(19) United States
(12) Patent Application Publication
(10) Pub. No.: US 2023/0337899 A1
(43) Pub. Date: Oct. 26, 2023

(54) MOVABLE ELONGATED STRUCTURE AND METHOD

(71) Applicant: NATIONAL UNIVERSITY CORPORATION SHIGA UNIVERSITY OF MEDICAL SCIENCE, Shiga (JP)

(72) Inventors: Atsushi YAMADA, Shiga (JP); Tohru TANI, Shiga (JP)

(21) Appl. No.: 18/143,451

(22) Filed: May 4, 2023

Related U.S. Application Data
(63) Continuation-in-part of application No. PCT/JP2021/040517, filed on Nov. 4, 2021.
(60) Provisional application No. 63/109,986, filed on Nov. 5, 2020.
(60) Provisional application No. 63/405,512, filed on Sep. 12, 2022.

(30) Foreign Application Priority Data
Aug. 27, 2021 (JP) 2021-138738
Feb. 28, 2023 (JP) 2023-029460

(51) Int. Cl. A61B 1/005; A61B 1/00; A61B 34/30

(52) U.S. Cl. CPC A61B 1/0052; A61B 1/00087; A61B 1/00133; A61B 34/30; A61B 1/0057; A61B 2034/302; A61B 2034/305; A61B 2034/301

(57) ABSTRACT

A movable elongated structure includes a distal end side tube, a base end side tube, traction wires, and a wiring aid routed by the traction wires arranged between the distal end side tube and the base end side tube, wherein the wiring aid guides the pair of traction wires from a pair of base end side wire lumens provided in the base end side tube to be introduced into a pair of distal end side wire lumens provided in the distal end side tube.

### CROSS REFERENCE TO RELATED APPLICATION

This application is a continuation-in-part application of PCT/JP2021/040517 (filed Nov. 4, 2021), which claim priorities to U.S. provisional Application No. 63/109,988 (filed Nov. 5, 2020) and Japanese Patent Application No. 2021-138738 (filed Aug. 27, 2021). Also, this application claims priority to Japanese Patent Application No. 2023-29460 (filed Feb. 28, 2023. Also, this application claims priority to U.S. Provisional Application No. 63/405,512 (filed Sep. 12, 2022). The entire contents of the above applications are incorporated herein by reference.

### BACKGROUND OF THE INVENTION

### 1. Field of the Invention

The present invention relates to a movable elongated structure and a method of operating the same, particularly relates to a movable elongated structure that is inserted into a duct such as a hollow organ, a vessel, or a blood vessel, and bent in a predetermined direction at a bifurcation or the like, wiring aids used for movable elongated structures, movable long structural instruments, flexible endoscopes and steering catheters, medical systems, tools, manipulators, robots, medical robots, insertion methods, robots and a method of operating a movable elongated structure.

### 2. Description of the Related Art

For example, elongated medical devices such as catheters and endoscopes that are inserted into body cavities are frequently used. Such an elongated medical device is inserted into a bifurcation or the like by bending the distal end portion like a medical manipulator.

Further, in an endoscope provided on a manipulator, a configuration has been proposed in which a bending portion provided in the middle of the distal end side bends in four directions, namely up, down, left, and right, so that the direction of the distal end may be changed up, down, left, and right. there is However, its structure Is complicated and its assembling property is low.

### SUMMARY OF THE INVENTION

Accordingly, the present invention provides a movable elongated structure that is easy to assemble, a movable elongated structure that may be bent and deformed in a desired direction with a simple structure, a movable elongated treatment instrument, and a movable elongated structure, a wiring aid used for a structure, a movable elongated structural instrument, a medical system, a tool, a manipulator, a robot, a medical robot, an insertion method, a robot operating method, and a movable elongated structure operating method.

This invention provides a movable elongated structure that includes a distal end side tubular body, a base end side tubular body, and a traction operation body, further including a wiring aid that is held by the traction operation body between the distal end side tubular body and the base end side tubular body.

Further, according to the present invention, a movable elongated structure having a distal end side tubular body, a base end side tubular body, and a traction operation body, further including a wiring aid that is removably held between the distal end side tubular body and the base end side tubular body, or configured without being easily detached.

As an aspect of a wiring aid configured to be detachable and retainable or to be removably held according to the present invention, there is provided the wiring aid, wherein the wiring aid arranged along the longitudinal direction between the flexible elongated distal end side tubular body and base end side tubular body arranged in series from the distal end side to the base end side along the longitudinal direction includes at least a cylindrical body, and is provided with a base end side flange protruding radially outward provided with an operation body arrangement part at the base end side end of the cylindrical body in which the interval in a circumferential direction is set wider than the interval in the circumferential direction between the pair of distal end side through holes provided inside the tube wall of the distal end side tubular body and penetrating in the longitudinal direction and a pair of traction operation bodies led out from a pair of base end side through holes provided inside the tube wall of the base end side tubular body and introduced into the distal side through hole are arranged.

The traction operation body may be string-shaped or band-shaped. Further, the pair of traction operation bodies may be separate traction operation bodies, or one traction operation body may be bent back to form a pair. Further, when pulling the pair of traction operation bodies, both of the traction operation bodies may be pulled, or one of the traction operation bodies may be pulled.

Further, according to the present Invention, the above-mentioned movable elongated structure and a traction drive unit for pulling the pair of traction operation bodies are provided, and a plurality of the traction drive units are provided, and a predetermined traction drive unit is provided to pull the pair of traction drive units (or units). It is characterized by being a movable elongated structural instrument that pulls the traction operation body to bend and deform the distal end side tubular body in a desired direction.

According to another aspect of the present Invention, there is provided a medical system comprising the movable elongated structural instrument described above, a drive unit for driving the traction drive, and a control unit connected to apply a drive signal to the drive unit.

In addition, the present invention provides the above-mentioned movable type elongated structure instrument, a mounting section for mounting the base end side tubular body In the movable type elongated structure to a distal end of a robot arm, and driving the traction drive unit on the robot arm side. The tool is provided with a driving mechanism for connecting and a connecting portion for connecting.

The present invention also provides a robot including a tool, a robot arm having the tool at its distal end, a drive unit for driving the traction drive unit and the robot arm, and a controller connected to apply a drive signal to the drive unit. The robot is characterized by having a unit.

Further, the present invention provides the movable elongated structural instrument described above, a main body portion provided at the base end of the base end side tubular body in the movable elongated structural body, and the traction drive portion operated in the main body portion. It is characterized by being a manipulator provided with an operation unit.

Further, the present invention provides a medical robot comprising the robot described above, wherein the output unit provides a drive signal to an externally provided drive unit that mechanically drives the movable elongated structure.

Further, the present invention provides an Insertion method, wherein the above-described movable elongated structure is inserted into a duct, the traction driving section is drive-controlled to bend and deform the distal end side tubular body, and the distal end side tubular body is inserted into the branched duct.

Further, the present invention provides an robot operation method, wherein an input/output unit connected by wire and/or wirelessly receives an operation signal in real time to a robot equipped with the above-described movable long structural instrument, and, when an arithmetic unit executes a predetermined operation program based on the received operation signal, the traction operation body is pulled by the pulling driving section to bend and/or stretch (extend) the distal tubular body in a desired direction based on the output from the arithmetic unit.

In addition, as an aspect of the wiring aid configured so as not to come off inadvertently according to the present Invention, there is provided a movable elongated structure in which at least two wires are formed in a flexible, elongated shape and have an internal space penetrating in the longitudinal direction, a tubular body, a flexible elongated traction operation body, and a cylindrical portion disposed between the two tubular bodies arranged in series and held by the traction operation body; a wiring aid that is not fixed to the tubular body; and a movement regulation part that restricts movement of the wiring aid In a radial direction that intersects the longitudinal direction with respect to at least one of the tubular bodies.

According to this aspect of the present invention, the wiring assist device has a tubular portion that is arranged between the two tubular bodies arranged in series and held by the traction operation body, and that is not fixed to the tubular bodies. Since the tool has a movement regulation part that restricts movement in a radial direction that intersects the longitudinal direction with respect to at least one of the tubular bodies, the wiring assisting tool does not come off inadvertently and may be assembled. movable elongated structures with high flexibility, wiring aids used for movable elongated structures, movable long structural instruments, medical systems, tools, manipulators, robots, medical robots, insertion methods, robot operation methods, Methods for manipulating movable elongated structures, flexible endoscopes and steering catheters may be provided.

As an embodiment of a movable elongated structure that may be bent and deformed in a desired direction with a simple structure of the present Invention, it is formed in a flexible elongated shape and has an internal space penetrating in the longitudinal direction. a plurality of tubular bodies, a flexible elongated traction body, and a plurality of tubular bodies arranged in series, disposed between two adjacent tubular bodies in the longitudinal direction, a wiring aid for maintaining the interval between the opposing ends of the tubular body, wherein the direction in which the traction body is pulled in the longitudinal direction is the base end side, and the opposite side is the distal end side; of the two tubular bodies adjacent to each other in the longitudinal direction, the tubular body arranged on the base end side is defined as the base end side tubular body, and the distal end side tubular body is defined as the base end side tubular body. The tubular body to be arranged in the head is a distal end side tubular body, the pair of traction bodies is a traction body set, four sets of the traction body are provided, and the traction body are arranged in the tubular body along the longitudinal direction. A pair of the lumens are provided as a lumen set, and four lumen sets are provided, and the four lumen sets are arranged in four directions in the cross section of the tubular body. The base end side tubular body and the distal end side tubular body, which are arranged in series, are arranged such that the lumen set provided in four directions is oriented along the longitudinal direction, and the traction body set is arranged in, in the distal end side tubular body, the lumen set is routed in one of the four directions of the cross section in which the lumen set is provided, and in the base end side tubular body, the distal end side tubular body The interval between the cross-sectional center of the tubular body in the one direction is shorter than that of the routed route set, and the route is routed in the route in which the interval between the routed traction bodies is wide, and the traction body is routed. is routed in the lumen of the base end side tubular body and the lumen of the distal end side tubular body, and fixed to the distal end side tubular body on the distal end side of the distal end side tubular body to be independent At least one of the base end side tubular body and the distal end side tubular body bends in a desired direction with respect to the other by pulling the plurality of traction bodies toward the base end side. It Is characterized by being a deformable movable elongated structure.

The tubular body may be a tubular body such as a flexible tube, or a flexible tubular body formed by a so-called skeleton structure in which a plurality of tubular bodies are connected so as to be bendable.

According to the present invention, there is provided a movable elongated structure including a movable elongated treatment instrument, and a movable elongated structure that may be bent and deformed in a desired direction with a simple structure by pulling a plurality of the towing bodies toward the base end, A method for inserting a movable elongated structure, a method for operating a movable elongated structure, a movable elongated structural instrument, a medical system, a tool, a robot, a method for operating a robot, a medical robot, a manipulator, a flexible endoscope, and a steering catheter may be provided.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of the illustrative embodiments when read in conjunction with the accompanying drawings, wherein:

FIG. 1 is an explanatory diagram of a movable elongated structure provided with a wiring aid that may be removed and held as an embodiment of the present invention; FIG. 1 at (A) is a perspective view of a movable elongated structure, and FIG. 1 at (B) is a perspective view of the movable elongated structure showing a distal end side flexible tube and a base end side flexible tube In a see-through state;

FIG. 2 is an explanatory diagram of a movable elongated structure; FIG. 2 at (A) is a front view of the movable elongated structure, and FIG. 2 at (B) is a cross-sectional view taken along line A-A in FIG. 2 at (A);

FIG. 3 is an explanatory diagram of a movable elongated structure; FIG. 3 at (A) is a cross-sectional view taken along line B-B in FIG. 2 at (A), FIG. 3 at (B) is a cross-sectional view taken along arrow C-C in FIG. 2 at (A), and FIG. 3 at (C) is a front view of the movable elongated structure with the spacer removed;

FIG. 4 is an explanatory diagram of a spacer; FIG. 4 at (A) Is a perspective view showing the front, right side and top of the spacer, FIG. 4 at (B) is a perspective view showing the front, left side and bottom of the spacer, FIG. 4 at (C) is a front view of the spacer, and FIG. 4 at (D) is a sectional view taken along an arrow D-D in FIG. 4 at (C);

FIG. 5 is an explanatory diagram of a movable elongated structure; taken along taken along taken along FIG. 5 at (A) is an exploded perspective view seen from the base end side of the movable elongated structure, FIG. 5 at (B) is an exploded perspective view seen from the distal end side of the movable elongated structure, FIG. 5 at (C) is an enlarged view of part “a” In FIG. 5 at (A), and FIG. 5 at (D) is an enlarged view of the “b” portion of FIG. 5 at (B).

FIG. 6 is a perspective view of the movable elongated structure with the distal end side flexible tube bent;

FIG. 7 is an explanatory view of the movable elongated structure with the distal end flexible tube bent; FIG. 7 at (A) is a cross-sectional view corresponding to the cross-sectional view of the movable elongated structure in a state where the distal end side flexible tube is bent, taken along line B-B in FIG. 2 at (A), and FIG. 7 at (B) is a sectional view corresponding to a cross-sectional view along the line C-C of FIG. 2 at (A);

FIG. 8 is an explanatory diagram of a movable elongated structure with a distal end flexible tube obliquely bent; FIG. 8 at (A) is a cross-sectional view corresponding to the cross-sectional view taken along the line B-B of FIG. 2 at (A) in the movable elongated structure with the distal end flexible tube obliquely bent, and FIG. 8 at (B) is a cross-sectional view corresponding to the cross-sectional view taken along line C-C of FIG. 2 at (A);

FIG. 9 is an image diagram of clinical use of the movable elongated structure;

FIG. 10 is an explanatory diagram of another example of a movable elongated structure in which the distal end flexible tube is bent; FIG. 10 at (A) is a cross-sectional view corresponding to the cross-sectional view of another example of the movable elongated structure taken along line B-B in FIG. 2 at (A), and FIG. 10 at (B) is a cross-sectional view of the movable elongated structure taken along arrows C-C of FIG. 2 at (A). FIG. 10 at (C) is a cross-sectional view corresponding to the cross-sectional view of another example of the movable elongated structure taken along line B-B in FIG. 2 at (A), and FIG. 10 at (D) is a sectional view of the movable elongated structure corresponding to C-C arrow sectional drawing of FIG. 2 at (A):

FIG. 11 is an explanatory diagram of a movable elongated structure with another arrangement pattern; FIG. 11 at (A) is a perspective view of a movable elongated structure with another arrangement pattern, and FIG. 11 at (8) is a perspective view of the movable elongated structure showing a distal end side flexible tube and a base end side flexible tube in a see-through state;

FIG. 12 is an explanatory diagram of a movable elongated structure with another arrangement pattern; FIG. 12 at (A) is a front view of the movable elongated structure, and FIG. 12 at (B) is a cross-sectional view taken along line A-A in FIG. 12 at (A);

FIG. 13 is an explanatory diagram of another example of a spacer; FIG. 13 at (A) Is a front, right side, and top perspective view of another example of a spacer; FIG. 13 at (B) is a front, left side, and top perspective view of the spacer; and FIG. 13 at (C) is a plan view of the spacer;

FIG. 14 is an illustration of another example of a movable elongated structure; FIG. 14 at (A) is a front view of the movable elongated structure, FIG. 14 at (B) is a cross-sectional view taken along line E-E in FIG. 15 at (A), and FIG. 14 at (C) is a rear view of the movable elongated structure;

FIG. 15 is an illustration of another example of a movable elongated structure; FIG. 15 at (A) is a cross-sectional view along line F-F in FIG. 14 at (A), FIG. 15 at (B) is a cross-sectional view along line G-G in FIG. 14 at (A), and FIG. 15 at (C) is a cross-sectional view along line H-H in FIG. 14 at (A);

FIG. 16 is an explanatory diagram of another example of a spacer; FIG. 16 at (A) is a front, right side, and top perspective view of the spacer, FIG. 16 at (B) is a back, right side, and top perspective view of the spacer, FIG. 16 at (C) is a front view of the spacer, and FIG. 16 at (D) is a top view of the spacer;

FIG. 17 is an explanatory diagram of another example of a spacer; FIG. 17 at (A) is a front, right side, and top perspective view of the spacer; FIG. 17 at (B) is a rear, right side, and top perspective view of the spacer; FIG. 17 at (C) is a front view of the spacer and FIG. 17 at (D) is a side view of the spacer;

FIG. 18 is an illustration of still another example of a movable elongated structure and spacers; FIG. 18 at (A) is a perspective view of the movable elongated structure, FIG. 18 at (B) is a cross-sectional view taken along line I-I in FIG. 18 at (A), and FIG. 18 at (C) is a cross-sectional view along arrow J-J in FIG. 18 at (A).

FIG. 19 is an illustration of still another example of a movable elongated structure and spacers; FIG. 19 at (A) is a perspective view of a spacer of yet another example, FIG. 19 at (B) is a side view of the spacer viewed from the distal end side, and FIG. 19 at (C) is a side view of the spacer viewed from the base end side;

FIG. 20 is an illustration of still another example of a movable elongated structure; FIG. 20 at (A) is a perspective view of the movable elongated structure before assembly, FIG. 20 at (8) is a perspective view of the movable elongated structure in the assembled state, and FIG. 20 at (C) Is a perspective view of the movable elongated structure with a traction wire routed;

FIG. 21 is an illustration of still another example of a movable elongated structure; FIG. 21 at (A) is a front view of the movable elongated structure before assembly, FIG. 21 at (B) is a front view of the movable elongated structure in the assembled state, and FIG. 21 at (C) is a front view of the movable elongated structure with a traction wire routed;

FIG. 22 is an explanatory diagram of an assembly spacer; FIG. 22 at (A) is an enlarged sectional view of the assembled spacer portion before assembly, and FIG. 22 at (8) is an enlarged sectional view of the assembled spacer portion in the assembled state;

FIG. 23 is a schematic explanatory diagram of a retractor in another example; 23 at (A) is a perspective view of the retractor, and FIG. 23 at (B) is a perspective view of the retractor showing the traction wire in a see-through state;

FIG. 24 is a schematic explanatory diagram of a retractor in another example; FIG. 24 at (A) is a top view of the retractor, FIG. 24 at (B) is a cross section through the upper wire lumen, FIG. 24 at (C) is a top view of the retractor with the elastic retractor in an open state;

FIG. 25 is a schematic diagram of a medical device In another example of this embodiment;

FIG. 26 is a schematic diagram of a telesurgery system in another example of this embodiment;

FIG. 27 is a schematic explanatory diagram of a surgical device in a telesurgery system; 27 at (A) is a plan view of a surgical device that may be loaded onto a robot arm assembly of the telesurgery system; FIG. 27 at (B) is a diagram showing the internal configuration of the surgical device of FIG. 27 at (A);

FIG. 28 is an explanatory diagram of a telesurgery system; FIG. 28 at (A) is a block diagram showing the connection relationship with each unit, and FIG. 28 at (B) is an operation flow diagram of the telesurgery system;

FIG. 29 is a diagram showing the results of a demonstration experiment of a movable elongated structure; FIG. 29 at (A) is a photograph of the result of the initial state, FIG. 29 at (8) is a photograph of the result of bending the movable elongated structure, and FIG. 29 at (C) is a photograph of the result in a bent state Imitating a conventional two-way movable elongated structure;

FIG. 30 is a diagram showing the results of a demonstration experiment of a movable elongated structure comparing bending radii;

FIG. 31 is an explanatory diagram of a movable elongated structure provided with a wiring aid configured so as not to come off inadvertently, as another embodiment of the present invention. FIG. 31 at (A) is a front, right side, and top perspective view of the movable elongated structure, and FIG. 31 at (B) is a perspective view showing a front, a right side, and a top of the movable elongated structure showing the distal end side flexible tube, the intermediate tube, and the base end side flexible tube in a see-through state;

FIG. 32 is an explanatory diagram of a movable elongated structure; FIG. 32 at (A) is a front view of the movable elongated structure, FIG. 32 at (B) is a cross-sectional view of FIG. 32 at (D) along arrows A-A, FIG. 32 at (C) is a rear view of the movable elongated structure, FIG. 32 at (D) is an arrow B-B in FIG. 32 at (A), FIG. 32 at (E) is a cross-sectional view along the C-C arrow in FIG. 32 at (A), and FIG. 32 at (F) Is a cross-sectional view along the D-D arrow in FIG. 32 at (A);

FIG. 33 is an explanatory diagram of an enlarged exploded perspective view of a main part of a movable elongated structure; FIG. 33 at (A) is an enlarged exploded perspective view showing the front, right side, and top of the main part of the movable elongated structure, and FIG. 33 at (8) is an enlarged exploded perspective view showing the rear, left side, and bottom of the main part of the movable elongated structure;

FIG. 34 is an explanatory diagram of a spacer or wiring aid; FIG. 34 at (A) Is a front view of the spacer. FIG. 34 at (B) is a plan view of the spacer, FIG. 34 at (C) is a cross-sectional view of FIG. 34 at (D) taken along line E-E, FIG. 34 at (D) is a left side view of the spacer, FIG. 34 at (E) is a perspective view showing front, right side, and plan the spacer, and FIG. 34 at (F) is a perspective view showing rear, left and bottom surfaces of the spacer;

FIG. 35 is an explanatory diagram of a main part of a movable elongated structure; FIG. 35 at (A) is an enlarged cross-sectional view of a main part of the movable elongated structure before assembly, and FIG. 35 at (B) is an enlarged cross-sectional view of a main part of the movable elongated structure in the assembled state.

FIG. 36 is an exploded perspective view of another example spacer;

FIG. 37 is an explanatory diagram of another example of a spacer; FIG. 37 at (A) Is a front view of the spacer, FIG. 37 at (B) is a plan view of the spacer, FIG. 37 at (C) is a cross-sectional view of FIG. 37 at (D) taken along line F-F, FIG. 37 at (D) is a left side view of the spacer, FIG. 37 at (E) is a perspective view showing front, right side, and plan of the spacer, and FIG. 37 at (F) is a perspective view showing the rear, left and bottom surfaces of the spacer;

FIG. 38 is an explanatory diagram of an enlarged exploded perspective view of a main part of a movable elongated structure of another example; FIG. 38 at (A) is an enlarged exploded perspective view showing the front, right side, and top of the main part of the movable elongated structure, and FIG. 38 at (8) is an enlarged exploded perspective view showing the rear, left side, and bottom of the main part of the movable elongated structure;

FIG. 39 is an explanatory diagram of a main part of another example of a movable elongated structure; FIG. 39 at (A) is an enlarged cross-sectional view of the essential parts of the movable elongated structure before assembly, and FIG. 39 at (B) Is an enlarged cross-sectional view of the essential parts of the movable elongated structure in the assembled state;

FIG. 40 is an explanatory diagram of yet another example of a spacer; FIG. 40 at (A) is a front view of the spacer, FIG. 40 at (B) is a plan view of the spacer, FIG. 40 at (C) is a cross-sectional view of FIG. 40 at (D) taken along line G-G. FIG. 40 at (D) is a left side view of the spacer, FIG. 40 at (E) is a perspective view showing front, right side, and plan of the spacer, and FIG. 40 at (F) is a perspective view showing the rear, left and bottom surfaces of the spacer;

FIG. 41 is an explanatory diagram of an enlarged exploded perspective view of a main part of a movable elongated structure of still another example; FIG. 41 at (A) is an enlarged exploded perspective view showing the front, right side, and top of the main part of the movable elongated structure, and FIG. 41 at (B) is an enlarged exploded perspective view showing the rear, left side, and bottom of the main part of the movable elongated structure;

FIG. 42 is an explanatory diagram of a main part of still another example of a movable elongated structure; FIG. 42 at (A) is an enlarged cross-sectional view of a main part of the movable elongated structure before assembly, and FIG. 42 at (8) is an enlarged cross-sectional view of a main part of the movable elongated structure in the assembled state.

FIG. 43 is an explanatory diagram of yet another example of a spacer; FIG. 43 at (A) is a front view of the spacer, FIG. 43 at (B) is a plan view of the spacer, FIG. 43 at (C) is a cross-sectional view of H-H in FIG. 40 at (D), FIG. 43 at (D) is a left side view of the spacer, FIG. 43 at (E) is a perspective view showing front, right side, and plan of the spacer, and FIG. 43 at (F) is a perspective view showing the rear, left and bottom surfaces of the spacer;

FIG. 44 is an explanatory diagram of an enlarged exploded perspective view of a main part of a movable elongated structure of still another example; FIG. 44 at (A) is an enlarged exploded perspective view showing the front, right side, and top of the main part of the movable elongated structure, and FIG. 44 at (B) is an enlarged exploded perspective view showing the rear, left side, and bottom of the main part of the movable elongated structure;

FIG. 45 is an explanatory diagram of a main part of still another example of a movable elongated structure; FIG. 45 at (A) is an enlarged cross-sectional view of a main part of the movable elongated structure before assembly, and FIG. 45 at (B) is an enlarged cross-sectional view of a main part of the movable elongated structure in the assembled state;

FIG. 46 is an explanatory diagram of another example of a spacer; FIG. 46 at (A) is a front view of the spacer, FIG. 46 at (B) is a plan view of the spacer, FIG. 46 at (C) is a cross-sectional view of FIG. 46 at (D) taken along line I-I, FIG. 46 at (D) is a left side view of the spacer, FIG. 46 at (E) is a perspective view showing front, right side, and plan of the spacer, and FIG. 46 at (F) is a perspective view showing the rear, left and bottom surfaces of the spacer;

FIG. 47 is an explanatory diagram of an enlarged exploded perspective view of a main part of a movable elongated structure of another example; FIG. 47 at (A) is an enlarged exploded perspective view showing the front, right side, and top of the main part of the movable elongated structure, and FIG. 47 at (B) is an enlarged exploded perspective view showing the rear, left side, and bottom of the main part of the movable elongated structure;

FIG. 48 is an explanatory diagram of a main part of another example of a movable elongated structure; FIG. 48 at (A) is an enlarged cross-sectional view of a main part of the movable elongated structure before assembly, and FIG. 48 at (B) is an enlarged cross-sectional view of a main part of the movable elongated structure in the assembled state;

FIG. 49 is an explanatory diagram of another example of a spacer; FIG. 49 at (A) is a front view of the spacer, FIG. 49 at (B) is a plan view of the spacer, FIG. 49 at (C) is a cross-sectional view of J-J in FIG. 49 at (D), FIG. 49 at (D) is a left side view of the spacer, FIG. 49 at (E) is a perspective view showing front, right side, and plan of the spacer, and FIG. 49 at (F) is a perspective view showing the right side, the top, and the rear, left side, and bottom of the spacer;

FIG. 50 is an explanatory diagram of an enlarged exploded perspective view of a main part of a movable elongated structure of another example; FIG. 50 at (A) Is an enlarged exploded perspective view showing the front, right side, and top of the main part of the movable elongated structure, and FIG. 50 at (B) is an enlarged exploded perspective view showing the rear, left side, and bottom of the main part of the movable elongated structure;

FIG. 51 is an explanatory diagram of a main part of another example of a movable elongated structure; FIG. 51 at (A) is an enlarged cross-sectional view of the essential parts of the movable elongated structure before assembly, and FIG. 51 at (B) is an enlarged cross-sectional view of the essential parts of the movable elongated structure in the assembled state;

FIG. 52 is an explanatory diagram of another example of a spacer; FIG. 52 at (A) is a front view of the spacer. FIG. 52 at (B) Is a plan view of the spacer, FIG. 52 at (C) is a cross-sectional view taken along line K-K of FIG. 52 at (D), FIG. 52 at (D) Is a left side view of the spacer, FIG. 52 at (E) is a perspective view showing front, right side, and plan of the spacer, and FIG. 52 at (F) Is a perspective view showing the back, left and bottom surfaces of the spacer;

FIG. 53 is an explanatory diagram of an enlarged exploded perspective view of a main part of a movable elongated structure of another example; FIG. 53 at (A) is an enlarged exploded perspective view showing the front, right side, and top of the main part of the movable elongated structure, and FIG. 53 at (B) is an enlarged exploded perspective view showing the rear, left side, and bottom of the main part of the movable elongated structure;

FIG. 54 is an explanatory diagram of a main part of another example of a movable elongated structure; FIG. 54 at (A) is an enlarged cross-sectional view of a main part of the movable elongated structure before assembly, and FIG. 54 at (8) Is an enlarged cross-sectional view of a main part of the movable elongated structure in the assembled state;

FIG. 55 is an explanatory diagram of another example of a spacer; FIG. 55 at (A) is a front view of the spacer, FIG. 55 at (B) is a plan view of the spacer, FIG. 55 at (C) is a cross-sectional view taken along line M-M of FIG. 55 at (D), and FIG. 55 at (D) is a left side view of the spacer;

FIG. 56 is an explanatory diagram of an enlarged perspective view of a main part of a spacer of yet another example; FIG. 56 at (A) Is an enlarged perspective view showing the front, right side, and top view of the main part of another example of the spacer. FIG. 56 at (B) is an enlarged disassembled perspective view showing the front, right side, and top view of the main part of another example of the spacer, FIG. 56 at (C) is an enlarged perspective view showing the back, left side, and bottom of the main part of another example spacer, and FIG. 56 at (D) Is an enlarged exploded perspective view showing the rear, left side, and bottom surfaces of the main parts of the spacer in an exploded state of another embodiment;

FIG. 57 is an explanatory diagram of a main part of another example of a movable elongated structure; FIG. 57 at (A) is an enlarged cross-sectional view of a main part of the movable elongated structure before assembly, and FIG. 57 at (8) Is an enlarged cross-sectional view of a main part of the movable elongated structure in the assembled state;

FIG. 58 is an explanatory diagram of another example of a spacer; FIG. 58 at (A) is a front, right and top perspective view of yet another example spacer, FIG. 58 at (B) is a back, left and bottom perspective view of yet another example spacer, FIG. 58 at (C) is a perspective view showing the back, left, and bottom of yet another example spacer, and FIG. 58 at (D) is a perspective view showing the back, left, and bottom of yet another example spacer;

FIG. 59 is an explanatory diagram of another example of a spacer; FIG. 59 at (A) is a front, right side and top perspective view of yet another example spacer, FIG. 59 at (B) is a back, left side and bottom perspective view of yet another example spacer, FIG. 59 at (C) is a perspective view showing the front, right side, and top of a spacer of yet another embodiment, and FIG. 59 at (D) is a perspective view showing the back, left, and bottom of a spacer of yet another embodiment;

FIG. 60 is a schematic explanatory diagram of a retractor in another example;

FIG. 60 at (A) is a perspective view of the retractor, and FIG. 60 at (B) is a perspective view of the retractor showing the traction wire in a see-through state;

FIG. 61 is a schematic explanatory diagram of a retractor in another example;

FIG. 61 at (A) is a top view of the retractor, FIG. 61 at (B) is a cross section through the upper wire lumen, and FIG. 61 at (C) is a top view of the retractor with the elastic retractor in the open state;

FIG. 62 is a schematic diagram of a medical device in another example of this embodiment;

FIG. 63 is a schematic diagram of a telesurgery system in another example of this embodiment;

FIG. 64 is a schematic illustration of a surgical device in a telesurgery system;

FIG. 64 at (A) is a plan view of a surgical device that may be loaded onto a robot arm assembly of the telesurgery system, and FIG. 64 at (B) is a diagram showing the internal configuration of the surgical device of FIG. 64 at (A);

FIG. 65 is an explanatory diagram of a telesurgery system; FIG. 65 at (A) is a block diagram showing the connection relationship with each unit, and FIG. 65 at (B) is an operation flow diagram of the telesurgery system;

FIG. 66 is an explanatory diagram of a movable elongated structure In another example of this embodiment FIG. 66 at (A) Is a front, right side, and top perspective view of the movable elongated structure, and FIG. 66 at (8) is a perspective view showing a front, a right side, and a top of the movable elongated structure showing the distal end side flexible tube and the base end side flexible tube in a see-through state;

FIG. 67 is an illustration of still another example of a movable elongated structure; FIG. 67 at (A) is a front view of the movable elongated structure, FIG. 67 at (B) is a cross-sectional view along line N-N in FIG. 67 at (A), FIG. 67 at (C) is a cross-sectional view along line O-O In FIG. 67 at (A), and FIG. 67 at (D) is a cross-sectional view along line P-P in FIG. 67 at (A);

FIG. 68 is an explanatory diagram of a movable elongated structure that may be bent and deformed in a desired direction as another embodiment of the present invention; FIG. 68 at (A) is a front, right side, and top perspective view of the movable elongated structure, and FIG. 68 at (8) is a perspective view showing the front of a structure, a right side, and a top of the movable elongated structure showing the distal end side flexible tube, the intermediate tube, and the base end side flexible tube in a see-through state;

FIG. 69 is an illustration of a movable elongated structure; FIG. 69 at (A) is a front view of the movable elongated structure, FIG. 69 at (B) is a rear view of the movable elongated structure, FIG. 69 at (C) is a top view of the movable elongated structure, and FIG. 69 at (D) is a bottom view of the movable elongated structure;

FIG. 70 is an explanatory diagram of a spacer; 70 at (A) is a perspective view showing the front, right side, and top of the spacer, FIG. 70 at (B) is a perspective view showing the rear, left side, and bottom of the spacer, FIG. 70 at (C) Is a front view of the spacer, and FIG. 70 at (D) is A-A arrow sectional view In FIG. 70 at (C);

FIG. 71 is an explanatory diagram of a movable elongated structure; FIG. 71 at (A) is an exploded view of the movable elongated structure, FIG. 71 at (B) is a cross-sectional view along line B-B in FIG. 69 at (C), FIG. 71 at (C) Is a cross-sectional view along line C-C in FIG. 71 at (B), and FIG. 71 at (D) is a cross-sectional view along line D-D in FIG. 71 at (B);

FIG. 72 is a perspective view of the movable elongated structure with the distal end side flexible tube bent;

FIG. 73 is an explanatory diagram of a movable elongated structure in another example; FIG. 73 at (A) is a front, right side, and top perspective view of the movable elongated structure, and FIG. 73 at (B) is a perspective view showing the front of a structure, a right side, and a top of the movable elongated structure showing the distal end side flexible tube, the intermediate tube, and the base end side flexible tube in a see-through state;

FIG. 74 is an explanatory diagram of a movable elongated structure in another example; FIG. 74 at (A) is a front view of the movable elongated structure, FIG. 74 at (B) is a rear view of the movable elongated structure, FIG. 74 at (C) is a top view of the movable elongated structure, and FIG. 74 at (D) is a bottom view of the movable elongated structure;

FIG. 75 is an explanatory diagram of a spacer in another example; FIG. 75 at (A) is a perspective view showing the front, right side and top of the spacer; FIG. 75 at (B) is a perspective view showing the rear, left side and bottom of the spacer; FIG. 75 at (C) is a front view of the spacer, and FIG. 75 at (D) is E-E arrow directional cross-sectional view in FIG. 75 at (C);

FIG. 76 is an explanatory diagram of a movable elongated structure in another example; FIG. 76 at (A) is an exploded view of the movable elongated structure, FIG. 76 at (B) is a cross-sectional view taken along line F-F in FIG. 74 at (C), FIG. 76 at (C) is a cross-sectional view taken along line G-G in FIG. 76 at (B), and FIG. 76 at (D) is a cross-sectional view taken along line H-H in FIG. 76 at (B);

FIG. 77 is a perspective view of a movable elongated structure with a distal end side flexible tube bent according to another example;

FIG. 78 is an explanatory diagram of a movable elongated structure in still another example; FIG. 78 at (A) is a front, right side, and top perspective view of the movable elongated structure, and FIG. 78 at (B) is a perspective view showing a front of a structure, a right side, and a top of the movable elongated structure showing the distal end side flexible tube, the Intermediate tube, and the base end side flexible tube in a see-through state;

FIG. 79 is an explanatory diagram of a movable elongated structure in still another example; FIG. 79 at (A) is a front view of the movable elongated structure, FIG. 79 at (B) is a rear view of the movable elongated structure, FIG. 79 at (C) Is a top view of the movable elongated structure, and FIG. 79 at (D) is a bottom view of the movable elongated structure;

FIG. 80 is an explanatory diagram of a spacer in still another example; FIG. 80 at (A) is a perspective view showing the front, right side and top of the spacer; FIG. 80 at (B) is a perspective view showing the rear, left side and bottom of the spacer; FIG. 80 at (C) is a front view of the spacer, and FIG. 80 at (D) is I-I arrow sectional view In FIG. 80 at (C);

FIG. 81 is an explanatory diagram of a movable elongated structure in still another example; FIG. 81 at (A) is an exploded view of the movable elongated structure, FIG. 81 at (B) is a cross-sectional view taken along J-J in FIG. 79 at (C), FIG. 81 at (C) is a cross-sectional view taken along K-K in FIG. 81 at (8), and FIG. 81 at (D) is a cross-sectional view taken along M-M in FIG. 81 at (a);

FIG. 82 is a perspective view of a movable elongated structure with a distal end side flexible tube bent in yet another example;

FIG. 83 is a schematic explanatory diagram of a retractor in another example;

FIG. 83 at (A) is a perspective view of the retractor, and FIG. 83 at (8) is a perspective view of the retractor showing the traction wire in a see-through state;

FIG. 84 is a schematic explanatory diagram of a retractor in another embodiment; FIG. 84 at (A) is a top view of the retractor, FIG. 84 at (8) is a cross section through the upper wire lumen, and FIG. 84 at (C) is a top view of the retractor with the elastic retractor in an open state;

FIG. 85 is a partial cross-sectional side view of a movable elongated structure in another embodiment;

FIG. 88 is a side view of a moveable elongated structure in another embodiment; and

FIG. 87 is a side view of a moveable elongated structure in another embodiment.

### DETAILED DESCRIPTION

Hereinafter, embodiments of the present invention will be described with reference to the drawings. In the following description, the same parts and components are given the same reference numerals. This embodiment includes, for example, the following disclosures.

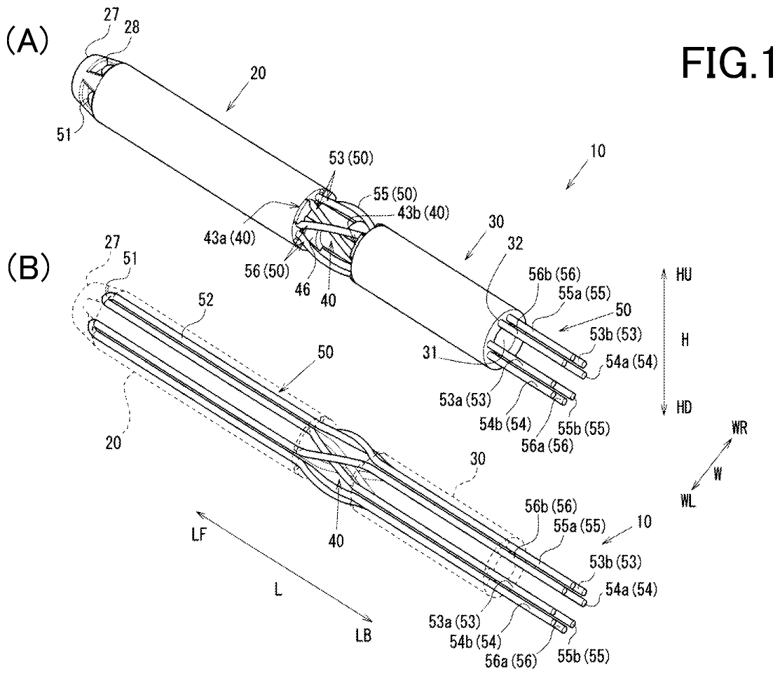

With reference to FIGS. 1 to 30, the following configurations are described relating to a movable elongated structure removably holding a wiring aid.

### [Configuration 1]

A movable elongated structure (10, 300) includes a distal end side tubular body (20, 60), a base end side tubular body (30), and a traction operation body (50), and provided with a wiring aid (40, 40a to 40k) held by a traction operation body (50) between the distal end side tubular body (20, 60) and the base end side tubular body (30). The wiring aid (40) may be removably held between the distal end side tubular body (20, 60) and the base end side tubular body (30) by a traction operation body (50).

As an example of the removably held wiring aid (40), the movable elongated structure (10, 300) includes a flexible distal end side tubular body (20, 60) having a through hole and a through hole a flexible base end side tubular body (30) having A tubular body (42), annular flanges (43) protruding radially outward from both ends of the tubular body (42), and recesses or cuts in the annular flanges (43) for arranging the traction operation body (50). An operation body arrangement part (44) having a cutout shape is provided, wherein the traction operation body (50) is inserted through the through hole of the base end side tubular body (30), removably hold the operation body arrangement part (44) by the operation body arrangement part (44), and is inserted through the through holes of the distal end side tubular bodies (20, 60).

The distal end side tubular body (20) and the base end side tubular body (30) are formed in a flexible and elongated shape, a pair of flexible traction operation bodies (50) are inserted through a pair of through holes along the longitudinal direction (L) provided Inside the tube wall of the distal end side tubular body (20) and the base end side tubular body (20), and a wiring aid (40) which is provided between the distal end side tubular body (20) and the base end side tubular body (30) to regulate the direction of the traction operation body (50). and penetrates through a through hole provided in the base end side tubular body (30) on the base end side. The through holes provided In the distal end side tubular body (20) are defined as the holes (32), and the through holes provided in the distal end side tubular body (20) are defined as the distal through holes (22). The interval Rf between the side wire lumen 36b and the base end side wire lumen 35a, and the interval Rg between the base end side wire lumen 36a and the base end side wire lumen 35b) extend in the circumferential direction between the pair of distal end side through holes (22). (interval Ra between distal end side wire lumen 23a and distal end side wire lumen 23b and interval Re between distal end side wire lumen 24a and distal end side wire lumen 24b). A cylindrical tubular body (42) arranged along the direction (L), and a base end side flange provided at the end of the tubular body (42) on the base end side (LB) and protruding radially outward. (43b), and a distal end side flange (43a) provided at the end of the tubular body (42) on the distal end side (LF) and projecting radially outward, and the base end side flange (43b) a proximity regulation part (45b) that regulates the proximity of the traction operation body (50) led out from the base end side through hole (32) and introduced into the distal end side through hole (22) in the circumferential direction; An operation body arrangement part (44) is provided that is open and in which a traction operating body (50) is placed, and is led out from the base end side through hole (32) to the distal end side flange (43a), leading to the distal end side. There may be provide a separation regulation part (45a) that regulates the circumferential separation of the traction operation body (50) introduced into the through hole (22), and an operation body arrangement part (44) in which the radial outer side Is open and the traction operation body (50) is arranged.

A distal virtual line (FVL) connecting the centers of the pair of distal through holes (22) and a distal end side parallel to the distal virtual fine (FVL) passing through the center of the distal end side tubular body (20) a base end side virtual line (BVL) connecting the centers of a pair of the base end side through holes (32) with a distal end side interval (X1), which is a interval from the central line (FCL), and the base end side tubular body; It may be set wider than the base-side interval (X2), which is the gap between the base-side center line (BCL) passing through the center of (30) and parallel to the base-side virtual line (BVL).

A pair of base end side through holes (32) and a pair of distal end side through holes (22) through which a pair of traction operation bodies (50) are inserted constitute a set of through holes (11), and a plurality of sets of through holes. (11) are arranged at different positions in the circumferential direction, and a plurality of proximity regulation parts (45b) and operation body arrangement parts (44) are provided on the base end side flange (43b), and the distal end side flange (43a) may be provided with a plurality of separation regulation portions (45a) and operation body arrangement parts (44).

Circumferentially adjacent traction operation bodies (50) of the pair of traction operation bodies (50) inserted through the circumferentially adjacent through hole sets (11) are close to each other at the base end side flange (43b). This is regulated by the common proximity regulation part (45b), and at the distal end side flange (43a), the pair of traction operation bodies (50) Inserted through the through hole sets (11) adjacent in the circumferential direction. The separation of the traction operation bodies (50) adjacent to each other in the circumferential direction is restricted by the separation regulation part (45a) common to the traction operation bodies (50) inserted into the through hole sets (11) adjacent to each other on the opposite side in the circumferential direction.

One traction operation body (50) and the other traction operation body (50) inserted through the through hole sets (11) adjacent in the circumferential direction intersect in the circumferential direction, and the tubular body (42) is provided with one traction operation body (50). A guide portion (46) may be provided to guide the body (50) to the outside of the other traction operation body (50).

An intermediate tubular body (60) having an elongated shape and having Intermediate through holes (63, 64, 65, 66) is arranged between the distal end side tubular body (20) and the base end side tubular body (30), a plurality of pairs of traction operation bodies (50) are provided, and at least one pair of traction operation bodies (50) among the plurality of pairs of traction operation bodies (50) has a distal end side through hole (22), an intermediate through hole (63, 64, 65, 66) and the base end side through hole (32), and at least one pair of the traction operation bodies (50) out of the plurality of pairs of traction operation bodies (50) are inserted through the intermediate through holes (63, 64, 65, 66) and the base end through hole (32).

The traction operation body (50) is a flexible wire, and the distal end side tubular body (20) and the base end side tubular body (30) are flexible tubes having main lumens (21, 31). The through hole may be a wire lumen formed inside the tube wall of the tube and through which a wire may be Inserted. The wire may be bent back at the distal end side (LF) of the distal end side tubular body (20) to form a pair of traction operation bodies (50).

### [Configuration 2]

A movable elongated structure (10, 300) having a distal end side tubular body (20, 60), a base end side tubular body (30), and a traction operation body (50), and a wiring aid (40) removably held between the distal end side tubular body (20, 60) and the base end side tubular body (30).

As an example, the wiring aid (40) has annular flanges (43) protruding radially outward from both ends of the tubular body (42), and the traction operation body (50) is arranged on the annular flanges (43). An operation body arrangement part (44) having a shape of a concave portion or a notch portion is provided.

In addition, the flexible elongated distal end side tubular body (20) arranged in series from the distal end side (LF) to the base end side (LB) along the longitudinal direction (L) and at least a cylindrical tubular body (42) arranged along the longitudinal direction (L) between the base end side tubular bodies (30) is provided, and the base end side (LB), a pair of distal end side through holes projecting radially outward and penetrating in the longitudinal direction (L) provided inside the tube wall of the distal end side tubular body (20) at intervals in the circumferential direction. A pair of base end side through holes (32) provided inside the tube wall of the base end side tubular body (30) and penetrating in the longitudinal direction (L), which are set wider than the interval in the circumferential direction between them.) and introduced into the distal end through hole (22), at the end of the tubular body (42) on the distal end side (LF), protrudes radially outward, is led out from the base end side through hole (32), and is introduced into the distal end side through hole (22), and a distal end side flange (43a) provided with an operation body arrangement part (44) in which the traction operation body (50) to be pulled is arranged.

In addition, the base end side flange (43b) is provided at the base end side (LB) end of the tubular body (42), and the distal end side flange (43a) is provided at the end on the distal end side (LF) of the tubular body (42), led out from the base end side through hole (32) to the base end side flange (43b), the distal end side through hole (22). A proximity regulation part (45b) is provided to restrict the proximity of the traction operation body (50) introduced into the peripheral direction in the circumferential direction, and the base end side through hole (32) is provided in the distal end side flange (43a). A separation regulation part (45a) for regulating the separation in the circumferential direction of the traction operation body (50) led out from and introduced into the distal end side through hole (22) is provided, and the operation body arrangement part (44) may be open radially outward.

A pair of base end side through holes (32) and a pair of distal end side through holes (22) through which a pair of traction operation bodies (50) are inserted constitute a set of through holes (11), and a plurality of sets of through holes (11) are arranged at different positions in the circumferential direction, and a plurality of proximity regulation parts (45b) and operation body arrangement parts (44) are provided on the base end side flange (43b), and the distal end side flange (43a) may be provided with a plurality of separation regulation portions (45a) and operation body arrangement parts (44).

Circumferentially adjacent traction operation bodies (50) of the pair of traction operation bodies (50) inserted through the circumferentially adjacent through hole sets (11) are close to each other at the base end side flange (43b). This is regulated by the common proximity regulation part (45b), and at the distal end side flange (43a), the pair of traction operation bodies (50) inserted through the through hole sets (11) adjacent in the circumferential direction The separation of the traction operation bodies (50) adjacent to each other in the circumferential direction is restricted by the separation regulation part (45a) common to the traction operation bodies (50) inserted into the through hole sets (11) adjacent to each other on the opposite side in the circumferential direction.

One traction operation body (50) and the other traction operation body (50) inserted through the through hole sets (11) adjacent in the circumferential direction intersect in the circumferential direction, and the tubular body (42) is provided with one traction operation body (50). A guide portion (46) may be provided to guide the body (50) to the outside of the other traction operation body (50).

### [Configuration 3]

The movable elongated structure (10, 300) and a traction drive unit (102, 221) for pulling the pair of traction operation bodies (50) are provided. A movable elongated structural Instrument (100, 217) for bending and deforming a distal end side tubular body (20) by pulling a traction operation body (50).

### [Configuration 4]

The movable elongated structure (10, 300) described above and a traction drive unit (102, 221) for pulling the pair of traction operation bodies (50) are provided, and a plurality of traction drive units (102, 221) are provided. A movable type that pulls a pair of traction operation bodies (50) with a predetermined traction drive unit (102, 221) to bend and/or stretch (extend) the distal end side tubular body (20) in a desired direction. An elongated structural instrument (100, 217).

### [Configuration 5]

A mobile elongated structural instrument (100, 217) as described above, a drive unit for driving a traction drive (102, 221), and a control unit (104, 202) connected to apply a drive signal to the drive unit. A medical system (200) comprising:

### [Configuration 6]

A movable elongated structural instrument (100, 217) as described above, a drive unit for selectively driving at least one of the plurality of traction drives (102, 221), and connected to apply a drive signal to the drive unit. a medical system (200) having an integrated control unit (104, 202). An operating portion for selectively operating at least one drive of the plurality of traction drives (102, 221) may be provided and connected to the control unit (104, 202),

### [Configuration 7]

The above-mentioned movable type elongated structure instrument (100, 217), the mounting part for mounting the base end side tubular body (30) in the movable type elongated structure (10, 300) to the distal end of the robot arm (212), and the robot A tool (217) provided with a drive mechanism for driving a traction drive (102, 221) and a connection (228) on the side of the arm (212).

### [Configuration 8]

The tool (217) described above, a robot arm (212) having the tool (217) at its distal end, a traction drive unit (102, 221) and a drive unit for driving the robot arm (212), and a drive signal to the drive unit. a control unit (104, 202) connected to a voltage.

### [Configuration 9]

A manipulator (100) provided with a main body including the movable elongated structural instrument (100) and the base end side tubular body (30) in the movable elongated structural body (10, 300), and an operating part to operate the traction drive unit (102, 221) on the main body side.

### [Configuration 10]

An input/output unit (210a) connected by wire and/or wireless to the movable elongated structural instrument (100, 217), an input unit for receiving an operation signal in real time, and a predetermined input unit based on the operation signal. A calculation unit (CPU) for executing an operation program, and a drawing drive section (102, 221) based on an output from the calculation unit (CPU) pulls a predetermined traction operation body (50) to pull at least the distal end side tubular body (20).) and an output unit for generating a drive signal for bending deformation and/or stretching (extending) deformation in a desired direction.

### [Configuration 11]

A medical robot comprising a robot as described above, wherein the output unit provides drive signals to an externally mounted drive unit for mechanically driving the movable elongated structure (10, 300).

### [Configuration 12]

An insertion method including the steps of inserting the above-described movable elongated structure (10, 300) into a duct, driving and controlling the traction drive unit (102, 221) to bend and deform the distal end side tubular body (20), and inserting the distal end side tubular body (20) into a branched duct.

The duct may be at least one of a hollow organ, a vessel, and a blood vessel.

### [Configuration 13]

The input/output unit (210a) connected by wire and/or wirelessly to the robot equipped with the movable long structural instrument (100, 217) receives an operation signal in real time, and the arithmetic unit (CPU) When a predetermined operation program is executed based on the received operation signal, the traction drive unit (102, 221) pulls the traction operation body (50) based on the output from the arithmetic unit (CPU) to pull the distal end side tubular body. A robot operating method for bending and/or stretching (extending) (20) in a desired direction.

### [Configuration 14]

An operation method of removing a wiring aid (40) from a movable elongated structure (10, 300) including a distal end side tubular body (20), a base end side tubular body (30) and a traction operation body (50) and provided with the wiring aid (40) removably held by the traction operation body (50) between the distal end side tubular body (20) and the base end side tubular body (30),

The distal end side tubular body (20) may be bent and deformed by pulling a pair of the traction operation bodies (50), or a plurality of pairs of the traction operation bodies (50) may be provided and a plurality of pairs of the traction operation may be provided. A predetermined pair of the traction operation bodies (50) of the body (50) may be pulled to bend and deform the distal end side tubular body (20) in a desired direction.

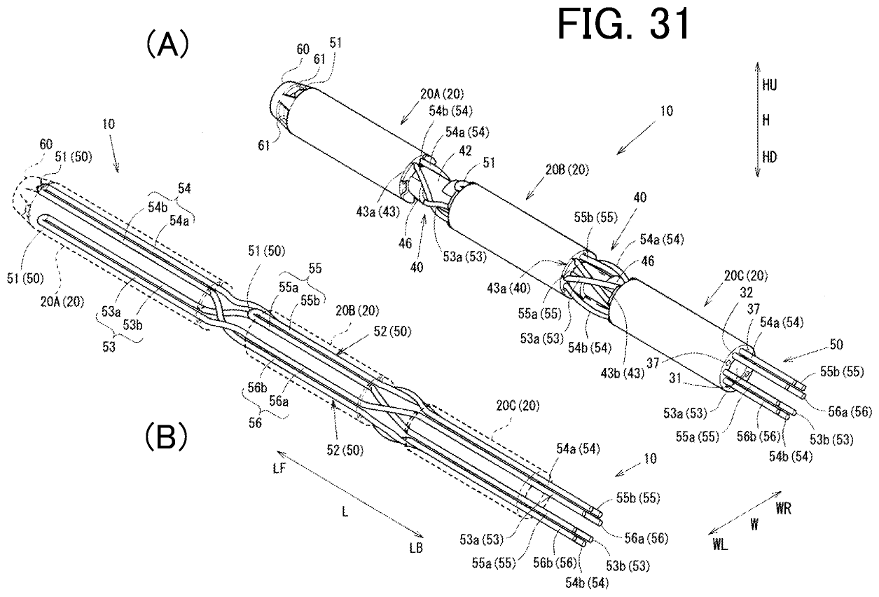

With reference to FIGS. 31 to 67, as examples of wiring aids to prevent accidental detachment, the following configurations are described.

### [Configuration 15]

The movable elongated structure (10) is formed in a flexible elongated shape and has at least two tubular bodies (20) having an inner space (31) penetrating in the longitudinal direction (L). and a flexible elongated traction operation body (50) and two tubular bodies (20) arranged in series, and held by the traction operation body (50). Wiring aids (40, 40a to 40k) having a cylindrical portion (42) and not fixed to the tubular body (20) and wiring aids (40, 40a to 40k) attached to at least one of the tubular body (20) 40k) is provided with movement regulation parts (47, 47a to 47f, 49) that restrict movement in at least the radial direction intersecting the longitudinal direction (L).

The traction operation body (50) may be inserted through a pair of through-passages (32) along the longitudinal direction (L) provided in the tube wall of the tubular body (20), and may be provided in plurality.

The wiring aids (40, 40a to 40k) are arranged at the ends of the cylindrical portion (42) and have flange portions (43, 43X) projecting radially outward, the wiring aids (40, 40a to 40k) arranged between the tubular bodies (20) are held via the projections (43, 43X), and the movement regulation parts (47, 47a) of the wiring aids 47f, 49) may be protrusions projecting from the end surface of the tubular part (42) toward at least one tubular body (20).

The protrusion (47, 47f) may be columnar, or may be columnar protruding from the ring-shaped member (48), or may be inserted Into a fitting recess (38) provided on the end face of the tube wall. Alternatively, they may be annular projections (47a, 47d, 49) fitted to at least one of the outer peripheral surface and the inner peripheral surface of the tubular body (20).

The protrusions may be small piece-like protrusions (47b, 47e) that contact at least one of the outer peripheral surface and the inner peripheral surface of the tubular body (20) at a plurality of points in the circumferential direction.

The protrusions may be tapered small piece protrusions (47c) that abut against the inner peripheral surface of the tubular body (20) at a plurality of points in the circumferential direction.

The tapered small piece protrusion (47c) may have a pull-out preventing portion (locking pawl) that regulates accidental pull-out from the inner peripheral surface in the inserted state.

The wiring aids (40, 40a to 40k) are arranged at the ends of the tubular portion (42) and have flanges (43, 43X) protruding radially outward. Wiring aids (40, 40a to 40k) arranged between bodies (20) are held via flanges (43, 43X), and the movement regulation part of the wiring aid is formed In at least one tubular shape. It may be a regulating tubular body (49) inserted across the Internal space (31) of the body (20) and the internal space (41) of the tubular portion (42).

The wiring aids (40, 40a to 40k) have a tubular portion (42) inserted into the internal space (31) of at least one tubular body (20), and the movement regulation part is located in the tubular portion (42), the portion inserted into the inner space (31) of the tubular body (20).

The movement regulation parts (47, 47a to 47f, 49) may be configured to regulate the wiring aids (40, 40a to 40k) from being unintentionally removed from being held by the traction operation body (50).

The movement regulation part may restrict radial movement of the wiring aids (40, 409 to 40k) with respect to both tubular bodies (20).

The traction operation body (50) may be a wire having flexibility, and the flanges projecting radially outward may be flanges (43, 43X).

A notch (44) for arranging the wire inside may be provided in the outer peripheral portion of the flange (43, 43X), or a hole (44a) through which the wire is inserted may be provided in the flange (43, 43X).

Of the two tubular bodies (20) arranged In series, the tubular body (20A) on the distal end side is an elastic retractor, elastic gripper, forceps or scissors having an internal space, and may be opened and closed with a wire (50).

### [Configuration 16]

A wiring aid (40) used for the movable elongated structure (10) described above.

### [Configuration 17]

The movable elongated structure (10) and the traction drive unit (102, 331) for pulling the pair of traction operation bodies (50) are provided. A movable elongated structural instrument (100, 317) that pulls the body (50) to bend and deform the distal end side tubular body (20A).

### [Configuration 18]

A movable elongated structural instrument (100, 317) Includes the movable elongated structure (10) and a traction drive unit (102, 331) for pulling the pair of traction operation bodies (50), wherein a plurality of traction drive units (102, 331) are provided, A pair of traction operation bodies (50) are pulled by a predetermined traction driving part (102, 331) to bend and/or stretch (extend) the distal end side tubular body (20A) in a desired direction.

### [Configuration 19]

A medical system (200) includes the movable elongated structural instrument (100, 317) as described above, a drive unit for driving a traction drive (102, 331), and a control unit (104, 202) connected to apply a drive signal to the drive unit.

### [Configuration 20]

A medical system (200) includes the movable elongated structural instrument (100, 317) as described above, a drive unit for selectively driving at least one of a plurality of traction drives (102, 331), and connected to apply a drive signal to the drive unit. a medical system (200) having an integrated control unit (104, 202).

An operating portion may be provided for selectively operating at least one drive of the plurality of traction drives (102, 331) and may be connected to the control unit (104, 202).

### [Configuration 21]

The above-mentioned movable type elongated structure instrument (100, 317), the mounting part for mounting the base end side tubular body (20C) in the movable elongated structure (10) to the distal end of the robot arm (312), and the robot arm (A tool (317) provided with a drive mechanism for driving the traction drive (102, 331) and a connection (361) on the 312) side.

### [Configuration 22]

A robot comprising the tool (317) described above, a robot arm (312) having the tool (317) at its distal end, and a drive unit driving the traction drive units (102, 331) and the robot arm (312), and a control unit (104, 202) connected to apply a drive signal to the drive unit, a drive unit.

### [Configuration 23]

A manipulator (100) comprising the above-mentioned movable type long structural instrument (100) and an operation part for operating a traction drive part (102, 331) on the main body side.

### [Configuration 24]

An input/output unit (310a) connected by wire and/or wireless to the movable elongated structural instrument (100, 317), an input unit for receiving an operation signal in real time, and a predetermined operation signal based on the operation signal. A calculation unit (CPU) that executes an operation program, and a drawing drive section (102, 331) based on an output from the calculation unit (CPU) pulls a predetermined traction operation body (50) to at least the distal end side tubular body (20A).) and an output unit for generating a drive signal for bending deformation and/or stretching (extending) deformation in a desired direction.

### [Configuration 25]

A medical robot comprising a robot as described above, wherein the output unit provides drive signals to an externally mounted drive unit for mechanically driving the movable elongated structure (10).

### [Configuration 26]

An insertion method of inserting the above-described movable elongated structure (10) into a duct, and driving/controlling the traction drive unit (102, 331) to bend and deform the distal end side tubular body (20A), and inserting the tubular body (20A) into a branching duct.

The duct may be at least one of a hollow organ, a vessel, and a blood vessel.

### [Configuration 27]

The input/output unit (310a) connected by wire and/or wirelessly to the robot equipped with the movable long structural instrument (100, 317) receives an operation signal in real time, and the arithmetic unit (CPU) When a predetermined operation program is executed based on the received operation signal, the traction drive unit (102, 331) pulls the traction operation body (50) based on the output from the arithmetic unit (CPU) to pull the distal end side tubular body. A robot operation method for bending and/or stretching (extending) (20A) in a desired direction.

### [Configuration 28]

An operation method of removing a wiring aid (40) from a movable elongated structure (10) including a distal end side tubular body (20A), a base end side tubular body (20C) and a fraction operation body (50) and provided with the wiring aid (40) removably held by the traction operation body (50) between the distal end side tubular body (20A) and the base end side tubular body (20C),

### [Configuration 29]

The distal end side tubular body (20A) may be bent and deformed by pulling a pair of the traction operation bodies (50), or a plurality of pairs of the traction operation bodies (50) may be provided and a plurality of pairs of the traction operation may be provided. A predetermined pair of the traction operation bodies (50) of the body (50) may be pulled to bend and deform the distal end side tubular body (20A) in a desired direction.

### [Configuration 30]

The movable elongated structure (10) described above and a plurality of traction operation units (103) for pulling the pair of traction operation bodies (50) are provided. A flexible endoscope or a steering catheter that pulls an operating body (50) to deform the tubular body (20A) on the distal end side (LF).

### [Configuration 31]

The tool (317) described above, a robot arm (312) having the tool (317) at its distal end, a drive unit for driving the traction drive units (102, 331) and the robot arm (312), and a drive signal to the drive unit. a control unit (104, 202) electrically connected, said control unit (104, 202) comprising artificial intelligence (AI).

### [Configuration 32]

A robot including the manipulator (100) described above, a robot arm (312) having the manipulator (100) at its distal end, and a drive unit driving the traction drive units (102, 331) and the robot arm (312), and a control unit (104, 202) connected to the drive unit to apply a drive signal, in which aid control unit (104, 202) includes an artificial intelligence (AI).

### [Configuration 33]

An input/output unit (310a) routed and/or wirelessly connected to the movable elongated structural instrument (100, 317), an input unit for receiving operation signals in real time, and a predetermined operation program based on the operation signals. and a traction drive unit (102, 331) based on the output from the computation unit (CPU) to pull a predetermined traction operation body (50) to at least the distal end side tubular body (20A) an output unit for generating drive signals for bending deformation and/or expansion/contraction (extension) deformation in a desired direction, and the arithmetic unit (CPU) provided with artificial intelligence (AI).

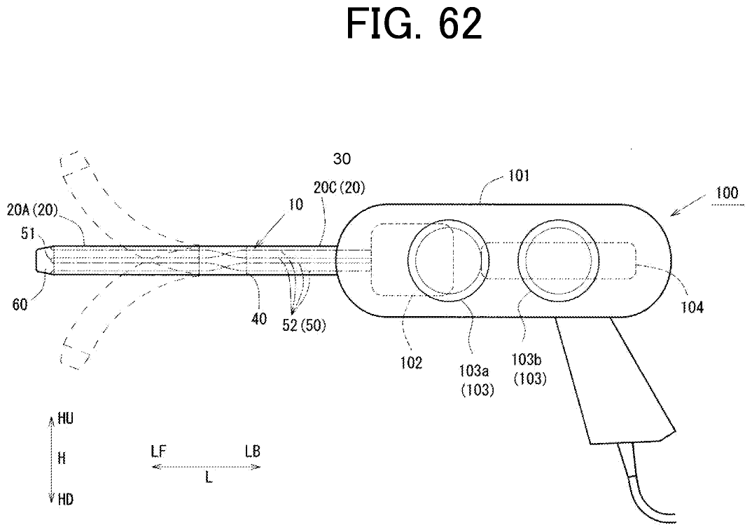

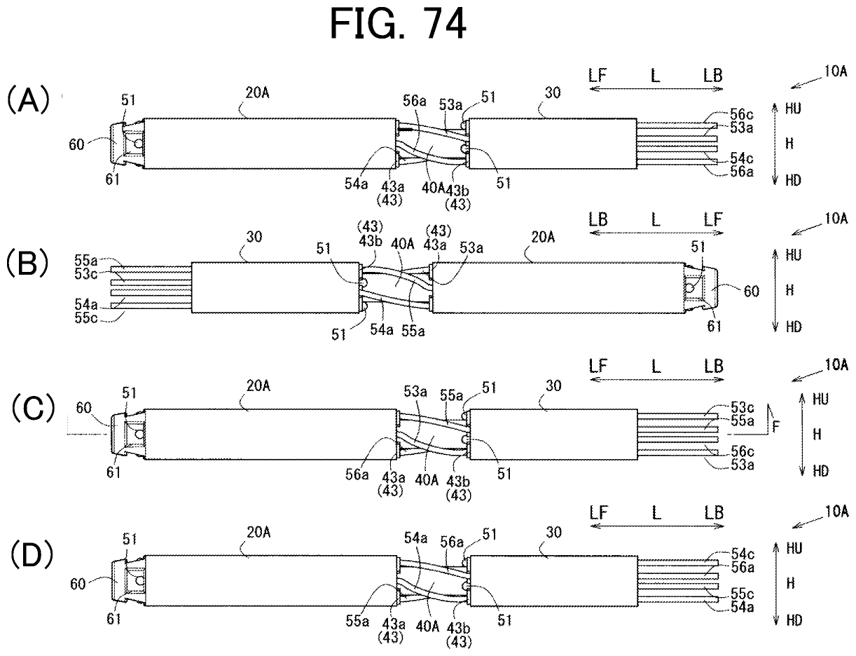

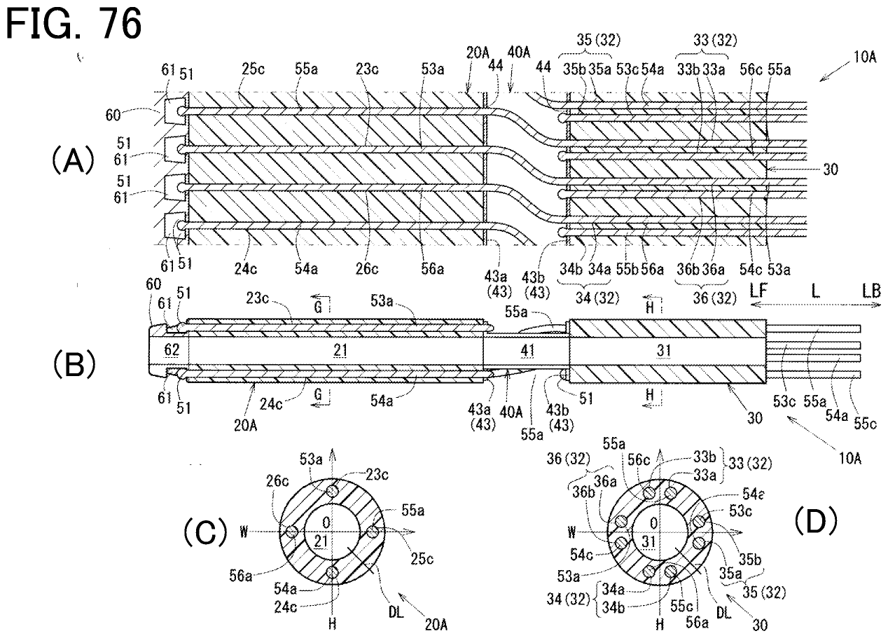

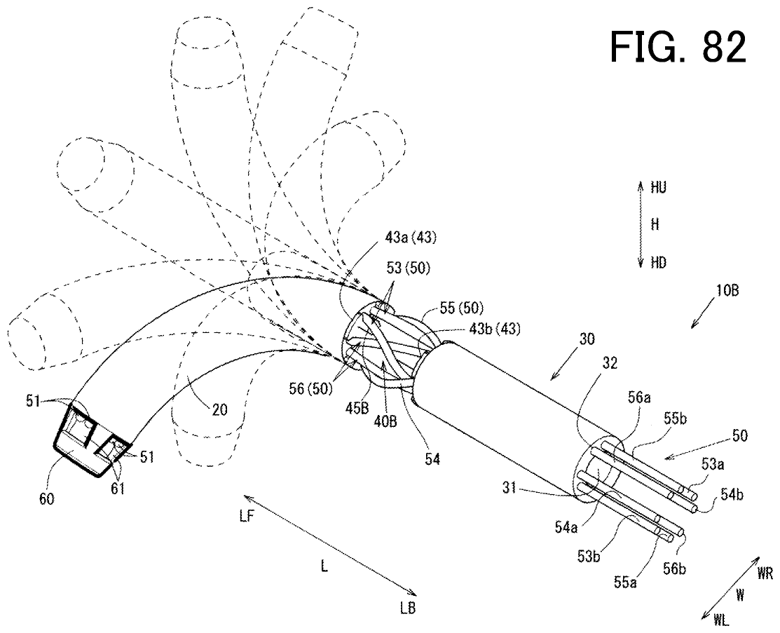

Next, configurations related to FIGS. 62 to 65 and FIGS. 68 to 84 will be described as aspects of the movable elongated structure that may be bent and deformed in a desired direction.

### [Configuration 34]

The movable elongated structure according to Configuration 1, wherein four pairs of traction bodies (50) are provided as traction body sets (53 to 56), the distal end side tubular body (20) and the base end side tubular body (30) are provided with wire lumens (22, 32) for routing the traction body (50) along the longitudinal direction (L),
four pairs of wire lumens (22, 32) are provided as wire lumen sets (23 to 26, 33 to 36),
the four wire lumen sets (23-26, 33-36) are arranged in four directions (HU, HO, WR, WL) in the cross section of the distal end side tubular body (20) and the base end side tubular body (30),
the base end side tubular body (30) and the distal end side tubular body (20) arranged in series are wire lumen sets (23 to 26, 33 to 36) provided in four directions (HU, HD, WR, WI), is arranged along the longitudinal direction (L), and

Distributed in the wire lumen set (23 to 26) in one of the four directions (HU, HD, WR, WL) of the cross section where the wire lumen set (23 to 26) is provided in the tubular body (20).

### [Configuration 35]

The movable elongated structure (10) according to Configuration 1, wherein m the base end side tubular body ((30), in one direction (HU, HD, WR, WI) from the route set (23a/23b: 23 to 26) routed in the distal end side tubular body (20), the distance to the center of the cross section (O) is close, the movable elongated structure is routed in the wire lumen (35b/36a: 33a to 36a, 33b to 36b) where the distance between the wired traction bodies (53a/53b: 53a to 56a, 53b to 56b) becomes wider

### [Configuration 36]

The movable elongated structure (10) according to Configuration 1, wherein in the base end side tubular body (30), the first traction body set (53a, 53b) routed in the distal end side tubular body (20) straddles the center of the cross section (O) and widens the interval. a second traction body set (54a/54b) routed to the wire lumen pair (35b/36b) and routed with the distal end tubular body (20) straddles the cross-sectional center (O) to widen the second and the virtual line connecting the first wire lumen pair crosses the virtual line connecting the second wire lumen pair near the cross-sectional center (O).

### [Configuration 37]

A movable elongated structures (108), wherein
a pair of traction bodies (50) are served as a traction body set (53, 54), and two traction body sets (53, 54) are provided;
tubular bodies (20, 30) are provided with wire lumens (22, 32) for routing the traction body (50) along the longitudinal direction (1), and the pair of wire lumens (2Z 92) are combined into a wire lumen set (23, 24, 35, 36);
in the tubular bodies (20, 30), two sets of wire lumens (23, 24, 35, 36) are provided, and two sets of lumen sets (23, 24, 35, 36) are provided in the tubular bodies (20, 30), and the distal end side tubular body (20) is arranged in the opposite direction and the base end in which the lumen set (23, 24, 35, 36) in the distal end side tubular body (20) is arranged in series with the base end side tubular body (30) in a direction that intersects the opposing direction in which the wire lumen set (23, 24, 35, 96) in the side tubular body (30) is arranged,
two sets of traction bodies (53 to 56) are routed to two sets of wire lumens (23, 24, 35, 36) in the distal end side tubular body (20), respectively, and the base end side tubular body (30), the traction body (50) of one traction body set (53, 54) of the two traction body sets (53, 54) is replaced with the two lumen sets (23, 24, 35, 36). each of the wire lumens (22, 32) in each of the two sets of wire lumens (23, 24, 35, 36). routed to the unrouted wire lumens (22, 32); and
in the base end side tubular body (30), the traction bodies (50) In the two sets of traction body sets (53, 54) are placed across the cross-sectional center of the tubular bodies (20, 30) in the cross direction that intersects one direction, either of the wire lumens (22, 32) in the two opposing wire lumen sets (23, 24, 35, 36) is routed, and in the base end side tubular body (30), the two wire lumen sets (the traction body (50) in the cross-sectional direction, and a second virtual fine (SL) connecting the traction bodies (50) of the other traction body set (53, 54) in the cross-sectional direction intersecting in direction.

### [Configuration 38]

A movable elongated structures (10B), wherein a plurality of tubular bodies (20, 30) formed in a flexible elongated shape and having internal spaces (21, 31) penetrating in the longitudinal direction (L) therein, and a flexible elongated shape The traction body (50) is arranged between two tubular bodies (20, 30) adjacent in the longitudinal direction among a plurality of tubular bodies (20, 30) arranged in series, and the tubular body (20, 30), and a wiring aid (40) that maintains the interval between the opposing ends of the traction body (50) in the longitudinal direction (L). The side is the distal end side (LF), and of the two tubular bodies (20, 30) arranged in series and adjacent in the longitudinal direction (L), the base end side tubular body (30) and a distal end side tubular body (20) arranged on the distal end side, and four pairs of traction bodies (50) are provided as traction body sets (53 to 56), and the tubular bodies (20, 30) are longitudinal. A lumen (22, 32) for wiring the traction body (50) is provided along (L), and a pair of lumens (22, 32) are arranged as a lumen set (23-26, 33-36). 4 sets are provided, and 4 sets of cableway sets (23 to 26, 33 to 36) are arranged in four directions (HU, HD, WR, WL) in the doss section of the tubular body (20, 30), and arranged in series. The base end side tubular body (30) and the distal end side tubular body (20) to be arranged are longitudinally arranged with the lumen sets (23 to 26, 33 to 36) provided in four directions (HU. HD, WR, WL), arranged along the direction (L), in one of the four directions (HU. HD, WR, WL) of the cross section in which the lumen set (23 to 26) is provided in the distal end side tubular body (20) while wiring the lumen set (23 to 26), the base end side tubular body (30) is arranged in one direction (HU, HD, WR, WL) is close to the cross-sectional center (O) of the tubular body (20, 30), and the interval between the routed traction bodies (53a to 56a, 53b to 56b) is widened, routed in the lumen (33a-36a, 33b-36b), multiple traction bodies (53a-56a, 53b-56b), traction bodies (53a-56a, 53b-56b) and traction sets (53-56) A movable elongated structure (10) that bends and deforms a distal end side tubular body (20) in a desired direction with respect to a base end side tubular body (30) by pulling it toward the base end side (LB).

### [Configuration 39]

A movable elongated structures (108), wherein a plurality of tubular bodies (20A, 30) formed in a flexible elongated shape and having internal spaces (21, 31) penetrating in the longitudinal direction (L) therein; Between a scale-shaped traction body (50, 50A) and two tubular bodies (20A. 30) adjacent in the longitudinal direction (L) among a plurality of tubular bodies (20A, 30) arranged in series and a wiring aid (40A) for maintaining the interval between the opposing ends of the tubular bodies (20A, 30), and the direction in which the traction bodies (50, 50A) are pulled in the longitudinal direction (L). The base end side (LB) and the opposite side thereof are defined as the distal end side (IF), and two tubular bodies adjacent to each other in the longitudinal direction (I) among the plurality of tubular bodies (20A, 30) arranged in series. Among (20A, 30), the tubular body (20A, 30) arranged on the base end side (LB) is defined as the base end side tubular body (30), and the tubular body (20A, 30) arranged on the distal end side (LF) 30) is a distal end side tubular body (20A), a pair of traction bodies (50, 50A) is a traction body set (53-56), and four traction body sets (53-56) are provided to form a pair of traction. One of the bodies (50) is a long traction body (50) that is routed over the base end side tubular body (30) and the distal end side tubular body (20A), and the other is the base end side. A short traction body (50A) is routed to the tubular body (30), and a lumen (22A, 32) are provided, and a pair of lumens (32) are served as lumen sets (33 to 36), four lumen sets (33 to 36) are provided, and four lumen sets (33 36) are arranged in four directions in the cross section of the base end side tubular body (30), and four lumens (22A) are arranged in four directions in the cross section of the distal end side tubular body (20A) and arranged in series. The base end side tubular body (30) and the distal end side tubular body (20A) are arranged so that four directions are aligned, and the long retractor (50) of the retractor set (53 to 56) is arranged in the distal end side tubular body. In the body (20A), the lumen (22A) Is routed in one of the four directions of the cross section where the lumen (22A) is provided, and at the distal end side (LF) of the distal end side tubular body (20A) it is fixed to the distal end side tubular body (20A), and the long retractor (50) and the short retractor (50A) of the retractor set (53-56) are attached to the base end side tubular body (30) at the distal end. The interval from the cross-sectional center (O) of the tubular bodies (20, 30) in one direction is closer than the lumen (22A) routed in the side tubular body (20A), and the wiring faces in the direction intersecting the one direction. The short traction body (50A) routed in the cableway (32) and routed in the cableway (32) of the base end side tubular body (30) is connected to the distal end side (LF) is fixed to the base end side tubular body (30), each retractor (50, 50A) is configured to be independently retractable, and a plurality of retractors (50, 50A) are configured to be retractable on the base end side (LB), at least one of the base end side tubular body (30) and the distal end side tubular body (20A) bends and deforms in a desired direction relative to the other (10A).

### [Configuration 40]

A movable elongated structures (10B), wherein a plurality of tubular bodies (20, 30) formed in a flexible elongated shape and having internal spaces (21, 31) penetrating in the longitudinal direction (L) therein, and a flexible elongated shape between the traction body (50) and two tubular bodies (20, 30) adjacent in the longitudinal direction (L) among the plurality of tubular bodies (20, 30) arranged in series, A wiring aid (40B) that maintains the interval between the opposing ends of the tubular bodies (20, 30), and the direction in which the traction body (50) is pulled in the longitudinal direction (L) is the base end side (LB), and the opposite side thereof is the leading end side (LF), and two tubular bodies (20, 30) adjacent in the longitudinal direction (L) among the plurality of tubular bodies (20, 30) arranged in series. Among them, the tubular body (20, 30) arranged on the base end side (LB) is defined as the base end side tubular body (30), and the tubular body (20, 30) arranged on the distal end side (LF) is defined as the distal end side tubular body. a body (20), a pair of traction bodies (50) as traction body sets (53-56), four traction body sets (53-56) are provided, and the tubular bodies (20, 30) are longitudinal. A lumen (22, 32) for wiring the traction body (50) is provided along (L), and a pair of lumens (22, 32) are arranged as a lumen set (23-26, 33-36). In addition, four cableway sets (23 to 26, 33 to 36) are provided, and four cableway sets (23 to 26, 33 to 36) are arranged in the cross section of the tubular body (20, 30). The base end side tubular body (30) and the distal end side tubular body (20), which are arranged in the direction and arranged in series, are arranged in four directions, and the lumen sets (23 to 26, 33 to 36) are arranged in the longitudinal direction. (L), and the traction body set (53-56) is placed in the distal end side tubular body (20) in one of the four directions of the cross section where the lumen set (23-26) is provided. While wiring the lumen set (23 to 26), in the base end side tubular body (30), in one direction than the lumen set (23 to 26) routed in the distal end side tubular body (20) The interval to the cross-sectional center (O) of the tubular bodies (20, 30) is short, and the interval between the routed traction bodies (50) is widened), the two traction body sets (53-56) are divided into two lumen sets (23-26) facing in one direction across the cross-sectional center (O) of the tubular bodies (20, 30). In the base end side tubular body (30), the traction bodies (50) in the two traction body sets (53 to 56) are cross-sectionally crossed in one direction. One of the two sets of lumens (33 to 36) facing each other across the center (O) is routed to one of the lumens (32), and the two sets of wiring are routed to the base end side tubular body (30). The traction body (50) in the cross-sectional direction, and a second virtual line (SL) connecting the traction bodies (50) of the other traction body set (53 to 56) in the cross-sectional direction pull the plurality of retractors (50) to base end side (LB) such that at least one of the base end (30) and distal (20) tubulars Is desired relative to the other. A movable elongated structure (10B) that bends and deforms in the desired direction.

### [Configuration 41]

A movable elongated structures (108), wherein a pair of traction bodies (50) are served as a traction body set (53, 54), and two traction body sets (53, 54) are provided, and the tubular bodies (20, 30) are longitudinal (L). A lumen (22, 32) for routing a traction body (50) is provided along the tubular body, and the pair of lumens (22, 32) constitutes a lumen set (23, 24, 35, 36). In (20, 30), two pairs of cableway sets (23, 24, 35, 36) are provided, and two pairs of cableway sets (23, 24, 35, 36) are provided in the tubular bodies (20, 30), and the distal end side tubular body (20) is arranged in a direction opposite to the lumen set (23, 24, 35, 36) in the distal end side tubular body (20) and the base end side It is arranged in series with the base end side tubular body (30) in a direction that intersects the opposing direction in which the lumen set (23, 24, 35, 36) in the tubular body (30) is arranged, and two sets of traction. The body assemblies (53 to 56) are routed to two sets of routing paths (23, 24, 35, 36) in the distal end side tubular body (20), respectively, and in the base end side tubular body (30), One of the two sets of traction bodies (53, 54), the first tow body (50) of one of the sets (53, 54) of the towing bodies (53, 54) is any, respectively, and the traction body (50) of the other traction body set (53, 54) is routed to the two routing path sets (23, 24, 35, 36) Each of the unrouted routing paths (22, 32) is routed, and the traction bodies (50) in the two traction body sets (53, 54) are routed in the base end side tubular body (30) in one direction. In either of the two pairs of cableway sets (23, 24, 35, 36) facing each other across the cross-sectional center of the tubular body (20, 30) in the crossing direction that intersects with two sets of traction routed and routed to one of the two sets of route routes (35, 36) in the base end side tubular body (30) (35a, 36a, 35b, 36b). A first virtual line (FL) connecting the traction bodies (50) of one traction body set (53 to 56) of the body sets (53, 54) in the cross-sectional direction, and the other traction body set (53, 54). The second virtual line (SL) connecting the traction bodies (50) in the cross-sectional direction intersects in the cross-sectional direction, and by pulling the multiple traction bodies (50) toward the base end side (LB), the base end, the movable elongated structures (10B) in which at least one of a side tubular body (30) and a distal end side tubular body (20) bends and deforms in a desired direction with respect to the other.

The first virtual fine (FL) and the second virtual line (SL) may pass through the cross-sectional center (O).

In addition, one traction body set (regulation part (458) may be provided for regulating the mutual relative positions of the traction body (50) of the traction body set (53-56) and the traction body (50) of the other traction body set (53-56).

The traction body (50, 50A) is a wire having flexibility, and the lumen may be a through path along the longitudinal direction provided inside the tube wall of the tubular body.

A treatment tool such as a refractor, a gripper, forceps, tweezers, or scissors is provided at the distal end of the tubular body (20, 20A) on the distal end side of the movable elongated structure (10, 10A, 10B), It may be a movable elongate treatment instrument in which a drive mechanism for the treatment instrument is arranged in the internal space (21).

Of the two tubular bodies (20, 20A, 30) arranged in series, the tubular body (20, 20A) on the distal end side is an elastic retractor, elastic gripper, forceps or scissors having an internal space, and a wire (50) to open and close it.

### [Configuration 42]

A insertion method of movable elongated structure: The above-described movable elongated structure (10, 10A, 10B) is inserted into the duct, and the traction drive unit (102, 331) for pulling the traction body (50, 50A) is driven and controlled to drive the distal end side tubular body (20, 20A) and the base end side tubular body (30) are bent and deformed in a desired direction, and the distal end side tubular body (20, 20A) is inserted into the branched duct. The duct may be at least one of a hollow organ, a vessel, and a blood vessel.

### [Configuration 43]

A method of operating the movable elongated structure (10, 10A, 10B) described above, including pulling the traction body set, and bending/deforming at least one of the distal end side tubular body (20, 20A) and the base end side tubular body (30) into a desired direction.

### [Configuration 44]

A method of operating the movable elongated structures (10, 10A, 10B) described above, wherein each of the plurality of traction body sets (53 to 56) is pulled for bending and deforming at least one of the distal end side tubular body (20, 20A) and base end side tubular body (30) in a desired direction.

### [Configuration 45]

An operation method of a movable elongated structure for bending and defaming a base end side tubular body (30) in a desired direction by pulling a traction body (50A), wherein of the traction body set, the lumen (32) of the base end side tubular body (30) and the lumen (22A) of the distal end side tubular body (20A) are routed, and the distal end side tubular body (20A) Is routed. One traction body (50) fixed to the distal end side tubular body (20A) on the distal end side (LF) is pulled to bend and deform the distal end side tubular body (20A) in a desired direction to form a base end side tubular body. The other of the traction body sets routed through the muting path (32) of the body (30) and fixed to the base end side tubular body (30) at the distal end side (LF) of the base end side tubular body (30).

### [Configuration 46]

A movable elongated structural instrument (100, 317) for bending and deforming at least one of a distal end side tubular body (20, 20A) and a base end side tubular body (30) in a desired direction, wherein The movable elongated structure (10, 10A, 10B) and a traction drive unit (102, 331) for pulling the traction body are provided, and the traction drive unit (102, 331) is designed to pull the traction body for bending and deforming.

### [Configuration 47]

A medical system (200) comprising: the movable elongated structural instrument (100, 317) as described above, a drive unit for driving a traction drive (102, 331), and a control unit (104, 302) connected to apply a drive signal to the drive unit.

### [Configuration 48]

A tool (317) including the movable elongated structural instrument (100, 317) described above, and a mounting part for mounting the base end side tubular body (30) of the movable elongated structural body (10, 10A, 10B) to the distal end of the robot arm (312), and a connecting part (361) that connects with a drive mechanism for driving a traction drive (102, 331) on the robot arm side (312).

### [Configuration 49]

A robot including the tool (317) described above and a drive unit driving a robot arm (312) having the tool (317) at its distal end, a traction drive unit (102, 331) and the robot arm (312) and a control unit (104, 302) connected to the drive unit to apply a drive signal to the drive unit.

### [Configuration 50]

A robot including an input/output unit (310a) connected by wire and/or wireless to the movable elongated structural instrument (100, 317), an input unit for receiving an operation signal in real time, and a predetermined operation signal based on the operation signal. A computation unit (CPU) that executes an operation program, and a traction drive unit (102, 331) based on an output from the computation unit (CPU) pulls a predetermined traction body (50, 50A) to pull at least the distal end side tubular body (20, 20A), and an output unit for generating drive signals for bending deformation and/or stretching (extension) deformation in a desired direction.

### [Configuration 51]

The tool (317) described above, a robot arm (312) having the tool (317) at its distal end, a drive unit for driving the traction drive units (102, 331) and the robot arm (312), and a drive signal to the drive unit. a control unit (104, 302) electrically connected, the control unit (104, 302) comprising artificial intelligence (AI).

### [Configuration 52]

A robot including the manipulator (100) as described above, the robot arm (312) mounting the manipulator (100) at its distal end, a traction drive unit (102, 331), and a drive unit driving the robot arm (312), a control unit (104, 302) electrically connected with the drive unit to apply a drive signal to the drive unit, wherein the control unit (104, 302) including an artificial intelligence (AI) for operation of the robot.

### [Configuration 53]

A robot including an input/output unit (310a) routed and/or wirelessly connected to the movable elongated structural instrument (100, 317), an input unit for receiving operation signals in real time, and a predetermined operation program based on the operation signals. and a fraction drive unit (102, 331) based on the output from the computation unit (CPU) to pull a predetermined traction body (50, 50A) to at least the distal end side tubular body (20, 20A), and an output unit for generating a drive signal for bending deformation and/or expansion/contraction (extension) deformation in a desired direction, and an arithmetic unit (CPU) equipped with artificial intelligence (AI).

### [Configuration 54]

A method of operating a robot that bends and/or stretches (extends) a distal end side tubular body (20, 20A) in a desired direction, wherein the input/output unit (310a) connected by wire and/or wirelessly to the robot equipped with the movable long structural instrument (100, 317) receives an operation signal in real time, and the arithmetic unit (CPU), when a predetermined operation program is executed based on the received operation signal, the traction drive unit (102, 331) pulls the traction body (50, 50A) based on the output from the arithmetic unit (CPU) to extend the distal end side tubular shape.

### [Configuration 55]

A medical robot comprising a robot as described above, wherein the output unit provides drive signals to an externally mounted drive unit for mechanically driving the movable elongated structure (10, 10A, 10B).

### [Configuration 56]

A manipulator (100) comprising the above-mentioned movable elongated structural instrument (100, 317) and an operation part for operating a traction drive part (102, 331) on the main body side.

### [Configuration 57]

The movable elongated structure (10) and a plurality of traction operation units (103) for pulling the pair of traction bodies (50, 50A) are provided (50, 50A) to deform the distal end (LF) tubular body (20A).

A flexible endoscope, wherein a movable elongated structure (10) as described above and a plurality of traction operation units (103) for pulling a pair of traction bodies (50, 50A) are provided, and the pair of traction bodies (50, 50A) are pulled by the traction operation part (103) to deform the tubular body (20A) on the distal end side (LF).

### [Configuration 58]

A steering catheter wherein the movable elongated structure (10) as described above and a plurality of traction operation units (103) for pulling a pair of fraction bodies (50, 50A) are provided, and the pair of traction bodies (50, 50A) are pulled by the traction operation part (103) to deform the tubular body (20A) on the distal end side

### [Configuration 59]

A method of manufacturing a movable elongated structure, including steps of
forming an elongated shape with a flexible distal end side tubular body and a flexible or inflexible base end side tubular body;
a step of inserting a pair of traction operation bodies having flexibility along the longitudinal direction through a pair of through holes provided inside the tube walls of the distal end side tubular body and the base end side tubular body;
arranging a wiring aid between the distal end side tubular body and the base end side tubular body, and
regulating (or restricting) the direction of the traction operation body. Similarly, the manufacturing method may be configured in accordance with the above described devices.

### [Configuration 60]

A method of designing a movable elongated structure, including steps of forming an elongated shape with a flexible distal end side tubular body and a flexible or inflexible base end side tubular body;
inserting a pair of traction operation bodies having flexibility along the longitudinal direction through a pair of through holes provided inside the tube walls of the distal end side tubular body and the base end side tubular body;
arranging a wiring aid between the distal end side tubular body and the base end side tubular body, and restricting the direction of the traction operation body.

Similarly, a method of designing a configuration suitable for the above described equipment and systems may be applied.

### [Configuration 61]

A movable elongated structure including at least to distal end side tubular body (20), a base end side tubular body (30), a traction operation body (53b 54a 56a 56b), and a tubular body (40L) connected with the distal end side tubular body (20) and the base end side tubular body (30), wherein the distal end side tubular body (20) and the base end side tubular body (30) each have a wire routine, and the traction operation body (53b 54a 56a 56b) is housed in a hollow portion (47S) of the tubular body (40L).

If necessary, the hollow portion (47S) of the tubular body (40L) may be filled with lubricant. The distal end side tubular body (20), the tubular body (40L), and the base end side tubular body (30) may be composed of a continuous tube, in which at least the distal end side tubular body (20) is composed of a flexible tube. Or, the distal end side tubular body (20) may be flexible and the base endside tubular body (30) may be flexible or non flexible.

### [Configuration 62]

A movable elongated structure including:
a flexible distal end side tubular body (20) having a through interior space disposed at a distal end,
a flexible or inflexible base end side tubular body (30) having a through Interior space disposed at a base end, and
at least a pair of flexible elongated traction bodies (53b, Ma, 56a, 56b) for pulling the flexible distal end side tubular body,
wherein the pair of fraction bodies are served as a traction body set, and a plurality of the traction body sets (53b. 54a, 56a, 56b) are provided,
wherein a lumen (22, 32) routing the traction body set along a longitudinal direction is provided in the distal end side tubular body (30) and the base end side tubular body (30),
wherein a pair of the lumens are served as a lumen set, and a plurality of the lumen sets are provided, and the plurality of lumen sets are formed in cross sections of the distal end side tubular body and the base end side tubular body, respectively arranged in multiple directions, and
wherein in the base end side tubular body, there are provided lumen sets in which the interval between the cross-sectional center of the base end side tubular body on one side is shorter than that of the lumen set routed in the distal end side tubular body, and the interval between the routed traction bodies is wider than that of the lumen set routed in the distal end side tubular body.

The plurality of sets may be 2 sets, 3 sets, 4 sets, or 6 sets according to design.

### [Configuration 63]

In the configuration described above, a movable elongated structure in which the distal end side tubular body (20) may be flexible and the base end side tubular body (30) may be inflexible.

Although the following examples are used for medical equipment as an example of the treatment tool of the present invention, the present invention provides a treatment tool that is not limited to medical equipment.

First, the drawings will be described in detail. For example, FIG. 1 shows, as an embodiment of the present invention, an explanatory view of a movable elongated structure 10 equipped with a wiring aid that may be removed and held. FIG. 1 at (A) shows a perspective view of movable elongated structure 10, and FIG. 1 at (B) shows distal end side flexible tube 20 and base end side flexible tube 30 (hereinafter flexible tubes 20, 30) in a see-through state.

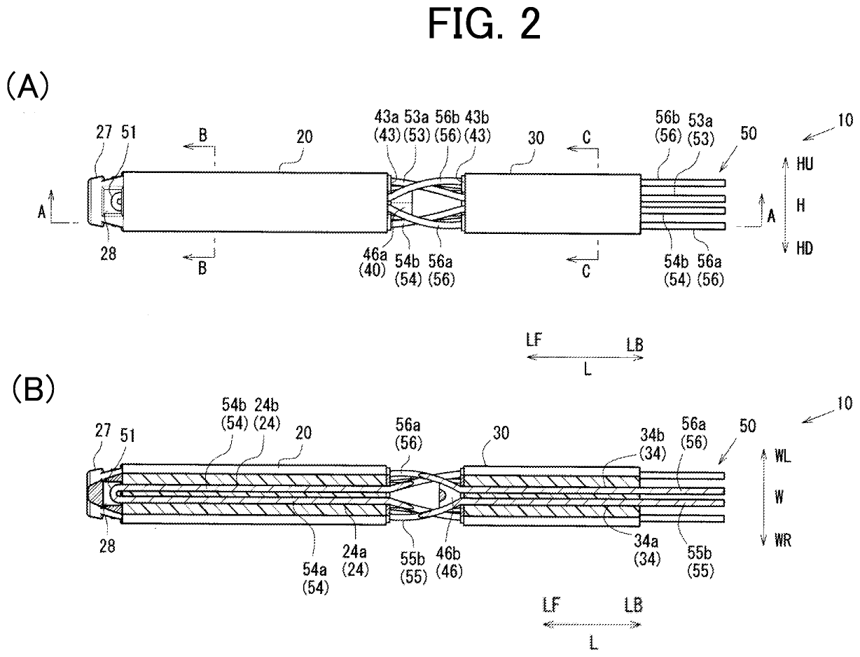

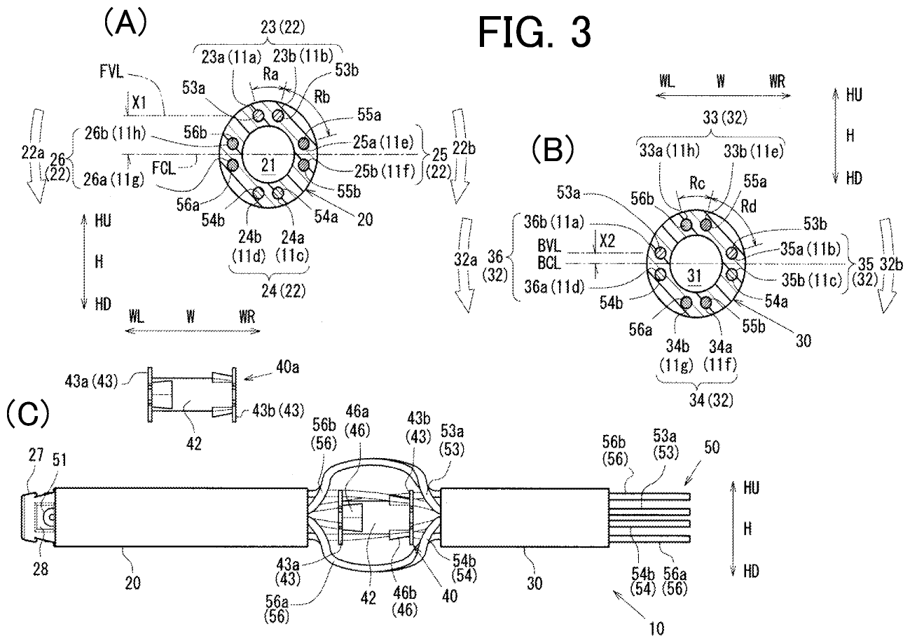

FIGS. 2 and 3 show illustrations of the movable elongated structure 10. FIG. 2A shows a front view of the movable elongated structure 10, FIG. 2 at (B) shows a cross-sectional view taken along line A-A in FIG. 2 at (A), FIG. 3 at (A) shows a cross-sectional view taken along line B-B in FIG. 2 at (A), and FIG. 3 at (B) shows a cross-sectional view taken along line C-C in FIG. 2 at (A), and FIG. 3 at (C) shows a front view of the movable elongated structure 10 with the spacer 40 removed.

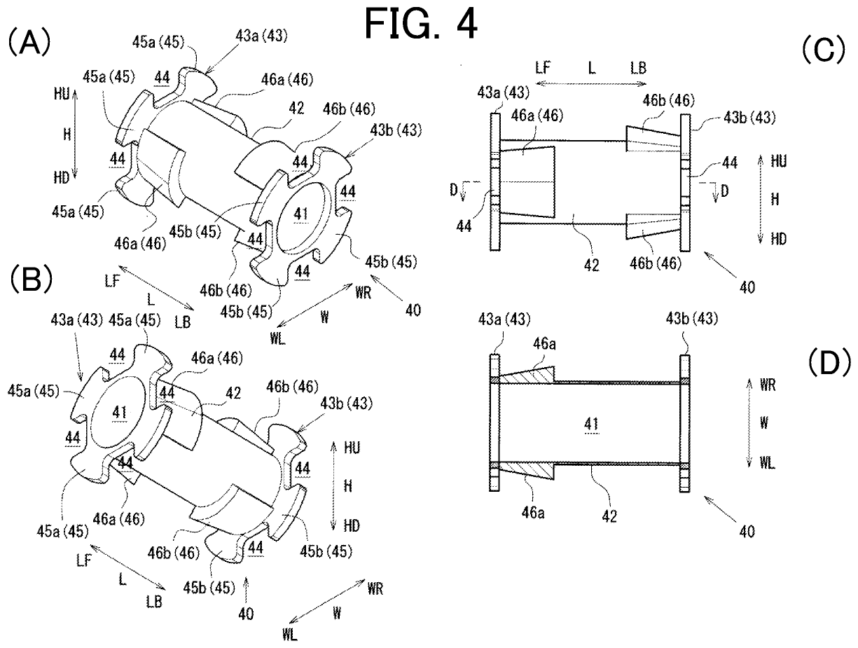

FIG. 4 shows an explanatory view of a spacer 40 which is a wiring aid explained in other examples. FIG. 4 at (A) shows a front, right side and top perspective view of spacer 40, FIG. 4 at (B) shows a front, left side and bottom perspective view of spacer 40, FIG. 4 at (C) shows a front view of spacer 40, and FIG. 4 at (D) shows a cross-sectional view taken along line D-D in FIG. 4 at (C).

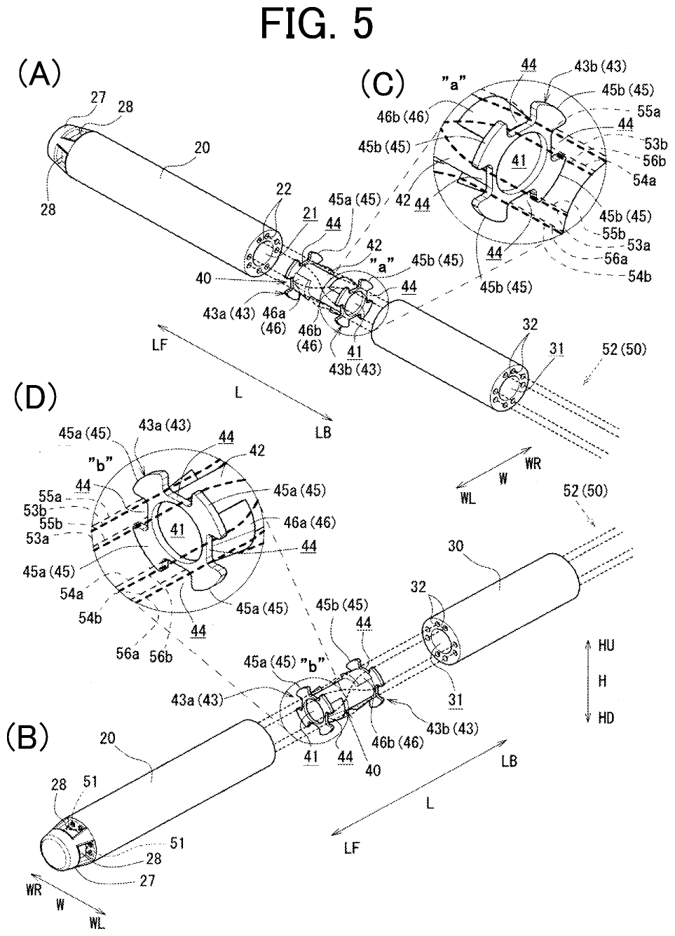

FIG. 5 shows an explanatory view of the movable elongated structure 10; FIG. 5 at (A) shows an exploded perspective view of the movable elongated structure 10 viewed from the base end side LB, and FIG. 5 at (B) shows an exploded perspective view of the movable elongated structure 10 viewed from the distal end side LF, FIG. 5 at (C) shows an enlarged view of part “a” in FIG. 5 at (A), and FIG. 5 at (D) shows an enlarged view of part “b” in FIG. 5 at (B).

It should be noted that the traction wire 50 is illustrated with a dashed line in FIG. 5.

FIG. 5 shows a perspective view of the movable elongated structure 10 with the distal end side flexible tube 20 bent.

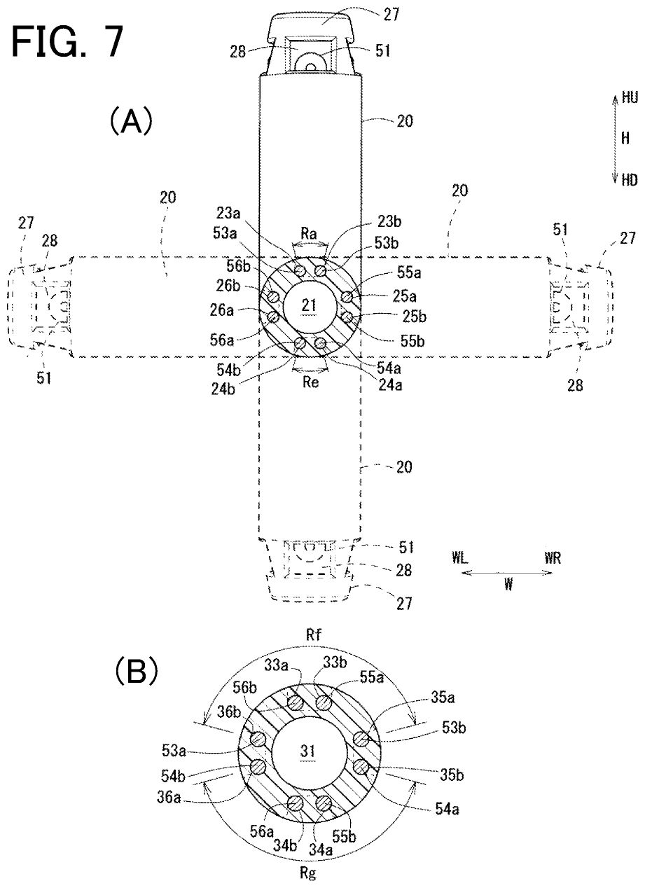

FIG. 7 shows an explanatory view of the movable elongated structure 10 with the distal end side flexible tube 20 bent. FIG. 7 at (A) shows a cross-sectional view corresponding to the cross-sectional view taken along the line 8-B of FIG. 2 at (A) in the movable elongated structure 10 with the distal end side flexible tube 20 bent, and FIG. 7 at (B) shows an enlarged cross-sectional view corresponding to the cross-sectional view taken along the line C-C of FIG. 2 at (A).

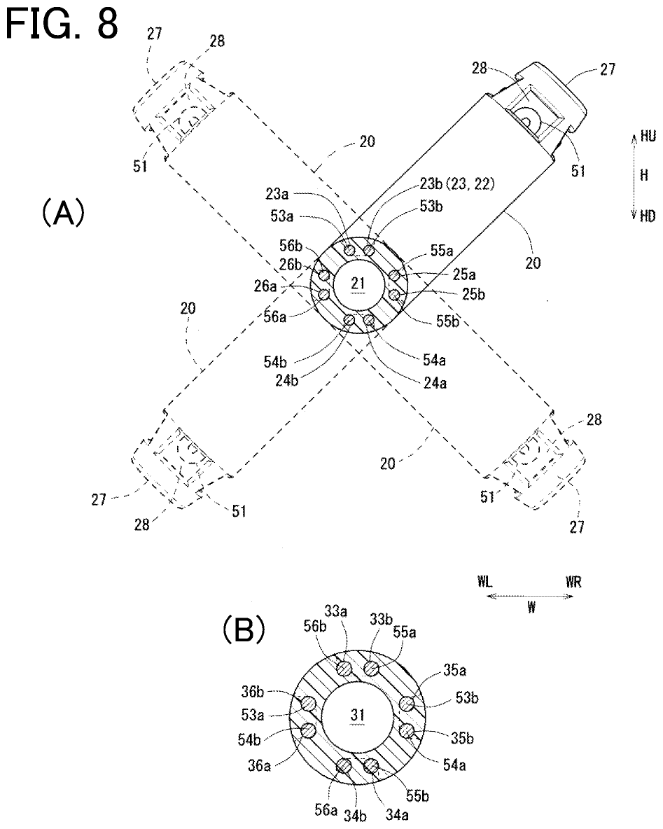

FIG. 8 shows an explanatory view of the movable elongated structure 10 with the distal end flexible tube 20 obliquely bent. FIG. 8 at (A) shows a cross-sectional view corresponding to the cross-sectional view taken along the line B-B in FIG. 2 at (A) in the movable elongated structure 10 with the distal end side flexible tube 20 obliquely bent, and FIG. 8 at (B) shows an enlarged cross-sectional view corresponding to the cross-sectional view taken along the line C-C of FIG. 2 at (A).

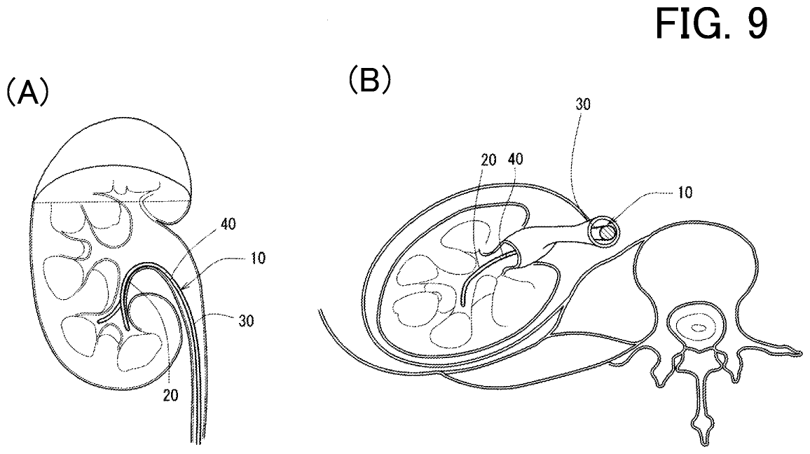

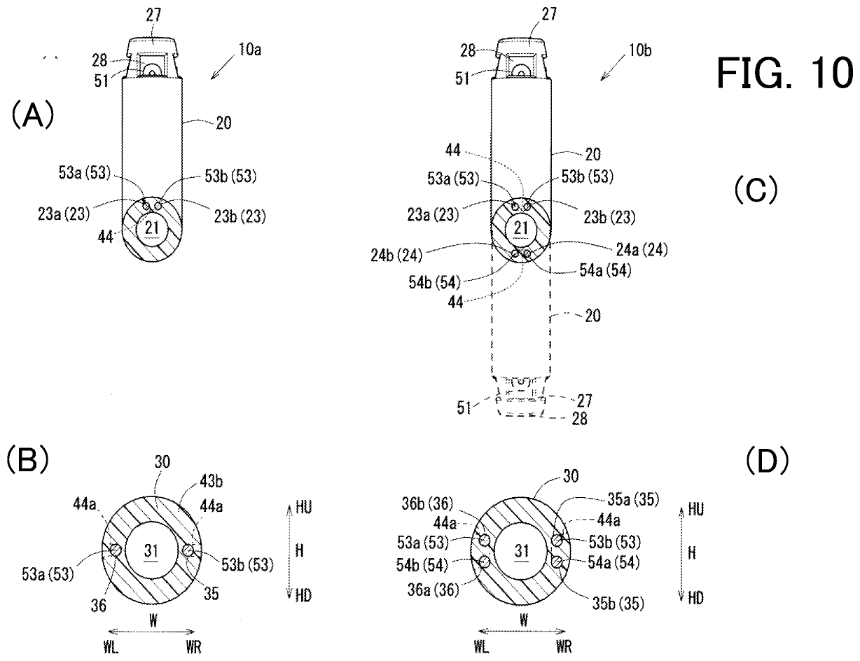

FIG. 9 shows an image diagram of clinical use of the movable elongated structure 10, and FIG. 10 shows an explanatory diagram of the movable elongated structures 10a and 10b with the distal end side flexible tube 20 which is bent. FIG. 10 at (A) shows a cross-sectional view corresponding to the cross-sectional view of the movable elongated structure 10a in a bent state taken along the line B-B in FIG. 2 at (A), and FIG. 10 at (B) shows an enlarged cross-sectional view corresponding to the arrow cross-sectional view taken along the line C-C in FIG. 2 at (A) in the movable elongated structures 10a, FIG. 10 at (C) shows a cross-sectional view of the movable elongated structure 10b in a bent state corresponding to the cross-sectional view of FIG. 2 at (A) taken along the line B-B, and FIG. 10 at (D) shows an enlarged cross-sectional view corresponding to the arrow cross-sectional view taken along the line C C in FIG. 2 at (A),

Note that the longitudinal direction of the movable elongated structure 10 is defined as a longitudinal direction L, and in the longitudinal direction L, the side of the distal end side flexible tube 20 with respect to the base end side flexible tube 30 is defined as a distal end side LF. The side of the base end side flexible tube 30 with respect to the tube 20 is defined as the base end side LB. For convenience of explanation of the movable elongated structure 10, the vertical direction in FIG. 1 is defined as the height direction H, the upward direction as the upward direction HU, and the downward direction as the downward direction HD. Further, the direction connecting the upper right and the lower left in FIG. 1 is defined as the width direction W, the upper right is defined as the right side WR, and the left lower side is defined as the left side WL.

Next, an embodiment of the movable elongated structure 10 will be described in detail with reference to FIGS. 1 to 5.

The movable elongated structure 10 includes a distal end side flexible tube 20 and a base end side flexible tube 30 arranged along the longitudinal direction L, and between the distal end side flexible tube 20 and the base end side flexible tube 30. It has a spacer 40 placed thereon and a traction wire 50 that passes through the inner walls of the flexible tubes 20 and 30. The movable elongated structure 10 is covered along the longitudinal direction L with an exterior cover (not shown). Also, only the outside of the spacer 40 may be covered with an exterior cover (not shown).

The distal end side flexible tube 20 is a cylindrical flexible tube elongated in the longitudinal direction 1, and has a distal end side main lumen 21 (FIG. 3 at (A): cross-sectional view taken along line 8-6 in FIG. 2 at (A)). The distal main lumen 21 is a space having a circular cross section along the longitudinal direction.

Further, as shown in FIG. 3 at (A), distal wire lumens 23a, 23b, 24a, 24b, 25a, 25b, 26a, 26b (hereinafter collectively referred to as 22).

The distal wire lumen 22 is a space with a circular cross section extending in the longitudinal direction L inside the tube wall, and is formed with a diameter that allows a traction wire 50 to be described later to be inserted therethrough,

The distal wire lumens 22 are provided in four directions on the tube wall having a ring-shaped cross section.

Specifically, as shown in FIG. 3 at (A), distal end side wire lumens 23a and 23b (hereinafter collectively referred to as 23) in the upward direction HU and distal end side wire lumens 24a and 24b in the downward direction HD in the pipe wall having a ring-shaped cross-section. (hereinafter collectively referred to as 24), right WR distal wire lumens 25a and 25b (hereinafter collectively referred to as 25), and left WL distal wire lumens 26a and 26b (hereinafter collectively referred to as 26).

In addition, as described above, the distal end side wire lumens 22 provided in the four directions in the tube wall having the ring-shaped cross section are spaced apart at predetermined intervals in the circumferential direction of the circular cross section so that the pair of traction wires 50 may be inserted therethrough are provided with two each.

In addition, the counterclockwise side of the wire lumen 22 on the distal end side constituted by a pair of two through holes is 22a (23a, 24a, 25a, 26a), and the clockwise side is 22b (23b, 24b, 25b, 28b).

In addition, the interval Ra between two distal end wire lumens 22 (22a, 22b) provided in each of the four directions on the pipe wan having a ring-shaped cross section (Ra is illustrated between 23a and 23b in FIG. 3 at (A)) is, the Interval Rb between the distal end side wire lumens 22 (23b and 25a, 25b and 24a, 24b and 26a, 26b and 23a) adjacent in the circumferential direction (Rb between 23b and 25a in FIG. 3 at (A)) set at intervals.

It should be noted that the distal end side wire lumens 22, which are provided in four directions and are provided two by two, may be integrally formed into an elliptical shape.

As shown in FIGS. 1, 2 and 5, a tip (or distal end) cap 27 is provided at the distal end LF of the distal end side flexible tube 20.

The distal end cap 27 has a bent recess 28 for forming a bent portion 51 of the traction wire 50, which will be described later. The bending recesses 28 are provided at four locations corresponding to the distal wire lumens 22 provided in four directions on the tube wall, and are provided with two insertion holes through which the traction wires 50 are inserted.

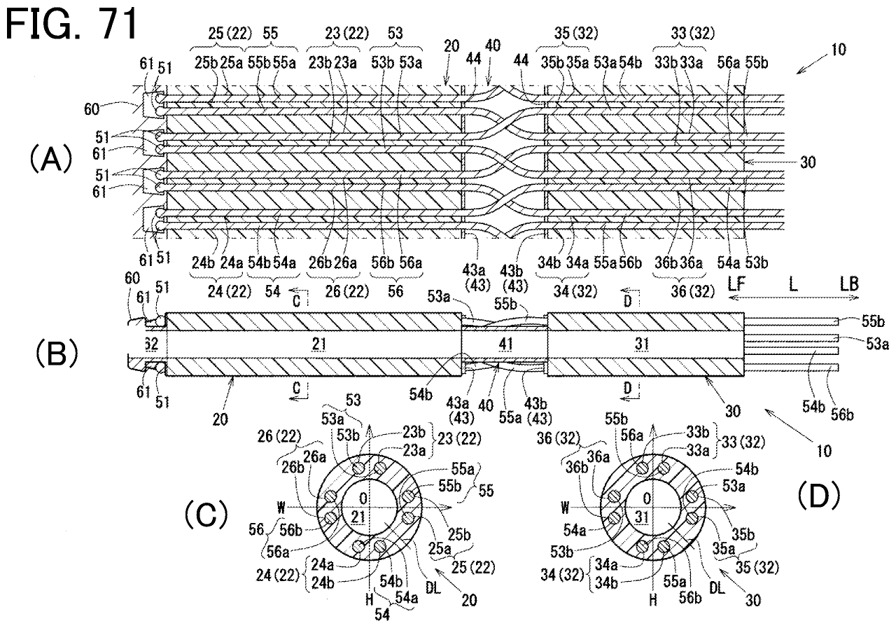

The distal end cap 27 may be removed by an instrument or the like inserted through a main lumen formed by connecting the distal end main lumen 21, the internal space 41 described later, and the base end main lumen 31 in a device using the movable elongated structure 10. A through hole penetrating in the longitudinal direction L may be provided as shown in FIG. 71 described later.

The distal end side flexible tube 20 configured as described above may be configured from a flexible tube such as polyamide elastomer, expanded polytetrafluoroethylene, polyurethane, or polytetrafluoroethylene.

As shown in FIGS. 1, 2 and 3, the base end side flexible tube 30 is a cylindrical flexible tube elongated in the longitudinal direction L, similar to the distal end side flexible tube 20, is provided with a base end side main lumen 31. The base end side main lumen 31 is a space having a circular crass section along the longitudinal direction L.

Further, as shown in FIG. 3 at (B) (cross-sectional view taken along line C-C in FIG. 2 at (A)), a base end side wire is inserted inside the tube wall between the base end main lumen 31 and the outer peripheral surface of the base end side flexible tube 30. It has lumens 33a, 33b, 34a, 34b, 35a, 35b, 36a, and 36b (hereinafter collectively referred to as 32).

Like the distal wire lumen 22, the base end wire lumen 32 is a circular cross-sectional space extending in the longitudinal direction L inside the tube wall, and is formed with a diameter that allows a traction wire 50, which will be described later, to pass through.

Like the distal wire lumen 22, the base end wire lumen 32 is provided in four directions on the tube wall having a ring-shaped cross section.

Specifically, as shown in FIG. 3 at (B), the base end side wire lumens 33a and 33b (hereinafter collectively referred to as 33) in the upward direction HU and the base end side wire lumen 34a in the downward direction HD in the pipe waft having a ring-shaped cross section 34b (hereinafter collectively referred to as 34), right WR base end side wire lumens 35a and 35b (hereinafter collectively referred to as 35), and left WL base end side wire lumens 36a and 36b (hereinafter collectively referred to as 36).

In addition, as described above, the base end wire lumens 32 provided In the four directions in the tube wall having a ring-shaped cross section are arranged two each at predetermined intervals in the circumferential direction of the circular cross section so that the pair of traction wires 50 may be inserted through them.

Note that the base end wire lumens 32, which are provided in four directions and are provided two by two, may be integrally formed into an elliptical shape.

In addition, the counterclockwise side of the base end side wire lumen 32 configured by a pair of two through holes is 32a (33a, 34a. 35a, 36a), and the clockwise side is 32b (33b, 34b, 35b, 36b).

In addition, the interval Rc between the base end side wire lumens 32 (32a, 32b) provided in each of the four directions on the pipe wall having a ring-shaped cross section (Rc is illustrated between 33a and 33b in FIG. 3 at (B)) is about half the interval between base end side wire lumens 32 (33b and 35a, 35b and 34a, 34b and 36a. 36b and 33a) adjacent in the circumferential direction (Rd is example written between 33b and 35a in FIG. 3 at (B)).

Like the distal end side flexible tube 20, the base end side flexible tube 30 configured as described above has flexibility such as polyamide elastomer, expanded polytetrafluoroethylene, polyurethane, or polytetrafluoroethylene. It may consist of a tube. The distal end side flexible tube 20 and the base end side flexible tube 30 may be made of the same material, or may be made of different materials.

As shown in FIGS. 4 and 5, the spacer 40 includes a cylinder 42 having an inner space 41 having a circular cross section penetrating in the longitudinal direction L. and flanges 43 (43a) provided at both ends of the cylinder 42 in the longitudinal direction L, 43b).

The internal space 41 is formed with a space having the same diameter as the main lumens 21 and 31 described above. The cylinder 42 has a diameter that is arranged inside the diameter of the traction wire 50 that is Inserted through the distal wire lumen 22 provided in the distal end side flexible tube 20 and the base end wire lumen 32 provided in the base end side flexible tube 30.

The flanges 43 (43a, 43b) are formed with an outer diameter that protrudes radially outward from the outer diameter of the cylinder 42, and are referred to as a distal wire lumen 22 and a base end wire lumen 33 (hereinafter referred to as wire lumens 22, 32). An arrangement recess 44 for arranging the traction wire 50 is provided at a corresponding location. The flange portion 43 is formed with an outer diameter equivalent to that of the distal end flexible tube 20 and the base end flexible tube 30.

The arrangement recess 44 is a recess for collectively arranging two traction wires 50 to be inserted into the two wire lumens 22 and 32, and is formed with a width and depth corresponding to the two wire lumens 22 and 32. are doing.

Of the protrusions 43 provided on both sides in the longitudinal direction L of the cylinder 42, the distal end side LF is defined as a distal end side flange 43a, and the base end side LB is defined as a base end side flange 43b.

Further, a spacing regulation part 45a is provided between a plurality of arrangement recesses 44 provided in the circumferential direction in the distal end side flange portion 43a. More specifically, separation regulation parts 45a provided on both sides of the placement recess (or arrangement) 44 restrict or regulate the traction wires 50 placed in the same placement recess 44 from being spaced apart in the circumferential direction at the distal end side flange portion 43a.

Specifically, the separation in the circumferential direction between the traction wires 50 arranged in the arrangement recesses 44 provided in the four directions in the distal end side flange 43a It is regulated by the regulation part 45a.

In addition, a proximity regulation part 45b is provided between a plurality of arrangement recesses 44 provided in the circumferential direction in the base-end flange 43b. More specifically, the proximity regulation portions 45b between the arrangement recesses 44 restrict the proximity of the traction wires 50 arranged in the arrangement recesses 44 adjacent in the circumferential direction in the base end side flange 43b to each other in the circumferential direction.

Specifically, the traction wire 50 arranged on the right side WR in the arrangement recess 44 in the upward direction HU in the base end flange portion 43b and the fraction wire 50 arranged in the upward direction HU in the arrangement recess 44 on the right side WR are close to each other. This is regulated by a separation regulation portion 45a between the arrangement recess 44 in the upward direction HU and the arrangement recess 44 in the right side WR.

Similarly, it is determined that the traction wire 50 arranged in the downward direction HD in the arrangement recess 44 of the right WR and the fraction wire 50 arranged in the right WR in the arrangement recess 44 in the downward direction HO are close to each other. The traction wire 50 is regulated by the separation regulation part 45a between the arrangement recess 44 in the direction HD, and is arranged on the left side WL in the arrangement recess 44 in the downward direction HD, and the traction wire 50 is arranged in the downward direction HD in the arrangement recess 44 in the left side WL 50 are regulated by the separation regulation part 45a between the arrangement recess 44 in the downward direction HD and the arrangement recess 44 in the left side WL, and the traction wire 50 arranged in the upward direction HU in the arrangement recess 44 in the left side WL. The proximity of the traction wire 50 arranged on the left side WL in the arrangement recess 44 in the upward direction HU is restricted by the separation regulation part 45a between the arrangement recess 44 in the left side WL and the arrangement recess 44 in the upward direction HU.

Furthermore, a guide convex part 46 for guiding the traction wire 50 radially outward is provided at the end of the cylinder 42 in the longitudinal direction L.

The guide convex part 46 is provided at a position corresponding to the placement recess 44 of the flange 43 at the end in the longitudinal direction L, with a corresponding width, and extends from the end in the longitudinal direction L to the center of the cylinder 42 in the longitudinal direction L. It is formed to have a trapezoidal vertical cross-sectional shape in which the protrusion height gradually increases (see FIG. 4 at (D)).

The guide convex parts 46 are provided at positions facing each other in the radial direction among the arrangement recesses 44 provided in the four directions at both ends in the longitudinal direction L, and the facing direction is the distal end side guide convex part 46a on the distal end side LF, and the base end side guide convex portion 46b of the base end side LB are provided so as to be perpendicular to each other.

Specifically, the distal end side guide convex portion 46a on the distal end side LF is provided in the upward direction HU and the downward direction HD so as to face each other in the height direction H, and the base end side guide convex portion 46b on the base end side LB is They are provided on the right side WR and the left side WL so as to face each other in the width direction W.

The spacer 40 configured as described above may be made of, for example, a metal material such as stainless steel, an elastic body such as polytetrafluoroethylene, an elastic body such as a spring, or a resin material.

Further, the spacer 40 may be configured by integrating the cylinder 42 and the flange portion 43, or may be configured separately and assembled. In addition, when the cylinder 42 and the flange portion 43 are configured separately, the cylinder 42 and the flange portion 43 may be configured with the same material, or may be configured with different materials.

The traction wire 50 is a flexible wire, and as shown in FIGS. 1 and 2, and formed with a length that is at least twice as long as the length in the longitudinal direction L of the movable elongated structure 10.

The traction wire 50 is composed of a bent portion 51 arranged in the bent concave portion 28 of the tip cap 27 and two traction portions 52 on the base end side LB from the bent portion 51.

As described above, the traction wire 50 that is bent back at the bending portion 51 and has a pair of traction portions 52 is provided with four pieces corresponding to the wire lumens 22 and 32 provided in the four directions. Specifically, traction portions 53a and 53b (hereinafter collectively referred to as 53) arranged generally in the upward direction HU, and traction portions 54a and 54b (hereinafter collectively referred to as 54) are arranged generally in the downward direction HD. There is a traction portion 55a, 55b (hereinafter collectively referred to as 55) located on the right side WR and a traction portion 56a, 56b (hereinafter collectively referred to as 56) located generally on the left side WL.

One of the portions corresponding to the traction portion 52 in each of the traction portions 53, 54, 55, 56 (counterclockwise side in the inserted state) is set as the pulled portion 53a, Ma, 55a, 56a, and the other (clockwise side in the inserted state) are served as traction portions 53b, 54b, 55b, and 56b.

The traction wire 50 may be made of a metal material such as stainless steel, nylon, fluorocarbon, or the like.

Assembly of the movable elongated structure 10 having the elements configured as described above will now be described.

First, the distal end side flexible tube 20, the spacer 40, and the base end side flexible tube 30 are arranged in series along the longitudinal direction L from the distal end side LF toward the base end side LB.

At this time, as shown in FIGS. 1, 2 and 5, the distal end side wire lumen 22 (23, 24, 25, 26), the four-direction placement recesses 44, the base end side wire lumen 32 (33, 34, 35, 36) are arranged to communicate with each other in the longitudinal direction L. Then, the traction portion 52 (FIG. 1) of the traction wire 50, which is bent around the center of the length to form the bent portion 51, is inserted through the distal wire lumen 22, the placement recess 44, and the base end wire lumen 32.

Specifically, first, the traction portion 53a of the traction portion 53 is inserted from the distal end side LF into the distal end side wire lumen 23a (FIG. 3 at (A)) of the left side WL in the distal end side wire lumen 23, and the traction portion 53b is inserted to the distal end side of the right side WR. It Is inserted through the wire lumen 23b to lead out the traction portion 53 from the base end side LB of the distal end side wire lumen 23, and the bent portion 51 is arranged in the bending concave portion 28 in the upward direction HU.

The traction portion 53 led out from the base end side LB of the distal end side wire lumen 23 is arranged in the arrangement recess 44 in the upward direction HU of the distal end side flange 43a, and obliquely downwards toward the base end side LB along the cylinder 42. It is stretched and arranged in the upward direction HU of the arrangement recess 44 in the width direction W of the base end side flange 43b.

Specifically, the traction portion 53a led out from the distal end side wire lumen 23a is arranged on the left side WL of the arrangement recess 44 in the upward direction HU in the distal end side flange 43a, and is arranged in the downward direction HD and the left side along the cylinder 42. It extends toward WL and is arranged in the upward direction HU of the placement recess 44 on the left side WL of the base-end flange 43b. Then, it is inserted into the base end side wire lumen 36b in the upward direction HU in the base end side wire lumen 36 on the left side WL of the base end side flexible tube 30, and led out from the base end side LB.

The traction portion 53b led out from the distal end side wire lumen 23b is arranged on the right side WR in the arrangement recess 44 in the upward direction HU in the distal end side flange portion 43a, and extends downward HD and right side WR along the cylinder 42, in the upward direction HU of the arrangement recess 44 on the right side WR of the base-end flange 43b. Then, it is inserted into the base end side wire lumen 35a in the upward direction HU in the base end side wire lumen 35 on the right side WR of the base end side flexible tube 30, and led out from the base end side LB.

As shown in FIGS. 2 and 3, the distal wire lumen 22 of the distal end side flexible tube 20 and the base end wire lumen 32 of the base end side flexible tube 30 through which the traction portion 52 is inserted as described above, are a set of through holes 11. The distal wire lumen 23a of the distal end side flexible tube 20 and the base end wire lumen 36b of the base end side flexible tube 30 are formed into a through hole set 11a through which the traction portion 53a is inserted, and the distal wire lumen 23b and the base end wire lumen 36b are formed into a pair of through holes 11a. The base end side wire lumen 35a is a through hole set 11b through which the traction portion 53b Is inserted.

Then, as shown in FIG. 3 at (8), a through hole set 11a is formed, and the center of the base end side wire lumen 36b through which the fraction portion 53 is inserted, and the base end through which the traction portion 53 is inserted are formed. A line passing through the center of the side wire lumen 35a is defined as a base end side virtual line BVL, and a line passing through the center of the base end side flexible tube 30 and parallel to the base end side virtual line BVL is defined as a base end side center line BCL, and the interval between the base end center line BCL and the base end virtual line BVL is defined as a base end interval X2.

As shown in FIG. 3 at (A), the center of the distal wire lumen 23a that constitutes the through hole set 11a and through which the traction portion 53 is inserted, and the distal wire lumen 23b that constitutes the through hole set 11b and through which the traction portion 53 is inserted. A line passing through the center of the distal end side is defined as a distal end side virtual line FVL, and a line passing through the center of the distal end side flexible tube 20 and parallel to the distal end side virtual line FVL is defined as a distal end side center line FCL. Assuming that the interval from the distal end side virtual line FVL is the distal end side interval X1, the distal end side interval X1 is wider than the base end side interval X2.

Next, as shown in FIGS. 1 and 3 at (A), the traction portion 54a of the traction portion 54 is inserted from the distal end side LF into the distal end side wire lumen 240 of the right side WR in the distal end side wire lumen 24, and the traction portion 54b is inserted into the left side WL. It is inserted into the distal end side wire lumen 24b to lead out the traction portion 54 from the base end side LB of the distal end side wire lumen 24, and the bent portion 51 is arranged in the bending concave portion 28 in the downward direction HO.

The traction portion 54 led out from the base end side LB of the distal end side wire lumen 24 is placed in the downward HD placement recess 44 of the distal end side flange 43a, and obliquely upwards toward the base end side LB along the cylinder 42. It is stretched and arranged in the downward direction HD of the arrangement recess 44 in the width direction W of the base-end flange 43b.

Specifically, the traction portion 54a led out from the distal end side wire lumen 24a is arranged on the right side WR of the arrangement recess 44 in the downward direction HD in the distal end side flange 43a, and is arranged in the upward direction HU and the right side along the cylinder 42. It extends toward the WR and is arranged in the downward direction HD of the arrangement recess 44 of the right WR in the base-end flange 43b. Then, it is inserted into the base end side wire lumen 35b in the downward direction HD in the base end side wire lumen 35 of the right side WR of the base end side flexible tube 30, and led out from the base end side LB.

The traction portion 54b led out from the distal end side wire lumen 24b is arranged on the left side WL of the arrangement recess 44 in the downward direction HD of the distal end side flange 43a, and extends along the cylinder 42 in the upward direction HU and the left side WL, in the downward direction HD of the arrangement recess 44 on the left side WL of the base-end flange 43b. Then, it is inserted into the base end side wire lumen 36a in the downward direction HD in the base end side wire lumen 36 on the left side WL of the base end side flexible tube 30, and led out from the base end side LB.

The distal wire lumen 24a of the distal end side flexible tube 20 and the base end wire lumen 35b of the base end side flexible tube 30 form a through hole set 11c through which the traction portion 54a is inserted. The base end side wire lumen 36a is a through hole set 11d through which the traction portion 54b is inserted (see FIG. 3 at (A) and (B)).

The center of the base end side wire lumen 35b that constitutes the through hole set 11 may through which the traction portion 54 is inserted, and the center of the base end side wire lumen 36a that constitutes the through hole set 11d and through which the traction portion 54 is inserted are separated. The through hole set 11c is configured with respect to the base end side interval (X2) between the base end side virtual line (BVL) passing through and the base end side center line (BCL) passing through the center of the base end side flexible tube 30, a distal virtual line (FVL) that passes through the center of the distal wire lumen 24a through which the traction portion 54 is inserted and the center of the distal wire lumen 24b that constitutes the through hole set 11d and through which the traction portion 54 is inserted, and the distal center line (FCL) passing through the center of the distal end side flexible tube 20 (X1) becomes wider.

Next, as shown in FIG. 3 at (A), the traction portion 55a of the traction portion 55 is inserted from the distal end side LF into the distal end side wire lumen 25a of the distal end side wire lumen 25 in the upward direction HU, and the traction portion 55b is inserted into the distal end side of the downward direction HD. It is inserted through the side wire lumen 25b to lead out the traction portion 55 from the base end side LB of the distal end side wire lumen 25, and the bent portion 51 is arranged in the bent concave portion 28 of the right side WR.

The traction portion 55 led out from the base end side LB of the distal end side wire lumen 25 is placed in the placement recess 44 on the right side WR of the distal end side flange 43a, and is pulled along the cylinder 42 toward the base end side LB toward the center in the width direction, and is arranged on the right side WR of the arrangement recess 44 in the height direction H of the base-end flange 43b.

Specifically, as shown in FIGS. 1, 3, and 5, the traction portion 55a led out from the distal end side wire lumen 25a is directed upward HU in the arrangement recess 44 of the right WR of the distal end side flange 43a. It extends in the left side WL and upward direction HU, and is arranged in the right side WR of the placement recess 44 in the upward direction HU in the base end flange 43b. Then, it Is inserted into the base end side wire lumen 33b of the right side WR in the base end side wire lumen 33 in the upward direction HU of the base end side flexible tube 30, and led out from the base end side LB.

In addition, it is arranged in the upward direction HU in the arrangement recess 44 on the right side WR of the distal end side flange 43a, extends toward the left side WL and upward HU, and extends toward the upward direction HU in the arrangement recess 44 in the base end side flange 43b. The traction portion 55a arranged on the right WR straddles the distal end side guide convex portion 46a of the right WR provided on the distal end side LF and the base end side guide convex portion 46b provided on the base end side LB in the upward direction HU. and is spaced apart from the outer surface of the cylinder 42 between the distal end side guide convex part 46a and the base end side guide convex part 46b.

The traction portion 55b led out from the distal wire lumen 25b is arranged in the downward direction HD in the arrangement recess 44 of the right side WR In the distal end side flange 43a, extends toward the left side WL and downward HD, and extends toward the base end side projection. It is arranged on the right side WR of the arrangement recess 44 in the downward direction HD in the edge 43b. Then, it is inserted into the base end side wire lumen 34a on the right side WR in the base end side wire lumen 34 in the downward direction HD of the base end side flexible tube 30, and led out from the base end side LB.

In addition, it is arranged in the upward direction HU in the arrangement recess 44 of the right side WR of the distal end side flange 43a, extends toward the left side WL and downward HD, and extends downward HD of the arrangement recess 44 of the base end side flange 43b. The traction portion 55b arranged on the right WR straddles the distal end side guide convex part 46a of the right WR provided on the distal end side LF and the base end side guide convex part 46b provided on the base end side LB in the downward direction HD. and is spaced apart from the outer surface of the cylinder 42 between the distal end side guide convex part 46a and the base end side guide convex part 46b.

The distal wire lumen 25a of the distal end side flexible tube 20 and the base end wire lumen 33b of the base end side flexible tube 30 are formed into a through hole set 11e through which the traction portion 55a is inserted, and the distal wire lumen 25b and the base end wire lumen 33b are formed into a pair of through holes 11e. The base end side wire lumen 34a is a through hole set 11f through which the traction portion 55b is inserted (see FIG. 3 at (A) and (B)).

The center of the base end side wire lumen 33b that constitutes the through hole set 11e and through which the traction portion 55 is inserted, and the center of the base end side wire lumen 34a that constitutes the through hole set 11f and through which the traction portion 55 is inserted are separated. The through hole set 11e is formed with respect to the base end side interval (X2) between the base end side virtual line (BVL) passing through and the base end side center line (BCL) passing through the center of the base end side flexible tube 30, a distal virtual line (FVL) passing through the center of the distal wire lumen 25a through which the traction portion 55 is inserted, and the center of the distal wire lumen 25b that constitutes the through hole set 11f and through which the traction portion 55 is inserted; and the distal center line (FCL) passing through the center of the distal end side flexible tube 20 (X1) becomes wider.

Next, the traction portion 56a of the traction portion 56 from the distal end side LF Is inserted into the distal wire lumen 26a of the distal wire lumen 26 in the downward direction HD, and the traction portion 564 is inserted into the distal wire lumen 26b of the upward direction HU. Then, the traction portion 56 is fed out from the base end side LB of the distal end side wire lumen 26, and the bent portion 51 is arranged in the bent concave portion 28 on the left side WL.

The traction portion 56 led out from the base end side LB of the distal end side wire lumen 26 is arranged in the arrangement recess 44 on the left side WL of the distal end side flange 43a, and along the cylinder 42, toward the base end side LB, the center in the width direction, and is arranged on the left side WL of the arrangement recess 44 in the height direction H of the base-end flange 43b.

Specifically, as shown in FIGS. 1 and 5 at (A), the traction portion 56a led out from the distal wire lumen 26a is arranged in the downward direction HD In the arrangement recess 44 on the Left side WL of the distal flange 43a, It extends to the right side WR and downward HD, and is arranged on the left side WL of the arrangement recess 44 in the downward direction HD in the base-end flange 43b. Then, it is inserted into the base end side wire lumen 34b on the left side WL in the base end side wire lumen 34 in the downward direction HD of the base end side flexible tube 30, and led out from the base end side LB.

In addition, it is arranged in the downward direction HD in the arrangement recess 44 on the left side WL in the distal end side flange 43a, extends toward the right side WR and in the downward direction HD, and extends toward the downward direction HD in the arrangement recess 44 in the base end side flange 43b. The traction portion 56a arranged on the left side WL straddles the distal end side guiding convex portion 46a of the left side WL provided on the distal end side LF and the base end side guiding convex portion 46b provided on the base end side LB in the downward direction HD. and is spaced apart from the outer surface of the cylinder 42 between the distal end side guide convex part 46a and the base end side guide convex part 46b.

The traction portion 56b led out from the distal wire lumen 26b is arranged in the upward direction HU In the arrangement recess 44 on the left side WL of the distal end side flange 43a, extends toward the right side WR and upward HU, and extends toward the base end side projection. It is arranged on the left side WL of the arrangement recess 44 in the upward direction HU In the edge 43b. Then, it is inserted into the base end side wire lumen 33a on the left side WL of the base end side wire lumen 33 in the upward direction HU in the base end side flexible tube 30, and led out from the base end side LB.

In addition, it is arranged in the downward direction HD in the arrangement recess 44 on the left side WL of the distal end side flange 43a, extends toward the right side WR and upward HU, and extends toward the upward direction HU in the arrangement recess 44 on the base end side flange 43b. The traction portion 56b arranged on the left side WL straddles the distal end side guiding convex portion 46a of the left side WL provided on the distal end side LF and the base end side guiding convex portion 46b provided on the base end side LB in the upward direction HU. and is spaced apart from the outer surface of the cylinder 42 between the distal end side guide convex part 46a and the base end side guide convex pert 46b.

The distal wire lumen 26a of the distal end side flexible tube 20 and the base end wire lumen 34b of the base end side flexible tube 30 are formed into a through hole set 11g through which the traction portion 56a is inserted, and the distal wire lumen 26b and the base end wire lumen 34b are formed into a pair of through holes 11g. The base end side wire lumen 33a is a through hole set 11h through which the traction portion 56b is inserted (see FIG. 3 at (A) and (B)).

The center of the base end side wire lumen 34b that constitutes the through hole set 119 and through which the traction portion 56 is inserted, and the center of the base end side wire lumen 33a that constitutes the through hole set 11h and through which the traction portion 56 is inserted are separated. The through hole set 11g is configured with respect to the base end side interval (X2) between the base end side virtual line (BVL) passing through and the base end side center fine (BCL) passing through the center of the base end side flexible tube 30, a distal virtual line (FVL) passing through the center of the distal wire lumen 26a through which the fraction portion 56 is inserted and the center of the distal wire lumen 26b that constitutes the through hole set 11h and through which the traction portion 56 is inserted, and the distal center line (FCL) passing through the center of the distal end side flexible tube 20 (X1) becomes wider.

In addition, in the spacer 40 of the movable elongated structure 10 assembled as described above, the traction portion 55 and the traction portion 56 are formed by the distal end side guide convex part 46a on the distal end side LF and the base end side guide convex part 46a on the base end side LB. Since it extends obliquely across the projection 46b and is spaced radially outward from the cylinder 42, it is disposed beyond the radially outer sides of the traction portions 53 and 54 extending obliquely along the cylinder 42.

When the traction wires 50 are Inserted through the wire lumens 22 and 32 and arranged as described above, the two traction portions 52 are respectively arranged in the arrangement recesses 44 of the spacer 40.

Specifically, as shown in FIG. 5 at (D), the fraction portions 53a and 53b are arranged in the arrangement recess 44 in the upward direction HU in the distal end side flange portion 43a, and the traction portions 54a and 54b are arranged in the arrangement recess 44 in the downward direction HQ. The fraction portions 55a and 55b are disposed in the placement recess 44 on the right side WR, and the traction portions 560 and 56b are placed in the placement recess 44 on the left side WL.

Then, as shown In FIG. 5 at (C), the traction portions 56b and 55a are arranged in the arrangement recess 44 in the upward direction HU in the base end side flange portion 43b, and the traction portions 56a and 55b are arranged in the arrangement recess 44 in the downward direction HG. The traction portions 53b and 54a are arranged in the placement recess 44 on the right side WR, and the traction portions 53a and 54b are placed in the placement recess 44 on the left side WL.

In the above description, the traction portions 53, 54, 55, and 56 are inserted in the order, but the insertion order is not limited and may be inserted in any order.

Also, from the base end side LS to the distal end side LF, it Is inserted through the base end side wire lumen 32, the arrangement recess 44, and the distal end side wire lumen 22 in this order, and is bent back at the bending recess 28 of the distal end side LF. The portion 51 may be formed, and the distal wire lumen 22, the arrangement recess 44, and the base end wire lumen 32 may be inserted in this order.

Furthermore, after the traction wire 50 is inserted through the distal wire lumen 22 of the distal end side flexible tube 20 and the base end wire lumen 32 of the base end side flexible tube 30 arranged along the longitudinal direction L, Loosening the traction wire 50 between the flexible tube 20 and the base end side flexible tube 30, placing the spacer 40 between the distal end side flexible tube 20 and the base end side flexible tube 30, and loosening the traction wire 50 may be placed in the placement recess 44 for assembly.

The movable elongated structure 10 configured in this way has the following features compared to conventional bidirectionally movable ureteroscopes and movable bile duct catheters. The movable elongated structure 10 may be formed with an outer diameter (1.8 to 3.2 mm) having a narrowest diameter smaller than that of a ureteroscope and a larger diameter than that of a movable bile duct catheter.

In addition, flexible tubes may be used for the distal end side flexible tube 20 and the base end side flexible tube 30 that constitute the movable elongated structure 10, and the maximum bending angle of 270 degrees, which is equivalent to that of a ureteroscope, is possible. It may freely move and bend in four directions (all directions) to a certain extent.

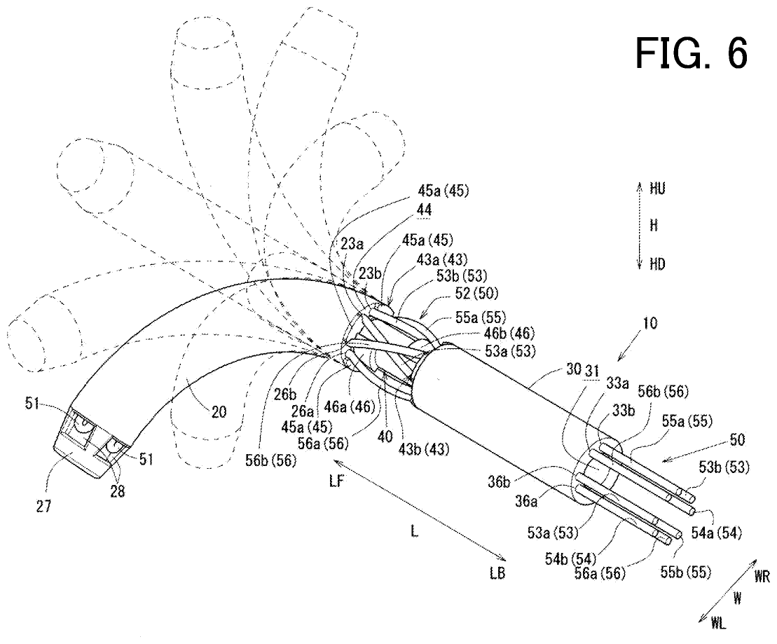

As shown in FIGS. 6 to 8, thus constructed movable elongated structure 10 may be smoothly bent in the longitudinal direction L by pulling the traction wire 50 toward the base end side LB.

Specifically, the distal end side flexible tube 20 is bent upward HU by pulling the traction portion 53 through which the distal wire lumen 23 in the upward direction HU is inserted in the distal end side flexible tube 20 toward the base end side LB. The distal end side flexible tube 20 may be bent downward HD by pulling the traction portion 54 inserted through the distal wire lumen 24 in the downward direction HD toward the base end side LB.

By pulling the traction portion 55 of the distal end side flexible tube 20 through which the distal wire lumen 25 of the right WR is inserted to the base end side LB, the distal end side flexible tube 20 may be bent to the right WR and left WL. The distal end side flexible tube 20 may be bent to the left side WL by pulling the traction portion 56 passing through the distal wire lumen 26 toward the base end side LB.

More specifically, as shown in FIG. 7, the traction portion 53a of the traction portion 53 that passes through the distal wire lumen 23a (FIG. 7 at (A)) in the upward direction HU in the distal end side flexible tube 20 is the base end side flexible tube 30. is inserted into the base end side wire lumen 36b (FIG. 7 at (B)) of the base end side wire lumen 36 of the left side WL (FIG. 7 at (B)), and the traction portion 53b is the base end side of the upward direction HU of the base end side wire lumen 35 of the right side WR. It is inserted through the wire lumen 35a.

A circumferential interval Rf between the base end wire lumen 35a and the base end wire lumen 36b, which is the upward direction HU of the base end wire lumens 35 and 36 in the width direction W, is the interval between the distal wire lumen 23 and the distal wire lumen. The distal end side wire lumens 23a and 23b are positioned in the upward direction HU with respect to the base end side wire lumens 35a and 36b.

Therefore, when the traction portion 53 is pulled toward the base end side LB, as shown in FIG. 7 at (A), the base end side wire lumens 35a and 36b, which are widely spaced in the circumferential direction, exert a puking force on the base end side flexible tube 30 in the bending direction, and the traction force acts eccentrically in the upward direction HU by the traction portion 53 inserted into the distal wire lumen arranged in the upward direction HU in the distal end side flexible tube 20, and the distal end side flexible tube 20 may be bent upward HU.

Similarly, the traction portion 54a of the traction portion 54 passing through the distal wire lumen 24 in the downward direction HD in the distal end side flexible tube 20 is the base end portion WR in the base end side flexible tube 30, as shown in FIG. 7 at (8y. It is inserted through the base end side wire lumen 35b in the downward direction HD of the base end side wire lumen 35, and the traction portion 54b is inserted through the base end side wire lumen 36a in the downward direction HD of the base end side wire lumen 36 of the left side WL.

A circumferential interval Rg between the base end wire lumens 35b and 36a, which is the downward direction HD of the base end wire lumens 35 and 36 in the width direction W, is the interval between the distal wire lumen 24a of the distal wire lumen 24 and the distal wire lumen 24a. The wire lumens 24a, 24b on the distal end side are positioned downward HD with respect to the wire lumens 35b, 36a on the base end side, and are wider than the circumferential interval Re with respect to the lumen 24b.

Therefore, when the traction portion 54 is pulled toward the base end side LB, the base end side wire lumens 35b and 36a, which are widely spaced in the circumferential direction, hardly exert a pulling force on the base end side flexible tube 30 in the bending direction, the traction portion 54 inserted into the distal wire lumen 24 arranged in the downward direction HD in the distal end side flexible tube 20 acts eccentrically In the downward direction HD to bend the distal end side flexible tube 20 in the downward direction HD.

The traction portion 55a of the traction portion 55 through which the right WR distal wire lumen 25 of the distal end side flexible tube 20 is inserted is the right WR of the base end side wire lumen 33 of the base end side flexible tube 30 in the upward direction HU, and the traction portion 55b is inserted through the base end wire lumen 34a of the right side WR of the base end side wire lumen 34 in the downward direction HD.

The interval between the base end wire lumens 33b and 34a, which is the right WR of the base end wire lumens 33 and 34 in the height direction H, is the interval between the distal wire lumen 25a of the distal wire lumen 25 and the distal wire lumen 25a. 25b, and the distal wire Lumens 25a, 25b are positioned on the right side WR with respect to the base end wire lumens 33b, 34a.

Therefore, when the traction portion 55 is pulled toward the base end side LB, as shown in FIG. 7 at (A), the base end side wire lumens 33b and 34a, which are widely spaced in the circumferential direction, exert a pulling force on the base end side flexible tube 30 in the bending direction. The traction force acts eccentrically on the right side WR due to the traction portion 55 inserted into the distal end side wire lumen 25 arranged on the right side WR of the distal end side flexible tube 20, and the distal end side flexible tube 20 is pulled. It may be bent to the right WR.

Furthermore, the traction portion 56a of the traction portion 56 inserted through the distal wire lumen 26 on the left side WL of the distal end side flexible tube 20 is the left side WL of the base end wire lumen 34 in the downward direction HD in the base end side flexible tube 30. As shown in FIG. 7 at (B), the traction portion 561) is inserted through the base end wire lumen 33a on the left side WL of the base end wire lumen 33 in the upward direction HU.

A circumferential distance or interval between the base end wire lumens 33a and 34b, which is the left side WL of the base end wire lumens 33 and 34 in the height direction H, is wider than the circumferential interval between distal end side wire lumens 26a and 26b of the distal wire lumen 26, and the distal wire lumens 26a and 26b are positioned on the left side WL with respect to the base end wire lumens 33a and 34b.

Therefore, when the traction portion 56 is pulled toward the base end side LB, the base end side wire lumens 33a and 34b, which are widely spaced in the circumferential direction, hardly act on the base end side flexible tube 30 in the bending direction. A traction force acts eccentrically on the left side WL by the traction portion 56 inserted into the distal end side wire lumen 26 arranged on the left side WL in the distal end side flexible tube 20, and the distal end side flexible tube 20 may be bent to the left side WL.

Also, as described in detail above and shown in FIG. 7 at (A), the distal side flexile tube 20 is bent upward HU by pulling the pulling portion 53 to the proximal side LB, the distal flexible tube 20 is bent downward HD by pulling the pulling portion 54, the distal flexible tube 20 is bent rightward WR by pulling the pulling portion 55, the distal flexible tube 20 is bent leftward WL by pulling the pulling portion 56 in the movable elongated structure 10, whereby as shown in FIG. 8, the distal flexible tube 20 may be bent in an oblique direction crossing the height direction H and the width direction W by pulling the pulling wire 50.

Specifically, when the traction portion 53 that bends the distal end side flexible tube 20 upward HU and the traction portion 55 that bends it rightward WR are pulled toward the base end side LB with the same pulling force (FIG. 8 at (A)), the distal end side flexible tube 20 may be bent obliquely upward to the right at 45 degrees when viewed from the longitudinal direction L with respect to the upward direction HU and the right side WR.

If the traction force of the traction portion 55 is greater than that of the traction portion 53, the force for bending to the right side WR is increased, and the vehicle is bent obliquely upward to the right toward the direction of the right side WR, the force for bending in the upward direction HU increases, and the bending occurs in an oblique upward right direction approaching the upward direction HU.

Further, when the traction portion 53 that bends the distal end side flexible tube 20 upward HU and the traction portion 56 that bends it leftward WL are pulled toward the base end side LB with the same pulling force, as shown in FIG. 8 at (A). The tube 20 may be bent obliquely upward to the left at 45 degrees when viewed from the longitudinal direction L with respect to the upward direction HU and the left side WL.

If the traction force of the traction portion 56 is greater than that of the traction portion 53, the force of bending to the left side WL increases, and the vehicle is bent obliquely upward to the left toward the direction of the left side WL. If it is increased, the force for bending in the upward direction HU will be increased, and the bending will be In the left oblique upward direction approaching the upward direction HU direction.

Conversely, if the traction portion 54 that bends the distal end side flexible tube 20 downward HD and the traction portion 55 that bends the distal end side flexible tube 20 rightward WR are pulled toward the base end side LB with the same pulling force, the distal end side flexible tube 20 is pulled downward HD and rightward. It may be bent obliquely downward to the right at 45 degrees when viewed from the longitudinal direction L with respect to WR.

If the traction force of the traction portion 55 is greater than that of the traction portion 54, the force for bending to the right side WR is increased, and the vehicle is bent obliquely downward to the right toward the direction of the right side WR, the force for bending in the downward direction HD increases, and the sheet is bent obliquely downward to the right closer to the downward HD direction.

When the traction portion 54 bending the distal end side flexible tube 20 downward HD and the traction portion 56 bending the distal end side flexible tube 20 leftward WL are pulled toward the base end side LB with the same pulling force, the distal end side flexible tube 20 is pulled downward HD and leftward WL, it may be bent obliquely downward to the left at 45 degrees when viewed from the longitudinal direction L.

If the traction force of the traction portion 56 is greater than that of the traction portion 54, the force for bending to the left side WL increases, and the vehicle is bent obliquely downward to the left toward the direction of the left side WL, the force for bending in the downward direction HD increases, and the sheet is bent in an obliquely downward left direction approaching the downward HD direction.

Furthermore, when the traction portions 53, 54, 55, 56 are pulled with the same pulling force, the flexible tubes 20 and 30 are compressed in the longitudinal direction L, whereby the length in the longitudinal direction L of the movable elongated structure 10 may also be shortened.

As described above, the moveable elongated structure 10 may be pulled distally relative to the base end side flexible tube 30 as shown in FIG. 6, the side flexible tube 20 may be bent or shortened in any desired direction.

However, as described above, the traction wire 50 arranged in the arrangement recess 44 of the spacer 40 extends in an oblique direction that intersects with the longitudinal direction 1. Although the forte acts, the movement is restricted by the restriction portions 45 (45a, 45b), which are the edges of the arrangement recess 44 in the flange portion 43. Therefore, it is possible to prevent a circumferential force from acting on the distal wire lumen 22 and the base end wire lumen 32 through which the traction wire 50 is Inserted.

In addition, the movable elongated structure 10 that may bend the distal end side flexible tube 20 in a desired direction by pulling the traction wire 50 is arranged such that the traction wire 50 is arranged at the flange 43 of the spacer 40. Since the concave portion 44 is a concave portion that is open radially outward, when the traction wire 50 is loosened between the distal end side flexible tube 20 and the base end side flexible tube 30 as shown in FIG. 3 at (C) The traction wire 50 is removed from the tube and the spacer 40 may be removed from between the distal end side flexible tube 20 and the base end side flexible tube 30. Therefore, for example, spacers 40a having different lengths in the longitudinal direction L or spacers having different deformability due to materials may be easily exchanged.

As described above, the elongated flexible distal end side flexible tube 20 and the base end side flexible tube 20 are arranged in series along the longitudinal direction L from the distal end side LF toward the base end side LB. A pair of flexible traction wires 50 passing through a tube 30, a pair of wire lumens 22, 32 provided inside the tube walls of the flexible tubes 20, 30 along the longitudinal direction L, and a distal end side flexible tube 20 and the base end side flexible tube 30 and provided with a spacer 40 for regulating the direction of the traction wire 50, and a pair of base end wire lumens 32 provided in the base end side flexible tube 30. is set wider than the interval in the circumferential direction between a pair of distal wire lumens 22 provided in the distal end side flexible tube 20 and through which the same traction wire 50 is inserted. A cylindrical cylinder 42 arranged along the base end side flange 43b provided at the end portion of the base end side LB of the cylinder 42 and protruding radially outward, and an end portion of the distal end side LF of the cylinder 42 and a distal end side flange 43a protruding radially outward is provided, and the traction wire led out from the base end side wire lumen 32 to the base end side flange 43b and introduced into the distal end side wire lumen 22 50 in the circumferential direction, and an arrangement recess 44 that is open on the radially outer side and in which the traction wire 50 is arranged. 32 and introduced into the wire lumen 22 on the distal end side, and a placement recess 44 that is open radially outward and in which the traction wire 50 is placed. Since the movable elongated structure 10 is configured so that the spacer 40 may be removed from between the distal end side flexible tube 20 and the base end side flexible tube 30, the movable elongated structure 10 may be assembled. This improves usability and versatility.

More specifically, by removing the spacer 40 from between the distal end side flexible tube 20 and the base end side flexible tube 30, the ease of assembly of the movable elongated structure 10 is improved and the movable elongated structure is unproved. The spacer 40 may be replaced according to the application and specifications of the body 10.

In addition, when disposing of the movable elongated structural body 10, the spacer 40 may be removed for separate disposal.

In addition, a flexible elongated distal end side flexible tube 20 and a base end side flexible tube 30 are arranged in series along the longitudinal direction L from the distal end side LF toward the base end side LB. A cylindrical cylinder 42 arranged along the longitudinal direction t, in the spacer 40 arranged therebetween, and a base end side flange provided at the end of the base end side LB of the cylinder 42 and protruding radially outward. 43b, and a distal end side flange 43a provided at the end of the distal end side LF of the cylinder 42 and protruding radially outward, a proximity regulation portion 45b for regulating proximity in the circumferential direction of the traction wire 50 introduced into the distal wire lumen 22, and an arrangement recess 44, which is open radially outward and in which the traction wire 50 is arranged, are provided. A separation regulation part 45a that regulates the separation in the circumferential direction of the traction wire 50 led out from the base end side wire lumen 32 and Introduced into the distal end side wire lumen 22 is provided on the side flange portion 43a. An arrangement recess 44 is provided in which a wire 50 is arranged, and a pair of wire lumens 22, 32 in which the spacer 40 is inserted along the longitudinal direction L provided inside the tube walls of the flexible tubes 20, 30. In order to regulate the direction in which the flexible traction wire 50 extends, the traction wire 50 led out from the base end side wire lumen 32 provided in the base end side flexible tube 30 is smoothly introduced into the distal end side wire lumen 22.

In addition, the interval (Rf, Rg) in the circumferential direction between the pair of base end side wire lumens 32 is the same interval (Ra, Re) in the circumferential direction between the pair of distal end side wire lumens 22 through which the traction wire 50 is inserted. set wider.

In addition, a base end virtual line BVL passing through the centers of the base end wire lumen 32 through which the traction portion 52 is inserted, and a base parallel to the base end virtual line BVL passing through the center of the base end side flexible tube 30. With respect to the base end side interval (X2) from the base end side center line BCL, the distal end side virtual line FVL passing through the centers of the distal end side wire lumens 22 through which the traction portion 52 is inserted and the center of the distal end side flexible tube 20 A distal end side interval X1 between a line parallel to the distal end side virtual line FVL and the distal end side center line FCL is widened.

Therefore, by pulling the pair of traction wires 50 inserted through the base end side wire lumen 32 with a wide circumferential interval and a narrow base end side interval X2, the circumferential interval is narrow and the distal end side interval X1 is wide. A distal end side flexible tube 20 having a distal wire lumen 22 may be bent.

The wire lumens 22, 32 are provided inside the tube walls of the flexible tubes 20, 30, and in the spacer 40, they are provided at the end of a cylindrical cylinder 42 and arranged on a flange 43 protruding radially outward. Since the recess 44 is provided, the traction wire 50 does not protrude Into the internal space 41 of the spacer 40, and the main lumens 21 and 31 may be effectively utilized.

Furthermore, since the arrangement recess 44 provided at the end portion of the cylindrical cylinder 42 and provided in the flange portion 43 protruding radially outward is open radially outward, the traction wire 50 may be inserted into the wire lumens 22 and 32. After insertion, the spacer 40 may be arranged between the base end side flexible tube 30 and the distal end side flexible tube 20, and the traction wire 50 may be arranged in the arrangement recess 44, so that the movable elongated structure 10 may be placed. may improve the assemblability.

Specifically, as shown in FIG. 5 at (C) and (D), the separation regulation portions 45a on both sides of the arrangement recess 44 in the upward direction HU regulates separation of the traction portions 53a and 53b inserted through the distal wire lumens 23a and 23b at the distal end side flange portion 3a.

Similarly, the separation regulation parts 45a on both sides of the arrangement recess 44 in the downward direction HD in the distal end side flange 43a restrict the separation of the traction portions 54a and 54b inserted through the distal end side wire lumens 24a and 24b. In the flange 43a, the separation regulation portions 45a on both sides of the arrangement recess 44 of the right side WR regulate the separation of the traction portions 55a and 55b inserted through the distal end side wire lumens 25a and 25b, and the left side of the distal end side flange 43a. Separation regulation parts 45a on both sides of the placement recess 44 of the WL regulate the traction portions 56a, 56b inserted through the distal end side wire lumens 26a, 26b from being separated from each other.

Then, in the base end side flange 43b, a traction portion 53b inserted through the base end side wire lumen 35a in the upward direction HU of the base end side wire lumen 35 of the right WR and the base end side wire lumen 36 of the left-side WL. The proximity of the fraction portion 53a inserted in the base end side wire lumen 36b in the upward direction HU is controlled by the proximity regulation portion 45b between the arrangement recess 44 in the upward direction HU and the arrangement recess 44 in the right side WR and the upward direction. This is regulated by the proximity regulation part 45b between the placement recess 44 of the HU and the placement recess 44 of the left WI.

Further, in the base end side flange portion 43b, the traction portion Ma inserted through the base end side wire lumen 35b in the downward direction HD of the right-hand side WR and the base end side wire lumen 36 of the left-hand side WL. The proximity of the traction portion 54b inserted in the base-side wire lumen 36a in the downward direction HD is controlled by the proximity regulation portion 45b between the arrangement recess 44 in the downward direction HD and the arrangement recess 44 in the right side WR, and the downward direction. This is regulated by a proximity regulation part 45b between the placement recess 44 of the HD and the placement recess 44 of the left side WL.

Similarly, in the base end side flange portion 43b, a traction portion 55a inserted through the base end side wire lumen 33b of the right WR of the base end side wire lumen 35 in the upward direction HU, and the base end side wire lumen 33b in the downward direction HD. The proximity of the traction portion 55b inserted through the base end wire lumen 34a of the right WR of 36 to the proximity regulation portion 45b between the arrangement recess 44 of the right WR and the arrangement recess 44 of the upward HU and the right side. This is regulated by the proximity regulation part 45b between the WR placement recess 44 and the downward placement recess 44 HD.

In addition, in the base end side flange portion 43b, a traction portion 56b inserted through the base end side wire lumen 33a on the left side WL of the base end side wire lumen 36 in the upward direction HU, and the base end side wire lumen 34 in the downward direction HD. The proximity of the traction portion 56a inserted in the base-end wire lumen 34b of the left WL of the left WL to the proximity regulation part 45b between the arrangement recess 44 of the left WL and the arrangement recess 44 of the upward direction HU and the left WI is regulated by the proximity regulation part 45b between the arrangement recess 44 in the direction HD and the arrangement recess 44 in the downward direction HD.

Further, as shown in FIG. 3 at (A) and (B), a pair of base end wire lumens 32 and a pair of distal wire lumens 22 through which the pair of traction wires 50 are inserted constitute a set of through holes 11, and a plurality of sets of through holes are formed. The through hole sets 11 are arranged at different positions in the circumferential direction, and a plurality of proximity regulation parts 45b and arrangement recesses 44 are provided on the base end side flange 43b, and the separation regulation parts 45a and 44 are provided on the distal end side flange 43a. Since a plurality of arrangement recesses 44 are provided, the distal end portion of the movable elongated structure 10 may be bent in a bending direction corresponding to the number of sets.

More specifically, a plurality of through hole sets 11 are arranged at different positions in the circumferential direction, a plurality of proximity regulation parts 45b and a plurality of arrangement recesses 44 are provided in the base-side flange 43b, and a plurality of recesses 44 are provided in the distal end side flange 43a. By providing a plurality of separation regulation parts 45a and arrangement recesses 44, at least a pair of the traction wires 50 out of the plurality of traction wires 50 inserted through the wire lumens 22, 32 may be pulled. The distal end portion of the movable elongated structure 10 may be bent in a bending direction corresponding to the traction wires 50.

In addition, in the base-end flange 43b, the common proximity regulation part 45b regulates the circumferentially adjacent traction wires 50 of the pair of traction wires 50 inserted through the through hole sets 11 that are adjacent in the circumferential direction from approaching each other, and the circumferentially adjacent traction wires 50 of the pair of traction wires 50 inserted through the through hole sets 11 adjacent in the circumferential direction are separated from each other at the distal end side flange 43a. Since the spacing regulation portion 45a is common to the traction wire 50, the directions of the plurality of pairs of traction wires 50 may be regulated by the spacer 40 having a simple structure.

More specifically, in the base end flange 43b, the common proximity regulation portion regulates the circumferentially adjacent traction wires 50 of the pair of traction wires 50 inserted through the circumferentially adjacent through hole sets 11 from approaching each other. 45b, that is, the traction wire 50 inserted through the through hole sets 11 adjacent in the circumferential direction may also be served as the proximity regulation portion 45b, the proximity of different pairs of traction wires 50 adjacent in the circumferential direction may be restricted.

In addition, in the distal end-side flange portion 43a, the separation of the traction wires 50 that are adjacent in the circumferential direction among the pair of traction wires 50 that are inserted through the through hole sets 11 that are adjacent in the circumferential direction may be restricted by the separation regulation part 45a. In other words, since the separation regulation portion 45a may be shared by the traction wires 50 inserted through the through hole sets 11 adjacent to each other on the opposite side in the circumferential direction, the structure is simpler than the case where the separation regulation portions 45a are provided in the respective traction wires 50, the separation of different pairs of traction wires 50 adjacent in the circumferential direction may be regulated.

As described above, the proximity regulation part 45b and the separation regulation part 45a function as the regulation parts 45 for different pairs of traction wires 50 that are adjacent in the circumferential direction, so that the construction may be made simple. It should be noted that a simpler structure may be achieved by arranging different pairs of traction wires 50 that are adjacent in the circumferential direction in the arrangement recesses 44 in the base end side flange 43b and the distal end side flange 43a.

For a specific example, as shown in FIG. 3 at (A) and (B), a through hole set h a constituted by a distal wire lumen 23a through which a traction portion 53a is inserted and a base end wire lumen 36b, and a distal wire through which a traction portion 56b is inserted. A through hole set 11h composed of the lumen 26b and the base end side wire lumen 33a will be described below.

Common proximity regulation part that the circumferentially adjacent traction portions 53a and 56b of the pair of traction portions 53 and 56 inserted through the circumferentially adjacent through hole sets 11a and 11h of the base-end flange 43b are close to each other which may be regulated by the regulation part 45b.

That is, the circumferentially adjacent towed portions 53a and 56b of the pair of towed portions 53 and 56 inserted through the circumferentially adjacent through hole sets 11a and 11h may also serve as the proximity regulation part 45b.

Therefore, compared with the case where the proximity regulation part 45b is provided in each of the towed portions 53a and 56b, the towed portions 53a and 56b of the towed portion 53 and the towed portion 56 of the through hole sets 11a and 11h adjacent in the circumferential direction are simpler in structure, so that proximity may be regulated.

In addition, in the distal end side flange portion 43a, the right side WR of the arrangement recess 44 in the upward direction HU indicates that the traction portions 53a and 53b of the pair of traction portions 53 that are inserted through the through hole sets 11a and 11b that are adjacent in the circumferential direction are separated from each other, and the separation regulation portion 45a on the left side WL.

In addition, in the distal end side flange portion 43a, the upward direction HU of the arrangement recess 44 of the right side WR indicates that the traction portions 55a and 55b of the pair of traction portions 55 inserted through the through hole sets 11e and 11f that are adjacent in the circumferential direction are separated from each other, and the separation regulation portion 45a in the downward direction HD.

That is, of the pair of traction portions 53 and 55, the through hole sets 11b and 11e, which are circumferentially adjacent to each other, are inserted through the separation regulation part 45a between the arrangement recess 44 in the upward direction HU and the arrangement recess 44 in the right side WR. The traction portions 53b and 55a adjacent to the may also be used.

Therefore, compared with the case of providing the separation regulation part 45a in each of the traction portions 53b, 55a, the traction portions 53b. 55a of the traction portion 53 and the traction portion 55 of the through hole sets 11a, 11h adjacent in the circumferential direction are simpler in structure. may be regulated.

In addition, one traction wire 50 and the other traction wire 50 inserted through the through hole sets 11 adjacent in the circumferential direction intersect in the circumferential direction, and the one traction wire 50 is inserted into the cylinder 42 to the outside of the other traction wire 50. Since the guide convex portion 46 is provided to guide the traction wires 50 to each other, the predetermined traction wire 50 is pulled smoothly without interfering with each other, and the distal end portion of the movable elongated structure 10 is bent. be able to.

More specifically, one traction wire 50 and the other traction wire 50 are inserted through the through hole sets 11 that intersect in the circumferential direction and are adjacent to each other in the circumferential direction. Since the guiding protrusion 46 is provided on the cylinder 42, it is possible to regulate one traction wire 50 from overpassing the other fraction wire 50 and causing interference. Therefore, the predetermined traction wire 50 may be smoothly pulled without the plurality of pairs of traction wires 50 interfering with each other, and the distal end portion of the movable elongated structure 10 may be bent.

Also, the traction wire 50 is a flexible wire, and the flexible tubes 20, 30 are composed of flexible tubes having main lumens 21, 31, and the wire lumens 22, 32 are formed inside the wall of tubes. Since there is a wire lumen formed inside the wall through which a wire may be inserted, a movable elongated structure composed of a tube having main lumens 21 and 31 is pulled by pulling a traction wire 50 composed of a flexible wire. At least the distal end portion of the body 10 may be bent, and a predetermined treatment may be performed by inserting an instrument or medicine through the main lumens 21 and 31 of the movable elongated structure 10 that may bend at least the distal end portion.

Since the pair of pulling wires 50 are famed by bending back the wires at the distal end side LF of the distal end side flexible tube 20, the pair of pulling wires 50 may be formed from one wire, thus a movable elongated structure 10 having a simple structure may be constructed.

In addition, even if the duct is at least one of hollow organs, vessels, and blood vessels that have a narrow and complicated branch structure, it may be easily inserted to a predetermined position.

Specifically, a two-way movable bendable neproscope is used clinically. As shown in FIG. 9, the kidney has a three-dimensional shape, and it has been found that there are parts that may not be reached by bidirectional bending. For example, in the three-dimensional shape of the upper small renal cup of the kidney, various inner surfaces may be accessed through a combination of bidirectional bending and axial rotation, but in the middle and lower small renal cups, some inner surfaces are inaccessible. On the other hand, as shown in FIG. 9 at (A), a movable elongated structure 10 that may move in four directions (omnidirectionally) has two vertical directions (two directions on the drawing of FIG. 9 at (A)) and two horizontal directions (It may be bent in all directions by two directions on the drawing of FIG. 9B) and combinations thereof.

In addition, since the plurality of traction wires 50 are routed crosswise using the spacer 40, even if the base end side flexible tube 30 is bent along the path as shown in FIG. 9. Deterioration of the movable bending performance of the tube 20 is small. Therefore, all inner surfaces of the upper, middle and lower calyces are freely accessible.

In this way, since the movable elongated structure 10 is movable In four directions (all directions), it is possible to make the distal end reach a desired position in three-dimensional tubular organs such as kidneys, vessels, and blood vessels.

The movable elongated structure 10 described above is provided with four traction wires 50 (53, 54, 55, 56) and configured to bend the distal end side flexible tube 20 in a desired direction. The configuration is not limited, and the traction wire 50 may be arranged according to the bending direction.

Specifically, as shown In FIG. 10 at (A) and (B), for example, in the case of a movable elongated structure 10a that bends the distal end-side flexible tube 20 in one upward direction HU, the above movable elongated structure Only 10 traction portions 53 are provided, and the base end flange portion 43b is provided with placement recesses 440 in which only one traction portion 53a, 53b may be placed on the right side WR and the left side WL.

Then, the traction portion 53 is inserted into the distal wire lumen 23 (23a, 23b) of the distal end side flexible tube 20, and the traction portion 53 is arranged in the arrangement recess 44 in the upward direction HU of the distal flange 43a, The traction portions 53a and 53b are respectively arranged in the arrangement recesses 44a of the base-end flange 43b.

Also, the traction portion 53 is inserted through the base end wire lumen 35 and the base end wire lumen 36 of the base end side flexible tube 30. The distal wire lumen 22 other than the distal wire lumen 23 in the distal end side flexible tube 20 and the base end wire lumen 32 other than the base end wire lumens 35 and 36 in the base end side flexible tube 30 may be provided. It may be provided or may not be provided.

In the movable elongated structure 10a configured in this manner, the distal wire lumen 23 is closer in the circumferential direction than the base end wire lumens 35 and 36 through which the traction portion 53 is inserted into the wire lumen 36 in the upward direction HU, the traction portion 53 is inserted through the wire lumen 23 on the distal end side HU may be bent in the upward direction HU.

Further, as shown in FIG. 10 at (C) and (D), for example, in the case of a movable elongated structure 10b that bends the distal end side flexible tube 20 in two directions, the upward direction HU and the downward direction HD, the above movable elongated structure A traction portion 53 and a traction portion 54 of the body 10 are provided.

Then, the traction portion 53 is inserted through the distal wire lumen 23 of the distal end side flexible tube 20, and the fraction portion 53 is disposed in the arrangement recess 44 in the upward direction HU of the distal end side flange 43a. The fraction portions 53a and 53b are arranged in the upward direction HU in the recesses 44 arranged in the width direction W of the portion 43b.

Also, the traction portion 53 is inserted through the base end wire lumen 35 a and the base end wire lumen 36b of the base end side flexible tube 30.

Furthermore, the pulling part 54 is inserted through the distal wire lumen 24 of the distal end side flexible tube 20, and the pulling part 54 is arranged in the arrangement recess 44 in the downward direction HD of the base end wire lumen 34a. Traction portions 54a and 54b are arranged downward HD in each recess 44 arranged in the width direction W of the lumen 34b. Then, the traction portion 54 is inserted through the base end wire lumen 35 b and the base end wire lumen 36 a of the base end side flexible tube 30.

In the movable elongated structure 10b configured in this way, as shown in FIG. 10 at (C) and (D), the distal wire lumen 24 is positioned in the circumferential direction from the base end wire lumens 35a and 36b through which the fraction portion 53 is inserted. The traction portion 53 is inserted through the distal end side wire lumen 23 which is narrowly spaced and arranged in the upward direction HU from the base end side wire lumens 35a and 36b, and the base end side wire lumens 35b and 36a through which the traction portion 54 is inserted is extended in the circumferential direction, is narrow, and the traction portion 54 is inserted through the distal end side wire lumen 24 arranged in the downward direction HD from the base end side wire lumens 35b and 36a. As shown in FIG. 10 at (C), the distal end side flexible tube 20 may be bent upward HU, and when the traction portion 53 is pulled toward base end side LB, the distal end side flexible tube 20 may be bent downward is HD.

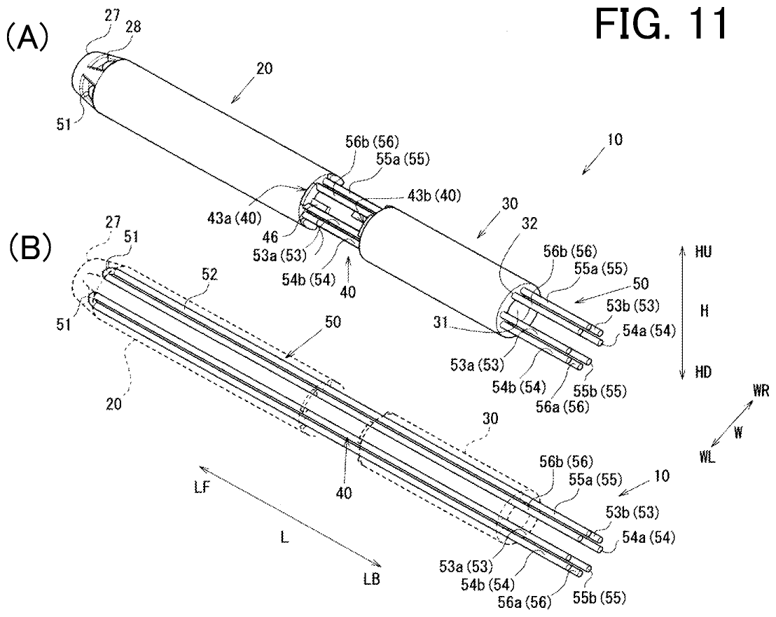

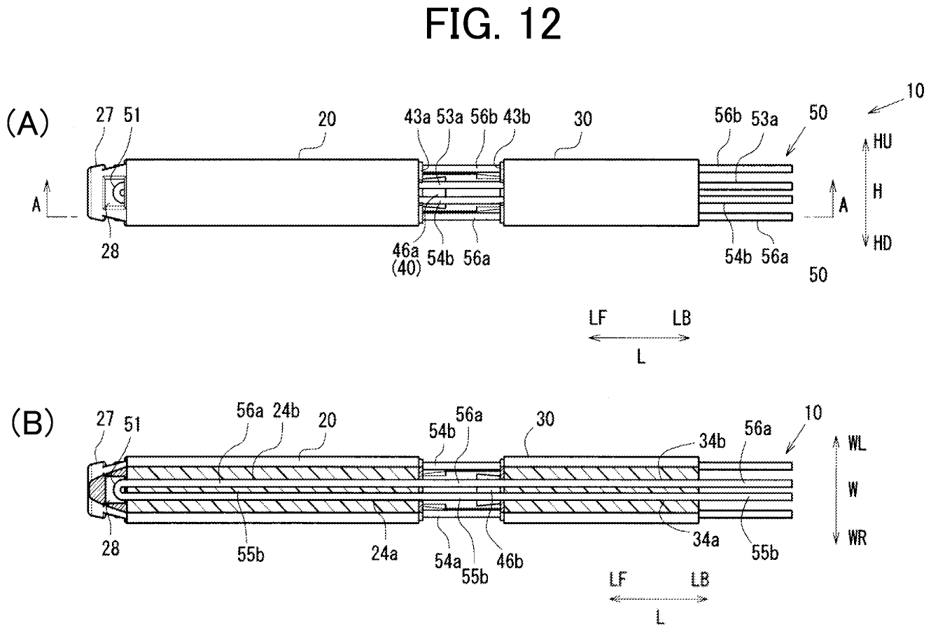

Regarding the movable elongated structure 10 using the spacer 40, in the above description, the traction wire 50 is obliquely extended in the spacer 40 and Inserted through the wire lumens 23, 33. However, as shown in FIGS. 11 and 12, the traction wire 50 may be arranged straight in the spacer 40.

FIG. 11 shows an explanatory view of the movable elongated structure 10 with another arrangement pattern. FIG. 11 at (A) shows a perspective view of the movable elongated structure 10 in another arrangement pattern, and FIG. 11 at (B) shows the movable elongated structure showing the distal end side flexible tube 20 and the base end side flexible tube 30 in a see-through state.

FIG. 12 shows an explanatory view of the movable elongated structure 10 with another arrangement pattern. FIG. 12 at (A) shows a front view of the movable elongated structure 10, and FIG. 12 at (B) shows a cross-sectional view taken along line A-A in FIG. 12 at (A).

The movable elongated structure 10 with an arrangement pattern is a distal end side wire in which the fraction portions 52 (53 to 56) of the traction wire 50, which are bent back near the center of the length to form a bent portion 51, are arranged in four directions. It is inserted along the longitudinal direction L through the lumen 22, the placement recess 44, and the base end wire lumen 32

For example, the traction portion 53a of the traction portion 53 from the distal end side LF is inserted into the distal end side wire lumen 23a of the left side WL in the distal end side wire lumen 23, the traction portion 53b is inserted through the distal end side wire lumen 23b of the right side WR. The traction portion 53 is led out from the base end side LB of the side wire lumen 23, and the bending portion 51 is arranged in the bending concave portion 28 in the upward direction HU. The other traction sections 54, 55, 56 are routed similarly.

The traction portion 53 led out from the base end side LB of the distal end side wire lumen 23 is arranged in the upward direction HU of the distal end side flange 43a and the base end side flange 43b, and the upward direction of the base end side flexible tube 30 It is inserted through the base end side wire lumen 33 of the HU and led out from the base end side LB.

In this way, the movable elongated structure 10 in which the straight traction portion 52 is inserted through the wire lumens 23 and 33 along the longitudinal direction L extends obliquely in the spacer 40 and the traction wire 50 is arranged as described above. Compared to the movable elongated structure 10, the effect of suppressing the bending effect on the base end side flexible tube 30 when the traction wire 50 is pulled is reduced, but the base end side flexible tube 30 is The flexible tube 20 on the distal end side may be bent in a desired direction by pulling all the four sets of traction wires 50, or by pulling two sets of traction wires 50 facing each other in the radial direction, the movable elongated tube 20 may be pulled. The structure 10 may be shortened (compressed) without bending.

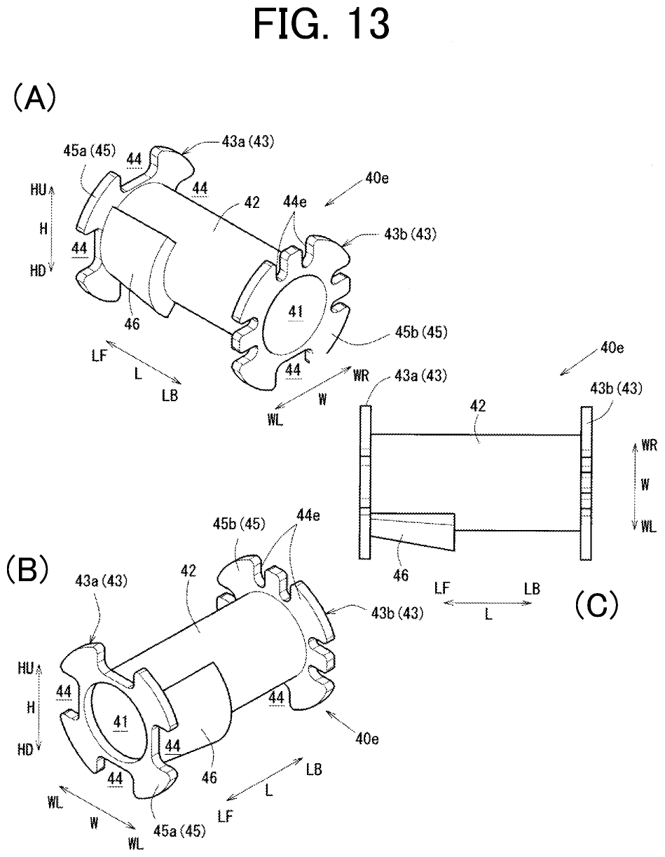

Further, the spacer 40 in the movable elongated structural body 10 described above is provided with the guide convex portion 46 on each of the distal end side LF and the base end side LB, but as shown in FIG. 13, only one side of the longitudinal direction L may be provided with the guiding convex portion 46.

Note that FIG. 13 shows an explanatory diagram of the spacer 40e. FIG. 13 at (A) shows a front, right side, and top perspective view of spacer 40e, FIG. 13 at (B) shows a front, left side, and top perspective view of spacer 40e, and FIG. 13 at (C) shows a top view of spacer 40e.

As shown in FIG. 13, the spacer 40e has a guide convex part 46 only on the distal end side LF. Further, in the spacer 40 described above, the guide convex parts 46 are provided so as to face each other in the circumferential direction.

In addition, among the arrangement recesses 44 of the base end flange 43b in which the guide convex part 46 is not provided, the arrangement recesses 44e arranged in the upward direction HU, the right side WR and the left side WL are arranged with two traction wires 50, respectively, it is formed in a substantially W shape with an open radial outer side when viewed from the longitudinal direction L. That is, the placement recess 44e is provided corresponding to each traction wire 50, and the side walls of the placement recess 44 e function as the regulation parts 45a and 45b for each traction wire 50.

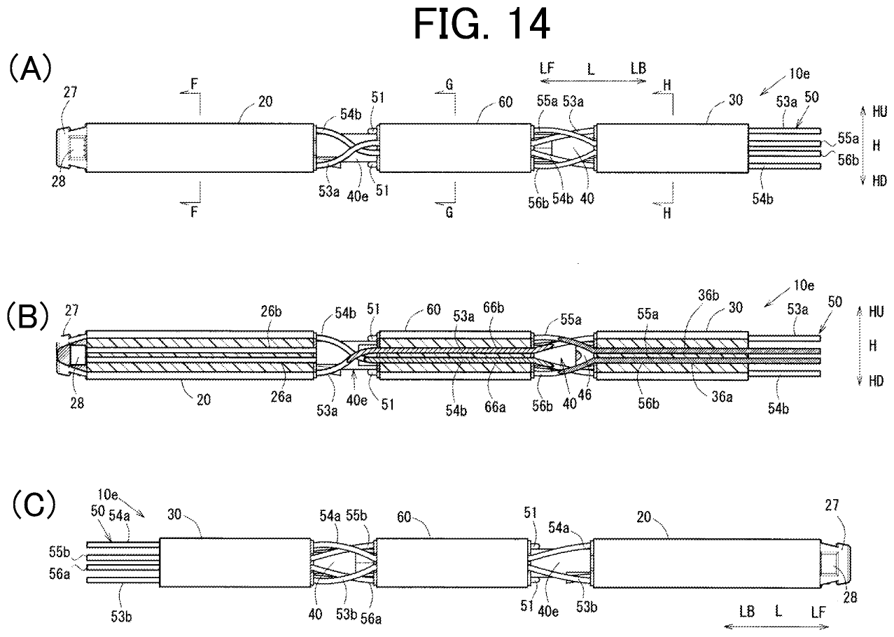

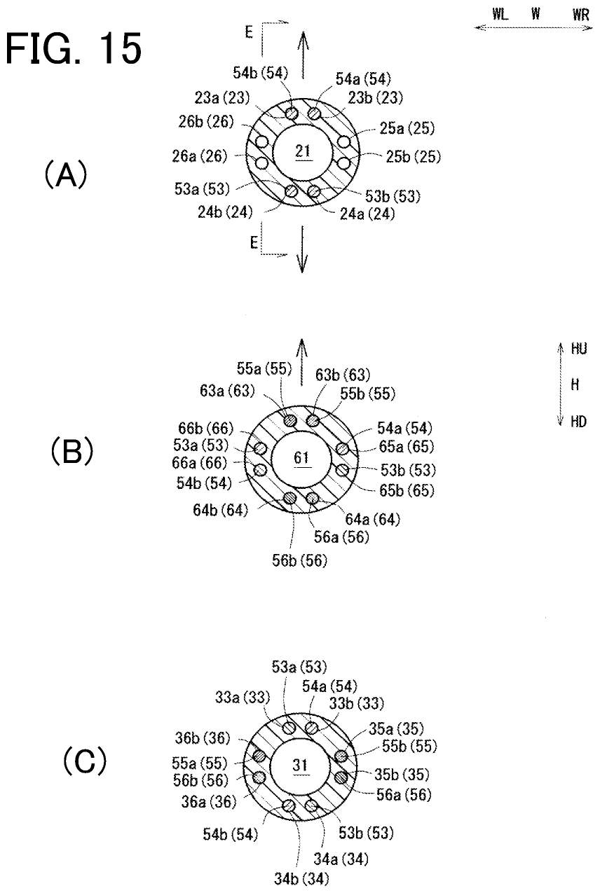

A movable elongated structure 10e using the spacer 40e configured in this way will be described with reference to FIGS. 14 and 15.

FIGS. 14 and 15 are explanatory diagrams of the movable elongated structure 10e. FIG. 14 at (A) shows a front view of the movable elongated structure 10e, FIG. 14 at (B) shows a cross-sectional view of FIG. 15 at (A) along line E-E, and FIG. 14 at (C) shows a rear view of the movable elongated structure 10e, FIG. 15 at (A) shows a cross-sectional view along the F-F arrow in FIG. 14 at (A), FIG. 15 at (B) shows a cross-sectional view along the G-G arrow in FIG. 14 at (A), and FIG. 15 at (C) shows a cross-sectional view along the H-H arrow in FIG. 14 at (A).

The movable elongated structure 10e shown in FIGS. 14 and 15 differs from the movable elongated structure 10 in which the spacer 40 is arranged between the distal end side flexible tube 20 and the base end side flexible tube 30, an intermediate tube 60 is arranged between the distal end side flexible tube 20 and the base end side flexible tube 30, a spacer 40 is arranged between the base end side flexible tube 30 and the intermediate tube 60, and a distal end side flexible tube is arranged. Between the flexible tube 20 and the intermediate tube 60, the spacer 40e described above is arranged.

The intermediate tube 60 is a cylindrical flexible tube elongated in the longitudinal direction 1, similarly to the distal end side flexible tube 20 and the base end side flexible tube 30, and has a main lumen 61 therein. Also, the intermediate tube 60 is provided with two intermediate wire lumens 63, 64, 65, 66 in each of the four directions of the upward direction HU, the downward direction HD, the right side WR and the left side WL inside the tube wall.

The intermediate tube 60 is a flexible tube made of polyamide elastomer, expanded polytetraftuoroethylene, polyurethane, or potytetrafluoroethylene, like the distal end side flexible tube 20 and the base end side flexible tube 30 may be configured. The intermediate tube 60 may be made of the same material as the distal end side flexible tube 20 and the base end side flexible tube 30, or may be made of different materials.

The traction portion 54b that passes through the left WL distal wire lumen 23a of the distal wire lumen 23 in the upward direction HU of the distal end side flexible tube 20 is below the intermediate wire lumen 66 provided on the left WL of the intermediate tube 60. The intermediate wire lumen 66a in the direction HD is inserted, and the base end wire lumen 34b on the left side WL of the base end wire lumen 34 provided in the downward direction HD in the base end side flexible tube 30 is inserted.

The traction portion 54a through which the right WR distal wire lumen 23b of the distal wire lumen 23 in the upward direction HU in the distal end side flexible tube 20 is inserted is above the intermediate wire lumen 65 provided In the right WR of the Intermediate tube 60. The intermediate wire lumen 65a in the direction HU is inserted, and the base end wire lumen 33b on the right side WR of the base end wire lumen 33 provided in the upward direction HU in the base end side flexible tube 30 is inserted.

In addition, the traction portion 53b through which the right. WR distal wire lumen 24a of the downward HD distal wire lumen 24 of the distal end side flexible tube 20 is inserted is below the intermediate wire lumen 65 provided on the right WR of the intermediate tube 60. The intermediate wire lumen 65b in the direction HD is inserted, and the base end wire lumen 34a on the right side WR of the base end wire lumen 34 provided in the downward direction HD in the base end side flexible tube 30 is inserted.

The traction portion 53a through which the left WL distal wire lumen 24b of the downward HD distal wire lumen 24 of the distal end side flexible tube 20 is inserted is located above the intermediate wire lumen 66 provided on the left WL of the intermediate tube 60. The intermediate wire lumen 66b in the direction HU is inserted, and the base end wire lumen 33a on the left side WL of the base end wire lumen 34 provided in the upward direction HU in the base end side flexible tube 30 is inserted.

On the other hand, the traction portions 55 and 56 are not Inserted into the distal wire lumens 25 and 26 in the width direction W of the distal end side flexible tube 20, and the left side of the intermediate wire lumen 63 provided in the upward direction HU in the intermediate tube 60. The traction portion 55a is inserted through the intermediate wire lumen 63a of the WL, and is inserted through the upward HU base end wire lumen 36b of the base end wire lumen 36 provided on the left WL of the base end side flexible tube 30.

Then, the fraction portion 55b is inserted into the intermediate wire lumen 63b of the right WR of the intermediate wire lumen 63 provided in the upward direction HU in the intermediate tube 60, and the base end side wire lumen provided in the right WR of the base end side flexible tube 30 is inserted. 35 is inserted through the base end side wire lumen 35a in the upward direction HU.

In addition, the traction portion 56a is inserted into the intermediate wire lumen 64a of the right WR of the intermediate wire lumen 64 provided in the downward HD of the intermediate tube 60, and the base end side wire lumen provided in the right WR of the base end side flexible tube 30 is inserted. 35 is inserted through the base end side wire lumen 35b in the downward direction HD.

Then, the traction portion 56b is inserted into the Intermediate wire lumen 64b on the left side WL of the intermediate wire lumen 64 provided on the downward HD of the intermediate tube 60, and the base end side wire lumen provided on the left side WL of the base end side flexible tube 30 is inserted. 36 is inserted through the base end side wire lumen 36a in the downward direction HD.

The traction portions 55 and 56 are not inserted through the distal wire lumen 23 of the distal end side flexible tube 20 but are inserted through the intermediate wire lumen 63 of the intermediate tube 60 and the base end wire lumen 33 of the base end side flexible tube 30, a bent portion 51 is formed at the arrangement recess 44e of the flange 43 of the spacer 40e arranged between the distal end side flexible tube 20 and the intermediate tube 60, and is bent back.

In addition, in the spacer 40e arranged between the distal end side flexible tube 20 and the intermediate tube 60 in the movable elongated structure 10e, the traction portion 53a and the traction portion 54b intersect on the front side shown in FIG. 14 at (A), but on the back side shown in FIG. 14 at (C), the traction portion 53b and the traction portion 54a are routed so as not to cross each other.

In the movable elongated structure 10e configured in this manner, the traction portion 53 and the traction portion 54 are inserted through the distal end side flexible tube 20, the intermediate tube 60, and the base end side flexible tube 30, and the traction portion 55 and the traction portion 55 are inserted. Section 56 extends through intermediate tube 60 and base end side flexible tube 30.

Therefore, the spacer 40 positioned between the base end side flexible tube 30 and the intermediate tube 60 has the four traction sections 53, 54, 55, and 56 positioned thereon, while the distal end side flexible tube 20 and the intermediate tube 60 are spaced apart. Two fraction portions 53 and 54 are arranged on the spacer 40e arranged between the tube 60 and the spacer 40e.

Therefore, a spacer 40e is used in which there are few intersections between the traction wires 50 and the guide projections 46 are provided only on the distal end side LF.

As described above, traction portion 53 and traction portion 54 extend through distal end side flexible tube 20, intermediate tube 60 and base end side flexible tube 30, and fraction portion 55 and traction portion 56 extend through intermediate tube 60 and base end tube 60, respectively. The movable elongated structure 10e that is inserted through the flexible tube 30 is based on one of the traction portion 53 and the fraction portion 54 that are inserted through the distal end side flexible tube 20, the intermediate tube 60 and the base end side flexible tube 30. The distal end side flexible tube 20 may be bent upward HU or downward HD with respect to the intermediate tube 60 by puffing toward the end LB.

On the other hand, by pulling either the traction portion 55 or the traction portion 56 inserted through the intermediate tube 60 and the base end side flexible tube 30 to the base end side LB, the intermediate tube is pulled relative to the base end side flexible tube 30, the intermediate tube 60 may be bent either upward HU or downward HD. At this time, if the traction portion 53 and the traction portion 54 are not pulled, the distal end side flexible tube 20 does not bend with respect to the intermediate tube 60 and is pulled upward HU or downward with respect to the base end side flexible tube 30. It bends following the intermediate tube 60 that bends to HD. That is, the movable elongated structure 10e may be bent with the distal end side flexible tube 20 and the intermediate tube 60 substantially not interfering with each other.

As described above, an intermediate tube 60 having an elongated intermediate wire lumen 63, 64, 65, and 66 is disposed between the distal end side flexible tube 20 and the base end side flexible tube 30, and a plurality of a pair of traction wires 50 are provided, and at least one pair of traction wires 50 out of the plurality of pairs of traction wires 50 are inserted through the distal wire lumen 22, the intermediate wire lumens 63, 64, 65, and 66 and the base end wire lumen 32. At least one pair of traction wires 50 out of the plurality of pairs of traction wires 50 is inserted through the intermediate wire lumens 63, 64, 65, and 66 and the base end wire lumen 32. Therefore, it is possible to configure the movable elongated structure 10e that may bend the intermediate tube 60 and the distal end side flexible tube 20, respectively.

Specifically, among the plurality of pairs of traction wires 50, the traction wires 55 and 56 are inserted through the intermediate wire lumens 63 and 64 and the base end wire lumens 35 and 36, and the other traction wire 53 of the plurality of pairs of traction wires 50 is inserted, traction wires 53 and 54 are inserted through the distal wire lumens 23 and 24, the intermediate wire lumens 65 and 66, and the base end wire lumens 33 and 34, the intermediate wire lumens 63, 64 and the base end wire lumens 35, 36 are by pulling the inserted traction wires 55 and 56, the intermediate tube 60 in the intermediate portion of the movable elongated structure 10e may be bent, and the distal end side wire lumens 23 and 24, the intermediate wire lumens 65 and 66, and the base end side wire lumens 23 and 24 may be bent. By pulling the fraction wires 53, 54 inserted through the wire lumens 33, 34, the distal end side flexible tube 20, which is the distal end portion of the movable elongated structure toe, may be bent.

In this way, with the above-described configuration, the intermediate tube 60 at the intermediate portion of the movable elongated structure 10e and the distal end side flexible tube 20, which is the distal end portion of the movable elongated structure 10e, substantially interfere with each other. may be bent without

The guide projections 46 provided on the spacers 40 and 40e described above are formed in a trapezoidal vertical cross section that gradually protrudes from the ends in the longitudinal direction L toward the center. You may form the guidance convex part 46g in a shape.

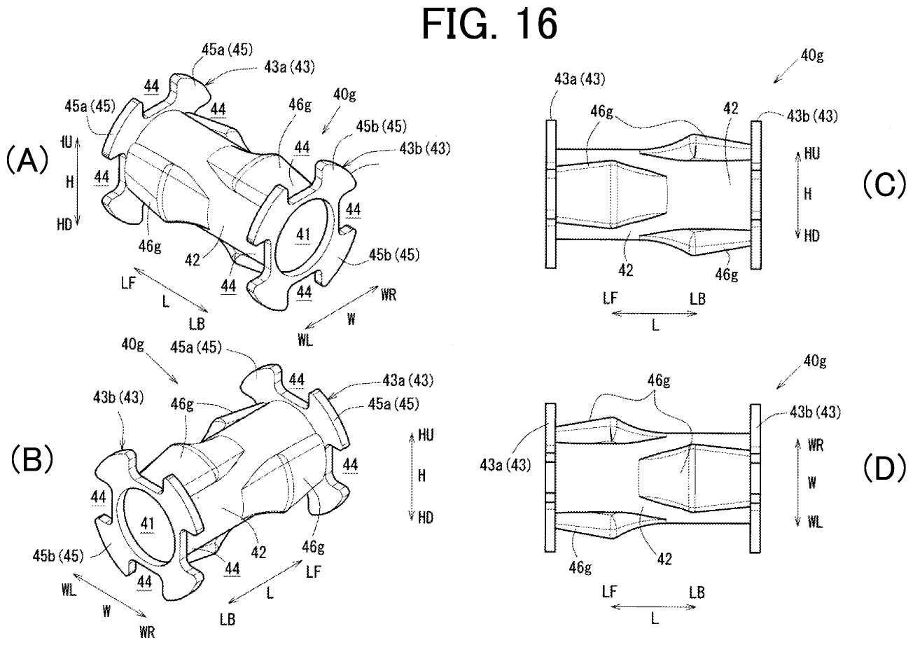

FIG. 16 shows an explanatory diagram of a spacer 40g having a guide convex part 46g. FIG. 16 at (A) shows a front, right side, and top perspective view of spacer 40g, FIG. 16 at (B) shows a rear, right side, and top perspective view of spacer 40g, and FIG. 16 at (C) shows a front view of spacer 40g. FIG. 16 at (D) shows a plan view of the spacer 40g.

The guiding projection 46g provided on the spacer 40g has a height that gradually increases from the end in the longitudinal direction L toward the center in the longitudinal direction L, and the width thereof also gradually widens. Then, it is formed in a substantially hexagonal shape in a front view in which the height gradually decreases from the widest point and the width gradually narrows.

The guide protrusion 46g formed in the shape described above has a gentler corner than the guide protrusion 46 provided on the spacer 40 or the spacer 40e, and stress concentration on the pulling wire 50 straddling the guiding projection 46 can be reduced.

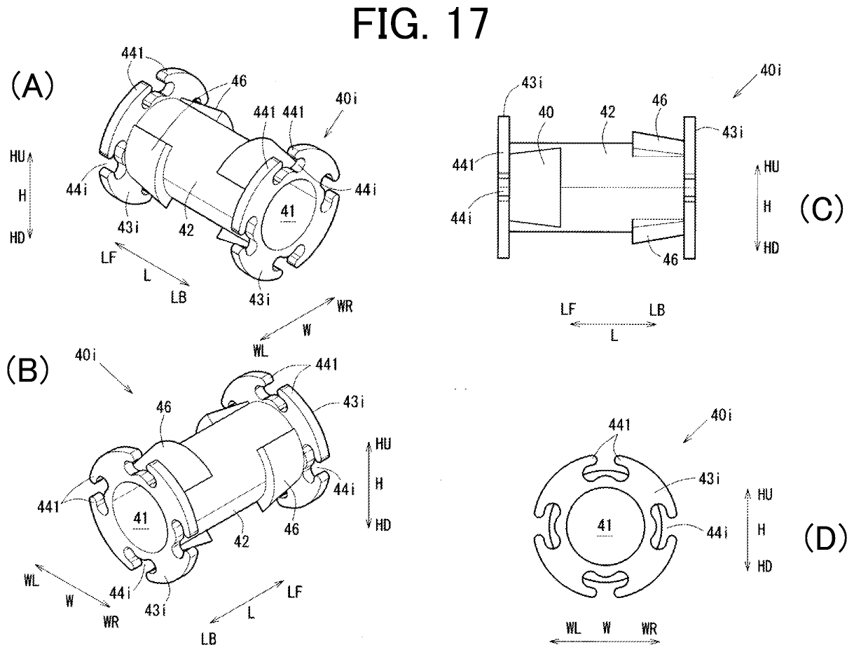

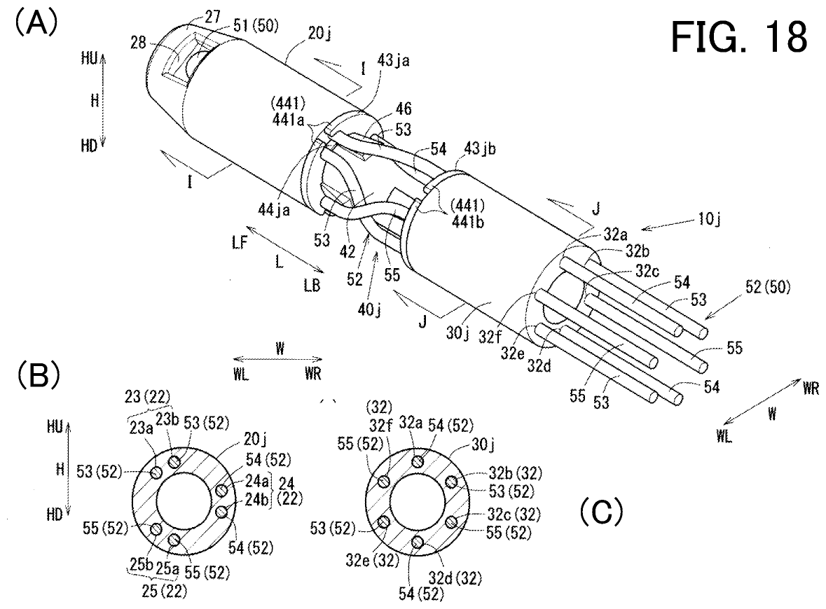

Further, the above-described spacer 40 is provided with a recessed placement recess 44 whose radial outer side is open in the flange portion 43. However, a spacer 40i having a placement recess 44i having a compression support portion 441 as shown in FIG. 17.

FIG. 17 shows an illustration of a spacer 40i having compression support portions 441 in the placement recesses 44i. FIG. 17 at (A) shows a front, right side and top perspective view of spacer 40i, FIG. 17 at (B) shows a back, right side and top perspective view of spacer 40i, FIG. 17 at (G) shows a front view of spacer 40i, and FIG. 17 at (D) indicates the side surface of the spacer 40i.

The flange 43i of the spacer 40i is provided with arrangement recesses 44i in four directions at equal intervals in the circumferential direction, and a compression support portion 441 is arranged in each arrangement recess 44i.

The arrangement recess 44i has circular arc recesses in which the traction wire 50 is arranged, which are arranged at predetermined intervals in the circumferential direction.

The compression support portions 441 are formed to protrude in the circumferential direction from both sides of the arrangement recess 44i on the radially outer side of the approximately bean-shaped arrangement recess 44i as described above, with a gap that allows the traction wire 50 to pass through in the radial direction.

The moveable elongated structure 10 in which the spacer 40i having the arrangement recess 44i configured in this manner is arranged between the distal end side flexible tube 20 and the base end side flexible tube 30 is configured such that the spacer 40 is arranged as described above. The same effect as the arranged movable type elongated structure 10 may be obtained, and the following effect may be obtained.

When the traction wire 50 is pulled to bend the flexible tubes 20,30, a compressive force is applied to the flexible tubes 20, 30. The compressive force also acts on the spacer 40i arranged between the flexible tubes 20, 30.

If the radially outer open portion of the arrangement recess 44i in the spacer 40i becomes large, the flexible tubes 20 and 30 to which the compressive force is applied may bite into the radially outer open portion of the arrangement recess 44i, regulating desired bending deformation. On the other hand, since the spacer 4i is provided with the compression support portion 441 that protrudes radially outward from the arrangement recess 44i, the compression force described above may be supported, and the desired bending deformation may be achieved.

In addition, the spacer 40i functions as a pull-out regulation part in which the compression support portion 441 regulates the traction wire 50 from slipping out of the placement recess 44i. It is possible to regulate unintentional detachment by moving radially outward from the.

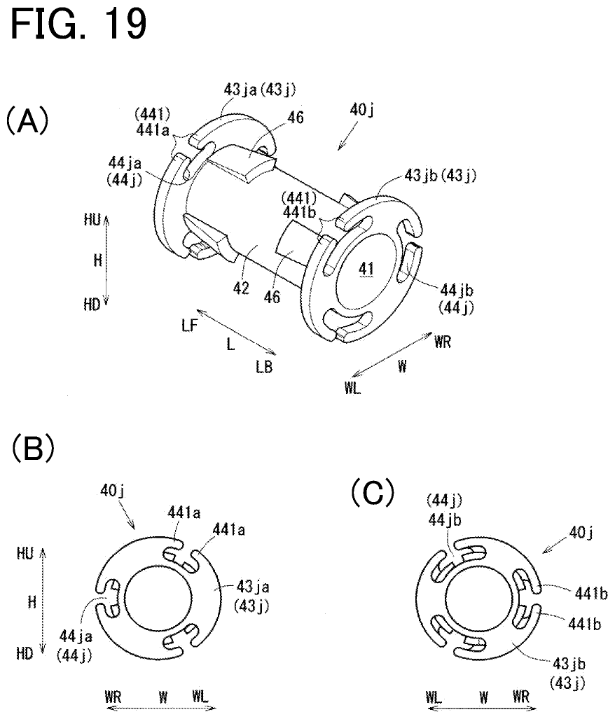

In the spacer 40i having the arrangement recesses 44i with the compression support portions 441, although not shown, the arrangement recesses 44i may be provided at three locations in the circumferential direction of the flange portion 43i. Specifically, as shown in FIGS. 18 and 19, a movable elongated structure 10j using a spacer 40j having three arrangement recesses 44j provided on a flange 43j will be described with reference to FIGS. 18 and 19.

FIGS. 18 and 19 are explanatory diagrams of the movable elongated structure 10j and the spacer 40j. FIG. 18 at (A) shows a perspective view of the movable elongated structure 10j, FIG. 18 at (B) shows a cross-sectional view taken along line I-I in FIG. 18 at (A), FIG. 18 at (C) shows a cross-sectional view taken along arrow J-J in FIG. 18 at (A), and FIG. 19 at (A). shows a perspective view of the spacer 40j, FIG. 19 at (B) shows a side view of the spacer 40j viewed from the distal end side LF, and FIG. 19 at (C) shows a side view of the spacer 40j viewed from the base end side LB.

Similar to the movable elongated structure 10 described above, the movable elongated structure 10j shown in FIGS. 18 and 19, the spacer 40j and the base end side flexible tube 30j are arranged, and the traction wire 50 is routed. In addition, the same reference numerals are given to the same configurations as those of the above-described movable elongated structure 10 and the like, and the description thereof will be omitted.

The spacer 40j used in the movable elongated structure 10j has three bean shaped placement recesses 44j similar to the placement recesses 44i of the spacer 40i shown in FIG. 17. A support portion 441 is provided to ether ach of placement recesses 44i. Therefore, three traction wires 50 (53, 54, 55) are routed in the movable elongated structure 10j.

However, in the arrangement recess 44i described above, the protrusion 43i on the distal end side LF and the protrusion 43i on the base end side LB are formed in the same shape, but the placement recess 44j has the distal end side flange on the distal end side LF. The placement recess 44ja and the compression support portion 441a provided in the portion 43ja are different in shape from the placement recess 44jb and the compression support portion 441b provided in the base end side flange portion 43jb of the base end side LB.

More specifically, although the circumferential position of the arrangement recess 44ja in the distal end side flange 43ja coincides with the circumferential position of the arrangement recess 44jb in the base end side flange 43jb, the base end side flange 43jb is located at the same position. The arrangement recess 44jb provided is formed to be longer in the circumferential direction than the arrangement recess 44ja provided in the distal end side flange 43ja. Therefore, the compression support portion 441b is also formed to protrude longer than the compression support portion 441a.

Specifically, while the arrangement recess 44ja provided in the distal end side flange 43ja has the length of the two traction wires 50 arranged at an appropriate interval, the arrangement recess 44jb provided in the base end side flange 43jb is formed such that the distance between the traction wires 50 arranged on both sides of the arrangement recess 44jb is famed to have the same length in the circumferential direction as the distance between the traction wires 50 arranged in the adjacent arrangement recess 440. Therefore, the traction portions 52 of the traction wires 50 arranged in the arrangement recesses 44jb arranged in three directions in the circumferential direction are arranged at intervals of 60 degrees in the cross-sectional circumferential direction.

The distal end side wire lumen 22 of the distal end side flexible tube 20j and the base end side wire lumen 32 of the base end side flexible tube 30j that constitute the movable elongated structure 10j using the spacer 40j configured in this way are different arrangement.

Specially, as shown in FIG. 18 at (B), two distal wire lumens 22 (23, 24, 25) provided in the distal end side flexible tube 20j are arranged in three directions in the circumferential direction of the cross section.

Among the distal end side wire lumens 22 arranged in three directions, the upward direction HU is the distal end side wire lumen 23, the counterclockwise side of the two distal end side wire lumens 23 is the distal end side wire lumen 23a, and the clockwise side is the distal end side wire lumen 23b.

Similarly, the distal wire lumen 22 arranged on the right WR is referred to as a distal wire lumen 24, the counterclockwise side of the two distal wire lumens 24 is referred to as a distal wire lumen 24a, and the clockwise side is referred to as a distal wire lumen 24a. The distal end side wire lumen 22 arranged on the left side WL and downward HD is the distal end side wire lumen 25, and the counterclockwise side of the two distal end side wire lumens 25 is the distal end side wire lumen 25a. side is the distal end side wire lumen 25b.

On the other hand, the base end side wire lumens 32 provided in the base end side flexible tube 30j are arranged in six directions at equal intervals in the circumferential direction of the cross section, as shown in FIG. 18 at (C). Therefore, among the six base end side wire lumens 32, the base end side wire lumen 32 in the upward direction HU is designated as the base end side wire lumen 32a, and clockwise from the base end side wire lumen 32b to the base end side wire lumen 32f in order.

The movable elongated structure 10) using the spacer 40j constructed in this manner is assembled as follows:

A traction wire 50 having a bent portion 51 formed by bending from a bending recess 28 of a distal end cap 27 provided at the tip of the distal end side flexible tube 20j is attached, and a traction portion 521s inserted through the distal end side wire lumen 22.

Then, the traction portion 52 led out to the base end side LB from the distal end side wire lumen 22 is arranged in the placement recess 44ja of the distal end side flange 43ja of the spacer 40j, and is extended obliquely to extend to the base end side flange 43jb is inserted into the base end side wire lumen 32 of the base end side flexible tube 30j, and led out from the base end side LB.

Specifically, the traction portion 53 is inserted from the distal end side LF into the distal end side wire lumen 23 (23a, 23b) of the distal end side flexible tube 20), is led out from the base end side LB, and is pulled out from the distal end side flange 43ja. It is arranged in the arrangement recess 44ja in the upward direction HU.

Then, along the cylinder 42, the traction portion 53 is extended obliquely toward the base end side LB and placed in the placement recess 440 of the base end side flange 43jb, and is placed in the base end side wire lumen of the base end side flexible tube 30j. It is inserted through 32e and 32b from the distal end side LF and led out from the base end side LB.

In addition, the traction portion 54 is inserted from the distal end side LF into the distal end side wire lumen 24 (24a. 24b) of the distal end side flexible tube 20j and is led out from the base end side LB, and the right side WR of the distal end side flange 43ja. It is placed in the placement recess 44ja.

Then, along the cylinder 42, the traction portion 541s extended obliquely toward the base end side LB and placed in the placement recess 44jb of the base end side flange 43jb, and is placed in the base end side wire lumen of the base end side flexible tube 30j. It is inserted through 32a and 32d from the distal end side LF and led out from the base end side LB.

Similarly, the traction portion 55 is inserted through the distal wire lumen 25 (25a, 25b) of the distal end side flexible tube 20j from the distal end side IF, is led out from the base end side LB, and is pulled out from the distal end side LB, and the left side WL of the distal end side flange 43ja. In addition, it is arranged in the arrangement concave portion 44ja in the downward direction HD.

Then, along the cylinder 42, the traction portion 55 is extended obliquely toward the base end side LB and placed in the placement recess 44jb of the base end side flange 43jb, and is placed In the base end side wire lumen of the base end side flexible tube 30j. It is inserted through 32 may 32f from the distal end side LF and led out from the base end side LB.

That is, in the base end side flexible tube 30j, the respective traction portions 52 are arranged in the base end side wire lumens 32 at opposing positions. The movable elongated structure 10j configured in this way has the same effects as the movable elongated structure 10 described above, and pulls all of the three traction wires 50 (53, 54, 55). Thus, the movable elongated structure 10j may be shortened (compressed) without being bent.

Subsequently, a movable elongated structure 10k and spacers 40k of further different embodiments will be described below.

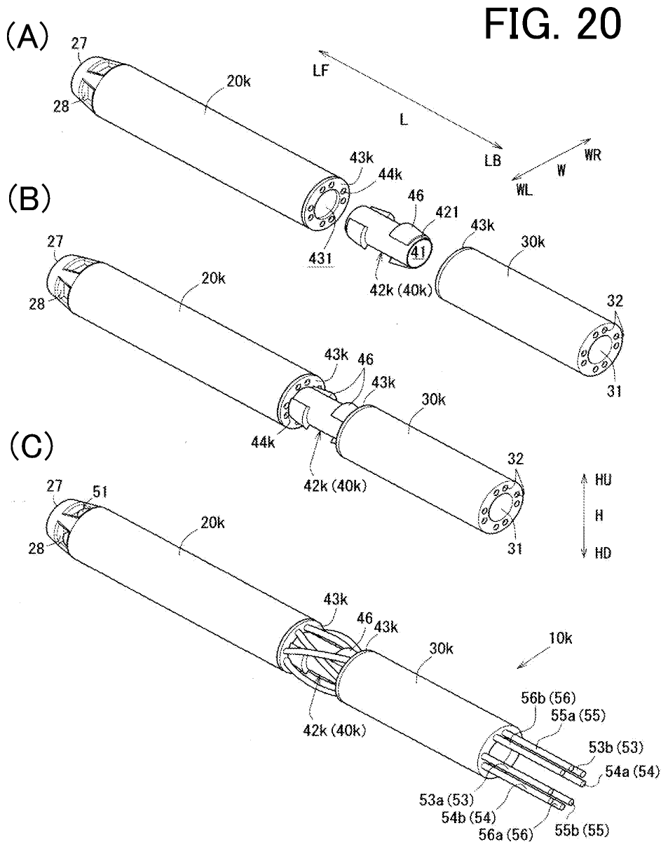

In the spacer 40 described above, the flange portions 43 are provided on both sides in the longitudinal direction L of the cylinder 42 having the internal space 41, and the assembly spacer 40k may be configured only by the cylinder 42k as shown in FIGS. 20 to 22.

FIG. 20 shows an explanatory view of the movable elongated structure 10k. FIG. 20 at (A) shows a perspective view of the movable elongated structure 10k before assembly, FIG. 20 at (B) shows a perspective view of the movable elongated structure 10k in an assembled state, and FIG. 20 at (C) shows a perspective view of a moveable elongated structure 10k.

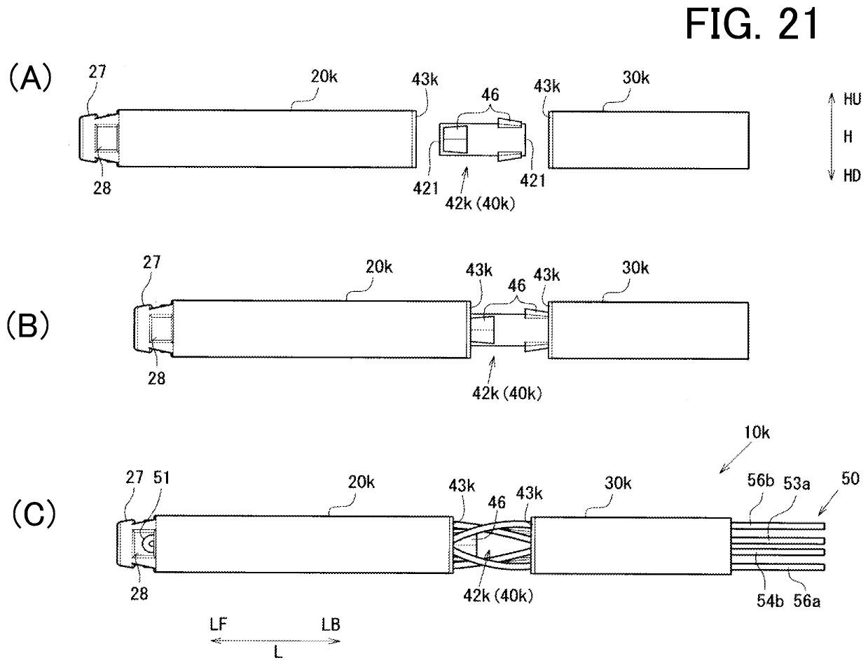

FIG. 21 shows an explanatory view of the movable elongated structure 10k. FIG. 21 at (A) shows a front view of the movable elongated structure 10k before assembly. FIG. 21 at (B) shows a front view of the movable elongated structure 10k in an assembled state, and FIG. 21 at (C) shows a front view of the movable elongated structure 10k with the traction wire 50 routed.

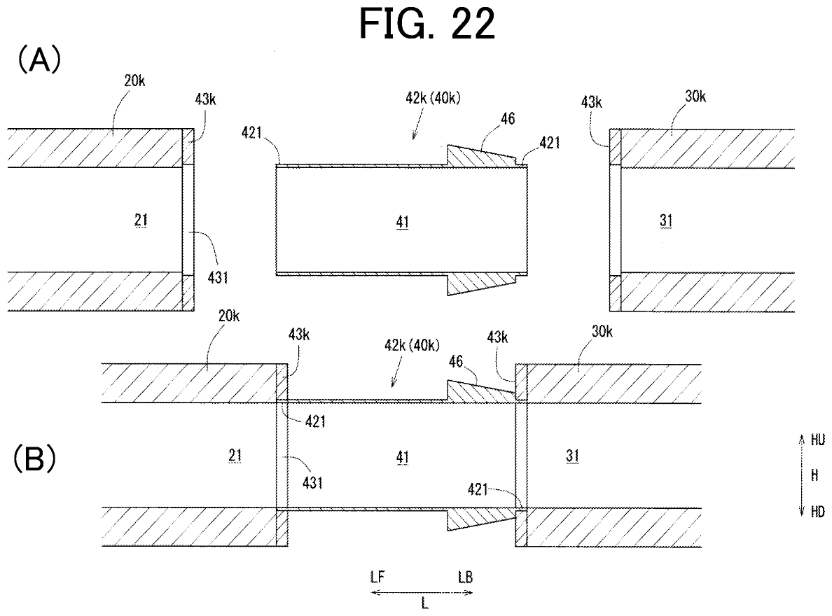

FIG. 22 shows an explanatory view of the assembled spacer 40k. FIG. 22 at (A) shows an enlarged-sectional view of a portion including the assembly spacer 40k before assembly, and FIG. 22 at (B) shows an enlarged cross-sectional view of a portion including the assembly spacer 40k in an assembled state.

In the following description of the movable elongated structure 10k and the assembly spacer 40k, the same components as in the description of the movable elongated structure 10 and the spacer 40 are denoted by the same reference numerals, and the explanation thereof is omitted.

The assembly spacer 40k is composed of a cylindrical cylinder 42k having a guide convex part 46 at the same position as the guide convex part 46 provided on the cylinder 42 of the spacer 40.

Mounting portions 421 for mounting flanges 43k3, which will be described later, are provided on both end side sides in the longitudinal direction L of the guide projection portion 46 of the cylinder 42k.

When using the cylinder 42k configured in this manner, the distal end side flexible tube 20k and the base end side flexible tube 30k having the flange portions 43k at the opposing ends are used.

Specifically, the flexible tubes 20k and 30k are cylindrical flexible tubes having main lumens 21 and 31 inside and elongated in the longitudinal direction L. It has an edge 43k.

As shown in FIG. 20 at (A), the flange portion 43k is a disc made of hard resin having a main through hole 431 provided in the main lumens 21 and 31 at its center, and corresponds to the wire lumens 22 and 32 and is composed of through holes. and a wire placement hole 44k.

The main through hole 431 is formed with a diameter that allows the attachment portion 421 of the cylinder 42k to be fitted therein.

The assembly spacer 40k composed of the distal end side flexible tube 20k, the base end side flexible tube 30k and the cylinder 42k thus configured is assembled to form the movable elongated structure 10k.

Specifically, as shown in FIGS. 20 at (A), 21 at (A), and 22 at (A), from the distal end side LF In the longitudinal direction L to the base end side LB, the distal end side flexible tube 20k, the assembly spacer 40k, and the base end side flexible. The tubes 30k are arranged in this order. At this time, the distal end side flexible tube 20k is arranged so that the flange 43k is on the base end side LB, and the base end side flexible tube 30k is arranged so that the flange 43k is on the distal end side LF.

Also, the assembly spacer 40k is arranged so that the guide convex part 46 is oriented in a desired direction in the circumferential direction.

Then, as shown in FIGS. 20 at (8), 21 at (B), and 22 at (B), the mounting portion 421 of the cylinder 42k constituting the assembly spacer 40k is inserted into the main through hole 431 of the flange 43k and fitted to the distal end side flexible portion to assemble tube 20k, assembly spacer 40k and base end side flexible tube 30k.

As shown in FIG. 20 at (C) and FIG. 21 at (C), the movable elongated structure 10k is configured by inserting the traction wire 50 through the wire lumens 22, 32 of the assembled distal end side flexible tube 20k and base end side flexible tube 30k.

At this time, the traction wire 50 is passed through the wire arrangement hoses 44k provided in the flange portions 43k provided at the ends of the distal end side flexible tube 20k and the base end side flexible tube 30k.

The traction wire 50 may be routed in the assembly spacer 40k so as to extend in an oblique direction in the same manner as in the movable elongated structure 10 described above, or as shown in FIGS. 11 and 12, it may route straight along the longitudinal direction L.

The assembly spacer 40k and the movable elongated structure 10k using the assembly spacer 40k configured in this manner are provided with flanges provided at the ends of the distal end side flexible tube 20k and the base end side flexible tube 30k. Since it is composed of the cylinder 42k assembled with the portion 43k, the same effect as that of the movable elongated structural body 10 provided with the spacer 40 integrated above and the spacer 40 may be obtained.

In addition, compared to the movable elongated structure 10 in which the distal end side flexible tube 20, the spacer 40 and the base end side flexible tube 30 are arranged along the longitudinal direction 1, and the traction wire 50 is routed and assembled, the structure is more flexible. By fitting the mounting portion 421 of the cylinder 42k into the main through hole 431 of the flange portion 43k provided at the ends of the tubes 20k. 30k, the distal end side flexible tube 20k, the assembly spacer 40k, and the base end side flexible tube are assembled. 30k may be assembled, and the assemblability is improved as compared with the movable elongated structure 10 described above.

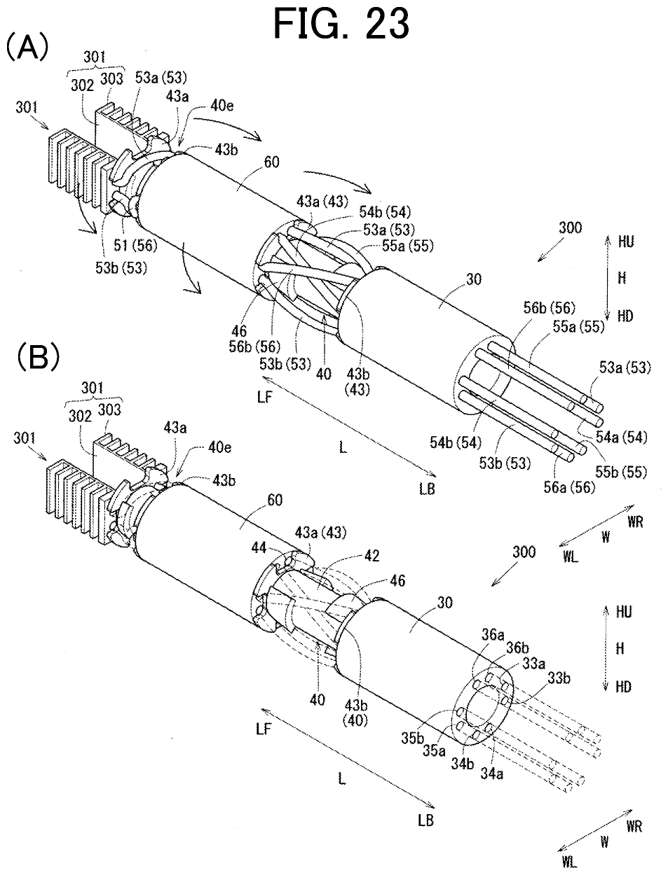

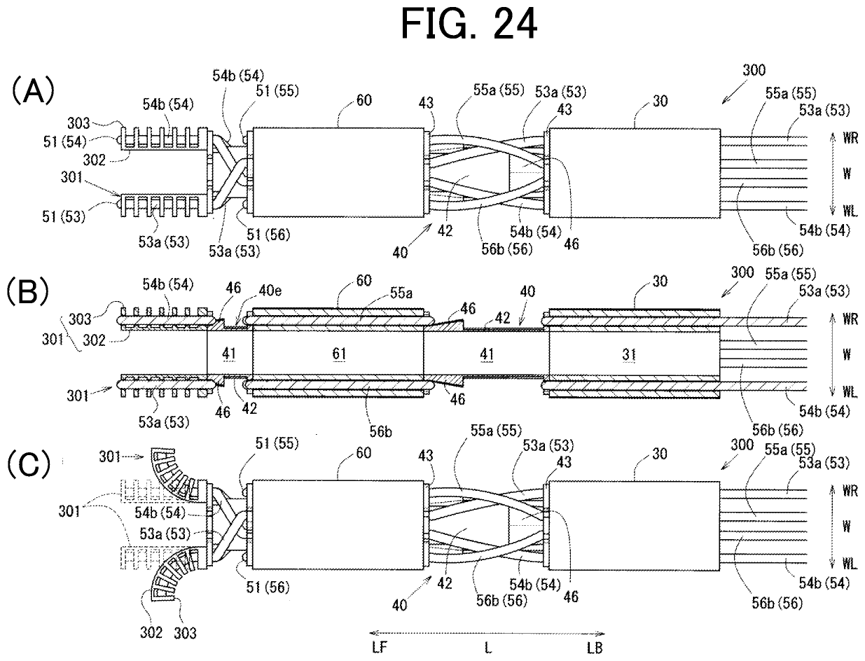

Also, as shown in FIGS. 23 and 24, the retractor 300 may be configured using the movable elongated structure 10e.

FIG. 23 shows a schematic illustration of the retractor 300. As shown in FIG. 23 at (A) shows a perspective view of retractor 300, and FIG. 23 at (B) shows a perspective view of retractor 300 with fraction wire 50 shown in transparent.

FIG. 24 shows a schematic illustration of the retractor 300. As shown in FIG. 24 at (A) shows a plan view of retractor 300, FIG. 24 at (B) shows a cross-section through the upper wire lumen, and FIG. 24 at (C) shows a plan view of retractor 300 with elastic retractor 301 in the open state.

The retractor 300 utilizes the movable elongated structure 10e described above, and has an elastic retractor 301 on the distal end side LF of the spacer 40e instead of the distal end side flexible tube 20 in the movable elongated structure 10e. Specifically, the movable elongated structure 10e shown in FIGS. 14 and 15 is used with the width direction W being the height direction H.

The length of the spacer 40e in the retractor 300 in the longitudinal direction L of the cylinder 42 is shorter than that of the spacer 40e on the front end side LF in the movable elongated structure 10e, but other configurations are the same.

Also, since the wiring of the traction wire 50 is the same as that of the movable elongated structure 10e, the description thereof is omitted.

Two of the elastic retractors 301 are arranged facing each other in the width direction W and are fixed to the front end side LF of the spacer 40e.

The elastic retractor 301 is composed of an elastic body 302 extending toward the distal end side LF and a plurality of plate portions 303 projecting outward In the width direction W from the elastic body 302.

The elastic body 302 is a rectangular plate having a predetermined thickness that is longer in the longitudinal direction L than in the height direction H, and the plate portion 303 is a rectangular plate having a predetermined thickness that is longer in the height direction H than in the width direction W.

A plurality of plate portions 303 are arranged at predetermined intervals in the longitudinal direction L, integrally configured with the elastic body 302, and are longer in the height direction H than in the width direction W and longer in the longitudinal direction L than in the height direction H. It is formed in a rectangular parallelepiped shape.

In a plurality of plate portions 303 arranged at predetermined intervals in the longitudinal direction L, an insertion hole 304 is formed through which the pulling wire 50 (54, 53) is inserted, corresponding to the distal wire lumen 22 (23, 24) of the distal flexible tube 20 in the movable elongated structure 10e.

The elastic retractor 301 configured in this way faces the front end side LF of the spacer 40e at a predetermined interval in the width direction W, that is, the direction in which the plate portion 303 protrudes from the elastic body 302 is in the width direction W. They are arranged facing outwards.

The fraction portion 53 is inserted through the insertion hole 304 of the elastic retractor 301 on the left side WL, and the traction portion 54 is inserted through the insertion hole 304 of the elastic retractor 301 on the right side WR.

The retractor 300 configured in this manner pulls the traction portions 55 and 56 arranged in the height direction H on the base end side LB to the base end side LB, thereby pulling the intermediate tube as indicated by the arrow in FIG. 23 at (A), the intermediate tube 60 may be bent in the width direction W.

Specifically, the intermediate tube 60 may be bent to the left side WL by pulling the traction portion 56 inserted through the base end side wire lumens 34b, 36a of the left side WL in the end side wire lumens 34, 36 to the proximal side LB.

Conversely, the intermediate tube 60 may be bent to the right side WR by pulling to the base end side LB the traction portion 55 inserted through the base end wire lumens 34a, 36b of the right side WR in the base end wire lumens 34, 36.

Then, by canceling the traction of the traction portions 55 and 56, the elastic force of the intermediate tube 60 cancels the bending of the traction portions 55 and 56 due to the traction.

The elastic retractor 301 may be opened by pulling the traction portions 53 and 54 inserted through the base end side wire lumens 33 and 35 in the width direction W on the base end side LB.

Specifically, by pulling the traction portion 53 inserted through the Insertion hole 304 of the elastic retractor 301 of the left WL, the elastic retractor of the left WL may be pulled as indicated by the arrow in FIG. 23 at (A) without bending the intermediate tube 60, 301 may be bent outward in the width direction W.

Conversely, by pulling the traction portion 54 inserted through the insertion hole 304 of the elastic retractor 301 of the right WR, the elastic retractor 301 of the right WR may be pulled as indicated by the arrow in FIG. 23 at (A) without bending the intermediate tube 60. It may be bent outward in the width direction W.

Therefore, when the traction portion 53 and the traction portion 54 are pulled at the same time, the elastic retractors 301 on both sides in the width direction W are bent outward in the width direction W as shown in FIG. 24 at (C). It may be served as a retractor to open the site.

By canceling the traction of the traction portion 53 and/or the traction portion 54, the bending due to the traction of the traction portion 53 and/or the traction portion 54 is canceled by the elastic force of the elastic retractor 341 (302).

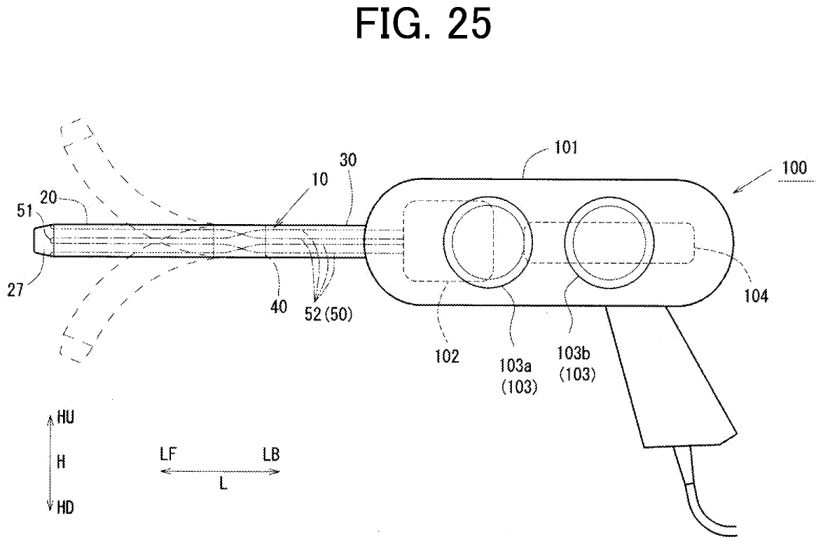

As another example of this embodiment shown in FIG. 25, a manipulator 100, which is a medical device using the movable elongated structure 10 described above, will be described below. Note that FIG. 25 shows a schematic diagram of a manipulator 100, which is a medical device in the other example.

The manipulator 100 is a medical device that is inserted into a duct such as a blood vessel that has a branched path, and performs a predetermined treatment after the tip reaches a predetermined location. A movable elongated structure 10 extending from the tip of the manipulator main body 101 to the distal end side LF is provided.

The manipulator main body 101 includes a traction drive unit 102 for pulling a traction portion 52 (not shown) of a traction wire 50 (not shown) extending from the base end side LB of the movable elongated structure 10, and a traction drive unit 102. The operation handle 103 (103a, 103b) for manually operating the bending direction of the movable elongated structure 10 by pulling the traction portion 52, and how the eight traction portions 52 are pulled by the manual operation of the operating handle 103. A control unit 104 is provided to control whether the driving unit 102 is towed.

The traction driving section 102 and the control unit 104 are arranged inside the manipulator main body 101, and the operating handle 103 is arranged outside the manipulator main body 101.

The operating handle 103 includes a vertical operating handle 103a for bending the movable elongated structure 10 in the height direction H, and a width direction operating handle 103b for bending it in the width direction.

The operator may bend the distal end side LF of the movable elongated structure 10 in the upward direction HU or the downward direction HD in the height direction H by manually operating the vertical direction operation handle 103a. It may be bent by a desired bending amount by the operation amount of 103a.

In addition, the operator may bend the movable elongated structure 10 to the right or left in the width direction by manually operating the width direction operation handle 103b, and the desired bending may be achieved by the operation amount of the width direction operation handle 103b. It may be bent in quantity.

Then, the operator manually operates both the vertical operation handle 103a and the width direction operation handle 103b at the same time or sequentially to move the distal end side LF of the movable elongated structure 10 in the height direction H and width direction. It may be bent in an oblique direction intersecting the direction, and may be bent in a desired bending direction by the amount of operation of the vertical direction operating handle 103a and the width direction operating handle 103b.

In the description of the manipulator 100 described above, the movable elongated structure 10 is used. may be used. In this case, it is preferable to provide a traction drive unit 102 corresponding to the bending direction, that is, the number of traction wires 50 (traction portions 52), and an operation handle 103 corresponding to the bending direction.

Further, if necessary, the operation handles 103a and 103b may be provided in separate units and connected to the manipulator main body 101 by wire or wirelessly.

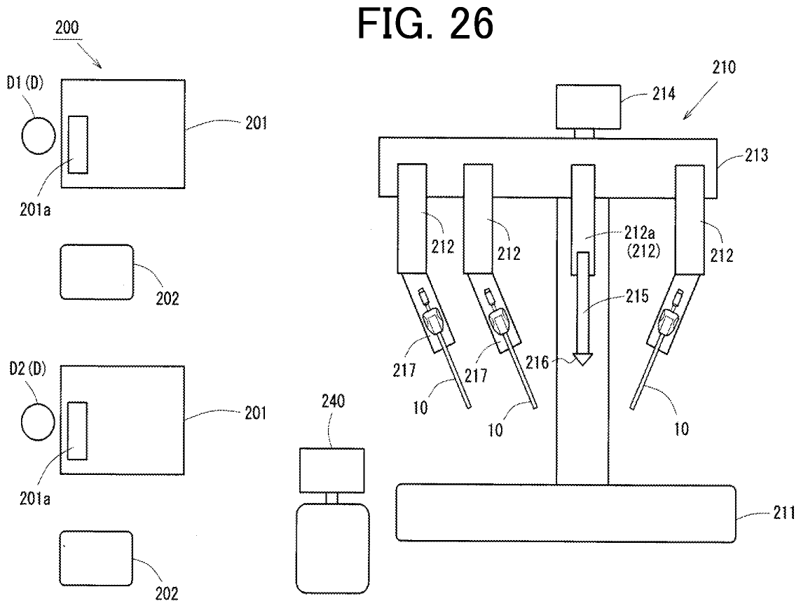

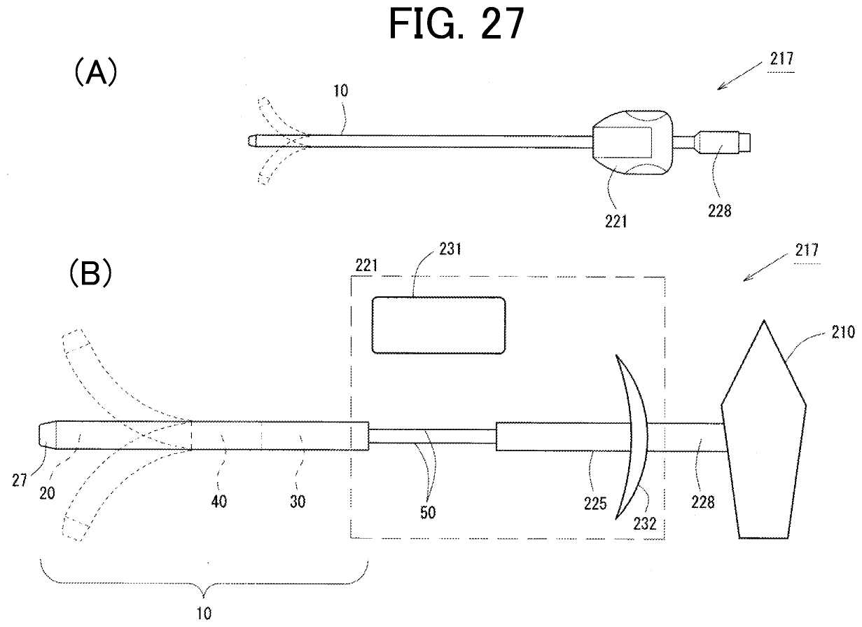

Next, as another example of this embodiment, a telesurgery system 200 will be described with reference to FIGS. 26 and 27.

FIG. 26 shows a schematic diagram of the telesurgery system 200 in the other example, and FIG. 27 shows a schematic explanatory diagram of the tool 217 in the telesurgery system 200. Specifically, FIG. 27 at (A) shows a plan view of a tool 217 that may be loaded onto the robotic arm assembly of telesurgical system 200, and FIG. 27 at (B) shows the internal configuration of tool 217.

The telesurgery system 200 includes a surgeon console 201 serving as a station for each of two operators D (D1, D2), a master control unit 202 operated by operator D, a viewing and core cart 240, and a patient side cart. It has a patient side cart 210 robot.

The surgeon's console 201 includes a viewer 201a on which an image of the surgical site is displayed to the operator D. When using the surgeon's console 201, the operator D1 and/or D2 typically sits in the chair of the surgeon's console, has their eyes in front of the viewer 201a, and holds the master control unit 202 in one hand.

Although the telesurgery system 200 may be operated by two operators simultaneously, it may also be operated by one operator. When two operators perform operations simultaneously, cooperative operations by the two operators are possible, and there is an advantage that the operation time for the entire patient may be shortened. The surgeon's console 201 and the master control unit 202 may be configured in a system in which three or more units each are provided, if necessary.

The patient-side cart 210 robot is placed adjacent to the patient. In use, the patient side cart 210 is placed near the patient requiring surgery. The patient-side cart 210 robot has casters on the pedestal 211 so that it is fixed but movable during the surgical procedure. Surgeon console 201 is used in the same operating room as the patient-side cart, but may be located remotely from patient-side cart 210.

The patient-side cart 210 includes four robotic arm assemblies 212, although the number of robotic arm assemblies 212 is arbitrary. Each robot arm assembly 212 is connected to a driving device 213 that enables three-dimensional movement and is driven and controlled.

A display 214 displays image data associated with the surgery. Drive 213 is controlled by master control unit 202 of surgeon console 201. Movement of tool 217 of robot arm assembly 212 is controlled by the operation of master control unit 202.

One robot arm assembly 212a of the four robot arm assemblies 212 is provided with an image capture device 215 such as an endoscope. A viewing camera 216 is included at the remote end of the image capture device 215. An elongated shaft-like image capture device 215 allows a viewing camera 216 to be inserted through a surgical entry port of a patient (not shown).

Image capture device 215 is operatively connected to viewer 201a of surgeon console 201 for displaying images captured by viewing camera 216 thereof. Each of the other robotic arm assemblies 212 is a linkage that respectively supports and Includes a removable surgical instrument, tool 217.

Tool 217 comprises an elongated movable elongated structure 10 to allow insertion through a patient's surgical entry port. Movement of movable elongated structure 10 is controlled by master control unit 202 of surgeon console 201. The movable elongated structure 10 utilizes the movable elongated structure 10 of the previous embodiment.

FIG. 27 shows a configuration of a tool 217 that may be loaded onto the robotic arm assembly 212 of the telesurgery system 200 of FIG. 26 as a representative example of a surgical device. Tools 217 attached to other robotic arm assemblies 212 may be of similar construction or may be surgical devices of other constructions.

A tool 217 shown in FIG. 27 at (A) has a movable elongated structure 10 having a traction wire 50, a surgical device 221 for driving control and monitoring of the tool 217, and a connector 228 for coupling to a robot. The surgical device 221 constitutes a traction driver that drives the traction wire 50 within the movable elongated structure 10.

As shown in FIG. 27 at (8) which illustrates the internal configuration of the tool 217, a surgical device 221 that drives the tool 217 directly connected to the robotic arm assembly 212 of FIG. 26, and a medical system consisting of 210 robots are shown.

The movable elongated structure 10 has a base end side tubular body 30 connected to a shaft 225, a wiring aid 40, and a distal end side tubular body 20 which is an end effector. Since the movable elongated structure 10 has a bent structure as in the above-described embodiment, the degree of freedom of the operation angle of the distal end side tubular body 20 is increased, and the accuracy of robot control is improved.

The surgical device 221 of the tool 217 has control circuitry 231 that controls signals within the surgical device and a signal interface 232 with the robot of the patient-side cart 210.

The control circuit 231 is configured too control a driving mechanism (not shown) that drives a predetermined traction wire of the movable elongated structure 10 based on a control signal from the robot 210.

As an operation is illustrated In FIGS. 26 to 28, the robot of the patient-side cart 210 is routed and/or wirelessly connected to the surgical device 221, the signal interface 232, and the connector 228, and the robot of the patient-side cart 210 Internally, there are an input unit that receives an operation signal from the master control unit 202, an arithmetic unit CPU that executes a predetermined operation program based on the operation signal, and a surgical device 221 based on the output from the arithmetic unit. and an output unit for generating a drive signal for driving the movable elongated structure 10 of the tool 217. The input unit and the output unit are composed of an input/output unit 210a (I/O).

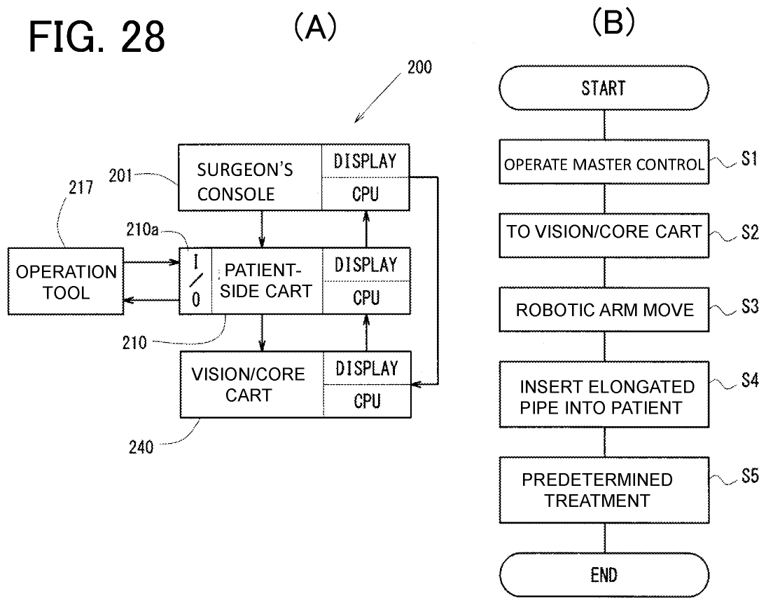

FIG. 28 is an explanatory diagram of the telesurgery system 200, FIG. 28 at (A) is a block diagram showing the connection relationship with each unit, and FIG. 28 at (B) is an operation flow diagram of the telesurgery system 200.

The vision and core cart 240 has functions associated with image capture equipment. When the telesurgical system 200 is activated for surgery, the surgeon operates the master control unit 202 of the surgeon's console 201, and if there are two surgeons, also operates the master control unit 202 of the surgeon's console 201 (step S1), the command generated by the operation is sent to the vision/core cart 240 (step S2).

The vision and core cart 240 then interprets the signals and causes movement of the desired robotic arm assembly 212 to the patients surgical area (step S3).

Next, the movable elongated structure 10 of the tool 217 attached to the selected robot arm assembly 212 is inserted into the patient through an elongated pipe (step S4), and after reaching a predetermined position by bending the distal end side tubular body 20, a predetermined treatment is performed (step S5), and the surgery on the living tissue is completed.

Although the movable elongated structure 10 was used in the description of the telesurgery system 200 and the tool 217 described above, instead of the movable elongated structure 10, the movable elongated structures 10a to 10e, 10j, 10k may be used. In this case, it is preferable to provide a traction drive unit 102 corresponding to the bending direction, that is, the number of traction wires 50 (traction portions 52), and an operation handle 103 corresponding to the bending direction.

Since the telesurgery system 200 including the manipulator 100, the retractor 300, the tool 217, and the tool 217 described above includes the movable elongated structure 10, in addition to the effects of the movable elongated structure 10 described above, the effect obtained by each structure may be produced.

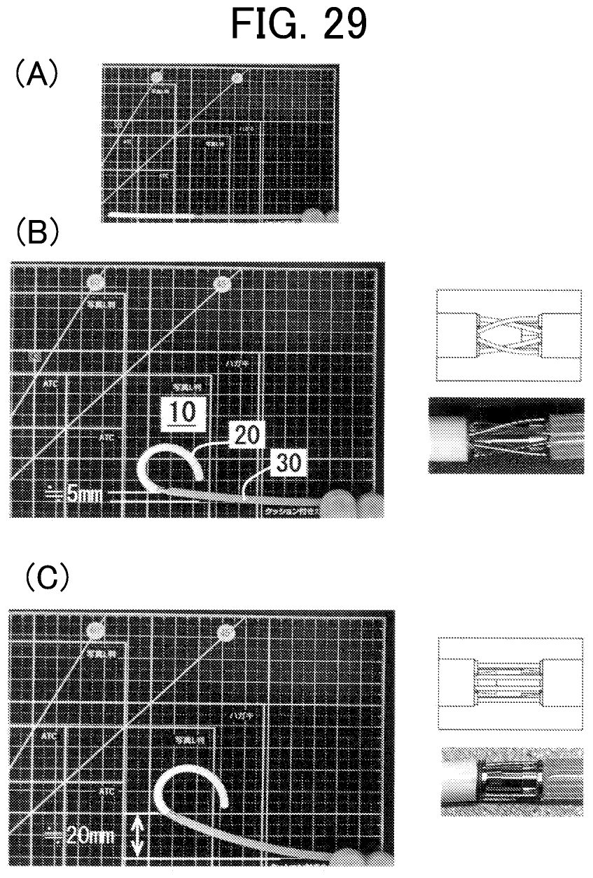

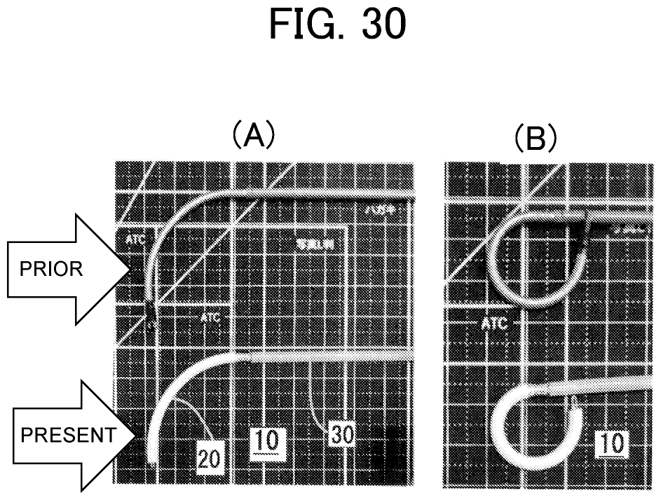

Demonstration experiments conducted on the movable elongated structure 10 (10a, 10b, 10e, 10j, 10k) configured as described above and used for the manipulator 100, the retractor 300, etc. will be described below with reference to FIGS. 29 and 30.

First, as shown in FIG. 29, the bending performance of a four-direction (omnidirectional) movable elongated structure 10 was confirmed as compared with a conventional two-direction movable elongated structure. 29 at (A) shows the initial state, FIG. 29 at (B) shows the state in which the movable elongated structure 10 is bent, and FIG. 29 at (C) shows the movable elongated structure simulating a conventional two-way movable elongated structure bent. 29 at (B) and 29 at (C) show the routed state of the traction portion 52 of the spacer 40 portion of the movable elongated structure 10.

In this demonstration experiment, in order to imitate a conventional two-way movable elongated structure, a two-way movable type was configured by linearly arranging the traction part 52 in the spacer 40 of the movable elongated structure 10, A bending shape with that of the movable elongated structure 10 was compared.

In the movable elongated structure 10 shown in FIG. 29 at (B), the outer diameter of the distal end side flexible tube 20 and the base end side flexible tube 30 is 3.2 mm, the distal spacing X1 is 1.157 mm, and the base end spacing X2 is 1.157 mm. It was designed to be 0.32 mm. When the pair of traction wires 50 are pulled, the movable elongated structure 10 is moved to the base end side flexible tube 30 in the movable elongated structure modeled after the conventional two-way movable elongated structure (see FIG. 29 at (C)). was displaced by about 20 mm, but only about 5 mm was displaced in FIG. 29 at (B). It has been confirmed that the deformation of the flexible tube 30 may reduce the interval of deviation from the horizontal direction by 75%.

As a result, the pulling of the traction wire 50 has little effect on the deformation of the base end side flexible tube 30 in the movable elongated structure 10, and even if the base end side flexible tube 30 is deformed due to a complicated path or external force, the deterioration of the movable bending performance of the distal end side flexible tube 20 is reduced. From this result, it was confirmed that the deterioration of the bending performance of the distal end side flexible tube 20 may be reduced by arranging the traction wire 50.

Next, as shown in FIG. 30, the bending radius of the movable elongated structure 10 was compared with the conventional two-way movable elongated structure.

In addition, FIG. 30 at (A) shows a diagram when bent by 90 degrees, and FAG. 30 at (B) shows a diagram when bent by 275 degrees. The upper part “PRIOR” of FIG. 30 at (A) shows a conventional two-way movable elongated structure, and the lower part “PRESENT” shows the movable elongated structure 10 of the present invention.

Although the movable elongated structure 10 shown in the lower part of FIG. 30 may be bent in four directions (all directions), compared to the conventional two-way movable elongated structure shown in the upper part of FIG. 30. It has been confirmed that the bending radius is reduced by about 13% in a bending state of about 10%.

In addition, in the conventional two-way movable elongated structure, one traction wire 50 is used for bending in one direction, whereas the loop-shaped traction wire 50 is used for bending in one direction. is equivalent (synonymous) to pulling two traction wires 50, the pulling force acting on the traction wires 50 is about half that of the conventional two-way movable elongated structure. Therefore, it is possible to use a thin traction wire 50 that is easily broken compared to a conventional two-way movable elongated structure. In addition, since the pulling force acting on the traction wire 50 is approximately halved, when the traction wire 50 is bent and deformed, the inner surface of the distal wire lumen 22 of the distal end side flexible tube 20 and the base end side flexible tube 30 may be moved. Since the force of contact with the inner surface of the base end side wire lumen 32 is also approximately halved, it was confirmed that a softer tube than the distal end side flexible tube 20 or the base end side flexible tube 30 may be used.

As described above, in the correspondence between the configuration of the present invention and the above-described embodiments, the longitudinal direction of the present invention corresponds to the longitudinal direction 1, and so on.
The distal end side corresponds to the distal end side LF,
The base end side corresponds to the base end side LB,
The distal end side tubular body corresponds to the distal end side flexible tubes 20, 20j, 20k,
the base end side tubular body correspond to the base end side flexible tubes 30, 30j, 30k;
The cylindrical bodies correspond to the cylinders 42, 42k,
Wiring aids correspond to spacers 40, 40a, 40e, 40g, 40i, 40j and assembly spacer
The base end side flanges correspond to the base end side flanges 43b and 43jb,
The distal end side flanges correspond to the distal end side flanges 43a and 43ja,
The distal end side through hole corresponds to the distal end side wire lumen 22,
The base end side through hole corresponds to the base end side wire lumen 32,
The traction operation body corresponds to the traction wire 50,
The distal end side virtual line corresponds to the distal end side virtual line FVL,
The distal end side center line corresponds to the distal end side center line FCL,
The distal end side interval corresponds to the distal end side interval X1,
the base end virtual line corresponds to the base end virtual line BVL,
the base end center line corresponds to the base end center line BCL,
The base end spacing corresponds to the base end spacing X2,
The proximity regulation portion corresponds to the proximity regulation portion 45b,
The traction operation body arrangement parts correspond to the placement recesses 44, 44a, 44e, 44i, 44j, 44ja, and 44jb.

The separation regulation portion corresponds to the separation regulation portion 45a,
The through hole sets correspond to through hole sets 11, 11a, 11b, 11c, 11d, 11e, 11f, 11g, and 11h,
The guiding portion corresponds to the guiding convex portions 46, 46g,
The intermediate tubular body corresponds to the intermediate tube 60,
the intermediate through holes correspond to the intermediate wire lumens 63, 84, 65, 66;
The main lumen corresponds to the main lumens 21 and 31,
The movable elongated structures correspond to movable elongated structures 10, 10a, 10b, 10e, 10j, and 10k,
The traction drive unit corresponds to the traction drive unit 102 and the drive device 213,
The movable elongated structural instrument corresponds to the manipulator 100 and the retractor 300,
The control unit corresponds to the control unit 104, the master control unit 202,
The medical system corresponds to the telesurgery system 200,
The robot arm corresponds to the robot arm assembly 212,
The connecting part corresponds to the connector 228,
The tool corresponds to tool 217,
The manipulator corresponds to manipulator 100,
The input/output unit corresponds to the input/output unit 210a,
The arithmetic unit corresponds to the arithmetic unit CPU,
Although the robot and the medical robot correspond to the patient-side cart 210, they are not limited to the above embodiments.

Although the traction wire 50 is served as the traction operation body, it may be belt-shaped. Moreover, although the single traction wire 50 is bent at the bent portion 51 to form a pair of traction portions 53, a pair of separate traction wires 50 may be used. Further, when pulling a pair of traction wires 50 configured separately, one traction wire 50 may be pulled, or both traction wires 50 may be pulled. Also, although both traction wires 50 are pulled, each traction wire 50 may be pulled with a different pulling force.

The flexible tubes 20 and 30 are circular tubes having a ring-shaped cross section and have the main lumens 21 and 31 inside, but they may be tubular bodies having an elliptical ring-shaped cross section or a polygonal ring-shaped cross section. 31 may have a similar shape to the outer diameter of the cross-sectional shape as described above, or may have a different cross-sectional shape. Also, the main lumens 21 and 31 in the flexible tubes 20 and 30 may be arranged at positions shifted from the centers of the flexible tubes 20 and 30.

Also, the flexible tubes 20 and 30 may be configured with a spine structure in which rigid bodies having main lumens therein are connected via rotary joints or elastic bodies.

Also, telesurgical system 200 may be configured using retractor 300 instead of moveable elongated structure 10 in tool 217.

As another embodiment of the present invention, a medical instrument will be described as an example of a surgical instrument having an auxiliary tool that does not come off accidentally, but the present invention provides a surgical instrument that is not limited to medical instruments.

FIGS. 31 to 35 show structural diagrams of the movable elongated structure 10 of this embodiment.

Note that the longitudinal direction of the movable elongated structure 10 is defined as a longitudinal direction L, and in the longitudinal direction L, the side of the distal end side flexible tube 20A with respect to the base end side flexible tube 20C is defined as a distal end side LF. The side of the base end side flexible tube 20C with respect to 20A is defined as the base end side LB. For convenience of explanation of the movable elongated structure 10, the vertical direction in FIG. 1 is defined as the height direction H, the upward direction as the upward direction HU, and the downward direction as the downward direction HD. Furthermore, the direction connecting the upper right and the lower left in FIG. 31 is defined as the width direction W, the upper right is defined as the right side WR, and the left lower side is defined as the left site WL.

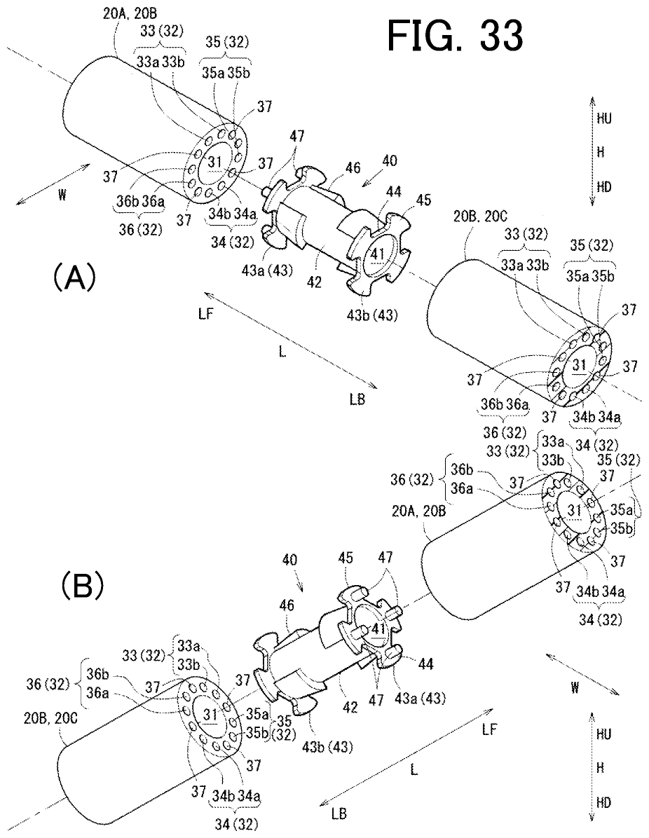

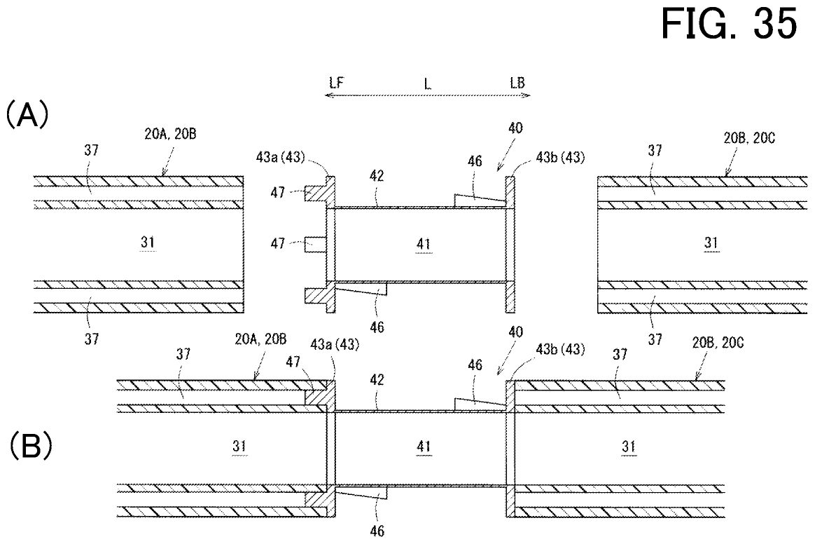

In FIGS. 33 and 35, when the illustrated spacer 40 is the spacer 40 on the distal end side LF in the movable elongated structure 10, the flexible tube 20 on the distal end side LF from the spacer 40 is the distal end side flexible tube 20A. Thus, the flexible tube 20 on the base end side LB becomes the intermediate flexible tube 20B. On the other hand, when the illustrated spacer 40 is the spacer 40 on the base end side LB in the movable elongated structure 10, the flexible tube 20 on the distal end side LF from the spacer 40 becomes the intermediate flexible tube 208, and the base end side. The LB flexible tube 20 becomes the base end side flexible tube 20C. It should be noted that the same applies to FIGS. 8, 9, 11, 12, 14, 15, 17, 18, 20, 21, 23, 24, and 27.

The movable elongated structure 10 shown in FIGS. 31 to 35 is arranged between the flexible tubes 20 (20A, 208, 20C) arranged in series with each other in the longitudinal direction L at predetermined intervals. and a fraction wire 50 inserted through the inside of the tube wall of the flexible tube 20. The movable elongated structure 10 is covered along the longitudinal direction L with an exterior cover (not shown). Also, only the outside of the spacer 40 may be covered with an exterior cover (not shown).

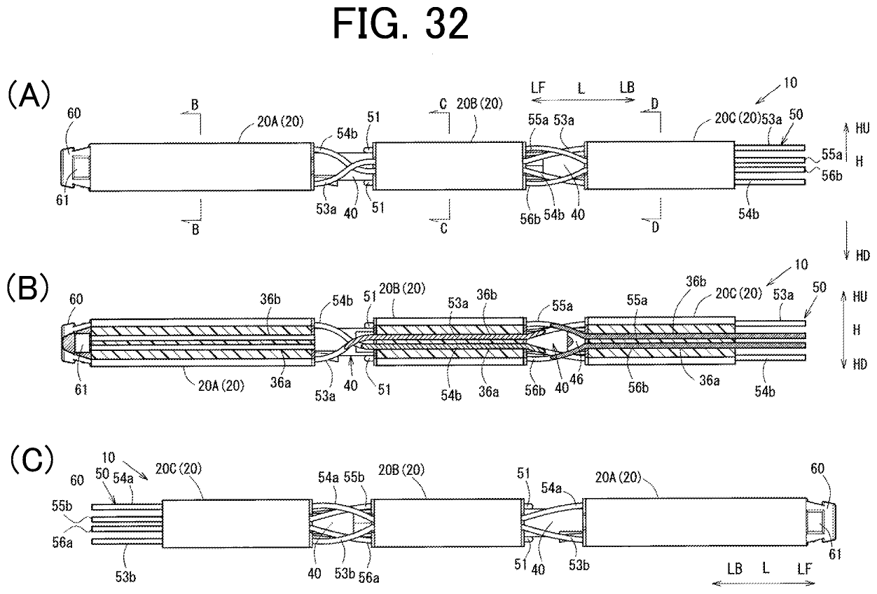

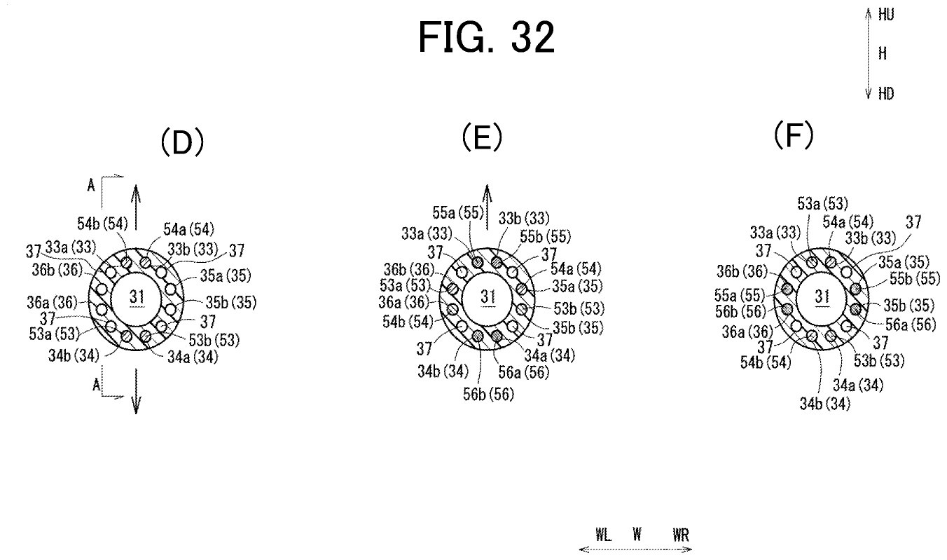

The flexible tube 20 is a cylindrical flexible tube elongated in the longitudinal direction L and provided with a main lumen 31 (FIG. 32 at (D) to (F)) inside. The main lumen 31 is a penetrating space having a circular cross section along the longitudinal direction 1, and is a lumen through which drugs and catheters may be inserted.

The number of main lumens 31 is not limited to one, and may be a plurality of partitioned main lumens 31.

Moreover, the main lumen 31 does not necessarily have to penetrate through the main lumen 31, as in a movable elongated structure In which an appropriate tool such as an electrode is inserted into the interior of the main lumen 31, and a filling material or the like is filled and fixed.

As shown in FIG. 32 at (D) to (F), wire lumens 32 (33, 34, 35, 36) and a position control lumen 37 are provided inside the tube wall between the main lumen 31 and the outer peripheral surface of the flexible tube 20.

The wire lumen 32 is a penetrating space with a circular cross section extending in the longitudinal direction L inside the tube wall, and is formed with a diameter that allows a traction wire 50 to be described later to be inserted therethrough.

The wire lumens 32 are provided in four directions on the pipe wall, which has a ring-shaped cross section.

Specifically, as shown in FIG. 32 at (Q) to (F), the wire lumen 33 (33a, 33b) in the upward direction HU, the wire lumen 34 (34a, 34b) in the downward direction HO, and the right WR in the pipe wall having a ring-shaped cross section wire lumens 35 (35a, 35b) on the left side WL and wire lumens 36 (36a, 36b) on the left side WL.

In addition, as described above, the wire lumens 32 provided in the four directions in the pipe wall having a ring-shaped cross section are spaced apart at predetermined intervals in the circumferential direction of the circular cross section so that the traction wires 50 configured as a pair are inserted. It has two of each.

In the pair of wire lumens 33, 34, 35, 36 formed by two through holes, the counterclockwise sides are 33a, 36a, 34a, 35a, and the clockwise skies are 33b, 35b, 34b, 36b.

In addition, as shown in FIG. 33 at (A), the intervals between the two wire lumens 32 provided in each of the four directions on the pipe wall having a ring-shaped cross section are the wire lumens 32 (33b and 35a, 35b and 34a) adjacent to each other In the circumferential direction. 34b and 36a, and 36b and 33a) are set at about half the intervals.

It should be noted that the wire lumens 32 provided two in each of the four directions may be integrally formed to form an elliptical shape.

The position regulating lumen 37 is disposed between the wire lumens 32 and is a penetrating space having a circular cross section extending in the longitudinal direction L inside the tube wall.

The position regulation lumens 37 arranged between the wire lumens 32 are arranged in four oblique directions. Specifically, between the wire lumen 33b and the wire lumen 35a, between the wire lumen 35b and the wire lumen 34a, between the wire lumen 34b and the wire lumen 36a, and between the wire lumen 36b and the wire lumen 33a. A position regulation lumen 37 is provided at one place.

It should be noted that the position control lumen 37 is formed in a through space having a circular cross section extending In the longitudinal direction L inside the pipe wall, similarly to the wire lumen 32.

The flexible tube 20 configured as described above may be composed of a flexible tube such as polyamide elastomer, expanded polytetrafluoroethylene, polyurethane, or polytetrafluoroethylene.

Among the flexible tubes 20, the middle flexible tube 20B and the base end side flexible tube 20C arranged in the middle in the longitudinal direction L and the base end side LB are different in flexibility from the distal end side flexible tube 20A. It may be made of another material.

A spacer 40 between the distal end side flexible tube 20A and the intermediate flexible tube 208 and between the intermediate flexible tube 208 and the base end side flexible tube 20C will be described.

As shown in FIG. 33, the spacer 40 includes a cylinder 42 having an internal space 41 having a circular crass section penetrating in the longitudinal direction L, and flanges 43 (43a, 43b) provided at both ends of the cylinder 42 in the longitudinal direction 1), a guide projection 46 and a position control projection 47.

The internal space 41 is formed with a space having the same diameter as the main lumen 31 described above. The cylinder 42 is formed with a diameter that does not interfere with the insertion of the traction wire 50 into the wire lumen 32 provided in the distal end side flexible tube 20A and the wire lumen 32 provided in the base end side flexible tube 20C.

The flanges 43 (43a, 43b) are formed so as to protrude from the outer surface of the cylinder 42 in the radial direction of the cylinder 42, and at a location corresponding to the wire lumen 32, an arrangement recess 44 for arranging the traction wire 50 is provided. A regulation part 45 is provided between the arrangement recesses 44. In addition, the flange portion 43 is formed with an outer diameter equivalent to the outer diameter of the flexible tube 20.

The arrangement recess 44 is a recess for collectively arranging two traction wires 50 to be inserted into two wire lumens 32, and is formed with a width and depth corresponding to the two wire lumens 32.

Of the protrusions 43 provided on both sides in the longitudinal direction L of the cylinder 42, the distal end side LF is defined as a distal end side flange 43a, and the base end side LB is defined as a base end side flange 43b.

In addition, the regulation parts 45 provided on both sides of the arrangement recess 44 restrict the traction wires 50 arranged in the same arrangement recess 44 from being separated from each other in the circumferential direction in the distal end side flange 43a. At 43b, the regulation parts 45 between the arrangement recesses 44 restrict the traction wires 50 arranged in the arrangement recesses 44 adjacent in the circumferential direction from coming close to each other in the circumferential direction.

Furthermore, a guide convex part 46 for guiding the traction wire 50 radially outward is provided at the end of the cylinder 42 in the longitudinal direction L.

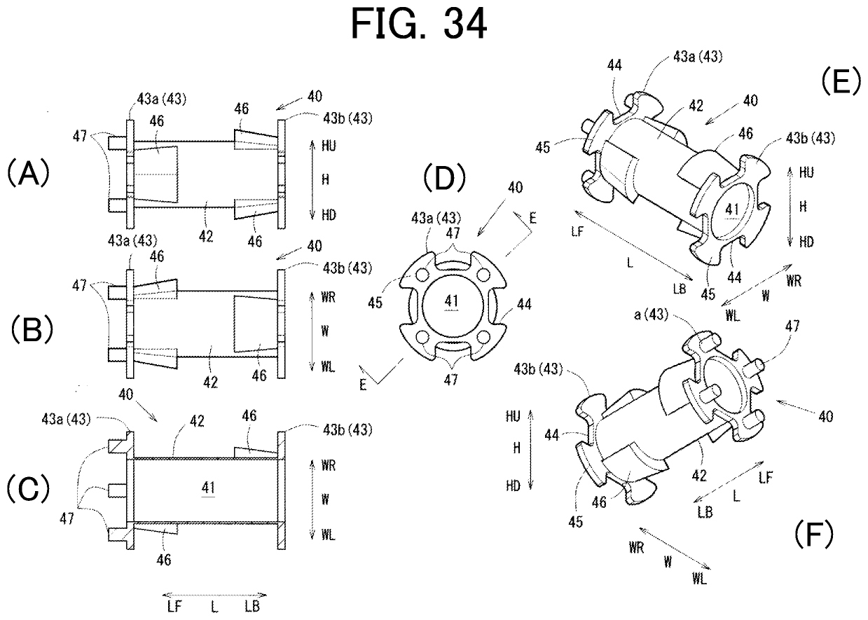

As shown in FIG. 34 at (A) to (C), the guide convex part 46 is provided at a position corresponding b the arrangement recess 44 of the protruded edge 43 at the end in the longitudinal direction L, and has a corresponding width. It is formed to have a trapezoidal vertical cross-sectional shape in which the projection height gradually increases toward the center in the longitudinal direction L of the cylinder 42.

The guide convex part s 46 are provided at positions facing the radial direction of the arrangement recesses 44 provided in the four directions at both ends in the longitudinal direction L, and the opposing direction is the guide convex part 46 on the distal end side LF. It is provided so as to be orthogonal to the guide convex portion 46 on the base end side LB.

Specifically, the guide convex part s 46 on the distal end side LF are provided on the right side WR and the left side WI so as to face each other in the width direction W, and the guide convex parts 46 on the base end side LB are provided so as to face each other in the height direction H. are provided in the upward direction HU and the downward direction HD.

A position regulating convex portion 47 that protrudes toward the distal end side LF is provided on the regulation part 45 of the distal end side flange portion 43a.

The position-regulating convex portion 47 is formed in a cylindrical shape with an appropriate height that may be inserted into the position-regulating lumen 37 of the flexible tube 20.

The spacer 40 configured as described above may be made of, for example, a metal material such as stainless steel, an elastic body such as polytetrafluoroethylene, an elastic body such as a spring, or a resin material.

Further, the spacer 40 may be configured by integrating the cylinder 42 and the flange portion 43, or may be configured separately and assembled. In addition, when the cylinder 42 and the flange portion 43 are configured separately, the cylinder 42 and the flange portion 43 may be configured with the same material, or may be configured with different materials.

Also, the traction wire 50 is a wire having flexibility, and is formed with a length that is at least twice the length in the longitudinal direction L of the movable elongated structure 10.

As shown in FIGS. 31 and 32, the traction wire 50 has two traction portions 52: a bent portion 51 arranged in a bent concave portion 61 of the distal end cap 60, which will be described later, and a base end side LB from the bent portion 51.

As described above, the four traction wires 50 that are bent at the bending portion 51 and have the pair of traction portions 52 are provided corresponding to the wire lumens 32 provided in the four directions.

Specifically, the traction wire 50 has a traction portion 53 (53a, 53b) and a traction portion 54 (54a, 54b) arranged in the height direction H in the base end side flexible tube 20 at (C) shown in FIG. 32 at (D) and (F), traction portions 55 (55a, 55b) arranged in the width direction W, and traction portions 56 (56a, 56b).

One of the portions corresponding to the towed portion 52 in each of the towed portions 53, 54, 55, and 56 is set to be towed portions 53a, 54a, 55a, and 56a, and the other is set to be towed portions 53b, 54b, 55b, and 56b.

The traction wire 50 may be made of a metal material such as stainless steel, nylon, fluorocarbon, or the like.

As shown in FIGS. 31 and 32, a distal cap 60 is provided at the distal end LF of the distal end side flexible tube 20A.

The distal end cap 60 has a bent recess 61 for forming a bent portion 51 of the traction wire 50, which will be described later. The bent recesses 61 are provided at four locations corresponding to the wire lumens 32 provided in the four directions on the tube wall, and two insertion holes through which the traction wires 50 are inserted are provided.

Note that the distal end cap 60 is penetrated in the longitudinal direction L by an instrument or the like that is inserted through the main lumen formed by communicating the main lumen 31, the internal space 41 described later, and the main lumen 31 in a device using the movable elongated structure 10. A through hole may be provided like through hole 62 in FIG. 71, or there may be no through hole.

Assembly of the movable elongated structure 10 having the elements configured as described above will now be described.

First, the distal end side flexible tube 20A, the spacer 40, the intermediate flexible tube 20B, the spacer 40, and the base end side flexible tube 20C are arranged in series along the longitudinal direction L from the distal end side LF toward the base end side LB. Arrange them in this order.

At this time, as shown in FIGS. 33 and 35, the position regulation convex portion 47 of the spacer 40 is inserted into the position regulation lumen 37 of the flexible tube 20 on the distal end side LF.

By arranging in this way, the wire lumen 32 (33, 34, 35, 36) of the distal end side flexible tube 20A, the four-direction arrangement recesses 44 of the spacer 40, the wire lumen 32 of the intermediate flexible tube 208, and the spacer 40, and the wire lumen 32 of the base end side flexible tube 20C communicate in the longitudinal direction L, respectively.

As shown in FIG. 32, the traction portion 54b that is inserted into the wire lumen 33a on the left side WL of the wire lumen 33 in the upward direction HU in the distal end side flexible tube 20A is a woes provided on the left side WL in the intermediate flexible tube 20B. It is inserted through the wire lumen 36a in the downward direction HD of the lumen 36, and is inserted through the wire lumen 34b on the left side WL of the wire lumen 34 provided in the downward direction HD in the base end side flexible tube 20C.

The traction portion 54a inserted into the right WR wire lumen 33b of the upward HU wire lumen 33 of the distal end side flexible tube 20A is the upward HU of the wire lumen 35 provided at the right WR of the intermediate flexible tube 208. It is inserted through the wire lumen 35a, and is inserted through the wire lumen 33b on the right side WR of the wire lumen 33 provided in the upward direction HU in the base end side flexible tube 20C.

In addition, the traction portion 53b that is inserted into the right WR wire lumen 34a of the downward HD wire lumen 34 of the distal end side flexible tube 20A extends downward HD of the wire lumen 35 provided on the right WR of the intermediate flexible tube 208. It is inserted through the wire lumen 35b, and is inserted through the wire lumen 34a on the right side WR of the wire lumen 34 provided in the downward direction HD in the base end side flexible tube 20C.

The traction portion 53a inserted into the left WL wire lumen 34b of the downward HD wire lumen 34 of the distal end side flexible tube 20A extends upward HU of the wire lumen 36 provided on the left WL of the intermediate flexible tube 20B. It is inserted through the wire lumen 36b, and is inserted through the wire lumen 33a on the left side WL of the wire lumen 34 provided in the upward direction HU in the base end side flexible tube 20C.

On the other hand, the traction portions 55 and 56 are not inserted into the wire lumens 35 and 36 in the width direction W of the distal end side flexible tube 20A, and the left side WL of the wire lumen 33 provided in the upward direction HU in the intermediate flexible tube 20B, and the wire lumen 36b in the upward direction HU of the wire lumen 36 provided on the left side WL of the base end side flexible tube 20C.

Then, it is inserted into the traction portion 55b of the wire lumen 33b of the right WR of the wire lumen 33 provided in the upward direction HU in the intermediate flexible tube 208, and is inserted above the wire lumen 35 provided in the right WR of the base end side flexible tube 20C. It is inserted through the wire lumen 35a in the direction HU.

In addition, it is inserted into the traction portion 56a of the wire lumen 34a of the right WR of the wire lumen 34 provided in the downward HD of the intermediate flexible tube 208, and is below the wire lumen 35 provided in the right WR of the base end side flexible tube 20C. It is inserted through the wire lumen 35b in the direction HD.

Then, it is inserted into the traction portion 56b of the wire lumen 34b on the left side WL of the wire lumen 34 provided in the downward direction HD in the Intermediate flexible tube 208, and below the wire lumen 36 provided in the left side WL in the base end side flexible tube 20C. It is inserted through the wire lumen 36a in the direction HD.

It should be noted that the traction portions 55 and 56 that do not pass through the wire lumen 33 of the distal end side flexible tube 20A but pass through the wire lumen 33 of the intermediate flexible tube 20B and the wire lumen 33 of the base end side flexible tube 20C are A bent-back portion 51 is formed al the arrangement recess 44e of the flange 43 of the spacer 40 arranged between the flexible tube 20A and the intermediate flexible tube 20B.

In addition, in the spacer 40 arranged between the distal end side flexible tube 20A and the intermediate flexible tube 208 in the movable elongated structure 10, the traction portion 53a and the traction portion 54b intersect on the front side shown in FIG. 32 at (A). However, on the back side shown in FIG. 32 at (C), the traction portion 53b and the traction portion 54a are routed so as not to cross each other.

In the movable elongated structure 10 configured as described above, the traction portion 53 and the traction portion 54 are inserted through the distal end side flexible tube 20A, the intermediate flexible tube 20B, and the base end side flexible tube 20C and the fraction portion 55 is inserted, and the traction portion 56 are threaded through the intermediate flexible tube 208 and the base end side flexible tube 20C.

Therefore, the spacer 40 disposed between the base end side flexible tube 20 and the intermediate flexible tube 206 has four traction portions 53, 54, 55, 56 disposed thereon, while the distal end side flexible tube 20A is disposed, and the Intermediate flexible tube 208.

Therefore, it is possible to use a spacer 40 in which the traction wires 50 are less likely to intersect with each other, and the guide convex portion 46 (FIG. 4) is provided only on the distal end side LF.

As described above, traction portion 53 and traction portion 54 extend through distal end side flexible tube 20A, intermediate flexible tube 208 and base end side flexible tube 20C and traction portion 55 and traction portion 56 extend through intermediate flexible tube 208. And the movable elongated structure 10 inserted through the base end side flexible tube 20C includes a traction portion 53 and a traction portion that are inserted through the distal end side flexible tube 20A, the intermediate flexible tube 20B and the base end side flexible tube 20C. 54 toward the base end side LB, the distal end side flexible tube 20A is lifted relative to the intermediate flexible tube 208 without substantially interfering with the intermediate flexible tube 20B and the base end side flexible tube 20C. It may be bent either in the direction HU or downward HD.

On the other hand, by pulling either the traction portion 55 or the traction portion 56 inserted through the intermediate flexible tube 20B and the base end side flexible tube 20C toward the base end side LB, it substantially interferes with the base end side flexible tube 20C. The intermediate flexible tube 208 may be bent either upward HU or downward HD with respect to the base end side flexible tube 20C. At this time, if the traction portion 53 and the traction portion 54 are not pulled, the distal end side flexible tube 20A does not bend with respect to the intermediate flexible tube 208, and the base end side flexible tube 20C is directed upward HU or It bends following the intermediate flexible tube 208 that bends downward HD. At this time, if the traction portion 53 and the traction portion 54 are pulled, the distal end side flexible tube 20A may be pulled out of the intermediate flexible tube 208 and the base end side flexible tube 20C without substantially interfering with the bent intermediate flexible tube 20B and the base end side flexible tube 20C. 208 is bent upward HU or downward HD. That is, the movable elongated structure 10 may be bent without substantially interfering with the distal end side flexible tube 20A, the intermediate flexible tube 208, and the base end side flexible tube 20C.

As described above, an elongated intermediate flexible tube 208 having a wire lumen 32 is positioned between the distal end side flexible tube 20A and the base end side flexible tube 20C, as well as pairs of traction tubes. Wires 50 are provided, and at least one of the plurality of pairs of traction wires 50 are connected to the wire lumen 32 of the distal end side flexible tube 20A, the wire lumen 32 of the intermediate flexible tube 208, and the base end side flexible tube 20C. At least one pair of the traction wires 50 out of the plurality of pairs of traction wires 50 is inserted through the wire lumen 32 of the intermediate flexible tube 208 and the wire lumen 32 of the base end side flexible tube 20C. Therefore, it is possible to configure the movable elongated structure 10 in which the intermediate flexible tube 208 and the distal end side flexible tube 20A may be bent with respect to the base end side flexible tube 20C.

Specifically, the fraction wires 55 and 56 of the plurality of pairs of traction wires 50 are inserted through the wire lumens 33 and 34 of the intermediate flexible tube 20B and the wire lumens 35 and 36 of the base end side flexible tube 20C, respectively. The other traction wires 53, 54 of the traction wires 50 are connected to the wire lumens 33, 34 of the distal end side flexible tube 20A, the wire lumens 35, 36 of the intermediate flexible tube 208, and the wire lumen 33 of the base end side flexible tube 20C, 34 of the intermediate flexible tube 20B and the wire lumens 35 and 36 of the base end side flexible tube 20C. The intermediate flexible tube 208 in the intermediate portion of the lengthy structure 10 may be bent without substantially interfering with the base end side flexible tube 20C and the wire lumens 33 and 34 of the distal end side flexible tube 20A and the intermediate flexible tube 208 may be bent and the wire lumens 33, 34 of the base end side flexible tube 20C are pulled by pulling the traction wires 53, 54 that are inserted through the wire lumens 35, 36, and the wire lumens 33, 34 of the base end side flexible tube 20C. Flexible tube 20A may be bent substantially without interfering with intermediate flexible tube 208 and base end side flexible tube 20C.

Thus, with the above-described configuration, the intermediate flexible tube 208 in the intermediate portion of the movable elongated structure 10 and the distal end side flexible tube 20A in the distal portion of the movable elongated structure 10 substantially interfere with each other. may be bent without

Further, as described above, by pulling the traction wire 50, the distal end side flexible tube 20A (the intermediate flexible tube 208) may be bent with respect to the intermediate flexible tube 208 (the base end side flexible tube 20C). In the movable elongated structure 10, the spacer 40 has two traction wires 50 arranged in each of the arrangement recesses 44 provided in four directions in the flange portion 43. The spacer 40 may be held between the flexible tubes 20 because the fraction wire 50 surrounds the flange 43.

However, when the traction wire 50 is pulled such that the distal end side flexible tube 20A is greatly bent with respect to the intermediate flexible tube 208, an outward load acts on the spacer 40 in the bending direction, The spacer 40, which is surrounded and held by the traction wire 50, tries to fall out from between the flexible tubes 20 inadvertently. Since it is inserted into the position regulating lumen 37 of the flexible tube 20 of the LF, it is possible to regulate the spacer 40 from falling off against the load directed outward in the bending direction acting on the spacer 40.

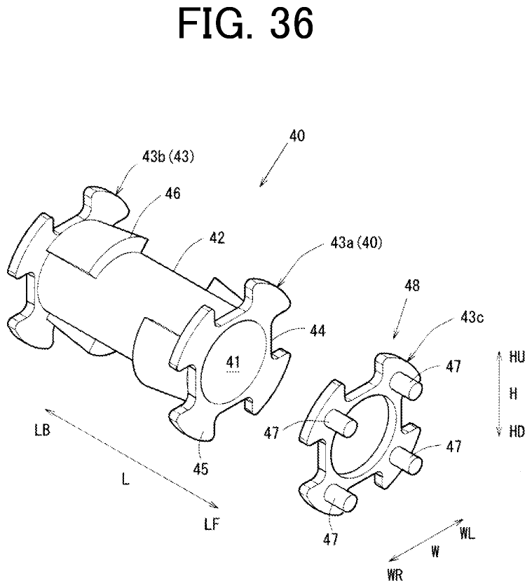

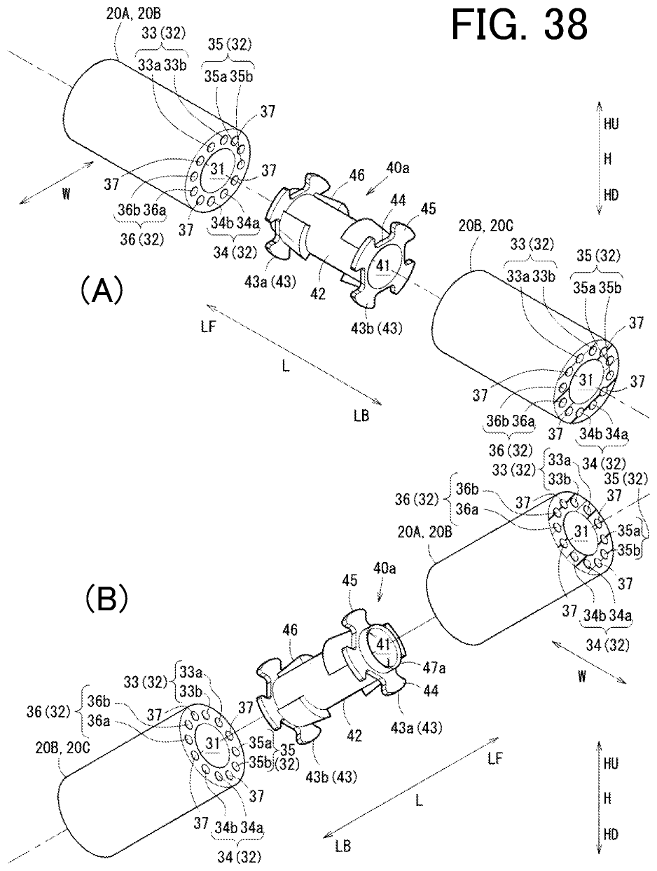

FIG. 38 shows an exploded perspective view of the spacer 40 of another embodiment. In the spacer 40 described above, the position regulation convex part 47 is provided so as to protrude from the distal end side flange 43a toward the distal end side LF. If required, a position regulation convex part 47 configured separately may be attached to the tip-side projecting edge 43a like the spacer 40 of FIG. 36.

Specifically, the spacer 40 is provided with a position regulation convex part 47 by attaching a mounting protrusion 48, which is formed separately from the spacer 40, to the tip end side flange 43a.

The mounting protrusion 48 includes a protrusion 43c having the same shape as the protrusion 43 and a position restricting projection 47, and the protrusion 43c is fixed to the distal end side LF of the distal end side flange 43a.

As a result, the spacer 40 having the attachment protrusion 48 attached to the distal end side flange 43a has the same effect as the spacer 40 described above. Furthermore, by attaching the mounting protrusion 48 to the distal end-side protruded edge 43a, even the spacer 40 that does not have the position regulation convex part 47 may be served as the spacer 40 that has the position regulation convex part 47.

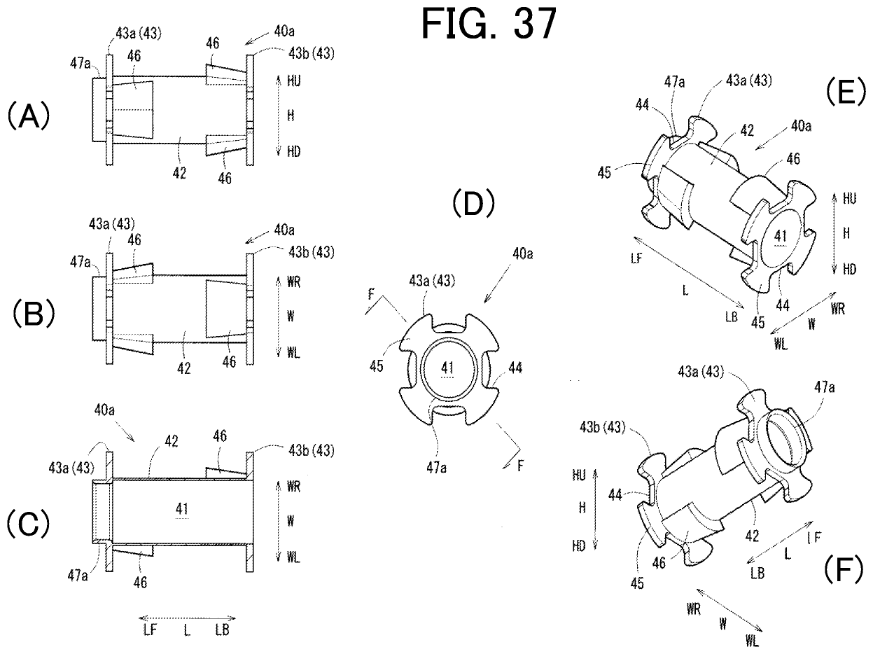

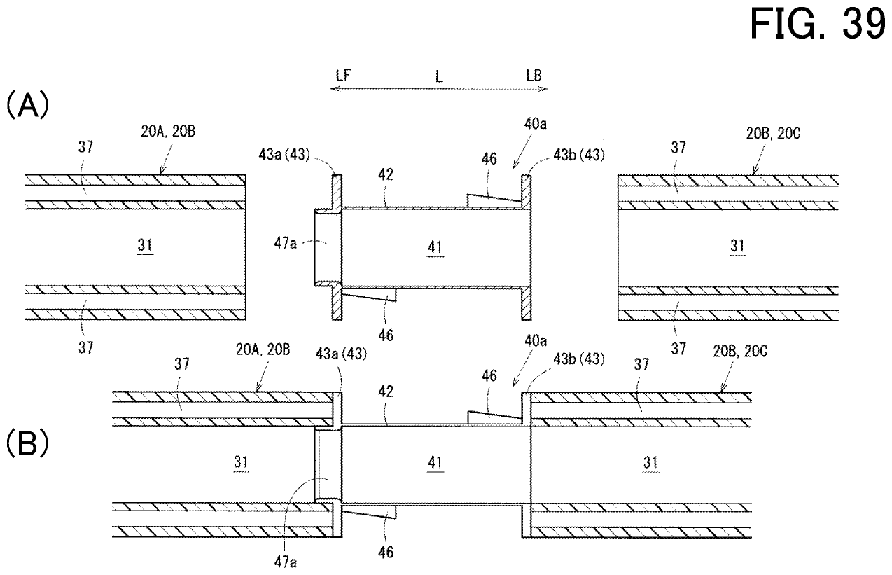

FIG. 37 shows another example of a spacer 40a, FIG. 38 shows another example of an enlarged exploded perspective view of the main part of the movable elongated structure 10, and FIG. 39 is an explanatory diagram of the essential part of the movable elongated structure 10 of still another embodiment.

In the spacer 40 described above, the position regulation convex part 47 protruding toward the distal end side LF from the distal end side flange 43a is inserted into the position-regulating lumen 37. Instead of the position regulation convex part 47, the spacer 40a as shown in FIGS. 37 to 39, a protruding ring 47a protruding toward the distal end side LF along the internal space 41 is provided from the distal flange 43a, and the protruding ring 47a is inserted into the main lumen 31 of the flexible tube 20 on the distal end side LF.

The movable elongated structure 10 using the spacers 40a in this manner has the same effects as the movable elongated structure 10 using the spacers 40 described above. Furthermore, since the protruding ring 47a of the spacer 40a is inserted into the main lumen 31, the position of the spacer 40 relative to the flexible tube 20 may be regulated at the cross-sectional center of the flexible tube 20.

Since the projecting ring 47a is inserted into the main lumen 31 of the flexible tube 20 on the distal end side LF and the flexible tube 20 on the distal end side LF and the spacer 40 are assembled, the flexible tube 20 has the main lumen 31 and the wire. Although it is possible to provide only the lumen 32 without providing the position control lumen 37, the position control lumen 37 may be provided as shown in the drawings.

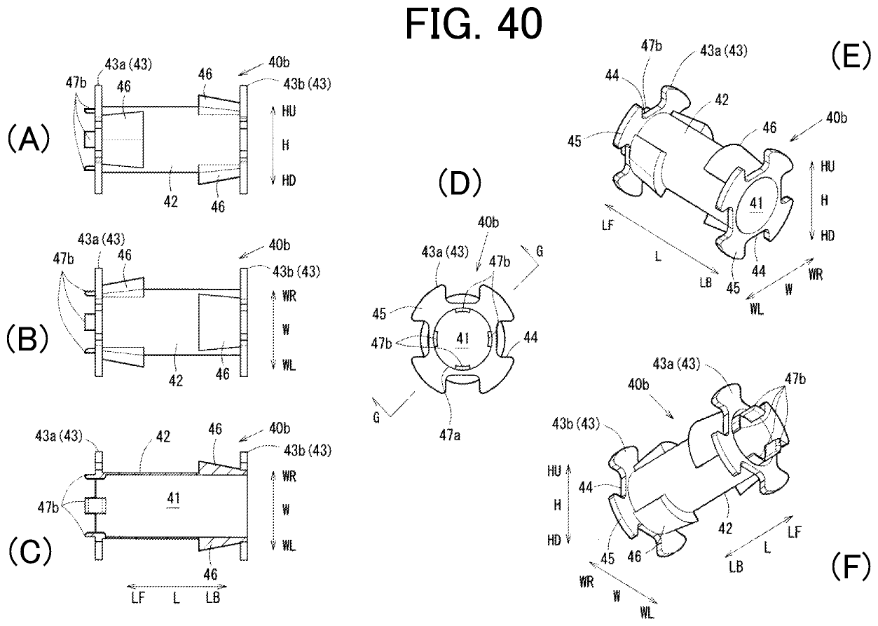

In the spacer 40a described above, the protruding ring 47a that protrudes toward the distal end side LF from the distal end side flange 43a is inserted into the position regulation lumen 37, but instead of the protruding ring 47a, as shown in FIGS. 40 to 42, a protruding piece 47b protruding toward the distal end side LF along the internal space 41 is provided from the distal end side flange portion 43a, and the projecting piece 47b is configured to be inserted into the main lumen 31 of the flexible tube 20 on the distal end side LF.

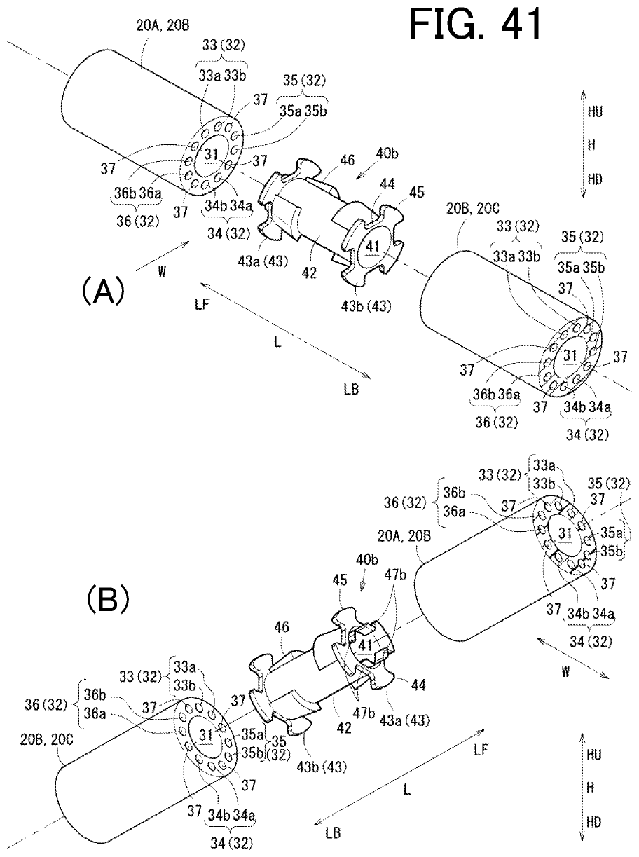

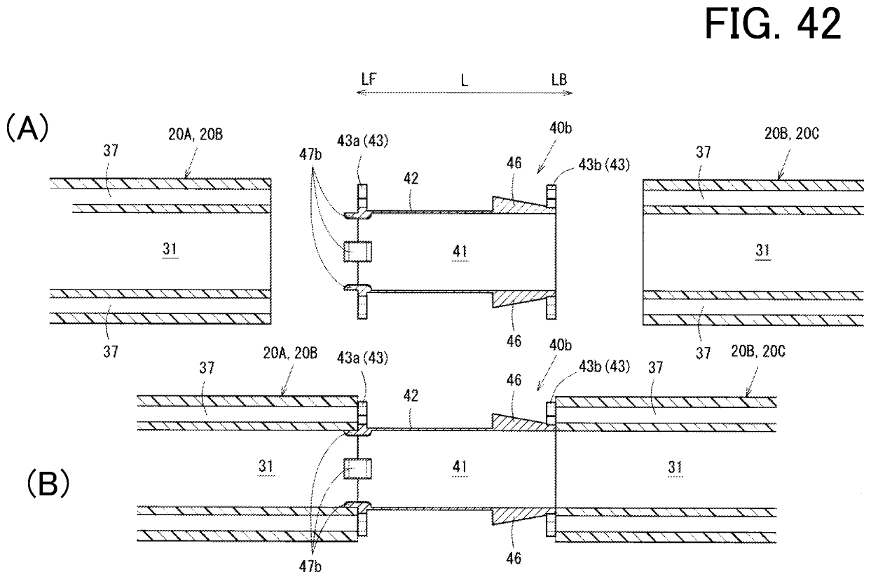

FIG. 40 shows an explanatory view of a spacer 40b of still another embodiment, FIG. 41 shows an explanatory view of an enlarged exploded perspective view of a main part of a movable long structural body 10 of still another embodiment, and FIG. 42 shows an explanatory diagram of a main part of the movable elongated structure 10 of another embodiment.

The protruding ring 47a of the spacer 40a described above is formed in a circular ring shape, but the spacer 40b forms the protruding piece 47b with a segment that protrudes toward the distal end side LF along the inner space 41, and extends in the circumferential direction. A plurality of them are provided at predetermined intervals. Although the spacer 40b illustrated in FIGS. 40 to 42 has four protruding pieces 47b, the number may be two or more.

The spacer 40b having the protruding piece 47b is assembled with the flexible tube 20 by inserting the protruding piece 47b into the main lumen 31 of the flexible tube 20 in the same manner as the spacer 40a having the protruding ring 47a described above. A body 10 is to be constructed.

The movable elongated structure 10 using the spacer 40b having the protruding piece 47b configured In this manner has the same effects as the movable elongated structure 10 using the spacer 40a having the protruding ring 47a.

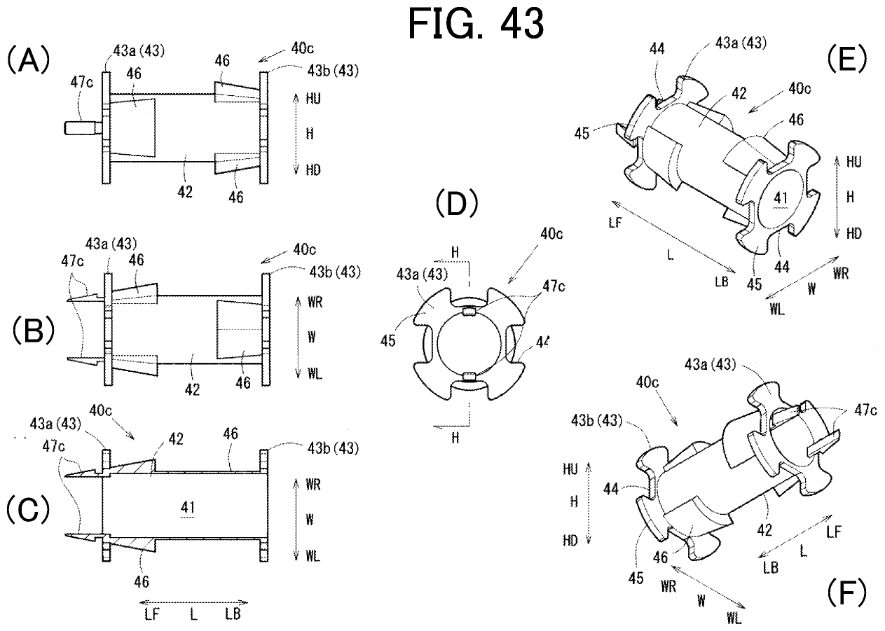

In the spacer 40b described above, four protruding pieces 47b protruding toward the distal end side LF along the Internal space 41 are provided from the distal end side flange 43a, and the protruding pieces 47b are provided in the main lumen 31 of the flexible tube 20 on the distal end side LF. However, instead of the protruding piece 47b, as shown in later-described FIGS. 43 to 45, a projecting locking piece 47c may be provided, and the projecting locking piece 47c may be inserted into the main lumen 31 of the flexible tube 20 on the distal end side LF for locking.

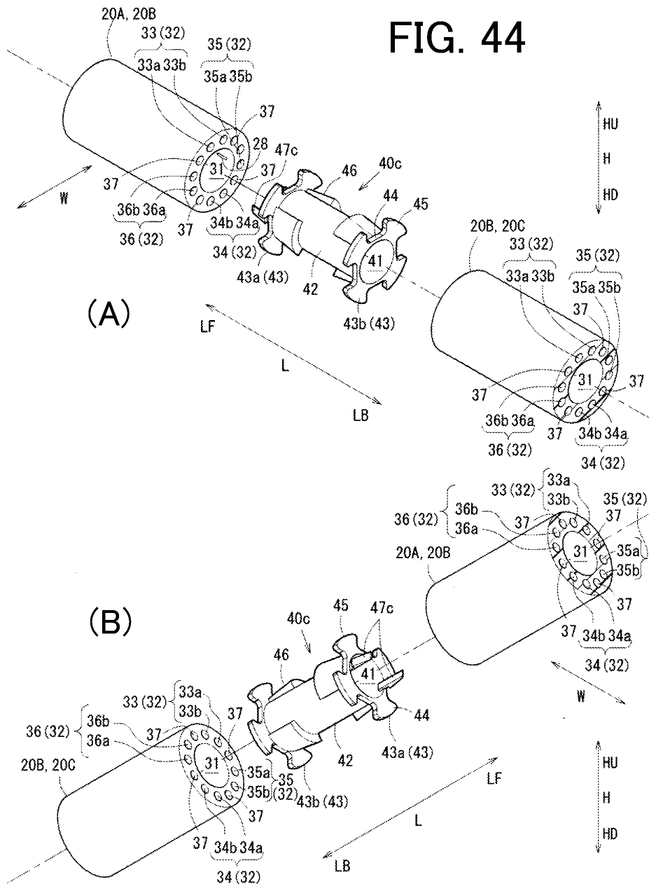

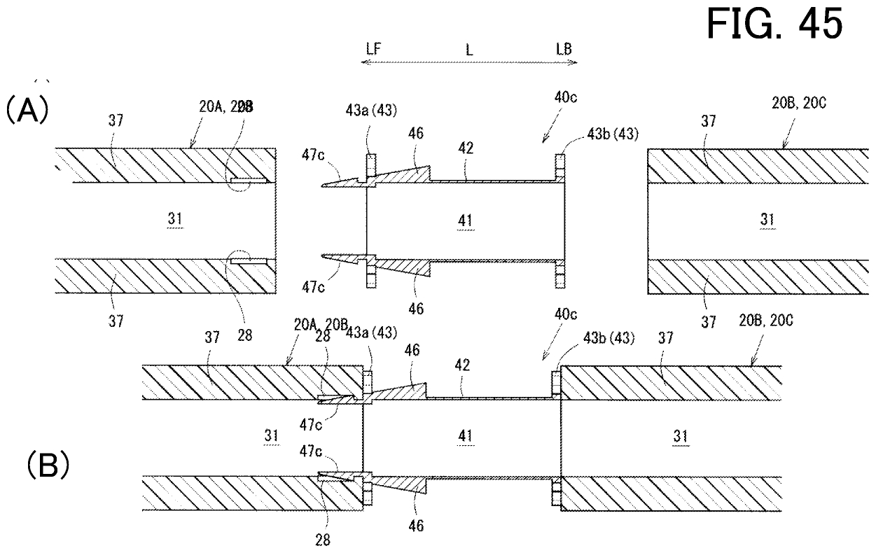

FIG. 43 shows an explanatory view of a spacer 40c of still another embodiment. FIG. 44 shows an explanatory view of an enlarged exploded perspective view of a main part of a movable long structural body 10 of still another embodiment, and FIG. 45 further shows an explanatory diagram of a main part of the movable elongated structure 10 of another embodiment.

Like the protruding piece 47b, the protruding locking piece 47c protrudes from the distal end side flange 43a along the internal space 41 toward the distal end side LF, but has a locking claw that may be locked outside the tip, are provided in opposite directions in the circumferential direction.

The flexible tube 20 to which the spacer 40c having the projecting locking piece 47c is mounted has the locking claw of the projecting locking piece 47c engaged with the inner surface of the base end side LB of the main lumen 31. As shown in FIG. 45, an engagement recesses 28 for stopping are provided at positions facing each other in the circumferential direction.

Similar to the spacer 40b having the projecting piece 47b, the spacer 40c having the projecting piece 47c is inserted into the main lumen 31 of the flexible tube 20 and the locking claw is engaged with the locking recess 28. It is stopped and assembled with the flexible tube 20 to form the movable elongated structure 10.

The movable elongated structure 10 using the spacer 40c having the projecting locking piece 47c configured in this way has the same effects as the movable elongated structure 10 using the spacer 40b having the projecting piece 47b. may be played. Further, in the movable elongated structure 10 using the spacer 40c having the projecting locking piece 47c, the locking claw of the projecting locking piece 47c is formed in the main lumen 31 of the flexible tube 20 on the distal end side LF. Since the spacer 40 is engaged with the recess 28, the position of the spacer 40 may be more reliably restricted with respect to the flexible tube 20.

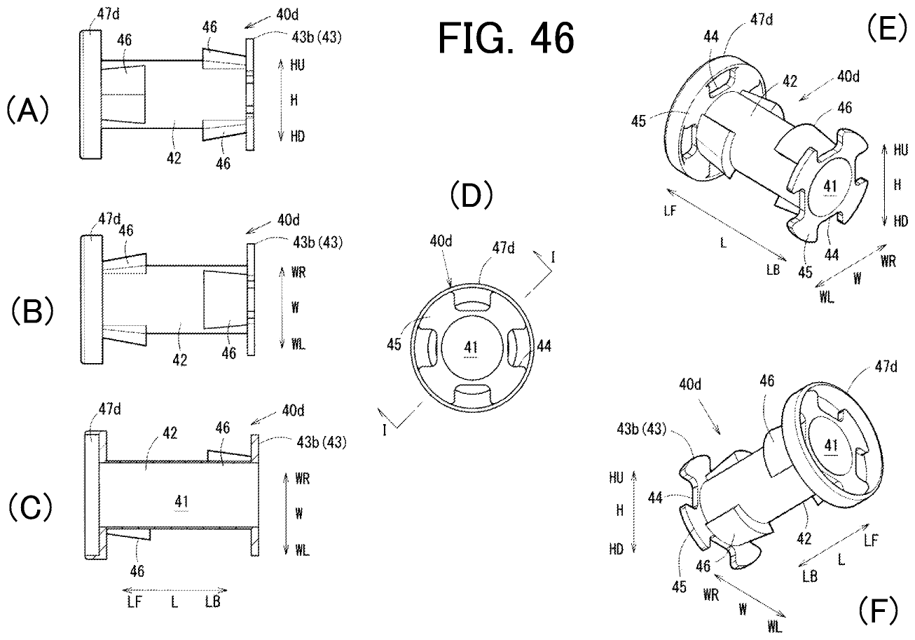

In the spacers 40a, 40b, and 40c described above, the protruding ring 47a, the protruding piece 47b, and the protruding locking piece 47c, which protrude toward the distal end side LF from the distal end side flange 43a, are inserted into the main lumen 31. Instead of the piece 47b and the protruding locking piece 47c, as shown in FIGS. 46 to 48 described later, a ring-shaped fitting ring 47d protruding toward the distal end side LF along the outer peripheral edge of the distal end side flange portion 43a is provided. A fitting ring 47d may be fitted to the outer periphery of the flexible tube 20 on the distal end side LF.

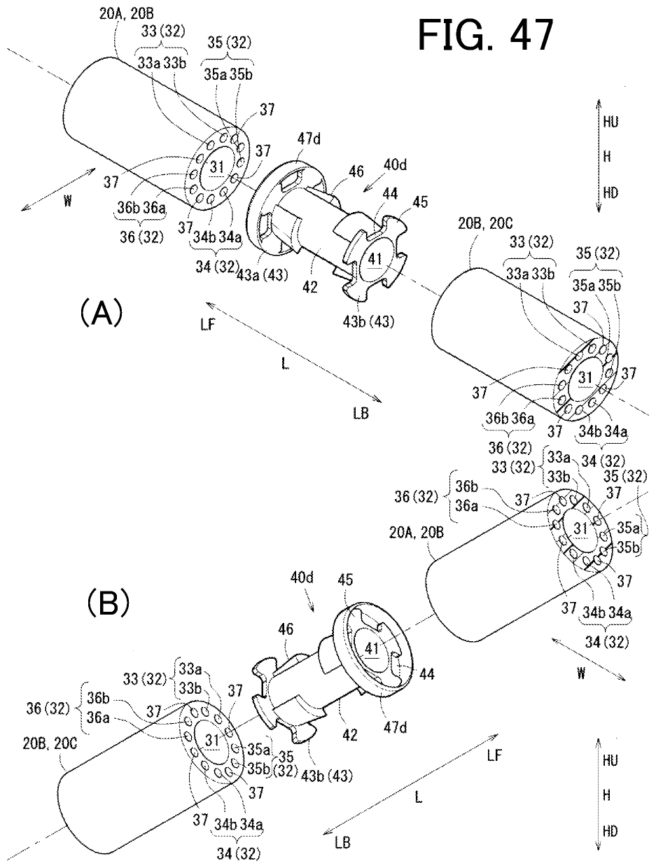

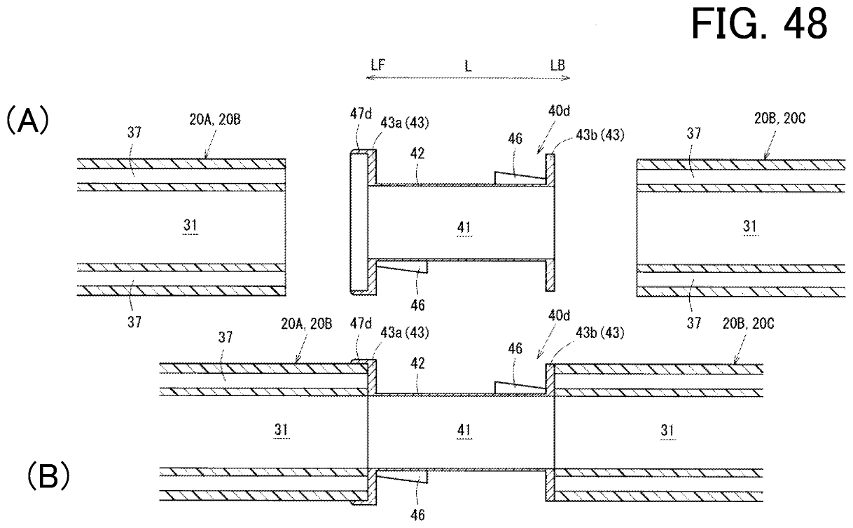

FIG. 46 shows another example of an explanatory view of a spacer 40d. FIG. 47 shows another example of an enlarged exploded perspective view of the main part of the movable elongated structure 10, and FIG. 48 shows another example of an explanatory diagram of a main part of the movable elongated structure 10.

As shown in FIG. 48, in the movable elongated structure 10 using the spacer 40d, the fitting ring 47d of the spacer 40d is attached to the outer circumference of the end of the flexible tube 20 on the distal end side LF on the base end side LB. is fitted from the base end side LB, and in a state where the flexible tube 20 on the distal end side LF and the spacer 40d are assembled, the distal end side flexible tube 20A, the spacer 40d, the intermediate flexible tube 20B, the spacer 40d and the base end side The flexible tube 20C is arranged in this order from the distal end side LF toward the base end side LB, and the traction wire 50 is inserted through the wire lumen 32 to complete the assembly.

Thus, the movable elongated structure 10 using the spacers 40d has the same effects as the movable elongated structure 10 using the spacers 40 described above. Furthermore, since the spacer 40 d fits the fitting ring 47 d to the end of the base end side LB of the flexible tube 20 on the distal end side IF, the position of the spacer 40 relative to the flexible tube 20 is regulated over the entire cross section of the flexible tube 20.

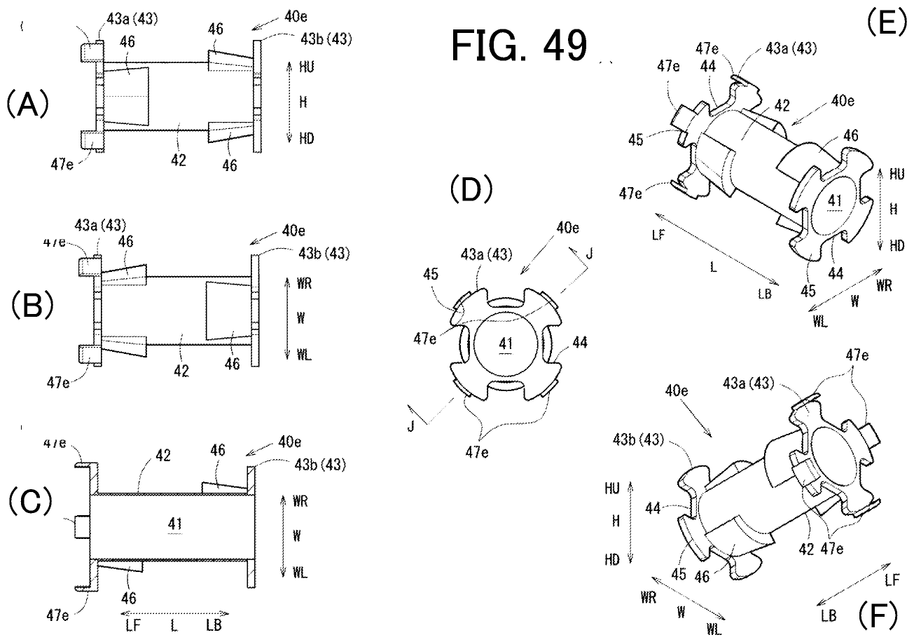

In the spacer 40d described above, a fitting ring 47d circularly protruding toward the distal end side LF from the outer peripheral edge of the distal end side flange portion 43a is fitted to the outer periphery of the base end side LB of the flexible tube 20 on the distal end side LF. However, instead of the fitting ring 47d, as shown in FIGS. 49 to 51, a plurality of fitting pieces 47e may be fitted to the outer circumference of the base end side LB of the flexible tube 20 on the distal end side LF.

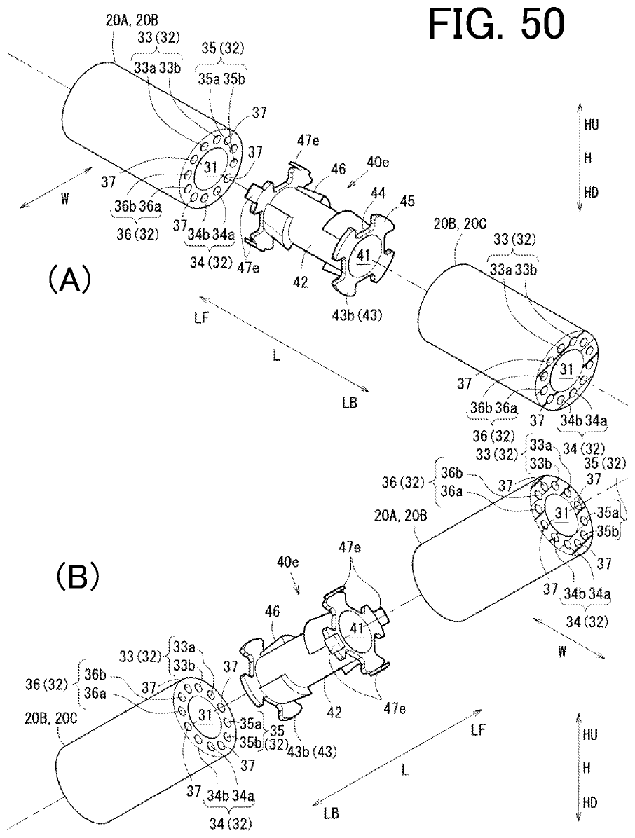

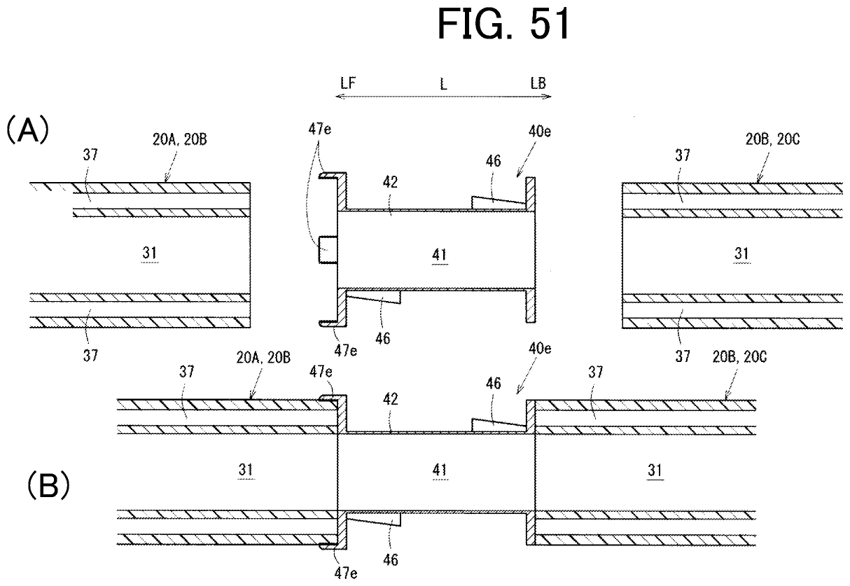

FIG. 49 shows another example of an explanatory view of a spacer 40e, FIG. 50 shows another example of an enlarged exploded perspective view of the main part of the movable elongated structure 10, and FIG. 51 shows an explanatory view of the main part of the movable elongated structure 10 of another example.

The fitting ring 47d of the spacer 40d described above is formed in a circular ring shape, but the spacer 40e is a segment protruding toward the distal end side IF along the outer peripheral edge of the regulation part 45 of the distal end side flange portion 43a. A plurality of fitting pieces 47e are provided at predetermined intervals in the circumferential direction. The spacer 40e illustrated in FIGS. 49 to 51 is provided with fitting pieces 47e for the four regulation parts 45, but any number of fitting pieces 47e may be provided as long as the number is two or more.

Similar to the spacer 40d having a fitting ring 47d, the spacer 40e having the fitting piece Ole is fitted to the outer circumference of the end of the flexible tube 20 on the base end side LB on the distal end side LF. Then, it is assembled with the flexible tube 20 to form the movable elongated structure 10.

The movable elongated structure 10 using the spacer 40e having the fitting piece 47e configured in this way has the same effects as the movable elongated structure 10 using the spacer 40d having the fitting ring 47d described above. may be played.

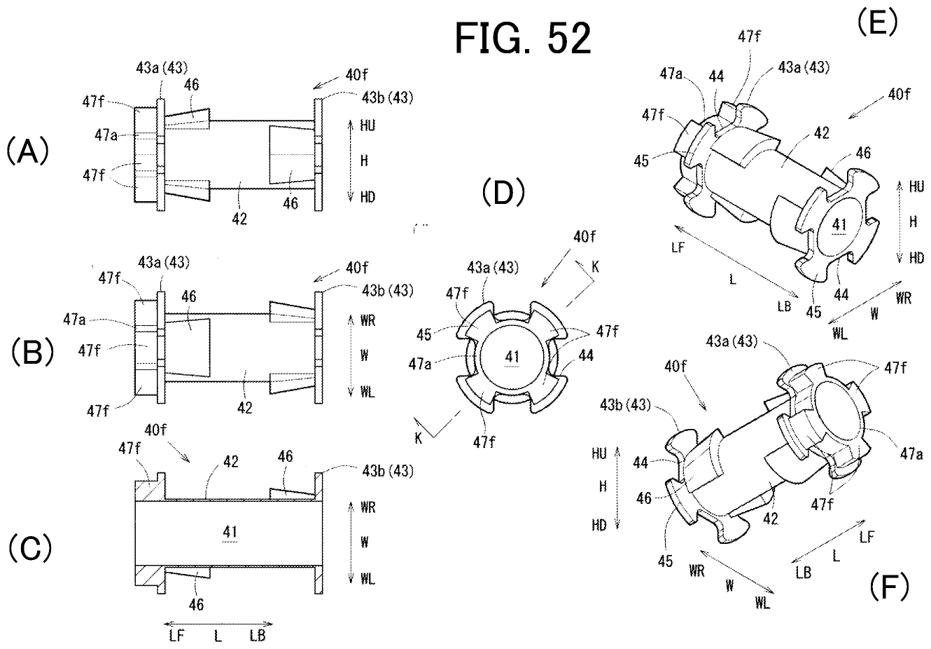

In the spacer 40 described above, the position regulation convex portion 47 protruding toward the distal end side LF from the distal end side flange 43a is inserted into the position regulation lumen 37. As shown in FIGS. 52 to 54 described later, a protruding block 47f is provided that protrudes toward the distal end side LF along the internal space 41 from the distal end side flange 43a, and the projecting block 47f is inserted into the insertion recess 38 provided at the end of the flexible tube 20 on the distal end side LF 47f may be inserted.

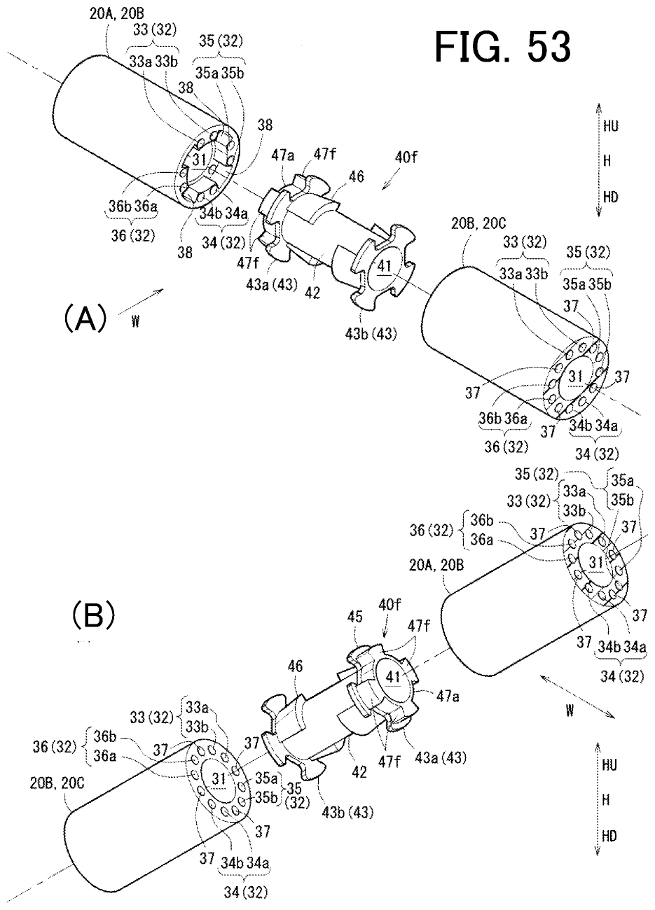

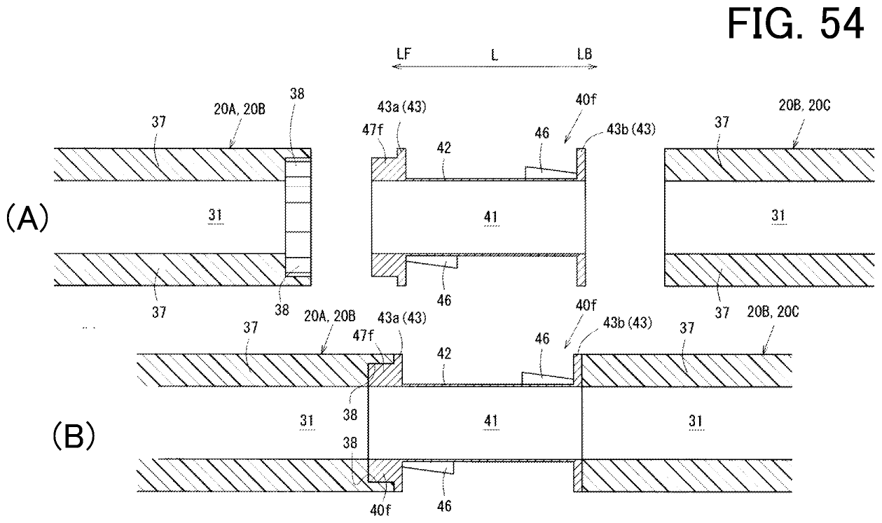

FIG. 52 shows another example of a spacer 40f, FIG. 53 shows another example of an enlarged exploded perspective view of the main part of the movable elongated structure 10, and FIG. 54 shows an explanatory diagram of a main part of the movable elongated structure 10.

The spacer 40f includes a protruding ring 47a similar to the spacer 40a and a protruding block 47f, which is provided radially outwardly of the protruding ring 47a and has a generally sectoral shape when viewed from the side and is slightly smaller than the regulation part 45, at the distal end side flange 43a.

An insertion recess 38 into which a protruding block 47f is fitted is provided between the wire lumens 32 at the end of the base end side LB of the flexible tube 20 on the distal end side LF to which the spacer 40f is assembled. The insertion recess 38 is a space having the same shape as the projecting block 47f.

The projecting block 47f and the insertion recess 38 are provided in four directions corresponding to the regulation part 45.

Similar to the spacer 40a having the protruding ring 47a, the spacer 40f having the protruding block 47f has the protruding ring 47a inserted into the main lumen 31 of the flexible tube 20 on the distal end side LF and the protruding block 471 inserted into the insertion recess 38. Then, it is assembled with the flexible tube 20 to form the movable elongated structure 10.

The movable elongated structure 10 using the spacers 40f having the projecting blocks 471 configured in this manner has the same effects as the movable elongated structure 10 using the spacers 40a having the projecting rings 47a described above.

In the spacer 40a described above, the protruding ring 47a protruding from the distal flange 43a toward the distal end side LF is inserted into the main lumen 31. In addition, an insertion pipe 49 is provided that protrudes toward the distal end side LF along the interior space 41 from the distal end side flange portion 43a, and part of the insertion pipe 49 is inserted into the main lumen 31 of the flexible tube 20 on the distal end side LF.

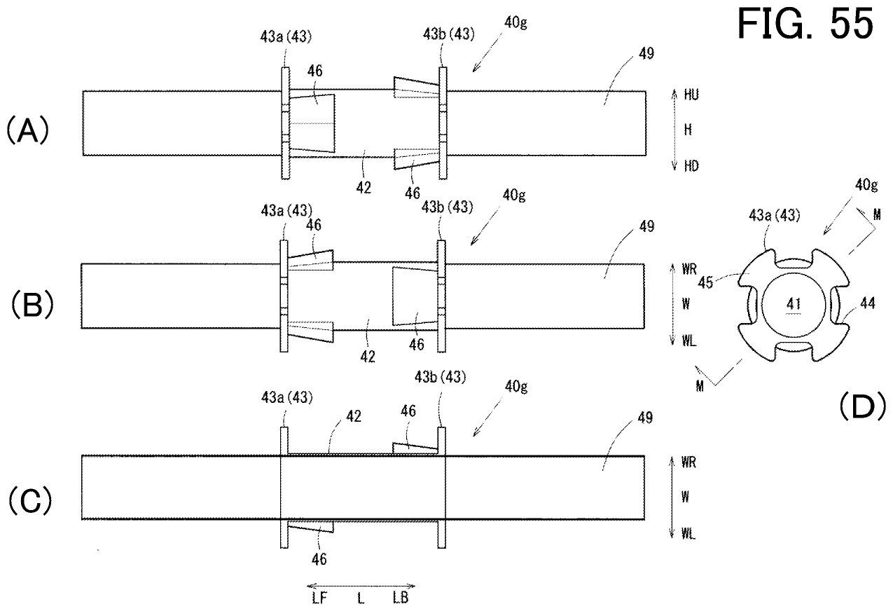

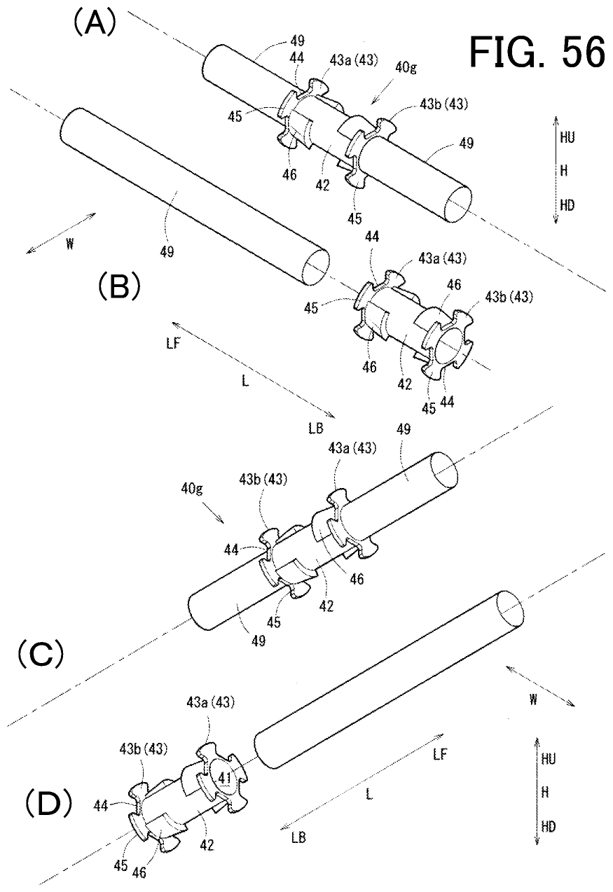

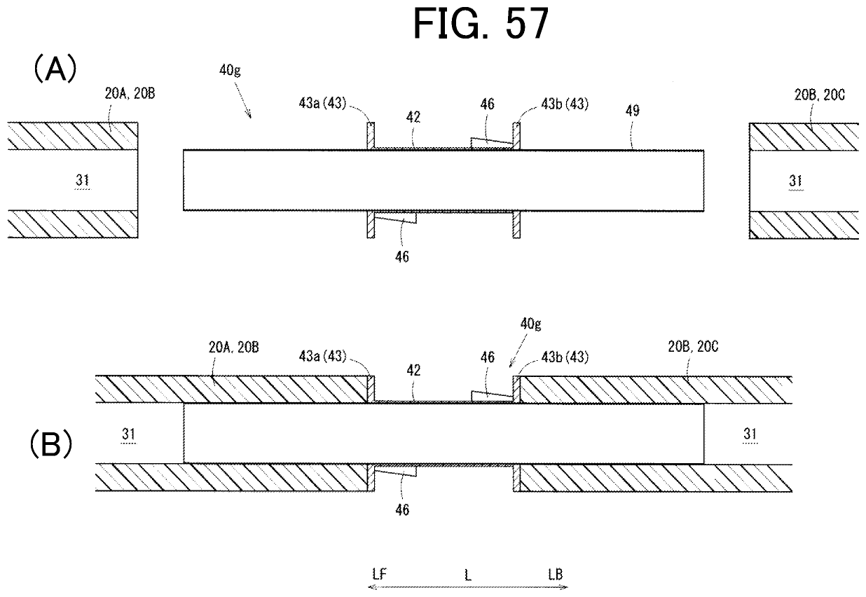

FIG. 55 shows another example of a spacer 40g, FIG. 56 shows another example of an enlarged perspective view of the essential parts of the spacer 40, and FIG. 57 shows an explanatory diagram of a main part of the elongated structure 10.

The spacer 40a is provided with a protruding ring 47a that protrudes toward the distal end side LF along the internal space 41, but the spacer 40g is provided with an insertion pipe 49 that is constructed separately instead of the above-described protruding ring 47a.

More specifically, the spacer 40g has a structure in which the projecting ring 47a is removed from the spacer 40a, and includes a cylinder 42 having an internal space 41 and flange portions 43 (43a, 43b) provided on both sides in the longitudinal direction L of the cylinder 42, and a guide convex part 46 provided on the outer periphery of the cylinder 42 are integrally configured, and an insertion pipe 49 configured separately is provided.

The insertion pipe 49 is a pipe body having an outer diameter that allows it to be inserted into the inner space 41 and the main lumen 31, and an inner space penetrating in the longitudinal direction L, and is made of an elastic material that is elastically deformed by a predetermined external force.

The spacer 40g is assembled so that the insertion pipe 49 is inserted into the internal space 41 and protrudes outward in the longitudinal direction L by approximately the same length from the distal end side flange 43a and the base end side flange 43b.

A spacer 40g having an insertion pipe 49 is assembled with the flexible tube 20 by inserting the projecting insertion pipe 49 into the main lumen 31 of the flexible tube 20 on the distal end side LF and the base end side LB to forma movable elongated structure 10.

The movable elongated structure 10 using the spacer 40g having the insertion pipe 49 configured in this manner has the same effects as the movable elongated structure 10 using the spacer 40a having the projecting ring 47a. Further, the movable elongated structure 10 using the spacer 40g having the insertion pipe 49 has an insertion pipe projecting from the flange portion 43 into the main lumen 31 of the flexible tube 20 on both the distal end side LF and the base end side LB. 49 is inserted, the position is restricted with respect to the flexible tubes 20 on both sides in the longitudinal direction L.

In the spacer 40g having the insertion pipe 49, the insertion pipe 49 is projected on one side in the longitudinal direction L from the flange portion 43, and the insertion pipe 49 is inserted into the main lumen 31 of the flexible tube 20 on the side where the insertion pipe 49 projects. may be inserted to regulate the position.

Further, when the length of the insertion pipe 49 protruding in the longitudinal direction L from the flange 43 is short, that is, when the length of the flexible tube 20 inserted into the main lumen 31 is short, the flexible tube 20 is not flexible. The inserting pipe 49 may be made of the same material as the spacer 40 so that the effect on the performance is reduced.

In addition, in the spacer 40g described above, the insertion pipe 49 is constructed separately and is inserted into the internal space 41. The flange 43 may be fixed. Even in this case, the same effect as the spacer 409 described above may be obtained, and the number of parts may be reduced from that of the spacer 40g.

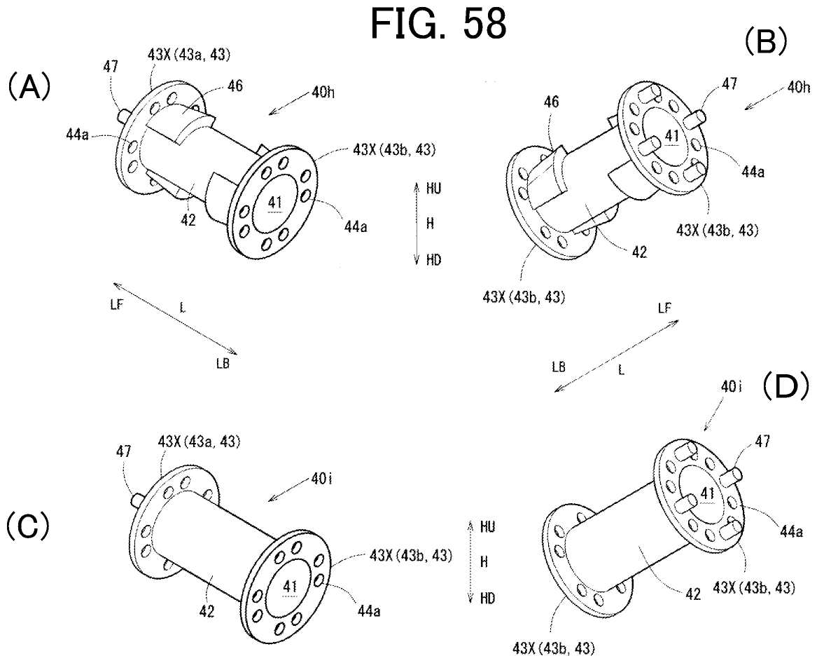

In the spacer 40 described above, the position regulating convex portion 47 protruding toward the distal end side LF from the distal end side flange 43a of the flange 43 in which the arrangement concave portions 44 and the regulation parts 45 are alternately arranged in the circumferential direction is positioned on the distal end side LF. However, as shown in FIG. 58 at (A) and (B), instead of the arrangement recess 44, the distal end side composed of the flange portion 43X having two through holes 44a. A spacer 40h having a position regulating convex portion 47 on the flange portion 43a may be used. Furthermore, as shown in FIG. 58 at (C) and (D), the spacer 40i may be a spacer 40h that does not include the guiding projections 46.

The spacers 40h and 40i configured in this way may achieve the same effect as the spacer 40 provided with the above-described position regulating convex portion 47 even if the flange 43 is the flange 43X.

Of course, the same effect may be obtained even if the spacer 40 is configured by the flange portions 43 in which the arrangement recesses 44 and the regulation parts 45 are alternately arranged In the circumferential direction, and the guide convex part s 46 are not provided.

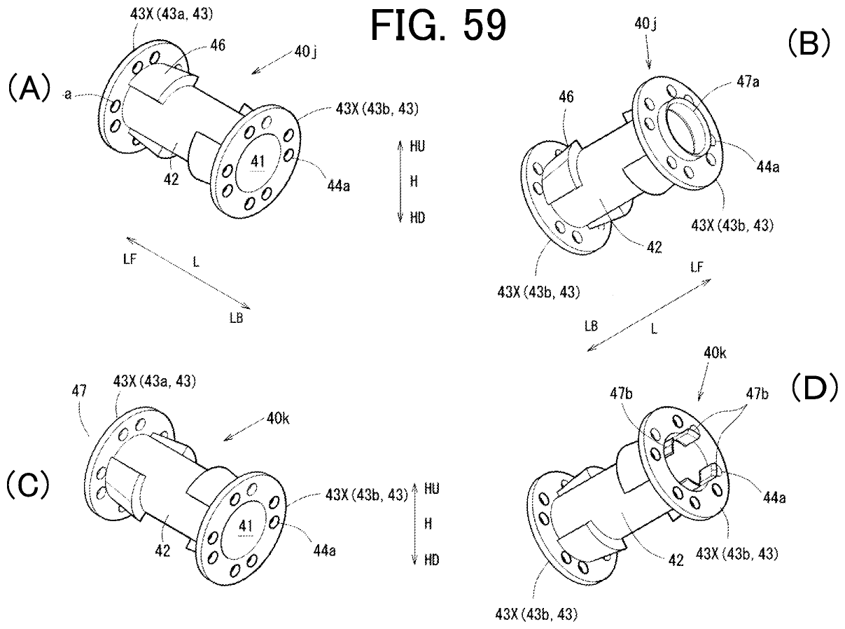

In the spacer 40 described above, the protruding ring 47a that protrudes toward the distal end side LF from the distal end side flange 43a of the flange 43 in which the arrangement recesses 44 and the regulation parts 45 are alternately arranged in the circumferential direction is the main portion of the distal end side LF. Although it was inserted into the lumen 31, as shown in FIG. 59 at (A) and (B), may be provided in the spacer 40j. Furthermore, as shown in FIG. 59 at (C) and (D), the spacer 40k may be a spacer 40k in which a protruding piece 47b is provided on the distal end side flange 43a constituted by a flange 43X having a through hole 44a.

The spacers 40j and 40k configured in this way may achieve the same effect as the spacer 40a having the projecting ring 47e described above even if the flange portion 43 is the flange portion 43X.

Of course, even if the spacers 40a, 40b. 40j, and 40k are not provided with the guide convex part s 46, the same effect may be obtained.

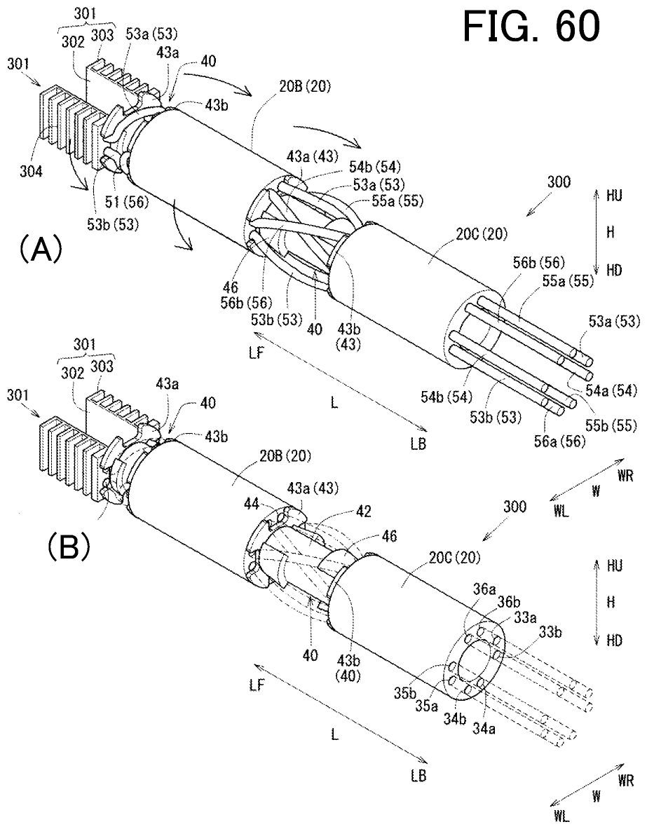

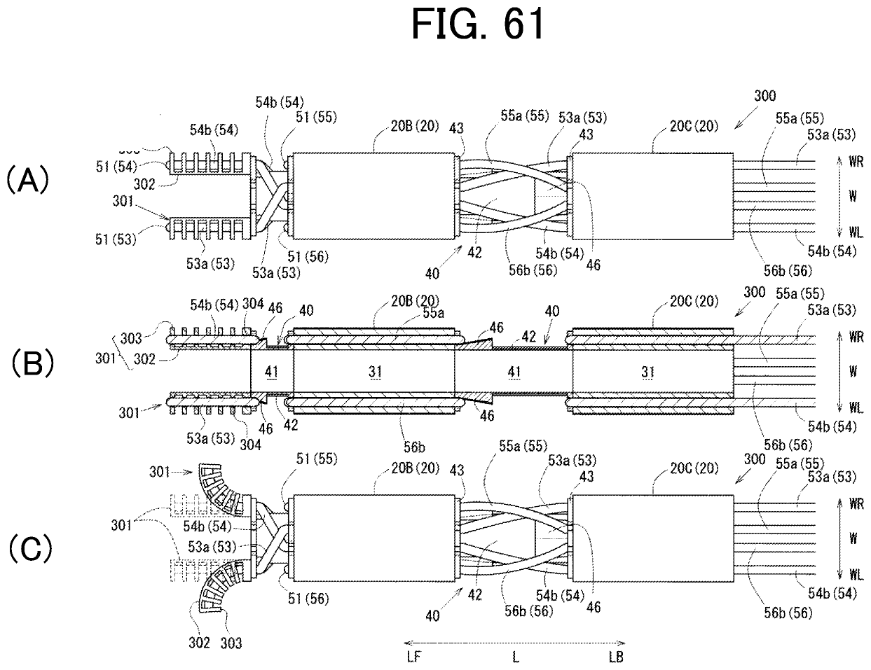

In addition, as shown in FIGS. 60 and 61 to be described later, the retractor 300 may be constructed using the movable elongated structure 10.

FIG. 60 shows a schematic illustration of the retractor 300. As shown in FIG. 60 at (A) shows a perspective view of retractor 300, and FIG. 60 at (B) shows a perspective view of retractor 300 with traction wire 50 shown in a see-through state.

FIG. 61 shows a schematic illustration of the retractor 300. As shown in FIG. 61 at (A) shows a plan view of retractor 300, FIG. 61 at (B) shows a cross section through the upper wire lumen, and FIG. 61 at (C) shows a plan view of retractor 300 with elastic retractor 301 in the open state.

The refractor 300 utilizes the movable elongated structure 10 described above, and has an elastic retractor 301 on the distal end side LF of the spacer 40 instead of the distal end side flexible tube 20A in the movable elongated structure 10.

Specifically, the movable elongated structure 10 shown in FIG. 32 is used with the width direction W being the height direction H.

Note that the spacer 40 in the retractor 300 has a shorter length in the longitudinal direction L of the cylinder 42 than the spacer 40 in the movable elongated structure 10 on the distal end side LF, but other configurations are the same. be. Also, the wiring of the traction wire 50 is the same as the wiring in the movable long structural body 10, so the description is omitted.

Two elastic retractors 301 are fixed to the front end side LF of the spacer 40 so as to face each other in the width direction W.

The elastic retractor 301 is composed of an elastic body 302 extending toward the distal end side LF and a plurality of plate portions 303 probing outward in the width direction W from the elastic body 302.

The elastic body 302 is a rectangular plate having a predetermined thickness that is longer in the longitudinal direction L than in the height direction H. and the plate portion 303 is a rectangular plate having a predetermined thickness that is longer in the height direction H than in the width direction W. It is a plate of a plurality of plate portions 303 are arranged at predetermined intervals in the longitudinal direction L, integrally configured with the elastic body 302, and are longer in the height direction H than in the width direction W and longer in the longitudinal direction L than in the height direction H. It is formed in a rectangular parallelepiped shape.

A plurality of plate portions 303 arranged at predetermined intervals in the longitudinal direction L correspond to the wire lumens 32 (33, 34) of the distal end-side flexible tube 20A in the movable elongated structure 10, and pull wires 50 (54, 53) are formed through holes 304.

The elastic retractor 301 configured in this way faces the front end side LF of the spacer 40 at a predetermined interval in the width direction W, that is, the direction in which the plate portion 303 protrudes from the elastic body 302 is the width direction W. It is arranged to be outside.

The traction portion 53 is inserted through the insertion hole 304 of the elastic retractor 301 on the left side WI, and the traction portion 54 is inserted through the insertion hole 304 of the elastic retractor 301 on the right side WR.

The retractor 300 configured in this manner pulls the retracting portions 55 and 56 arranged in the height direction H on the base end side LB to the base end side LB, thereby allowing the retractor 300 to be intermediately movable as indicated by the arrow in FIG. 30 at (A). The flexible tube 208 may be bent In the width direction W.

Specifically, the intermediate flexible tube 208 is pulled toward the base end side LB by puffing the traction portion 56 inserted through the wire lumens 34b, 36a on the left side Wt. of the wire lumens 34, 36 of the base end side flexible tube 20C. It may be bent to the left side WL.

Conversely, by pulling the traction portion 55 inserted through the right WR wire lumens 34a, 38b in the wire lumens 34, 36 of the base end side flexible tube 20C toward the base end side LB, the intermediate flexible tube 208 is pulled toward the right WR.

By canceling the pulling of the traction portions 55 and 56, the elastic force of the intermediate flexible tube 20B cancels the bending of the traction portions 55 and 56 due to the pulling.

The elastic retractor 301 may be opened by pulling the traction portions 53 and 54 inserted through the wire lumens 33 and 35 of the base end side flexible tube 20C in the width direction W on the base end side LB.

Specifically, by pulling the traction portion 53 inserted through the insertion hole 304 of the elastic retractor 301 of the left side WL, the left side WI is pulled as indicated by the arrow in FIG. 60A without bending the intermediate flexible tube 20B. The elastic retractor 301 may be bent outward in the width direction W to the left side is WL.

Conversely, by pulling the traction portion 54 inserted through the insertion hole 304 of the elastic retractor 301 of the right WR, the elastic retractor of the right WR may be pulled as Indicated by the arrow in FIG. 60 at (A) without bending the intermediate flexible tube 20B, 301 may be bent to the right side WR, which is the outside in the width direction W.

Therefore, when the traction portion 53 and the traction portion 54 are pulled at the same time, the elastic retractors 301 on both sides in the width direction W are bent outward in the width direction W as shown in FIG. 61 at (C). It may be served as a retractor to open the site.

By canceling or releasing the traction of the traction portion 53 and/or the traction portion 54, the bending due to the traction of the traction portion 53 and/or the fraction portion 54 is canceled by the elastic restoring force of the elastic retractor 301 (302).

Furthermore, in the retractor 300 described above, the elastic retractor 301 in which the plate portion 303 is provided outside the elastic body 302 in the width direction W is used. By using the elastic retractor 301 provided with the plate portion 303 on the inner side in the width direction W, the elastic retractor 301 may be served as a forceps that bends inward in the width direction W by pulling the traction portion 53 and/or the traction portion 54.

Further, in the movable elongated structure 10 described above, the distal end side flexible tube 20 at (A), the spacers 40 (40a to 40k), the intermediate flexible tube 20B, the spacers 40 (40a to 40k), and the base end side flexible tube 20C are Although it is arranged in this order from the distal end side LF to the base end side LB, the distal end side flexible tube 20 at (A), the spacers 40 (40a to 40k), and the base end side flexible tube 20C are arranged from the distal end side LF to the base end side. They may be arranged in this order toward the side LB. In this case, the wiring method of the traction wire 50 with respect to the wire lumen 32 is the same as that of the prior art, so detailed description thereof will be omitted. In this way, the movable elongated structure 10 composed of the distal end side flexible tube 20A, the spacers 40 (40a to 40k), and the base end side flexible tube 20C is the same as the movable elongated structure 10 described above, whereby effects similar to those of the movable elongated structure 10 described above may be obtained.

A manipulator 100, which is another example of a medical device using the movable elongated structure 10 of the present embodiment shown in FIG. 62, will be described below. Here, an example using the movable elongated structure 10 in which the distal end side flexible tube 20A, the spacer 40 and the base end side flexible tube 20C are arranged in this order will be described, but the present invention is not limited to this. Note that FIG. 62 shows a schematic view of a manipulator 100, which is a medical device in another example using the movable elongated structure 10 of the present invention.

The manipulator 100 is a medical device that is inserted into a branched channel such as a blood vessel, a lumen, or inside a hollow organ, and performs a predetermined treatment after the tip reaches a predetermined location. A manipulator body 101 having a grip and a movable elongated structure 10 extending from the tip of the manipulator body 101 to the distal end side LF are provided.

The manipulator main body 101 includes a traction drive unit 102 for pulling a traction portion 52 (not shown) of a traction wire 50 (not shown) extending from the base end side LB of the movable elongated structure 10, and a traction drive unit 102. The operation handle 103 (103a. 103b) for manually operating the bending direction of the movable elongated structure 10 by puling the traction portion 52, and how the eight traction portions 52 are pulled by the manual operation of the operating handle 103. A control unit 104 is provided to control whether the driving unit 102 is towed.

The traction driving section 102 and the control unit 104 are arranged inside the manipulator main body 101, and the operating handle 103 is arranged outside the manipulator main body 101.

The operating handle 103 includes a vertical operating handle 103a for bending the movable elongated structure 10 in the height direction H, and a width direction operating handle 103b for bending it in the width direction.

The operator may bend the distal end side LF of the movable elongated structure 10 in the upward direction HU or the downward direction HD in the height direction H by manually operating the vertical direction operation handle 103a. It may be bent by a desired bending amount by the operation amount of 103a.

In addition, the operator may bend the movable elongated structure 10 to the right or left in the width direction by manually operating the width direction operation handle 103b, and may bend it by a desired amount depending on the operation amount of the width direction operation handle 103b.

Then, the operator manually operates both the vertical operation handle 103a and the width direction operation handle 103b at the same time or sequentially to move the distal end side LF of the movable elongated structure 10 in the height direction H and width direction. It may be bent in an oblique direction crossing the direction, and may be bent in a desired bending direction in all directions depending on the amount of operation of the vertical operation handle 103a and the width direction operation handle 103b.

Any of the spacers 40 (40a to 40k) may be used for the movable elongated structure 10 used in the manipulator 100 described above. It is preferable to provide a traction drive unit 102 and an operation handle 103 corresponding to the bending direction.

Further, if necessary, the operation handles 103a and 103b may be provided in separate units and connected to the manipulator main body 101 by wire or wirelessly.

The length of the movable elongated structure 10 is longer than that of the manipulator main body 101, and the manipulator 100 may be operated by inserting a treatment tool or the like into the main lumen (31) and the internal space (41) as a channel. It may be served as a steering catheter operable with the operating handle 103.

In addition, the length of the movable elongated structure 10 is formed longer than the manipulator body 101, and a treatment instrument or the like is inserted into the main lumen 31 and the internal space 41 as a channel, and for example, the distal end side flexible tube 20A is inserted. The manipulator 100 may be served as a flexible endoscope that may be operated with the operation handle 103 by mounting a camera as an image receiving unit on the distal end side of the manipulator 100 or the distal end cap 60.

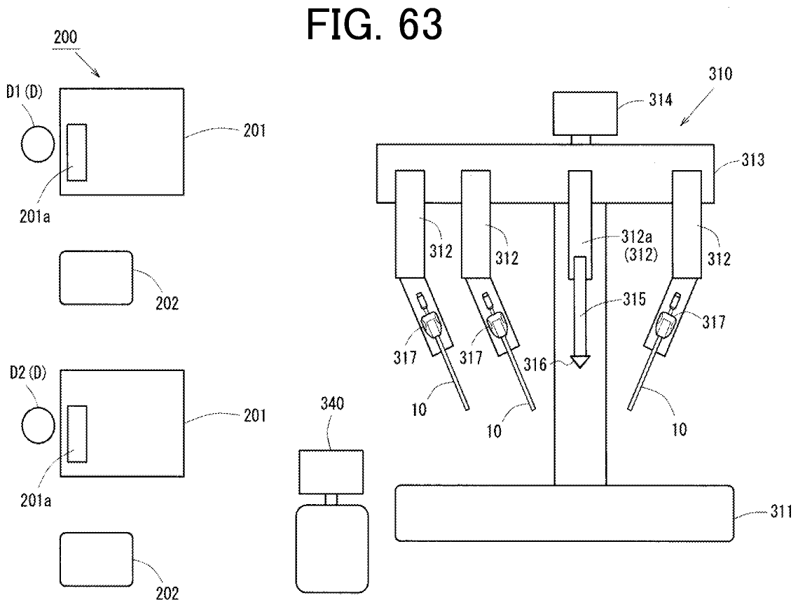

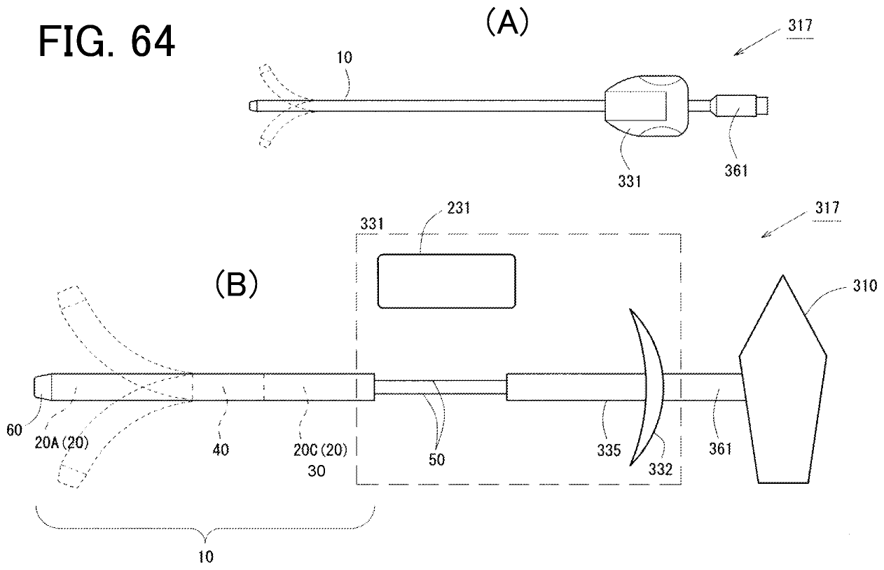

Next, a telesurgery system 200 according to this embodiment will be described with reference to FIGS. 63 and 64.

FIG. 63 shows a schematic diagram of the telesurgery system 200 in another example, and FIG. 64 shows a schematic explanatory diagram of the tool 317 in the telesurgery system 200. Specifically, FIG. 64 at (A) shows a plan view of a tool 317 that may be loaded onto the robotic arm assembly of telesurgical system 200, and FIG. 64 at (B) shows the internal configuration of tool 317.

The telesurgery system 200 includes a surgeon console 201 serving as a station for each of two operators D (D1, D2), a master control unit 202 operated by operator D, a viewing and core cart 340, and a patient side cart 310. Have a robot.

The surgeon's console 201 includes a viewer 201a on which an image of the surgical site is displayed to the operator D. When using the surgeon console 201, operators D1 and/or D2 typically sit in the chair of the surgeon console, have their eyes in front of the viewer 201a, and hold the master control unit 202 with one or both is hands.

Although the telesurgery system 200 may be operated by two operators simultaneously, it may also be operated by one operator. When two operators perform operations simultaneously, cooperative operations by the two operators are possible, and there is an advantage that the operation time for the entire patient may be shortened. The surgeon's console 201 and the master control unit 202 may be configured in a system in which three or more units each are provided, if necessary.

The patient-side cart 310 robot is placed adjacent to the patient. In use, the patient side cart 310 is placed near the patient requiring surgery. The patient-side cart 310 robot has casters on the base 311 so that it is fixed but movable during the surgical procedure. Surgeon console 201 is used in the same operating room as the patient-side cart, but may be located remotely from patient-side cart 310.

The patient-side cart 310 includes four robotic arm assemblies 312, although the number of robotic arm assemblies 312 is arbitrary. Each robot arm assembly 312 is connected to a driving device 313 that enables three-dimensional movement and is driven and controlled.

A display 314 displays image data associated with the surgery. Drive 313 is controlled by master control unit 202 of surgeon console 201. Movement of the tool 317 of the robot arm assembly 312 is controlled by manipulation of the master control unit 202.

One robot arm assembly 312a of the four robot arm assemblies 312 is provided with an image capture device 315 such as an endoscope. A viewing camera 916 is included at the remote end of the image capture device 315. An elongated shaft-like image capture device 315 allows a viewing camera 316 to be inserted through a surgical entry port of a patient (not shown).

Image capture device 315 is operatively connected to viewer 201a of surgeon console 201 for displaying images captured by viewing camera 316 thereof.

Each of the other robotic arm assemblies 312 is a linkage that respectively supports and includes a removable surgical instrument, tool 317. It should be noted that a viewing camera may be mounted In a portion of the main lumen 31 of the movable elongated structure 10 of the tool 317 of one or more robot arm assemblies 312 and used instead of the viewing camera 316 of the image capture device 315.

The tool 317 comprises an elongated movable elongated structure 10 to allow insertion through a patient's surgical entry port. Movement of movable elongated structure 10 is controlled by master control unit 202 of surgeon console 201. The movable elongated structure 10 utilizes the movable elongated structure 10 of the previous embodiment.

FIG. 64 shows a configuration of a tool 317 that may be loaded onto the robot arm assembly 312 of the telesurgery system 200 of FIG. 63 as a representative example of a surgical device. Tools 317 attached to other robotic arm assemblies 312 may be of similar construction or may be surgical devices of other constructions.

A tool 317 shown in FIG. 64 at (A) has a movable elongated structure 10 having a traction wire 50, a surgical device 331 for driving control and monitoring of the tool 317, and a connector 361 for coupling to a robot. The surgical device 331 constitutes a traction driver that drives the traction wire 50 within the movable elongated structure 10.

As shown in FIG. 64 at (8) illustrating the internal configuration of tool 317, a surgical device 331 that drives a tool 317 via a shaft 335 that Is directly coupled to the robotic arm assembly 312 of FIG. 63, and a robot on patient side cart 310 controlling surgical device 331 which constitute a medical system.

The movable elongated structure 10 has a base end side flexible tube 20C connected to the shaft 335, a wiring aid 40, and a distal end side flexible tube 20A as an end effector. Since the movable elongated structure 10 has a bent structure as in the above example, the degree of freedom In the operating angle of the distal end side flexible tube 20A is increased, and the applicable range of surgery is improved.

The surgical device 331 of the tool 317 has a control circuit 231 that controls signals within the surgical device and a signal interface 310a with the robot of the patient cart 310.

The control circuit 231 is configured to control a driving mechanism (not shown) that drives a predetermined traction wire 50 of the movable elongated structure 10 based on a control signal from the robot 310.

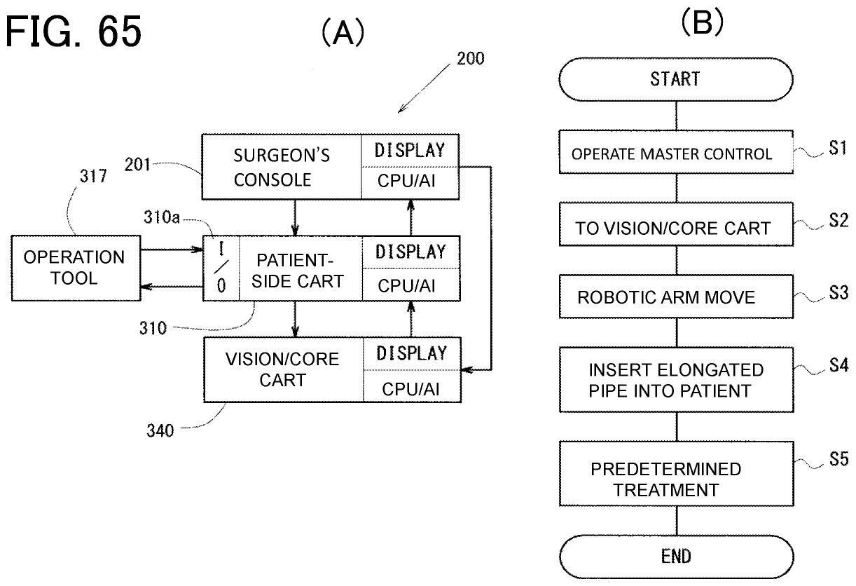

As shown in FIG. 65, the robot of the cart 310 on the patient side is routed and/or wirelessly connected to the surgical device 331 and the signal interface 310a via the connector 361. Internally, there are an input unit that receives an operation signal from the master control unit 202, an arithmetic unit CPU that executes a predetermined operation program based on the operation signal, and a surgical device 331 based on the output from the arithmetic unit. and an output unit for generating a drive signal for driving the movable elongated structure 10 of the tool 317. The input unit and the output unit are composed of an input/output unit 310a (I/O).

FIG. 65 is an explanatory diagram of the telesurgery system 200, FIG. 65 at (A) is a block diagram showing the connection relationship with each unit, and FIG. 65 at (B) is an operation flow diagram of the telesurgery system 200.

In addition, in the robot of the patient-side cart 310 shown in FIG. 65, artificial intelligence (AI) may be provided in the arithmetic unit CPU that executes a predetermined operation program based on the operation signal. The robot of the patient-side cart 310 equipped with artificial intelligence (AI) in the arithmetic unit CPU is provided with various sensors, accumulates and evaluates the detection results of the various sensors, and may control the tool 317 based on the evaluation results.

The vision and core cart 340 has functions associated with image capture equipment. When the telesurgery system 200 is activated for surgery, the surgeon operates the master control unit 202 of the surgeon's console 201, and if there are two surgeons, also operates the master control unit 202 of the surgeon's console 201 (step S1), the command generated by the operation is sent to the vision/core cart 340 (step S2).

The vision and core cart 340 then interprets the signals and causes movement of the desired robotic arm assembly 312 to the patient's surgical area (step S3).

Next, the movable elongated structure 10 of the tool 317 attached to the selected robot arm assembly 312 is inserted into the patient through an elongated pipe (step S4), and reaches a predetermined position by bending the distal end side flexible tube 20A. After that, predetermined treatment is performed (step S5) to complete the surgery on the living tissue.

It is preferable to provide a traction drive unit 102 corresponding to the bending direction, that is, the number of traction wires 50 (traction portions 52), and an operation handle 103 corresponding to the bending direction.

Since the telesurgery system 200 including the manipulator 100, the retractor 300, the tool 317, and the tool 317 described above includes the movable elongated structure 10, in addition to the effects of the movable elongated structure 10 described above. The effect obtained by each structure may be produced.

As described above, in the correspondence between the configuration of the present invention and the above-described embodiments, the longitudinal direction of the present invention corresponds to the longitudinal direction L, and so on.

Although the robot and the medical robot correspond to the patient-side cart 310, they are not limited to the above embodiments.

In addition, although the spacers 40 (40a to 40k) described above are provided with the position regulation convex parts 47 (47a to 47f, 47h to 47k) projecting toward the metal end side LF on the distal end side flange 43a of the distal end side LF, a position regulating convex portion 47 (47a to 47f, 47h to 47k) projecting toward the base end side LB may be provided on the base end side flange 43b of the base end side LB, or the tip of the distal end side LF may be provided. The position regulating projections 47 (47a to 47f, 47h to 47k) may be provided on both the side flange 43a and the base end flange 43b on the base end side LB.

Furthermore, when the position regulation convex parts 47 (47a to 47f, 47h to 47k) are provided on both the distal end side flange 43a of the distal end side LF and the base end side flange 43b of the base end side LB, Position control projections 47 (47a to 47f, 47h to 47k) of the same type or different types may be provided on the distal end side flange 43a and the base end side protrusion 43b of the base end side LB.

Although the traction wire 50 is served as the traction operation body, it may be belt-shaped. Moreover, although the single traction wire 50 is bent at the bent portion 51 to form a pair of try portions 53, a pair of separate traction wires 50 may be used. Further, when pulling a pair of traction wires 50 configured separately, one traction wire 50 may be pulled, or both traction wires 50 may be pulled. Also, although both traction wires 50 are pulled, each traction wire 50 may be pulled with a different pulling force.

As the flexible tube 20, a tube with a circular ring-shaped cross section having a main lumen (31) inside is used, but various ring shapes such as an elliptical ring-shaped cross section, a polygonal ring-shaped cross section such as a triangular diameter or a square, etc. are used. It may be a tubular body having a cross section, and the main lumen 31 may have a shape similar to the outer diameter of the cross-sectional shape as described above, or may have a different cross-sectional shape. Also, the main lumen 31 in the flexible tube 20 may be arranged at a position shifted from the center of the flexible tube 20.

Also, the flexible tube 20 may be configured with a backbone structure in which rigid bodies having a main lumen therein are connected via rotary joints or elastic bodies.

Also, telesurgical system 200 may be configured using retractor 300 instead of moveable elongated structure 10 in tool 317.

Furthermore, in the above description, the flexible tube 20, which is a tube having a cylindrical shape elongated in the longitudinal direction L and having a wire lumen 32 inside the tube wall, is used. A flexible tube made by weaving wear-resistant fibers, such as a flexible tube, may also be used.

In this case, two layers are formed of a small-diameter tube that forms the main lumen 31 and a large-diameter tube that forms the outer shape, and a wire lumen 32 is formed between the layers, and the gap between the layers is filled with a flexible resin. It will be configured as follows. Thus, even when a flexible tube constructed by weaving wear-resistant fibers is used, the same functions and effects as the movable elongated structure 10 using the flexible tube 20 described above may be obtained.

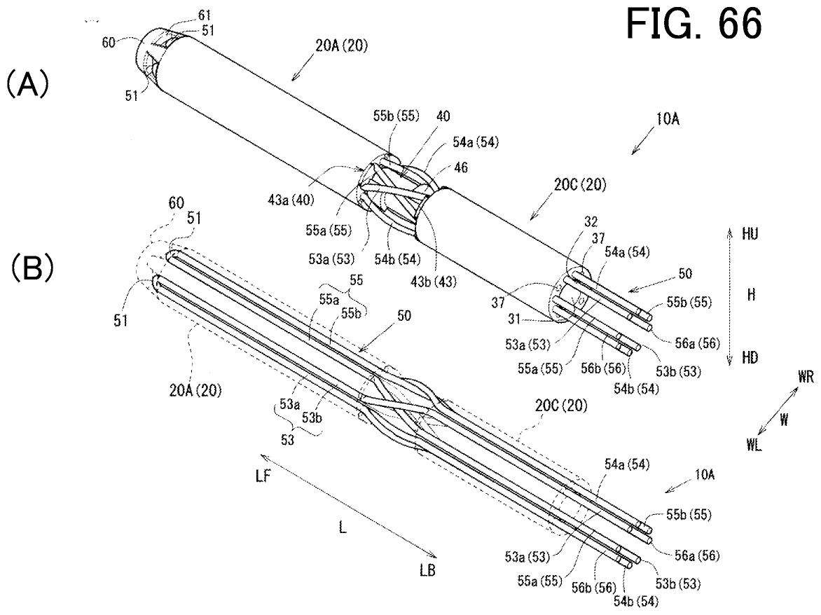

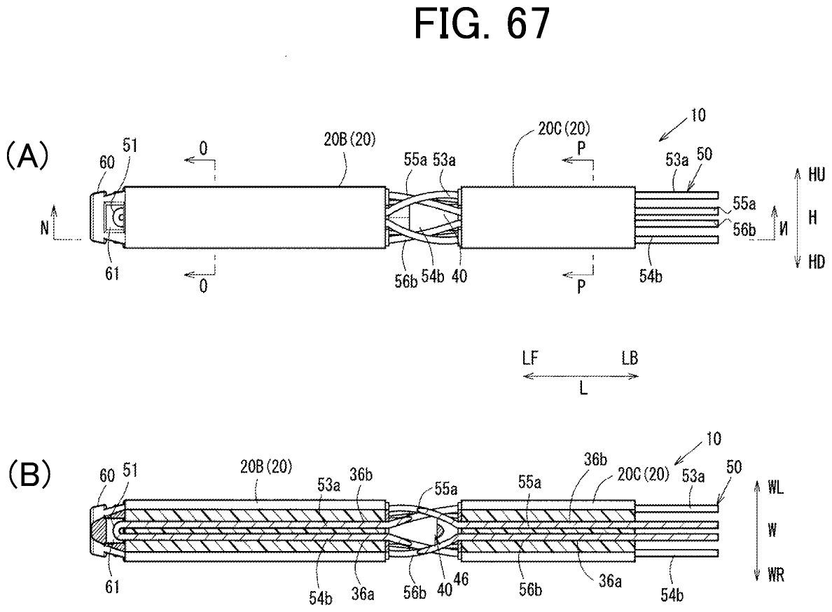

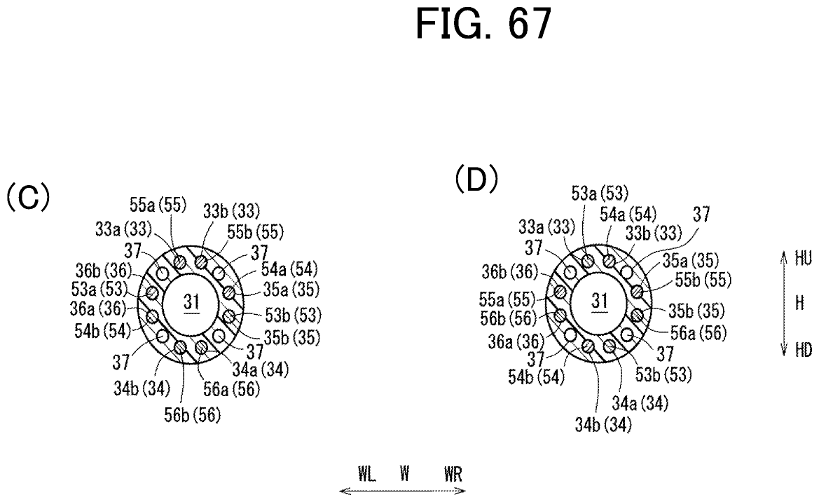

Further, the above-described movable elongated structure 10 may move the distal end side flexible tube 20A upward HU or downward HD with respect to the intermediate flexible tube 208 by pulling either the traction portion 53 or the traction portion 54, and by puling either the traction portion 55 or the traction portion 56 toward the base end side LB, the intermediate flexible tube 208 may be bent upward HU or downward with respect to the base end side flexible tube 20C. Although it may be bent in any of the directions HD, it may be bent in any of the four directions of the upward direction HU, the downward direction HO, the left side WL and the right side WR like the movable elongated structure 10A shown in FIGS. 66 and 67 which may be configured to bend.

The movable elongated structure 10A shown in FIGS. 66 and 67 is composed of the distal end side flexible tube 20A and the base end side flexible tube 20C in the movable elongated structure 10 described above, but the movable elongated structure It has the same configuration as the intermediate flexible tube 20B and the base end side flexible tube 20C in the elongated structure 10, and the distal end cap 6015 configured at the distal end of the distal end side LF of the intermediate flexible tube 208, The traction wires 50 are routed in the same manner as the wire lumens 32 of the intermediate flexible tube 20B and the base end side flexible tube 20C. The bent portions 51 are arranged in all of the bent concave portions 61 provided in the four directions in the height direction H and the width direction W of the tip end cap 60.

In the movable elongated structure 10A, one of the spacers 40, 40a to 40k is arranged between the distal end side flexible tube 20A and the base end side flexible tube 20C.

The movable elongated structure 10A configured in this way is pulled against the base end side flexible tube 20C by pulling either the traction portion 53 or the traction portion 54 of the traction wire 50 to the base end side LB. The dual end side flexible tube 20A may be bent to either right WR or left WL.

In addition, the movable elongated structure 10A may be flexible at the distal end side with respect to the base end side flexible tube 20C by pulling either the traction portion 55 or the traction portion 56 of the traction wire 50 to the base end side LB. The tube 20A may be bent either upward HU or downward HD.

In addition, the movable elongated structure 10A may be flexible at the distal end side with respect to the base end side flexible tube 20C by pulling either the traction portion 55 or the traction portion 56 of the traction wine 50 to the base end side LB. The tube 20A may be bent either upward HU or downward HD.

Furthermore, the movable elongated structure 10A may be pulled by pulling either the traction portion 53 or the traction portion 54 or the traction portion 55 or the fraction portion 56 to the base end side LB, thereby pulling the base end side flexible tube 20C. The distal end side flexible tube 20A may be bent obliquely in the height direction H and width direction W.

In this way, the movable elongated structure 10A in which any one of the spacers 40, 40a to 40k is arranged between the distal end side flexible tube 20A and the base end side flexible tube 20C is the movable elongated structure 10 in addition, as described above, the distal end side flexible tube 20A may be bent in the height direction H, width direction W, and oblique directions thereof.

As another embodiment of the present invention, a medical device will be described as an example of a surgical tool having a movable elongated structure that may be bent and deformed in a desired direction, but the present invention is not limited to medical devices.

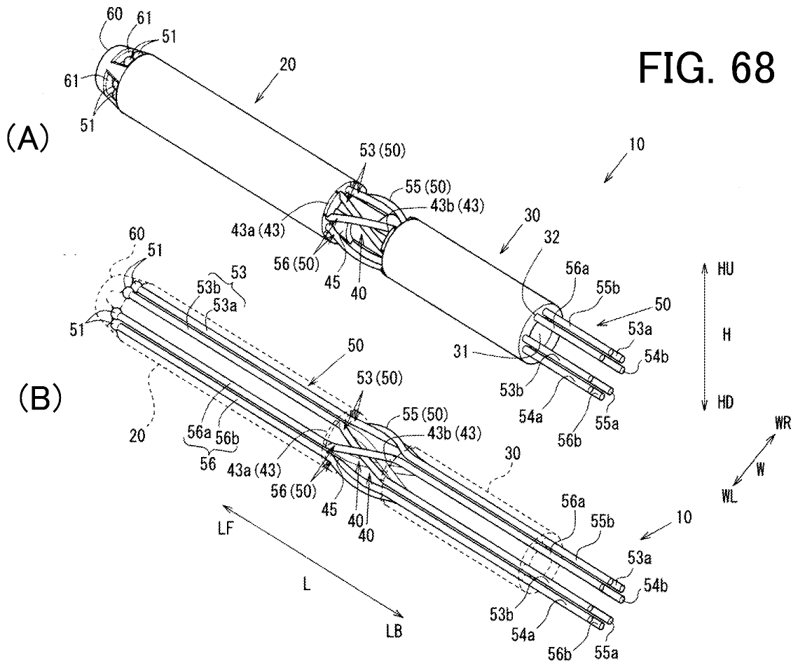

FIGS. 68 to 72 show structural diagrams of the movable elongated structure 10 of this embodiment.

Note that FIG. 71 at (A) is a developed view showing a portion passing through the center of the wire lumens 22, 32 by deploying the flexible tubes 20, 30 with annular cross sections along the virtual dividing line DL shown in FIG. 71 at (C) and (D).

The movable elongated structure 10 includes a distal end side flexible tube 20 and a base end side flexible tube 30 arranged along the longitudinal direction L, a distal end side flexible tube 20 and a base end side flexible tube 30 (to be summarized below). It is provided with a spacer 40 disposed between the flexible tubes 20 and 30 and a traction wire 50 inserted through the inside of the tube wall of the flexible tubes 20 and 30. The movable elongated structure 10 is covered along the longitudinal direction L with an exterior cover (not shown). Also, only the outside of the spacer 40 may be covered with an exterior cover (not shown).

The distal end side flexible tube 20 is a cylindrical flexible tube elongated in the longitudinal direction L, and has a main lumen 21 (FIG. 71 at (B) and (C)) inside. The main lumen 21 is a circular space along the longitudinal direction L in cross section.

In addition, as shown in FIG. 71 at (8) and (C), the distal end side wire lumen 22 (23a to 26a, 23b to 26b) is provided inside the tube wall between the main lumen 21 and the outer peripheral surface of the distal end side flexible tube 20.

The distal wire lumen 22 is a space with a circular cress section extending in the longitudinal direction L inside the tube wall, and is formed with a diameter that allows a traction wire 50 to be described later to be inserted therethrough.

Two distal wire lumens 22 are provided in each of the four directions (up, down, left, and right directions) on the tube wall having a ring-shaped cross section.

Specifically, as shown in FIG. 71 at (B) and (C), in the pipe wall having a ring-shaped cross-section, distal end side wire lumens 23a and 23b in the upward direction HU, distal end side wire lumens 24a and 24b in the downward direction HD, and distal end side of the right WR Wire lumens 25a, 25b and wire lumens 26a, 26b on the distal end side of the left WL are provided at predetermined intervals in the circumferential direction of the circular cross section.

When viewing the movable elongated structure 10 from the base end side LB to the distal end side IF, the clockwise sides of the distal end side wire lumen 22 formed by a pair of two through holes are indicated by 23a 24a. 25a, and 26a. and 23b, 24b, 25b, and 26b on the counterclockwise side.

Note that the distal end side wire lumens 22, which are provided in four directions (up, down, left, and right), may be integrally formed into an elliptical shape.

Among the distal end side wire lumens 22 configured in this way. the pair of distal end side wire lumens 23a and 23b provided in the upward direction HU on the tube wall having a ring-shaped cross section are collectively referred to as a distal end side upper lumen set 23, and similarly, The distal end side wire lumens 24a and 24b in U) the downward direction HD are the distal end side lower lumen set 24, the distal end side wire lumens 25a and 25b in the right side WR are the distal end side right lumen set 25, and the distal end side wine lumens 26a and 26b in the left side WL are the tip. A side left lumen set 26 is provided.

The distal end side flexible tube 20 configured as described above may be configured from a flexible tube such as polyamide elastomer, expanded polytetrafluoroethylene, polyurethane, or polytetrafluoroethylene.

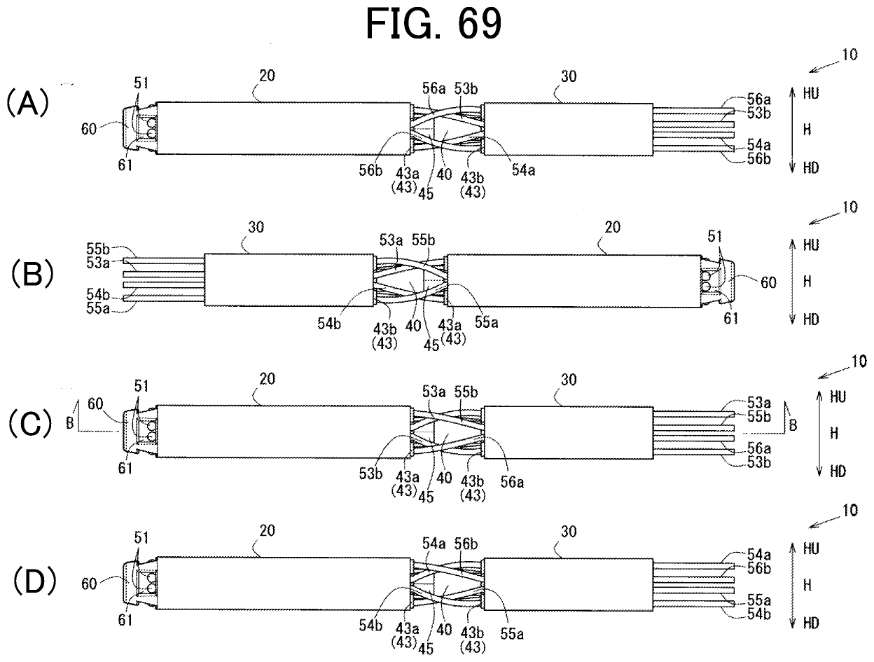

As shown in FIGS. 69 and 71, a distal cap 60 is provided at the distal LF end of the distal end side flexible tube 20.

The distal end cap 60 has a fixing recess 61 for forming an enlarged diameter portion 51 of the traction wire 50, which will be described later. The fixing recesses 61 are provided at four locations corresponding to the distal wire lumens 22 provided in four directions (up, down, left, and right) on the tube wall, and are provided with two insertion holes through which the traction wires 50 are inserted.

Note that, as shown in FIG. 71 at (8), the distal end cap 60 serves as a main lumen formed by connecting the main lumen 21, the internal space 41 described later, and the base end side main lumen 31 in a device using the movable elongated structure 10. A through hole 62 is provided in the longitudinal direction L by an instrument or the like to be inserted, but it may not be provided. An endoscope may be provided by inserting a camera as an insertion tool into the through hole 62.

Like the distal end side flexible tube 20, the base end side flexible tube 30 is a cylindrical flexible tube elongated in the longitudinal direction L and has a base end main lumen 31 therein. The base end side main lumen 31 is a space having a circular cross section along the longitudinal direction L.

As shown in FIG. 69 at (D), a base end wire lumen 32 (33 to 36) is provided inside the tube wall between the base end main lumen 31 and the outer peripheral surface of the base end side flexible tube 30.

Like the distal wire lumen 22, the base end wire lumen 32 is a circular cross-sectional space extending in the longitudinal direction L inside the tube wall, and is formed with a diameter that allows a traction wire 50, which will be described later.

Like the distal wire lumen 22, the base end wire lumen 32 is provided in four directions (up, down, left, and right) on the tube wall having a ring-shaped cross section.

Specifically, as shown in FIG. 71 at (D), wire lumens 33a and 33b in the upward direction HU, base end side wire lumens 34a and 34b in the downward direction HD, and base end side wire lumens 34a and 34b in the right side WR in the tube wall having a ring-shaped cross section 35a, 35b, and base end side wire lumens 36a, 36b on the left side WL are provided at predetermined intervals in the circumferential direction of the circular cross section.

When viewing the movable elongated structure 10 from the base end side LB to the distal end side LF, the clockwise sides of the base end side wee lumen 32 formed by a pair of two through holes are indicated by 33a, 34a, 35a, 33a. 34a. 35a, 36a, and the counterclockwise sides are 33b, 34b, 35b, and 36b.

Note that the base end side wire lumens 32, which are provided two by two in four directions (up, down, left, and right), may be integrally formed into an elliptical shape.

Among the base end side wire lumens 32 configured in this manner, the pair of wire lumens 33a and 33b provided in the upward direction HU on the tube wall having a ring-shaped cross section are collectively referred to as a base end side upper lumen set 33, and similarly, The base end side wire lumens 34a and 34b in the downward direction HD are the base end side lower lumen set 34, the base end side wire lumens 35a and 35b in the right side WR are the base end side right lumen set 35, and the base end side wire lumens in the left side WL. 36a and 36b are served as a base end left lumen set 36.

Like the distal end side flexible tube 20, the base end side flexible tube 30 configured as described above is a flexible tube made of polyamide elastomer, expanded polytetrafluoroethylene, polyurethane, polytetrafluoroethylene, or the like. may be configured with the distal end side flexible tube 20 and the base end side flexible tube 30 may be made of the same material, or may be made of different materials.

The arrangement of the wire lumens 22, 32 in the flexible tubes 20, 30 thus configured will be described in dotal with reference to FIG. 71 at (C) and (D).

Specifically, as shown in FIG. 71 at (C) and (D), in cross sections of the flexible tubes 20 and 30 viewed from the base end side LB to the distal end side LF, the width direction W passing through the center O of the cross section is set as the W coordinate, and the height Arrangement of the wire lumens 22 and 32 on virtual coordinates with H coordinate indicating the vertical direction H will be described.

In the virtual coordinates shown in FIG. 71 at (C) and (D), the right side WR in the W coordinate is on the + side, the left side WL is on the − side, the upward direction HU on the H coordinate is on the + side, and the downward direction HD is on the − side.

A set of lumens 23, 33 arranged in the upward direction HU in the flexible tubes 20, 30 is arranged on the + side of the H coordinate, wire lumens 23a, 33a are arranged on the + side of the W coordinate, and wire lumens 23b, 33b are arranged on the + side of the W coordinate. is arranged on the − side of the W coordinate.

A pair of lumens 24, 34 arranged in the downward direction HD in the flexible tubes 20, 30 are arranged on the − side of the H coordinate, wire lumens 24a, 34a are arranged on the − side of the W coordinate, and wire lumens 24b, 34b are arranged on the − side of the W coordinate. is arranged on the + side of the W coordinate.

The lumen set 25, 35 arranged on the right side WR of the flexible tubes 20, 30 is arranged on the + side of the W coordinate, the wire lumens 25a, 35a are arranged on the − side of the H coordinate, and the wire lumens 25b, 35b are arranged on the − side of the H coordinate, on the + side of the H coordinate.

The lumen set 26, 36 arranged on the left side WL in the flexible tubes 20, 30 is arranged on the − side of the W coordinate, the wire lumens 26a, 36a are arranged on the + side of the H coordinate, and the wire lumens 26b, 36b are arranged on the + side of the H coordinate, on the − side of the H coordinate.

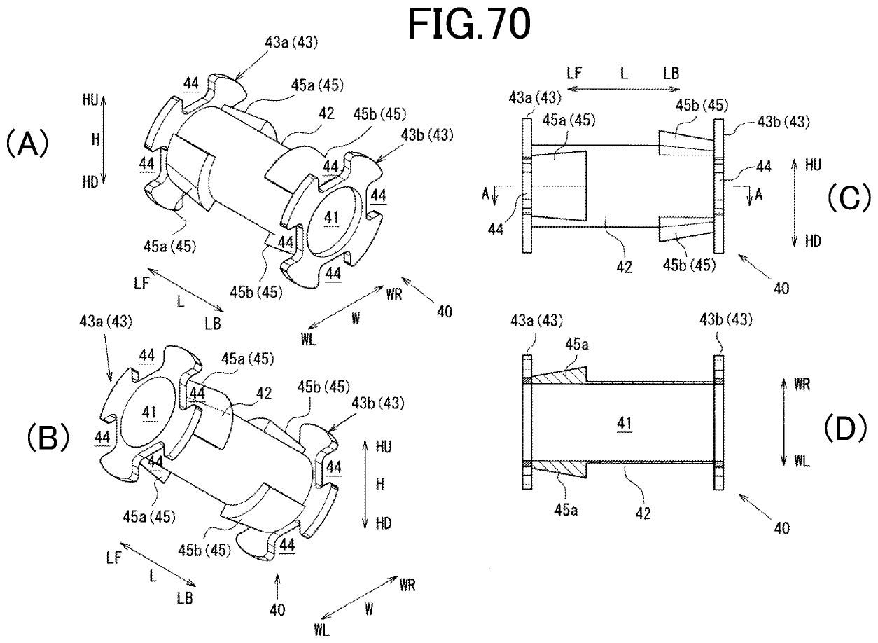

As shown in FIG. 70, the spacer 40 consists of a cylinder 42 having an inner space 41 having a circular cross section penetrating in the longitudinal direction L. and flange portions 43 (43a, 43b) provided at both ends of the cylinder 42 in the longitudinal direction L.

The internal space 41 is formed with a space having the same diameter as the main lumens 21 and 31 described above. The cylinder 42 has a diameter that is arranged inside the diameter of the traction wire 50 that is inserted through the distal wire lumen 22 provided in the distal end side flexible tube 20 and the base end wire lumen 32 provided in the base end side flexible tube 30.

The flanges 43 (43a, 43b) are formed with an outer diameter that protrudes radially outward from the outer diameter of the cylinder 42, and are referred to as a distal wire lumen 22 and a base end wire lumen 32 (hereinafter referred to as wire lumens 22, 32). An arrangement recess 44 for arranging the traction wire 50 is provided at a location corresponding to it. The flange portion 43 is formed with an outer diameter equivalent to that of the distal end flexible tube 20 and the base end flexible tube 30.

The arrangement recess 44 is a recess for collectively arranging two traction wires 50 to be inserted into the two wire lumens 22 and 32, and is formed with a width and depth corresponding to the two wire lumens 22 and 32.

Of the protrusions 43 provided on both sides in the longitudinal direction L of the cylinder 42, the distal end side LF is defined as a distal end side flange 43a, and the base end side LB is defined as a base end side flange 43b.

Furthermore, a guiding projection 45 for guiding the traction wire 50 radially outward is provided at the end of the cylinder 42 in the longitudinal direction L.

The guide convex part 45 is provided at a position corresponding to the arrangement recess 44 of the flange 43 at the end in the longitudinal direction L, with a corresponding width, and extends from the end in the longitudinal direction L to the center of the cylinder 42 in the longitudinal direction L It is formed to have a trapezoidal vertical cross-sectional shape in which the protrusion height gradually increases (see FIG. 70 at (D)).

The guide convex part s 45 are provided at positions facing each other in the radial direction among the arrangement recesses 44 provided in four directions (up, down, left, and right) at both ends in the longitudinal direction L, and the facing direction is the tip of the distal end side LF. The side guide convex portion 45a and the base end side guide convex portion 45b on the base end side LB are provided so as to be perpendicular to each other.

Specifically, the distal end side guide convex portion 45a on the distal end side LF is provided in the upward direction HU and the downward direction HD so as to face each other in the height direction H, and the base end side guide convex portion 45b on the base end side LB. They are provided on the right side WR and the left side WL so as to face each other in the width direction W.

The spacer 40 configured as described above may be made of, for example, a metal material such as stainless steel, an elastic body such as polytetrafluoroethylene, an elastic body such as a spring, or a resin material.

Further, the spacer 40 may be configured by integrating the cylinder 42 and the flange portion 43, or may be configured separately and assembled. When the cylinder 42 and the flange portion 43 are formed separately, the cylinder 42 and the flange portion 43 may be made of the same material.

In addition, the spacer 40 protrudes in the longitudinal direction L from the flange 43 and is fitted into the main lumens 21 and 31 of the distal end side flexible tube 20 and the base end side flexible tube 30 to provide the distal end side flexible tube 20 and the base end side flexible tube. The spacer 40 may be configured so as not to move relative to the end flexible tube 30 in the radial direction.

The traction wire 50 is a flexible wire, has an enlarged diameter portion 51 at its distal end, and is homed with a length slightly longer than the length of the movable elongated structure 10 in the longitudinal direction L.

Eight traction wires 50 each having an enlarged diameter portion 51 at its distal end are provided.

Specifically, eight traction wires 50 are provided corresponding to the wire lumens 22 and 32 provided in four directions (up, down, left, and right directions). Specifically, the traction wires 53a and 53b are routed in the distal upper lumen set 23 in the upward direction HU of the distal end side flexible tube 20, and the traction wires are routed in the distal lower lumen set 24 in the downward direction HD. 54a and Mb, traction wires 55a and 55b routed to the distal right lumen set 25 arranged on the right WR, and traction wires 56a and 56b routed to the distal left lumen set 26 on the left WL.

Traction wires 53a and 53b are routed respectively corresponding to the distal wire lumens 23a and 23b of the distal upper lumen set 23 in the upward direction HU.

Similarly, the traction wires 54a and 54b are routed respectively corresponding to the distal wire lumens 24a and 24b of the distal end side lower lumen set 24 in the downward direction HD, and the distal end side of the distal end side right lumen set of the right side WR. Traction wires 55a and 55b are routed respectively corresponding to the wire lumens 25a and 25b, and traction wires 56a and 56b are respectively arranged corresponding to the distal end side wire lumens 26a and 26b of the end side left lumen set 26 of the left WL.

Among the traction wires 50 configured as described above, the pair of fraction wires 53 a and 53 b is collectively referred to as a first wire set 53. Similarly, the pair of traction wires 54a and 54b are set together as a second wire set 54, the pair of traction woes 55a and 55b are set together as a third wire set 55, and the pair of traction wires 56a and 56b are set together. A fourth wire set 56 is provided.

The traction wire 50 may be made of a metal material such as stainless steel, nylon, fluorocarbon, or the like.

In addition, instead of providing eight traction wires 50, four long traction wires 50 may be bent.

Assembly of the movable elongated structure 10 having the elements configured as described above will now be described.

First, the distal end side flexible tube 20, the spacer 40, and the base end side flexible tube 30 are arranged in series along the longitudinal erection L from the distal end side LF toward the base end side LB. At this time, the distal end side flexible tube 20 and the base end side flexible tube 30 are arranged in directions corresponding to the wire lumens 22, 32 provided in four directions (up, down, left, and right directions).

At this time, as shown in FIGS. 68 and 71, the distal wire lumens 22 (23 to 26), the arrangement recesses 44 in four directions (vertical and horizontal directions), and the base end wire lumens 32 (33 to 36). They are arranged so as to communicate in the longitudinal direction L.

Then, the eight traction wires 50 are inserted through the distal wire lumen 22, the arrangement recess 44 and the base end wire lumen 32.

Specifically, in the distal end side flexible tube 20, the first wire set 53 is routed to the distal upper lumen set 23 in the upward direction HU among the four directions of the cross section where the distal wire lumen 22 is provided, and the base wire lumen 22 is provided. In the base end side flexible tube 30, the interval from the cross-sectional center O in the height direction H is closer than the distal end side upper lumen set 23 routed in the distal end side flexible tube 20, and the routed traction wires 53a and 53b are closer to each other. It is routed to the wire lumens 35b and 36a where the interval is widened. Wire lumens 35b and 36a of the lumen sets 35 and 36 facing each other in the width direction W are arranged on the + side of the H coordinate, like the distal upper lumen set 23 of the distal end side flexible tube 20.

the spacer 40 arranged between the distal end side flexible tube 20 and the base end side flexible tube 30, the protrusion 43 has a The traction wires 53a and 53b are arranged in the appropriate arrangement recesses 44.

In addition, the upward direction HU of the distal end cap 60 in the distal end side flexible tube 20 is fixed.

In the distal end side flexible tube 20, the second wire set 54 is routed to the distal lower lumen set 24 in the downward direction HD among the four directions of the cross section where the distal wire lumen 22 is provided. In the base end side flexible tube 30, the interval from the cross-sectional center O in the height direction H is shorter than the distal end side lower lumen set 24 routed in the distal end side flexible tube 20, and the routed traction wires 54a, It is routed to the base end side wire lumens 35a and 36b where the interval between 54b is widened. The base end wire lumens 35a and 36b of the lumen sets 35 and 36 facing each other in the width direction W are arranged on the minus side of the H coordinate, like the distal lower lumen set 24 of the distal end side flexible tube 20.

Similarly, in the spacer 40 arranged between the distal end side flexible tube 20 and the base end side flexible tube 30, the flange 43 is appropriately spaced so that the second wire set 54 may be routed as described above. The traction wires 54a and 54b are arranged in the arrangement recess 44 of it.

In addition, the expanded diameter portions 51 of the traction wires 54a and 54b are arranged and faced in the fixing concave portion 61 of the distal end cap 60 of the distal end flexible tube 20 in the downward direction HD.

In the distal end side flexible tube 20, the third wire set 55 is routed to the distal right lumen set 25 of the right WR among the four directions of the cross section where the distal wire lumen 22 is provided. In the base end side flexible tube 30, the interval from the cross-sectional center O in the width direction W is closer than the distal end side right lumen set 25 routed in the distal end side flexible tube 20, and the routed fraction wires 55a, It is routed to the base end side wire lumens 33a and 34b where the interval between 55b is widened. The base end wire lumens 33a and 34b of the lumen sets 23 and 24 facing each other in the height direction H are arranged on the + side of the W coordinate in the same manner as the distal right lumen set 25 of the distal end side flexible tube 20.

Similarly, in the spacer 40 arranged between the distal end side flexible tube 20 and the base end side flexible tube 30, the flange 43 is appropriately spaced so that the third wire set 55 may be muted as described above. The traction wires 55a and 55b are arranged in the arrangement recess 44.

In addition, the expanded diameter portions 51 of the traction wires 55a and 55b are arranged and faced in the fixing recess 61 of the right side WR of the distal end cap 60 of the distal end flexible tube 20.

In the distal end side flexible tube 20, the fourth wire set 56 is routed to the distal left lumen set 26 an the left side WL among the four directions of the cross section where the distal wire lumen 22 is provided. In the base end side flexible tube 30, the interval from the cross-sectional center O in the width direction W is shorter than the distal end side left lumen set 26 routed in the distal end side flexible tube 20, and the routed traction wires 56a, It is routed to the base end side wire lumens 33b and 34a where the interval between 56b is widened. The base end wire lumens 33a and 34b of the lumen sets 23 and 24 facing each other in the height direction H are arranged on the − side of the W coordinate in the same manner as the distal left lumen set 26 in the distal end side flexible tube 20.

Similarly, in the spacer 40 arranged between the distal end side flexible tube 20 and the base end side flexible tube 30, the flange 43 is appropriately spaced so that the fourth wire set 56 may be routed as described above. The traction wires 56a and 56b are arranged in the arrangement recesses 44 of it.

In addition, the enlarged diameter portions 51 of the traction wires 56a and 56b are placed and fixed in the fixing recess 61 on the left side WL of the tip end cap 60 of the distal end side flexible tube 20.

In the above description, the wire sets (53 to 56) are arranged in order, but the order of arrangement is not limited and may be arranged in any order.

Furthermore, after the traction wire 50 is routed through the distal wire lumen 22 of the distal end side flexible tube 20 and the base end wire lumen 32 of the base end side flexible tube 30 arranged along the longitudinal direction 1, loosen traction wire 50 between side flexible tube 20 and base end side flexible tube 30, place spacer 40 between distal end side flexible tube 20 and base end side flexible tube 30, and loosen pull. The wire 50 may be assembled by arranging it in the arrangement recess 44.

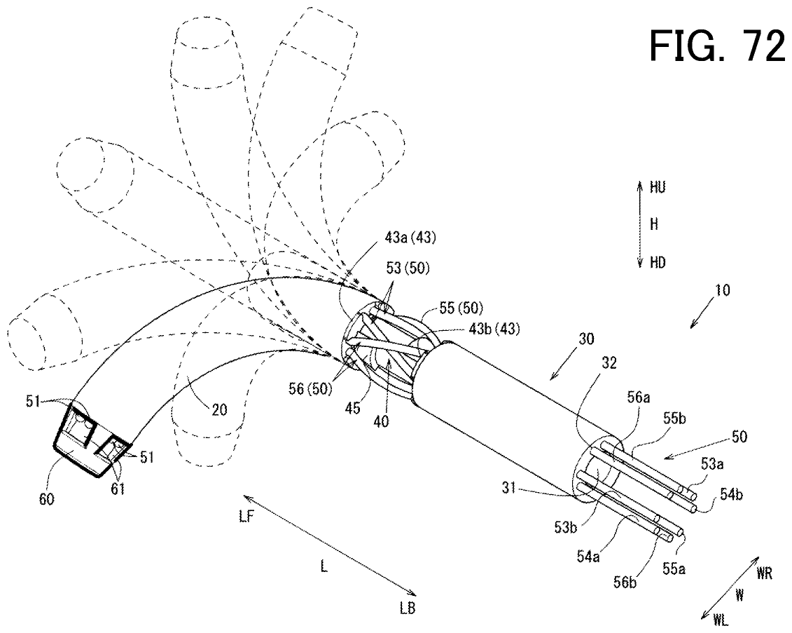

As shown in FIG. 72, the movable elongated structure 10 configured in this way may be smoothly curved and deformed in the longitudinal direction L by pulling the traction wire 50 toward the base end side LB.

Specifically, the distal end side flexible tube 20 is moved upward by pulling the first wire set 53 routed to the distal upper lumen set 23 in the upward direction HU in the distal end side flexible tube 20 toward the base end side LB. The distal end side flexible tube 20 may be bent downward HD by pulling the second wire set 54 routed to the distal lower lumen set 24 in the downward HD direction toward the base end side LB.

Then, the distal end side flexible tube 20 is bent and deformed to the right WR by pulling the third wire set 55 routed to the distal right lumen set 25 of the right WR in the distal end side flexible tube 20 to the base end side LB. The distal end side flexible tube 20 may be bent and deformed to the left side WI by pulling the fourth wire set 56 routed to the distal left lumen set 26 of the left side WL toward the base end side LB.

In this way, by pulling the wire sets (53-56) routed to the distal lumen sets (23-26) of the distal end side flexible tube 20, the wire sets (53-56) to be pulled are routed. The distal end side flexible tube 20 may be bent and deformed in four directions (up, W down, left, and right directions) of the set of distal end side lumens (23 to 26).

On the other hand, by pulling some of the traction wires (53a to 56a, 53b to 56b) constituting the wire set (53 to 56), the distal end side flexible tube 2 may be pulled.

For example, a traction wire 53a routed in the distal wire lumen 23a of the distal upper lumen set 23 of the upward direction HU, and a traction wire 55b routed in the distal wire lumen 25b of the distal right lumen set 25 of the right WR. By pulling the distal end side flexible tube 20 is bent obliquely upward and rightward to become the upward direction HU and the right side WR.

On the other hand, the first wire set 53 routed in the distal upper lumen set 23 of the upward direction HU and the traction wire 55b routed in the distal wire lumen 25b of the distal right lumen set 25 of the right WR are connected. By pulling, the distal end side flexible tube 20 may be bent and deformed not only in the upward direction HU and the right side WR but also in the upward right direction, which is the upward direction HU.

Conversely, the traction wire 53a routed in the distal wire lumen 23a of the distal upper lumen set 23 of the upward direction HU and the third wire set 55 routed in the distal right lumen set 25 of the right WR are pulled. By doing so, the distal end side flexible tube 20 may be bent and deformed in the oblique upper right direction, which is the upward direction HU and the right side WR, but further In the upper right direction, which Is the right side WR.

Similarly, the distal wire lumen 23b of the distal upper lumen set 23 in the upward direction HU and the distal wire lumen 26a of the distal left lumen set 26 in the left WL, the distal upper lumen set 23 in the upward direction HU and the left WL. or the distal wire lumen 23b of the distal upper lumen set 23 of the upward direction HU and the distal left lumen set 26 of the left WI, The distal end flexible tube 20 may be bent and deformed obliquely upward and to the left.

Also, the distal wire lumen 24b of the distal lower lumen set 24 in the downward direction HD and the distal wire lumen 25a of the distal right lumen set 25 in the right WR, the distal lower lumen set 24 in the downward direction HD and the right WR. By pulling the distal wire lumen 25a of the distal right lumen set 25, or the distal wire lumen 24b of the distal lower lumen set 24 in the downward direction HD and the distal right lumen set 25 of the right WR, wherein the distal end flexible tube 20 may be bent and deformed in the lower right direction.

Furthermore, the distal wire lumen 24b of the distal lower lumen set 24 in the downward direction HD and the distal wire lumen 26a of the distal left lumen set 26 in the left WL, the distal lower lumen set 24 in the downward direction HD and the left WL. or the distal wire lumen 24b of the distal lower lumen set 24 in the downward direction HD and the distal left lumen set 26 in the left WL. The distal end side flexible tube 20 may be bent and deformed obliquely downward and leftward.

Thus, in the movable elongated structure (10), the eight traction wires 50 are fixed to the fixing concave portion 61 of the distal cap 60 on the distal end side of the distal end side flexible tube (20) with the enlarged diameter portion 51, In the distal end side flexible tube (20), one of the four directions (upward HU, downward HD, right WR, left WL) of the cross section where the lumen set (23-28) is provided, lumen set (23-26), and in the base end side flexible tube (30), one direction (upward HU, downward HD. Base end side wire lumens (33a to 36a, 33b to 36b) that are closer to the cross-sectional center O on the right side WR and left side WL) and have wider intervals between the routed traction bodies (53a to 56a, 53b to 56b) Because it is routed in the LB, multiple traction bodes (53a to 56a, 53b to 56b), traction bodies (53a to 56a, 53b to 56b) and traction sets (53 to 56) are pulled to the base end side LB. As a result, the distal end side flexible tube 20 may be bent and deformed in a desired direction with respect to the base end side flexible tube 30.

It should be noted that the traction force when pulling the plurality of traction wires 50 may be the same, but the traction wires 50 may be pulled with different traction forces.

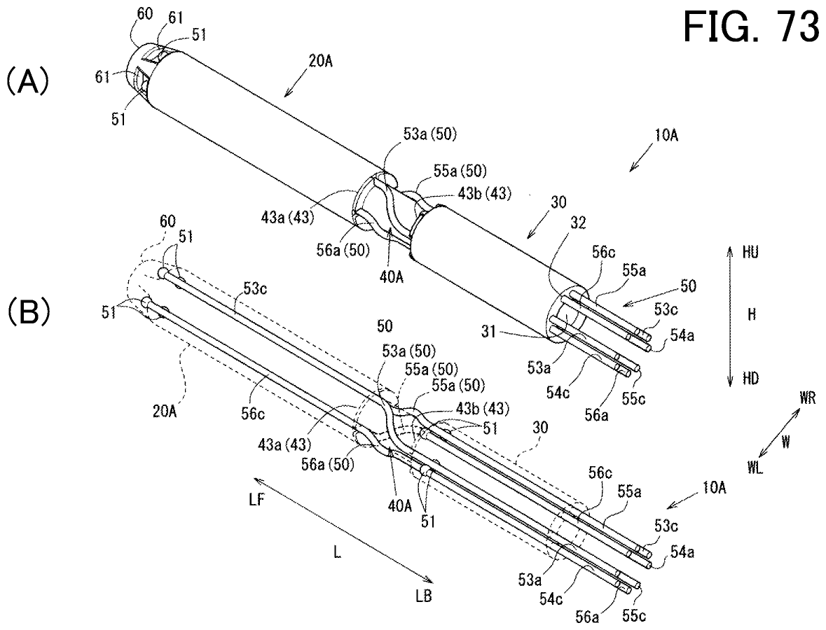

Next, a movable elongated structure 10A of a different embodiment will be described with reference to FIGS. 73 to 77.

In addition, the same reference numerals are assigned to the same configurations as those of the movable elongated structure 10 described above, and the description of the configurations is omitted. In the following, the configuration of the movable elongated structure 10A that is different from that of the movable elongated structure 10 described above will be described.

Different from the movable elongated structure 10 comprising the distal end side flexible tube 20, the base end side flexible tube 30, the spacer 40, and the eight traction wires 50 described above. In addition to a distal end side flexible tube 20A, a base end side flexible tube 30, a spacer 40A and four traction wires 50, four short traction wires 50A shorter than the fraction wires 50 are provided.

The distal end side flexible tube 20A has a main lumen 21 at the cross-sectional center O, and lumen sets 23, 24, 25, and 26 each having a pair of distal end side wire lumens 22 in four directions (up, down, left and right) inside the tube wall. Unlike the distal end side flexible tube 20, the distal end side wire lumens 22A (23c to 26c) are provided in four directions (up, down, left, and right directions).

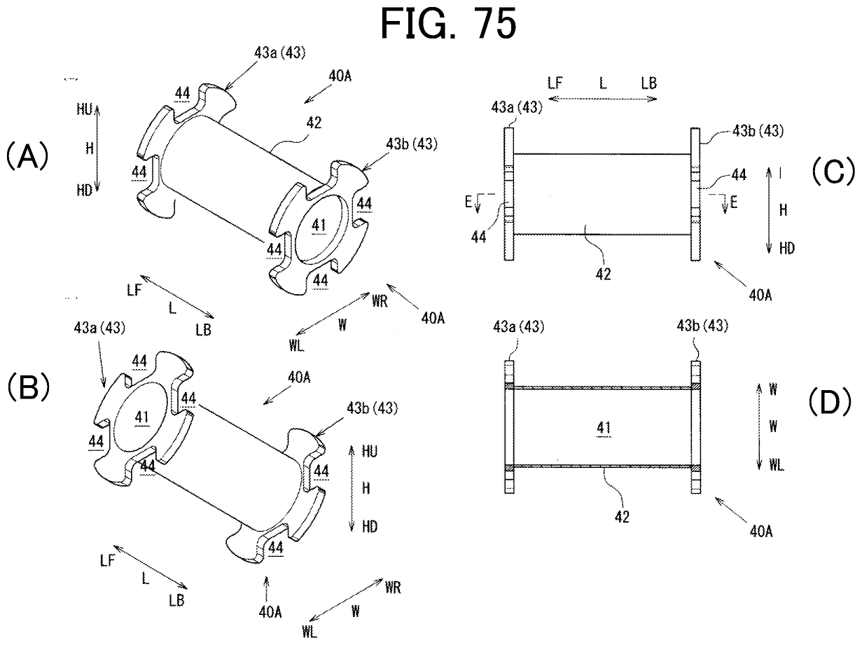

The base end side flexible tube 30 in the movable elongated structure 10A is a base end side lumen set (33 to 36) in four directions (up, down, left, and right). The spacer 40A has the same configuration as the spacer 40 in the movable elongated structure 10 except that it does not have the guide convex part 45 as shown in FIG. 75.

Further, as described above, in contrast to the movable elongated structure 10 having eight traction wires 50, the movable elongated structure 10A has four fraction wires 50 (53a, 54a, 55a, 56a), and four short traction wires 50A (53c, 54c, 55c. 56c) to provide eight traction wires.

As described above, the traction wire 50 (53a. 54a, 55a, 56a) is routed through the distal wire lumen 22A of the distal end side flexible tube 20 and the base end wire lumen 32 of the base end side flexible tube 30. Therefore, the short traction wires 50A (53c, 54c, 55c, 56c) are formed only in the base end wire lumen 32 of the base end side flexible tube 30, although they are formed longer than the movable elongated structure 10. For wiring, it is formed slightly longer than the length in the longitudinal direction L of the base end side flexible tube 30. An enlarged diameter portion 51 is also provided at the distal end of the short traction wire 50A (53c, 54c, 55c, 56c).

Then, the traction wire 50 and the short traction wire 50A form a pair and form a set. Specifically, a first wire set 53 is made up of a traction wire 53a and a short traction wire 53c, a second wire set 54 is made up of a traction wire 54a and a short traction wire 54 may a traction wire 55a and a short traction wire 55c. A third wire set 55 is formed by these and a fourth wire set 56 is formed by a traction wire 56a and a short traction wire 56c.

Assembling of the movable elongated structure 10A having each element configured as described above will be described below.

First, the distal end side flexible tube 20A, the spacer 40A, and the base end side flexible tube 30 are arranged in series along the longitudinal direction L from the distal end side LF toward the base end side LB. At this time, the distal end side flexible tube 20A and the base end side flexible tube 30 area pair of wire lumens 22A (23c to 26c) provided in four directions (vertical and horizontal directions) and a base end side wire lumen 32. [0429] to the base end side lumen set (33 to 36)

Then, the traction wire 50 is inserted through the distal wire lumen 22A, the arrangement recess 44 and the base end wire lumen 32, and the short traction wire 50A is inserted through the base end wire lumen 32.

Specifically, in the distal end side flexible tube 20A, the traction wire 53a of the first wire set 53 is routed in the distal wire lumen 23c in the upward direction HU among the four directions of the cross section where the distal wire lumen 22A is provided. Then, in the base end side flexible tube 30, the wire lumen that is closer to the cross-sectional center O in the height direction H than the distal end side wire lumen 23c routed in the distal end side flexible tube 20 and faces in the width direction W. A traction wire 53a is routed in the base end side wire lumen 36a of 35b and 36a, and a short traction wire 53c is routed in the base end side wire lumen 35b.

In addition, in the spacer 40A arranged between the distal end side flexible tube 20 and the base end side flexible tube 30, an appropriate placement recess in the flange portion 43 is provided so that the traction wire 53a may be routed as described above. At 44, a pull wire 53a is placed.

In addition, the enlarged diameter portion 51 of the traction wire 53a is arranged and fixed in the facing recess 61 of the distal end cap 60 in the distal end flexible tube 20 in the upward direction HU, and the short traction wire is routed to the base end wire lumen 35b. The expanded diameter portion 51 of the wire 53c is fixed in the vicinity of the base-end flange 43b of the spacer 40A.

In the distal end side flexible tube 20A, the traction wire 54a of the second wire set 54 is routed to the distal wire lumen 24c in the downward direction HD among the four directions of the cross section where the distal wire lumen 22A is provided. Then, in the base end side flexible tube 30, the interval from the cross-sectional center O in the height direction H is closer than the distal end side wire lumen 24c routed in the distal end side flexible tube 20, and the base end side facing the width direction W Of the side wire lumens 35a and 36b, the traction wire 54a is routed through the base end wire lumen 35a, and the short traction wire 54c is routed through the base end wire lumen 36b.

In addition, in the spacer 40A arranged between the distal end side flexible tube 20 and the base end side flexible tube 30, an appropriate placement recess in the flange 43 is provided so that the traction wire 54a may be routed as described above. At 44, a pull wire 54a is placed.

In addition, the enlarged diameter portion 51 of the traction wire 54a is arranged and fixed in the fixing recess 61 in the downward direction HD of the distal end cap 60 of the distal end flexible tube 20, and the short traction wire is routed to the base end wire lumen 36b. The enlarged diameter portion 51 of the wire 54c is fixed in the vicinity of the base-end flange 43b of the spacer 40A.

In the distal end side flexible tube 20A, the traction wire 55a of the third wire set 55 is routed to the distal wire lumen 25c of the right WR among the fax directions of the cross section where the distal wire lumen 22A is provided. Then, in the base end side flexible tube 30, the interval from the cross-sectional center O in the width direction W is closer than the distal end side wire lumen 25c routed in the distal end side flexible tube 20, and the base end facing in the height direction H of the side wire lumens 33a and 34b, a traction wire 55a is routed through the base end wire lumen 33a, and a short traction wire 55c is routed through the base end wire lumen 34b.

In addition, in the spacer 40A arranged between the distal end side flexible tube 20 and the base end side flexible tube 30, an appropriate placement recess in the flange 43 is provided so that the traction wire 55a may be routed as described above. At 44, a pull wire 55a is placed.

Further, the expanded diameter portion 51 of the traction wire 55a is arranged and fixed in the fusing recess 61 of the right side WR of the distal end cap 60 of the distal end flexible tube 20, and the short traction wire is routed to the base end wire lumen 34b. The enlarged diameter portion 51 of 55c is fixed in the vicinity of the base end flange 43b of the spacer 40A.

In the distal end side flexible tube 20A, the traction wire 56a of the fourth wire set 56 is routed to the distal wire lumen 26c on the left side WL among the four directions of the cross section where the distal wire lumen 22A is provided. Then, in the base end side flexible tube 30, the interval from the cross-sectional center O in the width direction W is closer than the distal end side wire lumen 26c routed in the distal end side flexible tube 20, and the base end facing the height direction H of the side wire lumens 33b and 34a, a traction wire 56a is routed through the base end wire lumen 34a, and a short traction wire 56c is routed through the base end wire lumen 33b.

In addition, in the spacer 40A arranged between the distal end side flexible tube 20 and the base end side flexible tube 30, an appropriate placement recess in the flange 43 is provided so that the traction wire 56a may be routed as described above. At 44 a traction wire 56a is placed.

Further, the enlarged diameter portion 51 of the traction wire 56a is arranged and fixed in the fixing recess 61 on the left side WL of the distal end cap 60 of the distal end flexible tube 20, and the short traction wire is routed In the base end wire lumen 33b. The enlarged diameter portion 51 of 56c is fixed in the vicinity of the base end flange 43b of the spacer 40A.

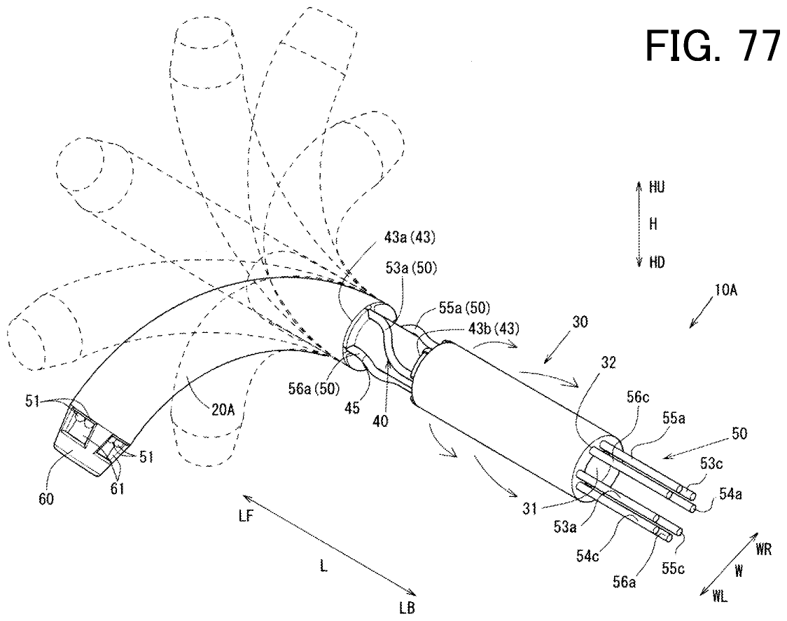

As shown in FIG. 77, the movable elongated structural body 10A configured in this way smoothly pulls the distal end side flexible tube 20A in the longitudinal direction L by pulling the traction wire 50 toward the base end side LB. The curve may be deformed. Furthermore, by pulling the short traction wire 50A to the base end side LB, the movable elongated structure 10A may smoothly move the base end side flexible tube in the longitudinal direction L as indicated by the arrow in FIG. 30 may be bent and deformed.

Specifically, the distal end side flexible tube 20A is bent in the upward direction HU by pulling the traction wire 53a routed in the distal end side wire lumen 23c in the upward direction HU in the distal end side flexible tube 20A toward the base end side LB. The distal end side flexible tube 20A may be bent and deformed downward HD by pulling the traction wire 54a routed in the distal wire lumen 24c downward HD toward the base end LB.

Then, the distal end side flexible tube 20A may be bent and deformed to the right WR by pining the traction wire 55a routed to the distal wire lumen 25c of the right WR in the distal end side flexible tube 20A toward the base end side LB. The distal end side flexible tube 20A may be bent and deformed toward the left side WL by pulling the traction wire 56a routed to the distal end side wire lumen 26c of the left side WI toward the base end side LB.

In addition, the base end side flexible tube 30 is bent to the right WR by pulling the short traction wire 53c routed to the base end wire lumen 35b of the right WR in the base end side flexible tube 30 to the base end side LB. The base end side flexible tube 30 may be bent and deformed to the left side WL by pulling the short traction wire 54c routed to the base end side wire lumen 36b of the left side WL toward the base end side LB.

Then, the base end side flexible tube 30 is moved downward HQ by pulling the short traction wire 55c routed to the base end wire lumen 34b in the downward HD direction in the base end side flexible tube 30 toward the base end side LB. The base end side flexible tube 30 may be bent in the upward direction HU by pulling the short traction wire 56c routed in the base end side wire lumen 33b in the upward direction HU toward the base end side LB.

In this way, when the traction wire 50 and the short traction wire 50A are independently pulled toward the base end side LB, one of the distal end side flexible tube 20A and the base end side flexible tube 30 may be bent and deformed. may. Then when the wire set (53 to 56) configured as a pair of the traction wire 50 and the short traction wire 50A is pulled to the base end side LB, the distal end side flexible tube 20A and the base end side flexible tube 30 are separated into four sections. It may be bent and deformed in an orthogonal direction among directions (up, down, left, and right).

Of course, in the movable elongated structure 10A as well, a plurality of traction wires 50, a plurality of short traction wires 50A, or a combination thereof may be used to move the distal end side flexible tube 20A and the base end side flexible tube 30 to desired positions. It may bend and deform in any direction.

As described above, the movable elongated structure 10A includes the long traction bodies (53a-56a) of the wire sets (53-56) in the distal end side flexible tube (20) through the distal wire lumen 22A (23c).-26c) are arranged in the distal end side wire lumen 22A (23c to 26c) in one of the four directions (upward HU, downward HD, right WR, left WL) of the cross section, and the distal end side is flexible. Fixed at the distal end side (61) of the tube (20), the long retractors (53a-56a) and short retractors (53c-56c) of the wire sets (53-56) are attached to the base end side flexible tube (30), the interval from the cross-sectional center O in one direction is closer than the distal wire lumen 22A (23c to 26c) routed by the distal end side flexible tube (20), and the base faces in the direction intersecting the one direction. A short traction body routed to the base end side wire lumens (33a to 36a, 33b to 36b) and routed to the base end side wire lumens (33a to 36a, 33b to 36b) of the base end side flexible tube (30) Since (50A) is fixed to the base end side tubular body on the distal end side of the base end side flexible tube (30), each traction body (50, 50A) may be pulled independently. At least one of the base end side flexible tube (30) and the distal end side tubular body (20, 20A) is pulled against the other by pulling the plurality of fraction bodies (50, 50A) to the base end side (LB), which may be bent and deformed in a desired direction.

The traction wire 50 and the short traction wire 50A may be composed of one long traction wire that is bent back at the base end side FB.

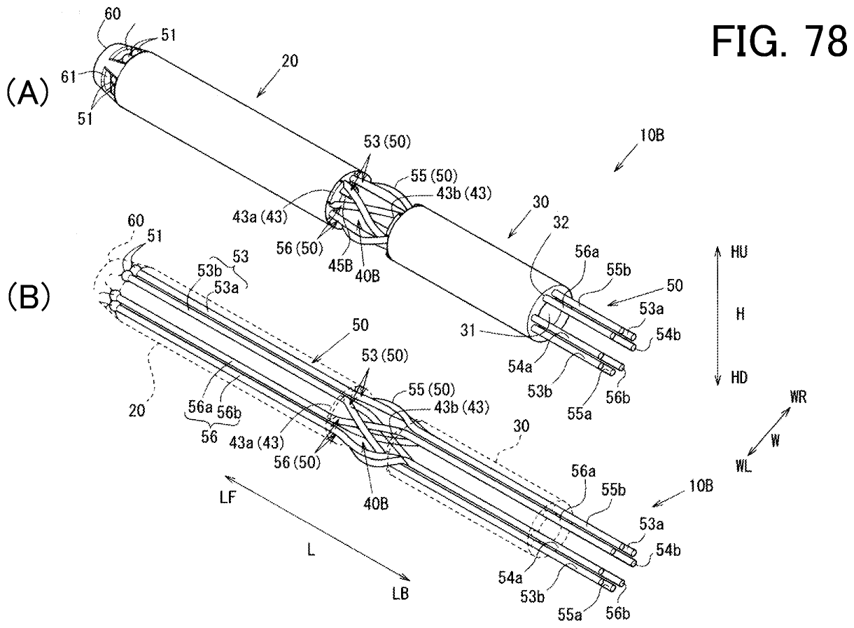

Next, a movable elongated structure 10B of a different embodiment will be described with reference to FIGS. 78 to 82.

Although the movable elongated structure 10B has a spacer 408 that is different from the spacer 40 in the movable elongated structure 10 described above, other elements have the same configuration. The wiring route of the base end side wire lumen 32 in the base end side flexible tube 30 of the wire set (53 to 56) routed to the side wire lumen 22 (23 to 26) is different.

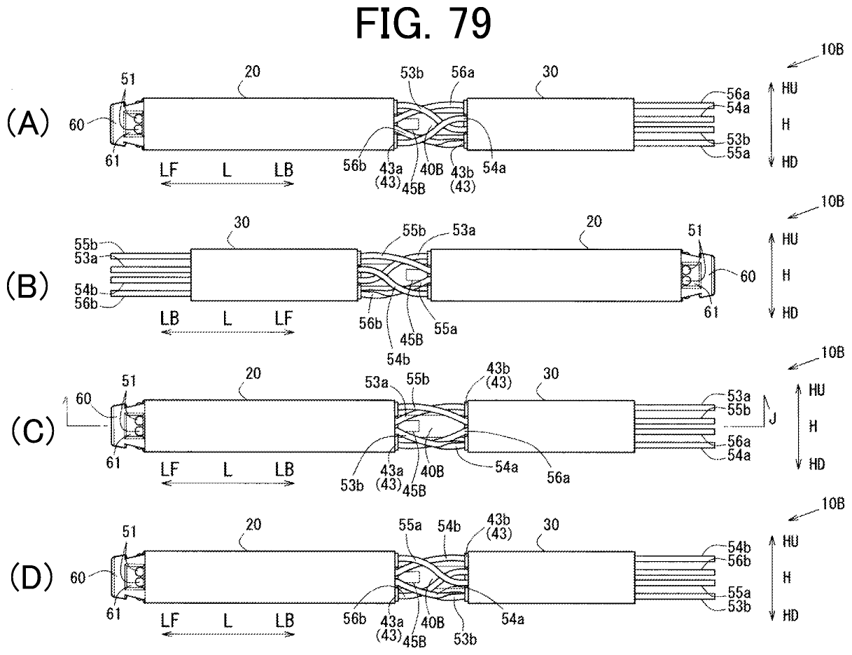

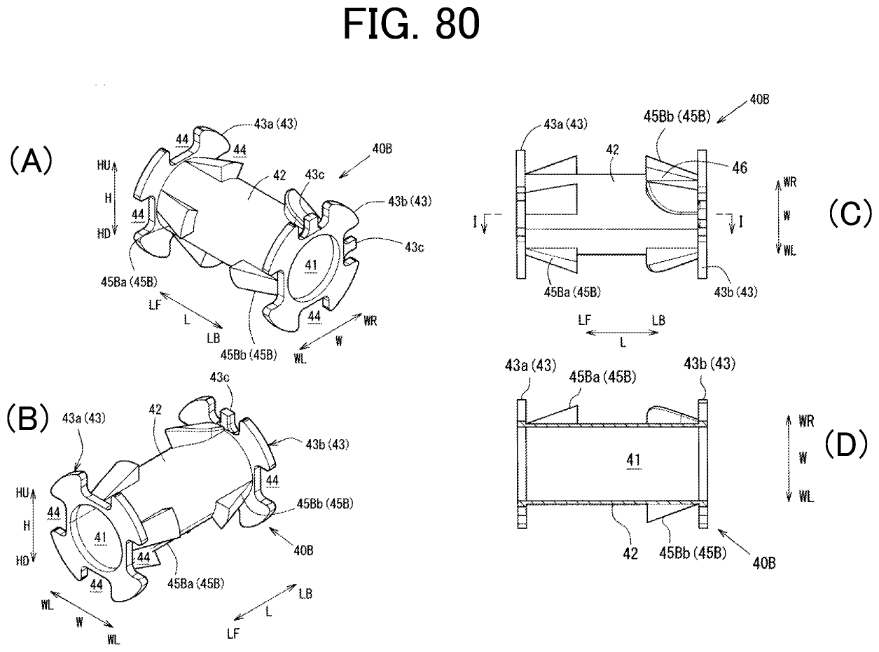

As shown in FIG. 80, the spacer 408, like the spacer 40 described above, has a distal end side flange 43a (43) having a placement recess 44 on the distal end side LF of a cylinder 42 having an internal space 41 inside, The base end side LB is provided with a base end side flange 43b (43). In addition, the upward direction HU and the right side WR of the base-end flange 43b are provided with partitioning projections 43c for partitioning the traction wires 50 to be arranged.

Further, in the case of the distal end side flange 43a in the cylinder 42, a guide projection portion 45Ba is provided at a position corresponding to the arrangement recessed portion 44 so that the projecting height gradually increases toward the base end side LB.

In the case of the base end side flange 43b in the cylinder 42, a guide convex part whose protruding height gradually increases toward the base end side LB is provided at a corresponding position between the arrangement recesses 44 adjacent in the circumferential direction 458b. The guide convex part 458b has a groove 46 through which the traction wire 50 may pass, and is formed in a shape curved in the circumferential direction. In addition, since the groove 48 is provided in the guide convex part 45Bb, as will be described later, it Is possible to arrange the crossing traction wires 53b. 54a and the traction wires 55a, 56b so as not to interfere with each other.

In the movable elongated structure 10 described above, in the distal end side flexible tube 20, four directions (upward HU, downward HO, right WR, left WL) of the cross section provided with the distal lumen set (23 to 26) of these, the distal end side lumen set (23 to 26) is routed in one direction, and the base end side flexible tube 30 is more flexible than the distal end side lumen set (23 to 26) routed by the distal end side flexible tube 20. The base end side where the interval from the cross-sectional center O in one direction (upward HU, downward HD, right WR, left WI) is short and the interval between the routed traction wires (53a to 56a, 53b to 56b) becomes wider.

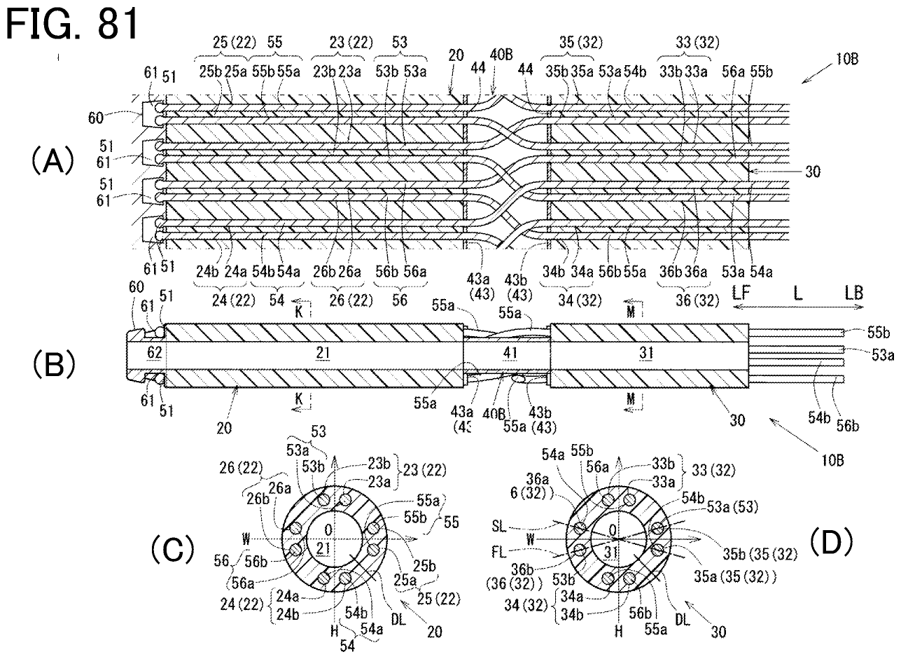

On the other hand, in the movable elongated structure 10B as well, as shown in FIG. 81 at (A), (C) and (D), the interval to the cross-sectional center O in one direction (upward HU, downward HO, right WR, left WL) is shorter, and the interval between the routed traction wires (53a to 56a, 53b to 56b) is wider. It was routed to the base end side wire lumen (33a to 36a, 33b to 36b).

Furthermore, in the movable elongated structure 10B, in the distal end side flexible tube 20, two wire sets (53 to 56) are arranged in two distal lumen sets (23 to 26).

In the base end side flexible tube 30, the traction wires (53a to 56a, 53b to 56b) in the two wire sets (53 to 56) are opposed to each other across the cross-sectional center O in an intersecting direction that intersects one direction. Any one of the base end side wire lumens (33a to 36a, 33b to 36b) in the two base end side lumen sets (33 to 36) is routed.

At this time, as shown in FIG. 81 at (D), in one of the base end side wire lumens (33a to 36e. 33b to 36b) in the two sets of lumens 33, 34, 35, 36 in the base end side flexible tube 30, A first virtual line FL connecting the traction bodies of one of the two routed wire sets (53 to 56) in the cross-sectional direction, and connecting the traction bodies of the other traction body set in the cross-sectional direction. It is routed so as to intersect with the second virtual line SL in the cross-sectional direction.

Specifically, in the distal end side flexible tube 20, the first wire set 53 is routed in the distal upper lumen set 23 in the upward direction HU among the four directions of the cross section where the distal wire lumen 22 is provided, and the lower A second wire set 54 is routed to the distal lower lumen set 24 in the direction HD.

The lumen sets 23 and 24 are opposed to each other in the width direction W perpendicular to the height direction H in cross section in the base end side flexible tube 30, and the interval between the lumen sets 23 and 24 in the width direction W is wider than that of the lumen sets 23 and 24. The lumen set 35, 36 is routed.

One of the traction wires 53a and 53b routed to the upper lumen set 23 on the distal end side of the upward direction HU is arranged in the routing path farther in the height direction H in the lumen set 35 and 36, that is, on the − side of the H coordinate. The other is routed to a route that is closer in the height direction H to the lumen set 35, 36, that is, to the route that is on the + side of the H coordinate.

On the other hand, one of the traction wires 54a and 54b routed to the distal lower lumen set 24 in the downward direction HD is arranged farther in the height direction H than the lumen set 35 and 36, that is, on the H coordinate + side. The other is routed to the lumen that is closer in the height direction H, that is, to the route that is on the H coordinate − side of the lumen set 35 and 36.

As an example, the traction wire 53a of the first wire set 53 routed in the distal wire lumen 23a on the H coordinate + side and the W coordinate + side of the distal upper lumen set 23 of the distal end side flexible tube 20 is located at the base end. In the side flexible tube 30, the base end wire lumen 35b on the H-coordinate + side and W-coordinate + side, which is closer in the height direction H. of the lumen pairs 35 and 36 is routed.

On the other hand, the traction wire 53b of the first wire set 53 muted in the distal wire lumen 23b on the H-coordinate + side and W-coordinate − side of the distal upper lumen set 23 of the distal end side flexible tube 20 is located at the base. In the base end side flexible tube 30, the base end side wire lumen 36a on the H-coordinate-side and W-coordinate-side, which is the furthest in the height direction H, of the lumen sets 35 and 36 is routed.

In addition, the traction wire 54b of the second wire set 54 routed in the distal end side wire lumen 24b on the H coordinate − side and the W coordinate + side of the distal end side lower lumen set 24 of the distal end side flexible tube 20 is located on the base end side. A base end side wire on the H coordinate − side and W coordinate + side, which Is closest in the height direction H in the lumen set 35, 36 in the flexible tube 30.

On the other hand, the traction wire 54a of the second wire set 54 routed in the distal wire lumen 24a on the H-coordinate − side and W-coordinate − side of the distal lower lumen set 24 of the distal end side flexible tube 20 is located at the base. In the end flexible tube 30, the base end wire lumen 36b on the H coordinate + side and W coordinate − side, which is farther in the height direction H from among the lumen sets 35 and 36, is routed.

]In this manner, the lumen pairs 23 and 24 facing each other in the height direction H of the distal end side flexible tube 20 are routed, and the lumen pairs 35 and 36 of each base end side flexible tube 30 facing each other in the width direction W are routed. Of the wire pairs 53 and 54, the traction wire 53b routed to the base end side wire lumen 36b farther in the height direction H, and the traction wire 54a routed to the base end side wire lumen 36a are arranged in the spacer 408. will cross over. However, the guiding projections 458 (45Ba, 45Bb) of the spacer 40B allow the fraction wire 53b and the traction wire 54a to operate smoothly by being pulled toward the base end side LB without interfering with each other.

Then, in the base end side flexible tube 30, a traction wire 53a routed to the base end side wire lumen 35b on the H coordinate + side and W coordinate + side, and a base end side wire on the H coordinate − side and W coordinate − side. A first virtual line FL connecting the traction wire 53b routed to the lumen 36a passes through the cross-sectional center O and connects the H coordinate − side and W coordinate − side and the H coordinate + side and W coordinate + side, FIG. 81 at (D), the direction is upward to the right.

On the other hand, in the base end side flexible tube 30, the traction wire 54b is routed in the base end wire lumen 35a on the H coordinate − side and W coordinate + side, and the bare end on the H coordinate + side and W coordinate − side. A second virtual line SL connecting the traction wire 54a routed to the side wire lumen 36b passes through the cross-sectional center O and connects the H coordinate + side and W coordinate − side and the H coordinate − side and W coordinate + side. In FIG. 81 at (D), the direction is downward to the right.

Therefore, the first virtual line FL passing through the traction wires 53a and 53b and the second virtual line L passing through the traction wires 54a and 54b intersect at the cross-sectional center O as shown in FIG. 81 at (D).

Similarly, in the distal end side flexible tube 20, the third wire set 55 is routed in the distal right lumen set 25 of the right. WR among the four directions of the cross section where the distal wire lumen 22 is provided, and the third wire set 55 is routed in the left WL. A fourth wire set 56 is routed to the distal left lumen set 26.

The lumen sets 25 and 26 are opposed to each other in the height direction H perpendicular to the width direction W in cross section in the base end side flexible tube 30, and the interval between the lumen sets 25 and 26 in the height direction H is larger than the interval between the lumen sets 25 and 26 in the height direction H. The wide lumen set 33, 34 is routed.

One of the traction wires 55b and 55a routed to the right lumen set 25 on the distal end side of the right WR is routed to a route farther in the width direction W of the lumen sets 33 and 34, that is, to the route on the − side of the W coordinate. Then, the other is routed to the route that is closer in the width direction W of the lumen pair 33, 34, that is, is on the + side of the W coordinate.

On the other hand, one of the traction wires 56b and 56a routed to the distal end side left lumen set 26 of the left WL is routed farther in the width direction W than the lumen sets 33 and 34, that is, to the W coordinate + side. The other is routed to the route which is closer in the width direction W of the lumen pair 33, 34, that is, to the route which is on the − side of the W coordinate.

As an example, the traction wire 55b of the third wire set 55 routed in the distal right wire lumen 25b on the W coordinate + side and the H coordinate + side in the distal right lumen set 25 of the distal end side flexible tube 20 is, in the base end side flexible tube 30, the base end wire lumen 33a on the W coordinate + side and the H coordinate + side, which is closer in the width direction W, of the lumen sets 33, 34 is routed.

On the other hand, the traction wire 55a of the third wire set 55 is routed in the distal right wire lumen 25a on the W coordinate + side and the H coordinate − side in the distal right lumen set 25 of the distal end side flexible tube 20. is routed to the base end side wire lumen 34a on the W coordinate − side and H coordinate − side, which is farther in the width direction W of the lumen sets 33 and 34 in the base end side flexible tube 30.

Further, the traction wire 56a of the fourth wire set 56 routed in the distal wire lumen 26a on the W coordinate − side and the H coordinate + side in the distal left lumen set 26 of the distal end side flexible tube 20 is located at the base end. In the side flexible tube 30, the base end wire lumen 33b on the W coordinate − side and the H coordinate + side, which is closer in the width direction W of the lumen pairs 33 and 34, is routed.

On the other hand, the traction wire 56b of the fourth wire set 56 routed in the distal end side wire lumen 26b on the W-coordinate − side and the H-coordinate − side of the distal end side left lumen set 26 of the distal end side flexible tube 20 is In the base end side flexible tube 30, the base end wire lumen 34b on the W coordinate + side and the H coordinate − side, which is farther in the width direction W of the lumen sets 33 and 34, is routed.

In this way, the lumen pairs 25 and 26 facing each other in the height direction H of the distal end side flexible tube 20 are routed, and the lumen pairs 33 and 34 of each base end side flexible tube 30 facing each other in the width direction W are muted. Of the wire sets 55 and 56, the traction wire 55a routed to the base end side wire lumen 34b farther in the width direction W, and the traction wire 56b routed to the base end side wire lumen 34a cross each other at the spacer 40B. will do. However, the guiding convex portion 45B of the spacer 40B allows the traction wire 55a and the traction wire 56b to operate smoothly by being pulled toward the base end side LB without interfering with each other.

Then, in the base end side flexible tube 30, a traction wire 55b routed to the base end side wire lumen 33a on the W coordinate + side and the H coordinate + side, and a base end side wire on the W coordinate − side and H coordinate − side. A first virtual line FL connecting the traction wire 55a routed to the lumen 34a passes through the cross-sectional center O and connects the W coordinate − side and H coordinate − side and the W coordinate + side and H coordinate + side, FIG. 81 at (D), the direction is upward to the right.

On the other hand, in the base end side flexible tube 30, the traction wire 56a routed to the base end side wire lumen 33b on the W coordinate − side and the H coordinate + side, and the base end on the W coordinate + side and H coordinate − side A second virtual line SL connecting the traction wire 56b routed to the side wire lumen 34b passes through the cross-sectional center O and connects the W coordinate + side and H coordinate − side and the W coordinate − side and H coordinate + side, downward to the right in FIG. 14 at (D).

Therefore, the first virtual line FL passing through the traction wires 55b and 55a and the second virtual line SL passing through the traction wires 56b and 56a intersect at the cross-sectional center O as shown in FIG. 14 at (D).

In the movable elongated structure 10B configured in this way, the base end side flexible tube 30 is pulled by pulling one of the wire sets (53 to 56) or a combination thereof toward the base end side LB. On the other hand, the distal end side flexible tube 20 may be bent and deformed in a desired direction.

In particular, as described above, both the first virtual line FL based on the wiring route of each traction wire 50 in two of the four wire sets (53 to 56) and the second virtual line SL passing therethrough are on the base end side. They pass through the cross-sectional center O of the flexible tube 30 and intersect at the cross-sectional center (O). Therefore, in the direction in which any one of the four wire sets (53 to 56) Is pulled to bend and deform the distal end side flexible tube 20, the pulling force exerts on the base end side flexible tube 30 in that direction. It is possible to substantially regulate the base end side flexible tube 30 from bending in that direction.

The movable elongated structure 10B configured in this manner is formed in a flexible elongated shape, and includes a plurality of flexible tubes having internal spaces (21, 31) penetrating in the longitudinal direction (L), (20, 30), a flexible elongated traction wire (50), and two of the plurality of flexible tubes (20, 30) arranged in series, which are adjacent in the longitudinal direction (L), and a wiring aid (408) arranged between the flexible tubes (20, 30) to maintain the interval between the opposing ends of the flexible tubes (20, 30), the direction in which the traction wire (50) is pulled is defined as the base end side (LB), and the opposite side is defined as the distal end side (LF), and among the plurality of flexible tubes (20, 30) arranged in series, Of the two flexible tubes (20, 30) adjacent in the longitudinal direction (L), the flexible tube (20, 30) arranged on the base end side (LB) is called the base end side flexible tube (30). In addition, the flexible tubes (20, 30) arranged on the distal end side (LF) are defined as the distal end side flexible tube (20), and the pair of traction bodies (50) are defined as a traction body set (53, 54), Two traction body sets (53, 54) are provided, and the tubular body (20, 30) is provided with a lumen (22, 32) for routing the traction body (50) along the longitudinal direction (L), A pair of lumens (22, 32) is defined as a lumen set (23, 24, 35, 36), and two lumen sets (23, 24, 35, 36) are formed in the tubular body (20, 30). Two sets of lumens (23, 24, 35, 36) are arranged at opposite positions in the cross section of the tubular bodies (20, 30), and the distal end side tubular body (20) has a distal end side tubular shape. Placing the route sets (23, 24, 35, 36) in the body (20) in opposite directions and the route sets (23, 24, 35, 36) in the base end side tubular body (30) It is arranged in series with the base end side tubular body (30) in a direction that intersects the opposing direction.

Then, the two sets of traction bodies (53 to 56) are routed to the two sets of lumens (23, 24, 35, 36) in the distal end side tubular body (20), respectively, and the base end tubular In the body (30), the first traction body (50) of one of the two traction body sets (53, 54) (53, 54) is connected to the two lumen sets (23, 24, 35), 36), and the tow body (50) of the other set of towing bodies (53, 54) is routed to two sets of lumens (23, 24), 35, 36), respectively, and in the base end side tubular body (30), the traction body (50) is any of the two pairs of lumens (23, 24, 35, 36) that face each other across the cross-sectional center of the tubular body (20, 30) in the crossing direction that intersects one direction. (22, 32), and in the base end side tubular body (30), routed to one of the two sets of wiring routes (35, 36) (35a, 36a, 35b, 36b). A first virtual line (FL) connecting the traction bodies (50) of one traction body set (53 to 56) of the two roped traction body sets (53, 54) in the cross-sectional direction, and the other fraction body A second virtual line (SL) connecting the traction bodies (50) of the set (53, 54) in the cross-sectional direction intersects In the doss-sectional direction, by pulling the plurality of traction bodies (50) to the base end side (18), at least one of the base end side tubular body (30) and the distal end side tubular body (20) may bend and deform in a desired direction with respect to the other.

Note that the movable elongated structure 10B may be configured with two sets of wires. In this case, the distal end side flexible tube 20 is arranged such that the two sets of lumens face each other in cross section. For example, the lumen set 23,24 and the wire set 53, 54 are provided, and in the distal end side flexible tube 20, the wire set 53, 54 is routed to the lumen set 23, 24 as described above. Then, wire sets 53 and 54 are routed to lumen sets 35 and 36 in the base end side flexible tube 30 in the same manner as described above.

In this manner, the movable elongated structure 10B, which includes two sets of lumens in the distal end side flexible tube 20 and two sets of wires, allows either one of the two sets of wires to be connected to the base end. By pulling to LB, the distal end side flexible tube 20 may be bent and deformed with respect to the base end side flexible tube 30 in the direction of the lumen set arranged with the wire set.

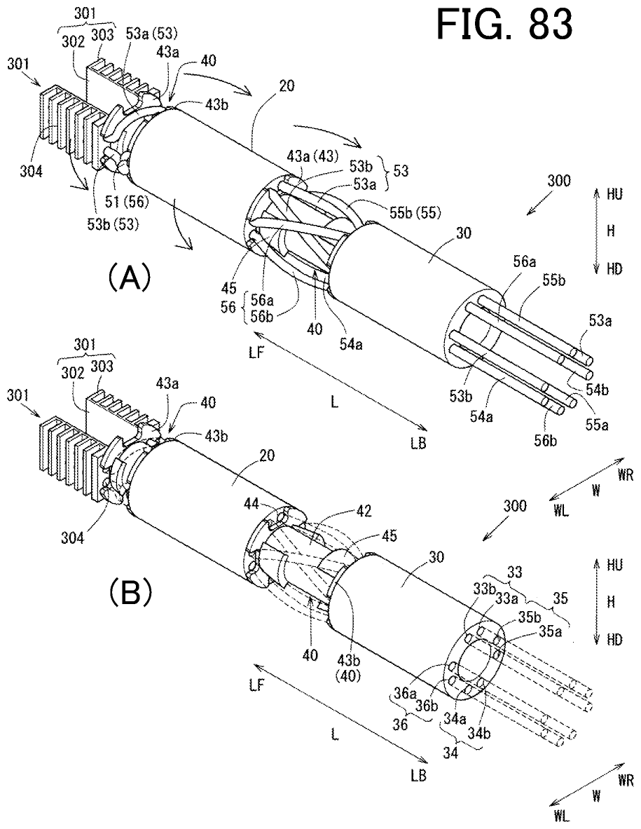

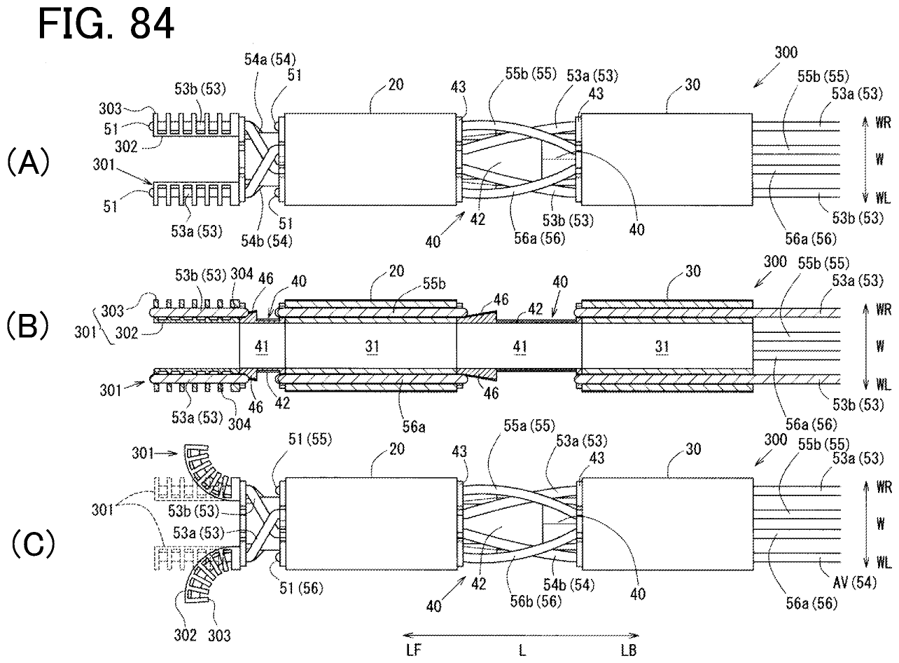

In addition, as shown in FIGS. 83 and 84 to be described later, the retractor 300 may be constructed using the movable elongated structure 10.

Note that the movable elongated structures 10, 10A, and 10B are collectively referred to as the movable elongated structure 10 below. Therefore, in the following description, the movable elongated structure 10 may be read as the movable elongated structures 10A and 10B. Similarly, the distal end side flexible tubes 20 and 20A are collectively referred to as the distal end side flexible tube 20, and in the following description, the description of the distal end side flexible tube 20 may be read as the distal end side flexible tube 20A.

FIG. 83 shows a schematic illustration of the retractor 300. FIG. 83 at (A) shows a perspective view of retractor 300, and FIG. 83 at (B) shows a perspective view of retractor 300 with traction wire 50 shown in transparent.

FIG. 84 shows a schematic illustration of the retractor 300. As shown in FIG. 84 at (A) shows a plan view of retractor 300, FIG. 84 at (8) shows a cross-section through the upper wire lumen, and FIG. 84 at (C) shows a plan view of retractor 300 with elastic retractor 301 in the open state.

The retractor 300 utilizes the movable elongated structure 10 described above and has an elastic retractor 301 on the distal end side LF of the spacer 40 instead of the distal end side flexible tube 20 in the movable elongated structure 10. Specifically, the movable elongated structure 10 described above is used with the width direction W being the height direction H.

Note that the spacer 40 in the retractor 300 has a shorter length in the longitudinal direction L of the cylinder 42 than the spacer 40 in the movable elongated structure 10 on the distal end side LF, but other configurations are the same. be. Also, the wiring of the traction wire 50 is the same as the wiring in the movable long structural body 10, so the description is omitted.

Two elastic retractors 301 are fixed to the front end side LF of the spacer 40 so as to face each other in the width direction W.

The elastic retractor 301 is composed of an elastic body 302 extending toward the distal end side LF and a plurality of plate portions 303 projecting outward in the width direction W from the elastic body 302.

The elastic body 302 is a rectangular plate having a predetermined thickness that is longer in the longitudinal direction L than in the height direction H, and the plate portion 303 is a rectangular plate having a predetermined thickness that is longer in the height direction H than in the width direction W.

A plurality of plate portions 303 are arranged at predetermined intervals in the longitudinal direction L, integrally configured with the elastic body 302, and are longer in the height direction H than in the width direction W and longer in the longitudinal direction L than in the height direction H. It is formed in a rectangular parallelepiped shape.

A plurality of plate portions 303 arranged at predetermined intervals in the longitudinal direction L correspond to lumen sets 33 and 34 of the distal end-side flexible tube 20 in the movable elongated structure 10, and wire sets 53 and 54 are inserted therethrough. An insertion hole 304 is formed to be inserted.

The elastic retractor 301 configured in this way faces the front end side LF of the spacer 40 at a predetermined interval in the width direction W, that is, the direction in which the plate portion 303 protrudes from the elastic body 302 is the width direction W. It is arranged to be outside.

The traction wires 53a and 54b are inserted through the insertion hole 304 of the elastic retractor 301 on the left side WL, and the traction wires 53b and 54a are inserted through the insertion hole 304 of the elastic retractor 301 on the right side WR.

The retractor 300 configured in this way pulls the wire sets 55 and 56 arranged in the height direction H on the base end side LB to the base end side LB, thereby pulling the wire sets 55 and 56 toward the distal end side as indicated by arrows in FIG. 16 at (A). The flexible tube 20 may be bent and deformed in the width direction W.

Specifically, the distal end side flexible tube 20 is bent and deformed to the left WL by puling the fourth wire set 56 inserted through the distal left lumen set 26 of the distal end side flexible tube 20 toward the base end side LB. By pulling the third wire set 55 inserted through the distal right lumen set 25 of the distal end side flexible tube 20 to the base end side LB, the distal end side flexible tube 20 is bent and deformed to the right WR.

By candling the pulling of the third wire set 55 and the fourth wire set 56, the elastic force of the distal end-side flexible tube 20 cancels the bending of the third wire set 55 and the fourth wire set 56 due to the pulling.

The elastic retractor 301 may be opened by pulling the wire set 53, 54 on the base end side LB.

Specifically, by pulling the traction wires 53a and 54b inserted through the insertion hole 304 of the elastic retractor 301 on the left side WL, the distal end side flexible tube 20 is not bent and deformed as shown by the arrow in FIG. 83 at (A). In addition, the elastic retractor 301 on the left side WL may be bent and deformed to the left side WL, which is the outside in the width direction W.

Conversely, by pulling the traction wires 53b and 54a inserted through the insertion hole 304 of the elastic retractor 301 of the right WR, the distal end side flexible tube 20 is not bent and deformed, as indicated by the arrow in FIG. 16 at (A). The elastic retractor 301 on the right side WR may be curvedly deformed to the right side WR, which is the outside in the width direction W.

Therefore, when the first wire set 53 and the second wire set 54 are pulled at the same time, the elastic retractors 301 on both sides in the width direction W are bent and deformed outward in the width direction W as shown in FIG. 84 at (C), the retractor 300 may be served as a retractor to open a site.

By canceling the pulling of the first wire set 53 and/or the second wire set 54, the first wire set 53 and/or the second wire set 54 are pulled by the restoring force due to the elasticity of the elastic retractors 301 (302), whereby bending by pulling is eliminated.

Furthermore, in the retractor 300 described above, the elastic retractor 301 in which the plate portion 303 is provided outside the elastic body 302 in the width direction W is used. By using the elastic retractor 301 provided with the plate portion 303 on the inner side in the width direction W, the elastic retractor 301 is bent inward in the width direction W by pulling the first wire set 53 and/or the second wire set 54, which may be served as forceps.

The above-described retractor 300 uses the above-described movable elongated structure 10, and instead of the distal end side flexible tube 20 in the movable elongated structure 10, the elastic retractor 301 is attached to the distal end side LF of the spacer 40. However, instead of the distal end side flexible tube 20, a retractor Is provided at the distal end LF of the distal end side flexible tube 20, and a drive mechanism for the retractor is provided in the main lumen 21 of the distal end side flexible tube 20. It may be a movable elongated treatment instrument. In place of the retractor provided at the end of the distal end side LF of the distal end side flexible tube 20, a treatment tool such as a gripper, forceps, forceps, a needle, a probe, or scissors is provided as a movable long treatment instrument, A driving mechanism for the treatment instrument may be arranged in the main lumen 21. In addition, in order to heat, cauterize, stop bleeding, and cut living tissue with these treatment tools, a high-frequency (radio wave) or microwave power transmission cable connected to the treatment tools may be routed.

In the following, as another embodiment, the medical equipment using the embodiments of FIGS. 68 to 84 will be described using the medical equipment based on FIGS. 62 to 65 of the previous embodiment. The configuration is similar to that of FIGS. 62 to 65, and the description of the same content will be omitted as appropriate.

A manipulator 100, which is the medical device, will be described as an example using the movable elongated structure 10 of the present embodiment. Here, an example using the movable elongated structure 10 in which the distal end side flexible tube 20, the spacer 40, and the base end side flexible tube 30 are arranged in this order will be described, but the present invention is not limited to this. Note that FIG. 62 shows a schematic diagram of a manipulator 100, which is a medical device in another embodiment using the movable elongated structure 10 of the present is invention.

The manipulator 100 is a medical device that is inserted into a branched channel such as a blood vessel, a lumen, or inside a hollow organ, and performs a predetermined treatment after the tip reaches a predetermined location. A manipulator body 101 having a grip and a movable elongated structure 10 extending from the distal end of the manipulator body 101 to the distal end side LF are provided. For details of the configuration of the manipulator main body 101, refer to the description of the above embodiment based on FIG. 62.

Next, as another example of this embodiment, a telesurgery system 200 will be described with reference to FIGS. 63 and 64.

FIG. 63 shows a schematic diagram of the telesurgery system 200 in the other example above, FIG. 64 shows a schematic illustration of the tool 317 in the telesurgical system 200.

Specifically, FIG. 64 at (A) shows a plan view of a tool 317 that may be loaded onto the robotic arm assembly of telesurgical system 200, and FIG. 64 at (B) shows the internal configuration of tool 317. For specific detailed configuration and operation, please refer to the description of the embodiment based on FIGS. 63 and 64 above.

Sine the operations of the telesurgery system 200 of FIGS. 63 and 64 are similar, please refer to the above description of operations based on FIG. 65.

In another embodiment of the present invention, forming a flexible distal end side tubular body and a flexible or non-flexible base end side tubular body into an elongated shape, a step of inserting a pair of traction operation bodies having flexibility along the longitudinal direction into a pair of through holes provided inside the tube wall of the base end side tubular body; and a step of regulating the direction of the traction operation body by placing it between the end tubular bodies. Similarly, the manufacturing method may be configured in accordance with the above equipment.

Further, in the embodiments based on FIGS. 1 to 87, movable elongated structures, movable elongated therapeutic instruments, movable elongated structural instruments, medical systems, tools, robots, medical robots, manipulators, flexible endoscopes, a method of manufacturing a speculum and steering catheter is disclosed and included in the present invention.

In another embodiment of the present invention, the step of forming an elongated tubular body with a flexible distal end side tubular body and a flexible or non-flexible base end side tubular body, a step of inserting a pair of traction operation bodies having flexibility along the longitudinal direction through a pair of through holes provided inside the tube walls of the distal end side tubular body and the base end side tubular body;

A method for designing a movable elongated structure, comprising a step of arranging a wiring aid between the distal end side tubular body and the base end side tubular body, and restricting the direction of the traction operation body. Similarly, a method of designing a configuration suitable for the above equipment may be used.

Further, in the embodiments based on FIGS. 1 to 87, the movable elongated structure, the movable elongated therapeutic Instrument, the movable elongated structural instrument, the medical system, the tool, the robot, the medical robot, the manipulator, the flexible Methods of designing endoscopes and steering catheters are disclosed and included in the present invention.

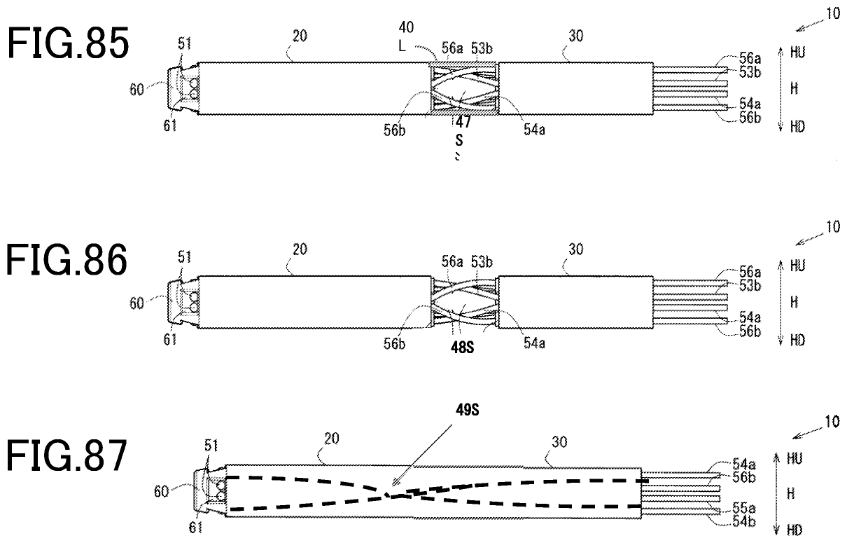

As an embodiment of the present invention. FIGS. 85 to 87, show a moveable elongated structure (10) including at least a distal end side tubular body (20), a base end side tubular body (30) and a traction operation body (53b, 54a, 56a, 56b), wherein a routing or wiring unit (40L, 48S, 49S) wires the traction operation bodies (53b, 54a, 56a, 56b) in the distal side tubular body (20) and the base end side tubular body (30) arranged in series for wiring.

FIG. 87 shows an example of a moveable elongated structure (10) made with multiple wire lumens, wherein the wiring unit (40L, 48S, 49S) attaches the movable elongated structure (10) to the tube walls of the distal end side tubular body (20) and the base end side tubular body (30). Traction operating bodies (53b, 54a, 56a, 56b) indicated by dashed lines are routed with a plurality of wire lumens and intersect at 49S.

The distal end side tubular body (20) and the base end side tubular body (30) are composed of one tube, the distal end side tubular body (20) is composed of a flexible tube portion, and the base end side. The tubular body (30) may be constructed from flexible or non-flexible tubing sections of one-piece, continuous construction. If necessary, the distal end side tubular body (20) and the base end side tubular body (30) may be divided as shown in FIG. 85 or FIG. 86.

As a modification, FIG. 85 shows a partial cross-sectional side view of a movable elongated structure 10 as another embodiment of the present invention, in which the wiring unit (40L) is a tubular body (40L) that connects the distal end side tubular body (20) and the base end side tubular body (30). The distal end side tubular body (20) and the base end tubular body (30) may be divided if necessary.

and the base end side tubular body (30) may be divided.

The movable elongated structure 10 includes at least a distal end side tubular body (20), a base end side tubular body (30), traction operating bodies (53b. 54a, 56a, 56b), and the distal end side tubular body (20). and the base end side tubular body (30), and the distal end side tubular body (20) and the base end side tubular body (30) each have a wire routine, the traction operation body (53b, 54a. 56a, 56b) is housed in the hollow portion (47S) of the tubular body (40L). If necessary, the hollow portion (47S) of the tubular body (40L) may be filled with a lubricant. The distal end side tubular body (20), the tubular body (40L), and the base end side tubular body (30) are continuous, and at least the distal end side tubular body (20) is composed of a flexible tube. The distal end side tubular body (20) may be flexible and the base end side tubular body (30) may be flexible or non-flexible.

As another example, an intermediate tubular body (20B) is provided between the distal end side tubular body (20) and the base end side tubular body (30) as shown in FIG. 68. It may be configured to be connected to the three tubular bodies (20, 208, 20). Also, a plurality of intermediate tubular bodies (206) may be provided to form a multi-stage movable elongated structure. In that case, the traction operation body (wire) may have different lengths according to the positions of the tubular bodies (20, 208). The connecting tubular body (40L) may be an outer seal, or may be made of plastic or metal. If plasticity is required for the connection tubular body (40L), bellows-shaped metal may be used instead of resin.

FIG. 86 shows a side view of movable elongated structure 10 according to another embodiment of the present invention. The movable elongated structure 10 does not include the tubular body (40L), and the distal end side tubular body (20) and the base end side tubular body (30) are each routed in a wire routine. It is held by the traction operation body (53b, 54a, 56a, 56b). The traction operation body has strength to withstand bending of the distal end side tubular body. The distal end side tubular body (20) may be flexible and the base end side tubular body (30) may be a flexible or non-flexible tubular body. One or more intermediate tubular bodies (20B) may be provided between the distal end side tubular body (20) and the base end side tubular body (30).

As another embodiment of the present invention, in FIGS. 85 and 86, a flexible distal end side tubular body (20) having a through internal space located distally and a through internal space located base end ly. and at least one pair of flexible elongated retractors (53b 54a 56a 56b) for retracting the distal end side tubular body (30). A movable elongated structure (10) having a pair of traction bodies as a traction body set, at least a plurality of the traction body sets (53b, 54e, 56e, 56b) provided, and the distal end side tubular body (20) and the base end side tubular body (30) are provided with lumens (22, 32) for routing the set of traction bodies along the longitudinal direction, and the pair of lumens is called a set of lumens. In addition, a plurality of pairs of the lumens are provided, and the plurality of pairs of the lumens are arranged in a plurality of directions in the cross sections of the distal end side tubular body and the base end side tubular body, a movable type provided with a lumen set in which the interval to the cross-sectional center of the base end side tubular body on one side is closer than that of the lumen set routed in the distal end side tubular body, and the interval between the routed traction bodies is wide.

The movable elongated structure described with reference to FIGS. 85 to 87 may be modified by adding other elements to the movable elongated structure shown in the example embodiment based on FIGS. 9 to 84, Movable elongate treatment instruments, movable elongated structural instruments, medical systems, tools, robots, medical robots, manipulators, flexible endoscopes and steering catheters may be provided.

In the above example, there is an example in which both the distal end side tubular body (20) and the base end side tubular body (30) are flexible tubes, but the flexible distal end side tubular body (20) and the non-flexible tubular body (20) are flexible. By forming a movable elongated structure having a flexible base end side tubular body (30), the inflexibility or rigidity of the base end side tubular body (30) makes the pulling force of the traction operation body even. may be made smaller.

Further, in the above-described embodiment, when a pair of lumens is one lumen set and a pair of traction bodies is one set of traction bodies, multiple sets such as 2 sets, 3 sets, 4 sets, or 6 sets are formed. Alternatively, the number of lumen sets may exceed the number of tow body sets.

The embodiments of the present invention are illustrative in all respects, and the scope of the present invention includes all modifications within the meaning and range of equivalents of the claims.

What is claimed is:

1. A movable elongated structure comprising at least a distal end side tubular body, a base end side tubular body, and a traction operation body, further comprising a wiring aid arranging the traction operation body, between the distal end side tubular body and the base end side tubular body.

2. The movable elongated structure according to claim 1, wherein the wiring aid is removably held by the traction operation body between the distal end side tubular body and the base end side tubular body.

3. The movable elongated structure according to claim 1, wherein the wiring aid provides with a traction operation body arrangement part receiving the traction operation body at both end portions facing the distal end side tubular body and the base end side tubular body.

4. The movable elongated structure of claim 3, wherein the traction operation body arrangement part has a flange projecting radially outward from both end parts of the wiring aid, and has a notch part receiving and engaging the traction operation body on the flange.

5. The movable elongated structure according to claim 1,
wherein the distal end side tubular body and the base end side tubular body are formed in a flexible and elongated shape,
wherein the traction operation body is a pair of flexible traction operation bodies that are inserted through a pair of longitudinal through holes provided inside tube walls of the distal end side tubular body and the base end side tubular body, and
wherein the through hole provided in the base end side tubular body is referred to as a base end side through hole, and the through hole provided in the distal end side tubular body is referred to as a distal end side through hole.

6. The movable elongated structure according to claim 5,
wherein an interval between the pair of base end side through holes in a circumferential direction in a cross-sectional plane of the base end side tubular body is set wider than an interval between the pair of distal end side through holes in a circumferential direction in a cross-sectional plane of the distal end side tubular body,
wherein a traction operation body arrangement part provided at the base end side and the distal end side have a base end side flange and a distal end side flange, respectively, protruding radially outward, and
wherein the traction operation body led out from the base end side through hole and introduced into the distal end side through hole is disposed in the traction operation body arrangement part.

7. The movable elongated structure according to claim 5,
wherein a distal end side interval which is an interval between a distal end side virtual line connecting the centers of the pair of distal end side through holes and a distal end side central line passing through the center of the distal end side tubular body and parallel to the distal end side virtual line, is set wider than a base end side interval which is an interval between a base end side virtual line connecting the centers of the pair of base end side through holes and a base end side central line passing through a center of the base end side tubular body and parallel to the base end side virtual line.

8. The movable elongated structure according to claim 6,
wherein the wiring aid includes at least a cylindrical body arranged along the longitudinal direction,
wherein the base end side flange is provided at a base end side end of the cylindrical body,
wherein the distal end side flange is provided at a distal end side end of the cylindrical body.
wherein the base end side flange portion is provided with a proximity regulation part that regulates proximity in a circumferential direction of the traction operation body led out from the base end side through hole and introduced into the distal end side through hole,
wherein the distal end side flange portion is provided with a separation regulation part that regulates separation in a circumferential direction of the traction operation body led out from the base end side through hole and introduced into the distal end side through hole, and
wherein the operation body arrangement part is open on a radial outer side.

9. The movable elongated structure according to claim 8, wherein at least one of the base end side flange and the distal end side flange is configured separately from the cylindrical body and configured to be assembled.

10. The movable elongated structure according to claim 8,
wherein the pair of base end side through holes and the pair of distal end side through holes through which the pair of traction operation bodies are inserted form a pair of through holes,
wherein a plurality of sets of the through hole sets are arranged at different positions in a circumferential direction,
wherein a plurality of the proximity regulation parts and the traction operation body arrangement parts are provided on the base end flange, and
wherein a plurality of the separation regulation parts and the traction operation body arrangement parts are provided on the distal end side flange portion.

11. The movable elongated structure according to claim 10,
wherein in the base end side flange, common the proximity regulation part regulates circumferentially adjacent traction operation bodies of a pair of the traction operation bodies inserted through the circumferentially adjacent through hole sets to come close to each other,
wherein in the distal end side flange, the separation of the traction operation bodies that are adjacent in the circumferential direction of the pair of traction operation bodies that are inserted through the through hole sets that are adjacent in the circumferential direction is regulated by the traction operation body inserted through a pair of through-holes adjacent to each other on the opposite side in the circumferential direction and common the separation regulation part.

12. The movable elongated structure according to claim 10,
wherein one of the traction operation bodies and the other of the traction operation bodies inserted through the through hole sets that are adjacent in the circumferential direction intersect in the circumferential direction, and
wherein the cylindrical body is provided with a guide portion for guiding one of the traction operation bodies to the outside of the other traction operation body.

13. The movable elongated structure according to claim 10,
wherein an elongated intermediate tubular body having an intermediate through hole is arranged between the distal end side tubular body and the end side tubular body,
wherein a plurality of pairs of the traction operation bodies are provided,
wherein at least one pair of the traction operation bodies out of the plurality of pairs of the traction operation bodies is inserted through the distal end side through hole, the intermediate through hole, and the base end side through hole, and
wherein at least one pair of the traction operation bodies out of the plurality of pairs of the traction operation bodies is inserted through the intermediate through hole and the base end through hole.

14. The movable elongated structure according to claim 5,
wherein the traction operation body is a flexible wire,
wherein the distal end side tubular body and the end side tubular body are composed of flexible tubes having a main lumen, and
wherein the through hole is a wire lumen formed inside the tube wall of the tube and through which the wire is available to be inserted.

15. A movable elongated structural instrument comprising the movable elongated structure according to claim 1 and a traction drive for pulling the pair of traction operation bodies, wherein the traction drive pulls the pair of traction operation bodies to bend and deform the distal end side tubular body.

16. A movable elongated structural instrument comprising the movable elongated structure according to claim 10 and a traction drive for pulling the pair of traction operation bodies, wherein a plurality of traction drives are provided, and the traction drives are constructed to pull the pair of traction operation bodies to bend and/or stretch (extend) the distal end side tubular body in a desired direction.

17. A medical system comprising the movable elongated structural instrument according to claim 15, a drive unit for driving the traction drive, and a control unit connected to apply a drive signal to the drive unit.

18. A medical system comprising the movable elongated structural instrument according to claim 16, a drive unit selectively driving at least one of the plurality of traction drives, and a control unit connected to apply a drive signal to the drive unit.

19. The medical system according to claim 18, wherein an operation unit selectively operating at least one of the plurality of traction drive units is provided, and is connected to the control unit.

20. A tool comprising:
the movable elongated structural instrument according to claim 15;
a mounting portion mounting the end side tubular body of the movable elongated structure to a distal end of a robot arm; and
a connecting portion connected to a driving mechanism for driving the traction driving portion is provided on the robot arm side.

21. A robot comprising:
the tool according to claim 20;
a robot arm having the tool at its distal end;
a drive unit that drives the traction drive and the robot arm; and
a control unit connected to apply a drive signal to the drive unit.

22. A manipulator comprising:
the movable elongated structural instrument according to claim 15;
a body portion provided at a base end of the base end side tubular body in the movable elongated structure; and
an operation unit operating the traction drive part in the main body part.

23. A robot comprising:
an input/output unit routed and/or wirelessly connected to the movable elongated structural instrument according to claim 15;
an input unit that receives operation signals in real time;
an arithmetic unit that executes a predetermined operation program based on the operation signal; and
an output unit that generates a driving signal for pulling a predetermined traction operation body by the traction drive unit and bending and/or expanding and contracting (extending) at least the distal end side tubular body in a desired direction based on an output from the arithmetic unit.

24. A medical robot comprising the robot according to claim 23, wherein the output unit provides drive signals to an externally mounted drive unit that mechanically drives the movable elongated structure.

25. An insertion method comprising:
Inserting the movable elongated structure according to claim 1 into a duct,
driving and controlling the traction drive unit that pulls the pair of traction operating bodies to bend and deform the distal end side tubular body, and
inserting the distal end side tubular body into a branched duct.

26. An insertion method comprising:
inserting the movable elongated structure according to claim 1 Including a distal end side tubular body, a base end side tubular body and a traction operation body, which includes the wiring aid removably held by the traction operation body between the distal end side tubular body and the base end side tubular body, into a duct,
driving and controlling the traction drive unit to bend and deform the distal end side tubular body, and
inserting the distal end side tubular body into a branched duct.

27. The insertion method according to claim 25, wherein the duct is at least one of a hollow organ, a vessel and a blood vessel.

28. An operation method of the robot equipped with the movable long structural instrument according to claim 15, comprising:
receiving an operation signal in real time by the input/output unit routed and/or wirelessly connected to the robot;
executing a predetermined operation program based on the received operation signal, by the arithmetic unit; and then,
pulling the traction operation body by the traction drive unit based on the output from the arithmetic unit to bend-deform and/or expand-contract (extension) deform the distal end side tubular body in a desired direction.

29. A method of operating a movable elongated structure of removing the wiring aid from the movable elongated structure of claim 1 provided with a distal end side tubular body, a base end side tubular body and a traction operation body which includes the wiring aid removably held by the traction operation body between the distal end side tubular body and the base end side tubular body.

30. A method of operating a movable elongated structure according to claim 29 comprising;
pulling the pair of traction operation bodies to bend and deform the distal end side tubular body.

31. A wiring aid. which is removably held between the distal end side tubular body and side tubular body of the movable elongated structure according to claim 1.

32. The wiring aid according to claim 31, wherein the wiring aid arranged along the longitudinal direction between the flexible elongated distal end side tubular body and base end side tubular body arranged in series from the distal end side to the base end side along the longitudinal direction includes at least a cylindrical body, and is provided with a base end side flange protruding radially outward provided with an operation body arrangement part at the base end side end of the cylindrical body in which the interval in a circumferential direction is set wider than the interval in the circumferential direction between the pair of distal end side through holes provided inside the tube wall of the distal end side tubular body and penetrating in the longitudinal direction and a pair of traction operation bodies led out from a pair of base end side through holes provided inside the tube wall of the base end side tubular body and introduced into the distal side through hole are arranged.

33. The movable elongated structure according to claim 1 in which the distal end side tubular body and the base end side tubular body are formed in a flexible, elongated shape and have an internal space penetrating in the longitudinal direction, and are composed of at least two tubular bodies, comprising:
a long traction operation body having flexibility of the traction operation body;
a wiring aid that is arranged between the two tubular bodies arranged in series, has a tubular part that is held by the traction operation body, and is not fixed to the tubular bodies;
a movement regulation part that regulates movement of the wiring aid in a radial direction that intersects the longitudinal direction with respect to at least one of the tubular bodies.

34. The movable elongated structure according to claim 33 in which the traction operation body is inserted through a pair of through-passages along the longitudinal direction provided in the tube wall of the tubular body, and a plurality of the traction operation bodies are provided.

35. The movable elongated structure according to claim 34,
wherein the wiring aid includes a flange disposed at the end of the cylindrical portion and protruding radially outward,
wherein the traction operation body holds the wiring aid arranged between the tubular bodies via the flange, and
wherein the movement regulation part is a protrusion protruding from an end surface of the cylindrical portion toward at least one of the tubular bodies.

36. The movable elongated structure according to claim 35, in which the protrusion is columnar.

37. The movable elongated structure according to claim 35, in which the protrusion is a columnar shape protruding from a ring-shaped member.

38. The movable elongated structure according to claim 35, in which the protrusion is an annular protrusion that fits into at least one of the outer peripheral surface and the inner peripheral surface of the tubular body.

39. The movable elongated structure according to claim 35, in which the protrusion is a small piece-like protrusion that contacts at least one of an outer peripheral surface and an inner peripheral surface of the tubular body at a plurality of points in the circumferential direction.

40. The movable elongated structure according to claim 35, in which the protrusion is tapered small piece protrusions that abut against the inner peripheral surface of the tubular body at a plurality of points in the circumferential direction.

41. The movable elongated structure according to claim 34,
wherein the wiring aid is provided with a flange disposed at the end of the cylindrical portion and protruding radially outward;
wherein the traction operation body holds the wiring aid arranged between the tubular bodies via the flange;
wherein the movement regulation part is an adhesive layer that bonds at least one end face of the tubular body and the flange.

42. The movable elongated structure according to claim 34,
Wherein the wiring aid is provided with a flange disposed at the end of the cylindrical portion and protruding radially outward,
wherein the traction operation body holds the wiring aid arranged between the tubular bodies via the flange;
wherein the movement regulation part is a regulatory tubular body that is inserted across the internal space of at least one of the tubular bodies and the internal space of the tubular part.

43. The movable elongated structure according to claim 34,
wherein the cylindrical portion of the wiring aid is inserted through the internal space of at least one of the tubular bodies; and
wherein the movement regulation part is composed of a portion of the cylindrical portion that is inserted into the internal space of the tubular body.

44. The movable elongated structure according to claim 33, wherein the movement regulation part is configured to prevent the wiring aid from being carelessly removed from being held by the traction operation body.

45. The movable elongated structure according to claim 33, wherein the movement regulation part regulates radial movement of the wiring aid with respect to both of the tubular bodies.

46. The movable elongated structure according to claim 35, in which the traction operation body is a flexible wire, and the flange protrude radially outward.

47. The movable elongated structure according to claim 46, wherein a hole through which the wire is inserted is provided on the outer periphery of the flange

48. The movable elongated structure according to claim 45, wherein the tubular body on the distal end side of the two tubular bodies arranged in series is an elastic retractor, elastic gripper, forceps or scissors having the internal space, which is opened and closed by the wire.

49. A wiring aid used in the movable elongated structure according to claim 33.

50. An insertion method comprising:
inserting the movable elongated structure according to claim 44 into a duct,
driving and controlling the traction drive unit and deforming the tubular body on the distal end side, and
inserting the tubular body on the distal end side into a branched duct.

51. The insertion method according to claim 50, wherein the aid duct is at least one of a hollow organ, a vessel and a blood vessel.

52. A method of operating a movable elongated structure comprising:
pulling the pair of traction operation bodies, and
deforming the tubular body on the distal end side of the movable elongated structure according to claim 44.

53. A flexible endoscope comprising;
the movable elongated structure according to claim 33;
a plurality of traction operation units for pulling the pair of traction operation bodies,
wherein the traction operation body pulls the pair of traction operation bodies to deform the tubular body on the distal end side.

54. A steering catheter comprising;
the movable elongated structure according to claim 33; and
a plurality of traction operation units for pulling a pair of traction operation bodies,
wherein the traction operation body pulls the pair of traction operation bodies to deform the tubular body on the distal end side.

55. The robot according to claim 21, wherein the control unit is equipped with artificial intelligence.

56. A robot comprising:
an input/output unit routed and/or wirelessly connected to the movable elongated structural instrument according to claim 15;
an input unit that receives operation signals in real time;
an arithmetic unit that executes a predetermined operation program based on the operation signal; and
an output unit generating a drive signal for pulling the predetermined traction operation body by the pulling driving section based on the output from the arithmetic unit and deforming at least the tubular body on the distal end side in a desired direction;
wherein the computing unit is equipped with artificial intelligence.

57. The movable elongated structure according to claim 1,
wherein a pair of traction bodies are served as a traction body set and four traction body sets are provided,
wherein wire lumens for routing the traction body along the longitudinal direction are provided in the distal end side tubular body and the base end side tubular body,
wherein a pair of wire lumens are served as a wire lumen set and four wire lumen sets are provided,
wherein the four wire lumen sets are arranged in four directions in cross sections of the distal end side tubular body and the base end side tubular body,
wherein the base end side tubular body and the distal end side tubular body arranged in series are arranged such that the wire lumen sets provided in four directions are oriented along the longitudinal direction,
wherein a wire lumen set is routed in one of the four directions of the cross sections in which the wire lumen sets are provided in the distal end side tubular body.

58. The movable elongated structure according to claim 1,
wherein the traction bodies route the wire lumens in the base end side tubular body which are closer to a cross-sectional center of the base end side tubular body and wider than the interval between the routed traction bodies with respect to the lumen set routed in the distal end side tubular body in one direction,
wherein body and the lumen of the distal end side tubular body, and fixed to the distal end side tubular body on the distal end side thereof ton be independently towable, and
wherein at least one of the base end side tubular body and the distal end side tubular body is bent and deformed in a desired direction with respect to the other by pulling the plurality of traction bodies toward the base end side.

59. The movable elongated structure according to claim 1,
wherein, in the base end side tubular body, a first traction body set routed in the distal end side tubular body is routed in a first pair of wire lumens with a wider distance across a center of a cross section, and a second traction body set routed in the distal end side tubular body is routed in a second pair of wire lumens with a wider distance across the center of the cross section, and
wherein a virtual line connecting the first pair of wire lumens and a virtual line connecting the second pair of wire lumens intersects near the center of the cross section.

60. The movable elongated structure according to claim 1,
wherein a pair of traction bodies are served as a traction body set, and two traction body sets are provided,
wherein a tubular body is provided with a wire lumen for routing the traction body along a longitudinal direction, and a pair of the wire lumens are served as a wire lumen set,
wherein In the tubular body, two sets of wire lumen set are provided, and two sets of lumens are arranged at opposite positions in the cross section of the tubular body, and the lumen set in the distal end side tubular body is arranged in the tubular body on the distal end side in series with the end side tubular body in a direction that intersects the opposing direction and the opposing direction in which the wire lumen set in the end side tubular body is arranged;
wherein two sets of traction bodies are routed respectively to two sets of wire lumens in the distal end side tubular body, and the retractor of one of the two traction body sets in the end side tubular body is routed to one of the wire lumens in the two sets of wire passages, and the tow body of the other tow body set is routed to an unrouted wire lumen in the two sets of wire lumens,
wherein in the base end side tubular body, the traction bodies in the two traction body sets are arranged in one of the wire lumens in the two wire lumen sets that face each other across the cross-sectional center of the tubular body in the crossing direction that intersects one direction.
wherein a first imaginary line connecting, in the cross-sectional direction, the traction bodies of one of the two traction body sets routed to one of the wire lumens in the two wire lumen sets in the base end side tubular body and a second imaginary line connecting the traction bodies of the other traction body set in the cross-sectional direction intersect in the cross-sectional direction.
whereby at least one of the base end side tubular body and the distal end side tubular body is bent and deformed in a desired direction with respect to the other by pulling the plurality of traction bodies toward the base end side.

61. The movable elongated structure according to claim 1,
wherein the base end side tubular body is a flexible tubular body having penetration internal space arranged on a base end side,
wherein the distal end side tubular body is a flexible tubular body having penetration internal space arranged on a distal end side,
wherein the wiring aid is a wiring aid that maintains a distance between the opposing ends of the tubular body,
wherein the traction operation body is a flexible elongated traction body,
wherein a pair of traction bodies are served as a traction body set, and four sets of the traction body sets are provided,
wherein one of the pair of traction bodies is a long traction body that is routed across the base end side tubular body and the distal end side tubular body, and the other is a short traction body that is routed into the base end side tubular body,
wherein a lumen for routing the traction body along a longitudinal direction is provided in the tubular body,
wherein a pair of the lumens are served as a lumen set, and four pairs of the lumen sets are provided,
wherein the four sets of the lumen sets are arranged in four directions of a cross section of the base end side tubular body and the four sets of the lumen sets are arranged in four directions of a cross section of the distal end side tubular body,
wherein the base end side tubular body and the distal end side tubular body arranged in series are arranged such that the four directions are aligned,
wherein the long traction body of the traction body set is routed in one of the four directions of the cross section of the distal end side tubular body, and fixed to the distal side of the distal end side tubular body as to the distal end side tubular body
wherein the long traction body and the short traction body of the traction body set in the base end side tubular body are routed into the lumen that is closer to the cross-sectional center of the tubular body in the one direction than the lumen that is routed in the distal side end tubular body, and that is opposed in a direction that intersects the one direction.
wherein the short traction body routed in the lumen of the base end side tubular body is fixed to the base end side tubular body on the distal side of the base end side tubular body, and each of the towing bodies is configured to be towable independently, and
whereby at least one of the base end side tubular body and the distal end side tubular body is bent and deformed in a desired direction with respect to the other by pulling the plurality of traction bodies toward the base end side.

62. The movable elongated structure according to claim 1,
wherein the base end side tubular body is a flexible tubular body having penetration internal space arranged on a base end side,
wherein the distal end side tubular body is a flexible tubular body having penetration internal space arranged on a distal end side,
wherein the wiring aid is a wiring aid that maintains a distance between the opposing ends of the tubular body,
wherein the traction operation body is a flexible elongated traction body,
wherein a pair of traction bodies are served as a traction body set, and four sets of the traction body sets are provided,
wherein a lumen for routing the traction body along the longitudinal direction is provided in the tubular body,
wherein a pair of the lumens are served as a lumen set, and four pairs of the lumen sets are provided, and the four sets of the lumens are arranged in four directions in the cross section of the tubular body,
wherein the base end side tubular body and the distal end side tubular body that are arranged in series are arranged such that the lumen sets provided in four directions are oriented along the longitudinal direction,
wherein in the distal end side tubular body, the traction body set is routed in one of the four directions of the cross section in which the lumen set is provided, and in the base end side tubular body, the interval between the cross-sectional center of the tubular body in the one direction is shorter than the lumen set routed in the distal end side tubular body, and the interval between the routed traction bodies is greater,
wherein in the distal end side tubular body, two sets of the traction body sets are routed to two sets of the lumen sets that face each other in one direction across the center of the cross section of the tubular body,
wherein in the base end side tubular body, any one of the two lumen sets that face each other across the cross-sectional center of the tubular body in the crossing direction that intersects one direction is the traction body in the two traction body sets,
wherein in the base end side tubular body, a first imaginary line that connects the traction bodies of one of the two traction body groups routed in one of the two lumen sets in the cross-sectional direction and a second virtual line connecting the traction bodies of the other traction body set in the cross-sectional direction intersect in the cross-sectional direction, and
wherein at least one of the base end side tubular body and the distal end side tubular body is bent and deformed in a desired direction with respect to the other by pulling the plurality of traction bodies toward the base end side.

63. The movable elongated structure according to claim 62, in which the first virtual line and the second virtual line pass through the cross-sectional center

64. The movable elongated structure according to claim 63, wherein the wiring aid is provided with a regulation part that regulates the relative positions of the traction body of one of the traction body set and the traction body of the other traction body set about two sets of the traction bodies intersecting between the longitudinally adjacent tubular bodies.

65. A movable long treatment tool,
wherein a distal end side of the distal end side tubular body in the movable elongated structure according to claim 1 is provided with a treatment tool such as a retractor, a gripper, forceps, forceps, or scissors, and
wherein drive mechanism for the treatment tool is arranged in an internal space of the movable elongated structure.

66. The movable elongated structure according to claim 1, wherein the distal end side tubular body includes elastic retractors, elastic grippers, forceps, needles, probes, or scissors having am internal space, which are opened and closed with wires.

67. A method of inserting a movable elongated structure comprising,
inserting the movable elongated structure according to claim 1 into a duct,
driving and controlling a traction drive unit to pull the traction body to curvedly deform at least one of the distal end side tubular body and the base end side tubular body in a desired direction, and
inserting the distal end side tubular body is into a branched duct.

68. A method of operating the movable elongated structure according to claim 1 comprising:
routing the lumen of the base end side tubular body and the lumen of the distal end side tubular body of the traction body set,
pulling one of the traction bodies fixed to the distal end side tubular body on a distal end side of the distal end side tubular body,
bending and deforming the distal end side tubular body in a desired direction;
routing in the lumen of the base end side tubular body,
pulling the other traction body of the traction body set fixed to the base end side tubular body on a distal side of the base end side tubular body to bend and deform the base end side tubular body,

69. A movable elongated structural instrument comprising the movable elongated structure according to claim 1 and a traction drive unit pulling the traction body, wherein at least one of the distal end side tubular body and the base end side tubular body is bent and deformed in a desired direction by pulling the traction body with the traction drive unit.

70. A robot comprising:
an input/output unit wired and/or wirelessly connected to the movable elongated structure according to claim 1;
an input unit that receives an operation signal in real time;
an arithmetic unit that executes a predetermined operation program based on the operation signal;
pulling a predetermined towing body by the tow drive unit based on an output from the arithmetic unit, and
an output unit that generates a drive signal for bending and deforming at least one of the distal end side tubular body and the base end side tubular body, wherein the computing unit is equipped with artificial intelligence.

71. A flexible endoscope comprising the movable elongated structure according to claim 1 and a plurality of traction operation units pulling the traction body, wherein at least one of the distal end side tubular body and the base end side tubular body is bent and deformed in a desired direction by pulling the traction body with the traction operation unit.

72. A method of manufacturing the movable elongated structure according to claim 1 comprising:
forming the distal end side tubular body flexible and the base end side tubular body flexible or non-flexible into an elongated shape;
inserting a pair of the traction operation bodies having flexibility in a longitudinal direction through a pair of through holes provided inside tube walls of the distal end side tubular body and the base end side tubular body;
arranging the wiring aid between the distal end side tubular body and the base end side tubular body; and
regulating the direction of the traction operation body.

73. A method of designing the movable elongated structure according to claim 1 comprising:
forming the distal end side tubular body flexible and the base end side tubular body flexible or non-flexible into an elongated shape;
inserting a pair of the traction operation bodies having flexibility in a longitudinal direction through a pair of through holes provided inside tube walls of the distal end side tubular body and the base end side tubular body;
arranging the wiring aid between the distal end side tubular body and the base end side tubular body; and
regulating the direction of the traction operation body.

74. A movable elongated structure comprising at least a distal end side tubular body, an base end side tubular body, a traction operation body, and routing unit routing the traction body. wherein the distal end side tubular body and the base end side tubular body are arranged in series,

75. The movable elongated structure according to claim 74,
wherein the distal end side tubular body and the base end side tubular body include a plurality of wire lumens in a tube wall, and
wherein the plurality of wire lumens constitute the routing unit routing the traction operation body.

76. The movable elongated structure according to claim 74,
wherein the routing unit is a tubular body that connects the distal end side tubular body and the base end side tubular body,
wherein the distal end side tubular body and the base end side tubular body each includes a wire lumen, and the traction operation body is housed in a hollow portion of the tubular body.

77. The movable elongated structure according to claim 76, wherein the distal end side tubular body, the tubular body and the base end side tubular body are continuous and at least the distal end side tubular body is made of a flexible tube.

78. A movable elongated structure comprising:
a flexible distal end side tubular body having a through interior space disposed at a distal end,
a flexible or inflexible base end side tubular body having a through interior space disposed at a base end, and
at least a pair of flexible elongated traction bodies for pulling the flexible distal end side tubular body,
wherein the pair of traction bodies are served as a traction body set, and a plurality of the traction body sets are provided,
wherein a lumen routing the traction body set along a longitudinal direction is provided in the distal end side tubular body and the base end side tubular body,
wherein a pair of the lumens are served as a lumen set, and a plurality of the lumen sets are provided, and the plurality of lumen sets are formed in cross sections of the distal end side tubular body and the base end side tubular body, respectively arranged in multiple directions, and
wherein in the base end side tubular body, there are provided lumen sets in which the interval between the cross-sectional center of the base end side tubular body on one side is shorter than that of the lumen set routed in the distal end side tubular body, and the interval between the routed traction bodies is wider than that of the lumen set routed in the distal end side tubular body.

79. The movable elongated structure according to claim 1, wherein the distal end side tubular body is flexible and the base end side tubular body is non-flexible.
